SEQUENCE LISTING (88 sequences)

SEQ ID NO: 1 - protein, 331 aa, Rhinovirus C
MGAQVSKQNVGSHESGISASSGSVIKYFNINYYKDSASSGLSKQDFSMDPEKFTKPLADVMTNPALMSPSIEACGFSDRLKQITIGSSTITTQDTLNTVVAYGEWPEYLRDTDASAVDKPTHPETSTDRFYTLTSVIWNGSSKGWWWKIPDCLKDMGMFGQNMYHHALGRSGYIFHIQCNATKFHSGLLLVAIVPEHQLAYVGGTYANVGYNHTHPGEGGHEIREPTGRDDKKPDEDPLFNCNGTLLGNLTIFPHQLINLRTNNSVTIVVPYINCVPMDSMLKHNNISLVIIPLVPLRSGSTQAPQTLPITISIAPDKSEFSGARQSNKTQ

SEQ ID NO: 2 - protein, 330 aa, Rhinovirus C
MGAQVSRQTTGSHESAVNATNGGIIKYFNINYYRDSASSGLTKQDFSQDPSKFTQPLVDTLTNPALMSPSVEACGFSDRLKQITMGNSTITTQDALHTVLAYGEWPQYLSDLDATSVDKPTHPETSSDRFYTLSSVSWTNTSKGWWWKLPDALKDMGVFGQNLYYHAMGRAGYIIHTQCNATKFHSGALLVVLIPEHQLAYIGAEKVNIAYDLTHPGETGHVIGRNTSRGNNNPDEDPFFNCNGTLFGNLTIFPHQIINLRTNNSSTIITPYINCQPMDNMLKHNNLTLLIVPLVRLRFGTEASPTVSITVTIAPYKSEFSGAMETQKHQ

SEQ ID NO: 3 - protein, 329 aa, Rhinovirus C
MGAQVSKQNVGSHESGISASSGSVIKYFNINYYKDSASSGLSKQDFSMDPEKFTKPIAETLTNPALMSPSIEACGFSDRLKQITIGNSTITTQDALNTVVAYGEWPQYLSDMDASAIDKPTHPETSTDRFYTLTSVIWDTTSKGWWWKIPDCLKEMGMFGQNMYHHALGRSGFIIHVQCNATKFHSGLLIVAVVPEHQLAYIGGTNVSVGYNHTHPGENGHTIGLNDQRGDRQPDEDPFFNCNGTLLGNLTIFPHQLINLRTNNSATIVVPYINCVPMDSMLRHNNLSLVIIPMVDLRFGTTGVTTLPITISIAPVKSEFSGARQSRTQ

SEQ ID NO: 4 - protein, 329 aa, Rhinovirus A
MGTQVSRQNVGTHSTQNSVSNGSSLNYFNINYFKDAASSGASKLEFSQDPSKFTDPVKDVLEKGIPTLQSPTVEACGYSDRIIQITRGDSTITSQDVANAVVGYGVWPHYLTPQDATAIDKPSRPDTSSNRFYTLESKMWTSDSKGWWWKLPDALKNMGIFGENMFYHFLGRSGYTVHVQCNASKFHQGTLIVVMIPEHQLASASTGNVTAGYNLTHPGEQGRDVGITRVEDLLKQPSDDSWLNFDGTLLGNITIFPHQFINLRSNNSATIIVPYVNAVPMDSMPRHNNWSLVIIPICPLESDGQTPVPITISISPMCAEFSGARAKSQ

SEQ ID NO: 5 - protein, 330 aa, Rhinovirus A
MGAQVSRQNVGTHSTQNSVSNGSSLNYFNINYFKDAASSGASKLEFSQDPSKFTDPVKDVLEKGIPTLQSPSVEACGYSDRIIQITRGDSTITSQDVANAVVAYGVWPHYLTPQDATAIDKPSRPDTSSNRFYTLESKTWTGSSKGWWWKLPDALKGMGIFGENMFYHFLGRSGYTVHVQCNASKFHQGTLIVAMIPEHQLASALAGNVTAGYNLTHPGEGGRTVDQTNRENQSKQPSDDSWLNFDGTLLGNITIFPHQFINLRSNNSATIIVPYVNAVPMDSMLRHNNWSLVIIPICPLESMQVPNTVPITISISPMCAEFSGARARSQ

SEQ ID NO: 6 - protein, 560 aa, Rhinovirus A
MGPSDMYVHVGNLIYRNLHLFNSEMHDSILISYSSDLVIYRTNTTGDDYIPTCDCTEATYYCKHKNRYFPIKVTSHDWYEIQESEYYPKHIQYNLLIGEGPCEPGDCGGKLLCKHGVIGMITAGGDGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKVKYLSELKKLNLLEKQIEHLRAADSATQEKIKCEIDTLHDLSCKFLPLYASEAKRIKVLHNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSLLTPPTITSLPAMNRRFFMDLDIIVCDKYKDAQGKLNVSAAFKPCDVDTKIGNARCCPFICGKAVMFKDRNTCRTYTLAQIYNQILEEDKRRRQVIDVMSAIFQ

SEQ ID NO: 7 - protein, 560 aa, Rhinovirus A
MGPSDMYVHVGNLIYRNLHLFNSDMHDSILVSYSSDLVIYRTNTTGDDYIPTCDCTEATYYCRHKNRYYPIKVTSHDWYEIQESEYYPKHIQYNLLIGEGPCEPGDCGGKLLCKHGVIGIITAGGEGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKIKYLSELKKLNLLEKQIEHLRVADSATQEKIKCEIDTLHDLSCKFLPLYASEAKRIKVLYNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSMLAPPTITSLPAMNRRFFMDLDIIVNDKYKDSQGKLNVSAAFKPCDVDTKIGNARCCPFICGKAVTFKDRNTCHTFSLAQVYNQILDEDKRRRQVIDVMSAIFQ

SEQ ID NO: 8 - protein, 568 aa, Rhinovirus C
MGPSDMYVHTKEAIYKNAHLTSANEQTILIALTADLQVDAADHPGDDVIPDCDCTTGTYYCKSKDRYYPVEVVSHAWYPIEETCYYPKHIQYNILLGEGPCVPGDCGGKLLCKHGVIGIVTAGGENHVAFTDLRPYSNLAHTQGPISDYVTQLGNAFGTGFTQTLETNLRETCSGMFDAITSKTVKWVVRIISALTIVIRNSSDIPTILATMALLGCTGSPWQYLKSKLCNWLGVQKPPSKQSDSWLKKFTEWCNAAKGLEWIGYKISKFIDWLKEKLIPTVQRKKDTLLECKKLTLYEDQVRAFPQSPEAFQNELTTKLQILKKNLDDMCPLYAAENKRVTNMLRDIKTMTAYKKTHRTEPVCVLIHGGPGCGKSLATTVIARGLTDSGNVYSLPPSPKHFDGYCQQQVVMMDDLGQNPDGQDLAMFCQMVSTTDFIVPMAALEDKGKSFTSDFVLASTNLNQLSPPTVTIPEAITRRFFLDVDLKIMSGYRTHAGLLDTAKALQACPDCAKPPYYKQCCPLLCGKAVVLQNRRTSASLSLNMVVSQLREESNTRKRIHTNLNAIFQ

SEQ ID NO: 9 - protein, 567 aa, Rhinovirus C
MGPSDQCVHTKDAIYTCAHLTEPNSNTILLAITADLQVDSTDTPGPDFIPTCDCVQACYYAKHAQRYYPITVTPHDWYEIQESQYYPKHIQYNILIGEGPCEPGDCGGKLLCRHGVIGIITAGGDGHVAFTDLRPYACLSHQGLVSDYVNQLGAAFGDGFSSNIKDHLTGLCTTVSDKITGKVIKWLVRVISALTIMIRNSSDTATVLATLALLGCHGSPWSFLKEKICQWLGIPRPPTRQGESWLKKFTECCNAAKGLEWVAQKIGKFIDWLKEKLIPTVQRKKETLDQCKKIGLYEEQTKGFSHSEAEAQQSLILEVAKLKRGLDDLAPLYASENKRVTIIQKELQRLSAYQKTHRHEPVCCLLRGPPGCGKSLVTSIIAHGLTNEANIYSLPPDPKHFDGYNQQTVVIMDDVGQNPDGKDLSMFCQMVSTTEFIVPMASIEDKGRAFTSQYVLASTNLDSLSPPTVTIPEAISRRFYLDADLQVTSKFKAHNGLLDVAKALQPCAKCPKPNHYKQCCPILCGQALVLRDRRTSANYPLLAVVEQLRMENNTRDKVKSNLRAIFQ

SEQ ID NO: 10 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 11 - RNA, 99 nt, synthetic construct
  source 1..99: mol_type = other RNA
gggtggcatccctgtgacccctccccagtgcctctcctggccctggaagttgccactccagtgcccaccagccttgtcctaataaaattaagttgcatc

SEQ ID NO: 12 - RNA, 105 nt, synthetic construct
  source 1..105: mol_type = other RNA
gggtggcatccctgtgacccctccccagtgcctctcctggccctggaagttgccactccagtgcccaccagccttgtcctaataaaattaagttgcatcaaagct

SEQ ID NO: 13 - RNA, 105 nt, synthetic construct
  source 1..105: mol_type = other RNA
cgggtggcatccctgtgacccctccccagtgcctctcctggccctggaagttgccactccagtgcccaccagccttgtcctaataaaattaagttgcatcaagct

SEQ ID NO: 14 - protein, 17 aa, Influenza A
MKAKLLVLLCTFTATYA

SEQ ID NO: 15 - protein, 17 aa, Influenza A
MKAILVVLLYTFATANA

SEQ ID NO: 16 - protein, 16 aa, Influenza A
MKTIIALSYILCLVFA

SEQ ID NO: 17 - protein, 15 aa, Influenza B
MKAIIVLLMVVTSNA

SEQ ID NO: 18 - protein, 12 aa, SARS CoV-2
MFVFLVLLPLVS

SEQ ID NO: 19 - protein, 21 aa, SARS CoV-2
MFLLTTKRTMFVFLVLLPLVS

SEQ ID NO: 20 - protein, 71 aa, VSV
MSPCGYYSKWRNRDRPEYRRNLRFRRFFSSIHPNAAAGSGFNGPGVFITSVTGVWLCFLCIFSMFVTAVVS

SEQ ID NO: 21 - protein, 30 aa, VSV
MGTVNKPVVGVLMGFGIITGTLRITNPVRA

SEQ ID NO: 22 - protein, 20 aa, VSV
MFLIQCLISAVIFYIQVTNA

SEQ ID NO: 23 - protein, 21 aa, VSV
MQALGIKTEHFIIMCLLSGHA

SEQ ID NO: 24 - protein, 23 aa, Measles morbillivirus
MGLKVNVSAIFMAVLLTLQTPTG

SEQ ID NO: 25 - protein, 21 aa, Rubella
MGAAAALTAVVLQGYNPPAYG

SEQ ID NO: 26 - protein, 23 aa, Rubella
MGAPQAFLAGLLLAAVAVGTARA

SEQ ID NO: 27 - protein, 19 aa, Mumps orthorubulavirus
MKVFLVTCLGFAVFSSSVC

SEQ ID NO: 28 - protein, 32 aa, Ebola virus
MGVTGILQLPRDRFKRTSFFLWVIILFQRTFS

SEQ ID NO: 29 - protein, 16 aa, Smallpox
MRSLIIFLLFPSIIYS

SEQ ID NO: 30 - protein, 576 aa, synthetic construct
MKAILVVLLYTFATANAGPSDMYVHVGNLIYRNLHLFNSEMHDSILISYSSDLVIYRTNTTGDDYIPTCDCTEATYYCKHKNRYFPIKVTSHDWYEIQESEYYPKHIQYNLLIGEGPCEPGDCGGKLLCKHGVIGMITAGGDGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKVKYLSELKKLNLLEKQIEHLRAADSATQEKIKCEIDTLHDLSCKFLPLYASEAKRIKVLHNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSLLTPPTITSLPAMNRRFFMDLDIIVCDKYKDAQGKLNVSAAFKPCDVDTKIGNARCCPFICGKAVMFKDRNTCRTYTLAQIYNQILEEDKRRRQVIDVMSAIFQ

SEQ ID NO: 31 - protein, 576 aa, synthetic construct
MKAILVVLLYTFATANAGPSDMYVHVGNLIYRNLHLFNSDMHDSILVSYSSDLVIYRTNTTGDDYIPTCDCTEATYYCRHKNRYYPIKVTSHDWYEIQESEYYPKHIQYNLLIGEGPCEPGDCGGKLLCKHGVIGIITAGGEGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKIKYLSELKKLNLLEKQIEHLRVADSATQEKIKCEIDTLHDLSCKFLPLYASEAKRIKVLYNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSMLAPPTITSLPAMNRRFFMDLDIIVNDKYKDSQGKLNVSAAFKPCDVDTKIGNARCCPFICGKAVTFKDRNTCHTFSLAQVYNQILDEDKRRRQVIDVMSAIFQ

SEQ ID NO: 32 - protein, 348 aa, synthetic construct
MKAILVVLLYTFATANADTLGTQVSRQNVGTHSTQNSVSNGSSLNYFNINYFKDAASSGASKLEFSQDPSKFTDPVKDVLEKGIPTLQSPTVEACGYSDRIIQITRGDSTITSQDVANAVVGYGVWPHYLTPQDATAIDKPSRPDTSSNRFYTLESKMWTSDSKGWWWKLPDALKNMGIFGENMFYHFLGRSGYTVHVQCNASKFHQGTLIVVMIPEHQLASASTGNVTAGYNLTHPGEQGRDVGITRVEDLLKQPSDDSWLNFDGTLLGNITIFPHQFINLRSNNSATIIVPYVNAVPMDSMPRHNNWSLVIIPICPLESDGQTPVPITISISPMCAEFSGARAKSQ

SEQ ID NO: 33 - protein, 349 aa, synthetic construct
MKAILVVLLYTFATANADTLGAQVSRQNVGTHSTQNSVSNGSSLNYFNINYFKDAASSGASKLEFSQDPSKFTDPVKDVLEKGIPTLQSPSVEACGYSDRIIQITRGDSTITSQDVANAVVAYGVWPHYLTPQDATAIDKPSRPDTSSNRFYTLESKTWTGSSKGWWWKLPDALKGMGIFGENMFYHFLGRSGYTVHVQCNASKFHQGTLIVAMIPEHQLASALAGNVTAGYNLTHPGEGGRTVDQTNRENQSKQPSDDSWLNFDGTLLGNITIFPHQFINLRSNNSATIIVPYVNAVPMDSMLRHNNWSLVIIPICPLESMQVPNTVPITISISPMCAEFSGARARSQ

SEQ ID NO: 34 - protein, 888 aa, synthetic construct
MGPSDMYVHVGNLIYRNLHLFNSEMHDSILISYSSDLVIYRTNTTGDDYIPTCDCTEATYYCKHKNRYFPIKVTSHDWYEIQESEYYPKHIQYNLLIGEGPCEPGDCGGKLLCKHGVIGMITAGGDGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKVKYLSELKKLNLLEKQIEHLRAADSATQEKIKCEIDTLHDLSCKFLPLYASEAKRIKVLHNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSLLTPPTITSLPAMNRRFFMDLDIIVCDKYKDAQGKLNVSAAFKPCDVDTKIGNARCCPFICGKAVMFKDRNTCRTYTLAQIYNQILEEDKRRRQVIDVMSAIFQGTQVSRQNVGTHSTQNSVSNGSSLNYFNINYFKDAASSGASKLEFSQDPSKFTDPVKDVLEKGIPTLQSPTVEACGYSDRIIQITRGDSTITSQDVANAVVGYGVWPHYLTPQDATAIDKPSRPDTSSNRFYTLESKMWTSDSKGWWWKLPDALKNMGIFGENMFYHFLGRSGYTVHVQCNASKFHQGTLIVVMIPEHQLASASTGNVTAGYNLTHPGEQGRDVGITRVEDLLKQPSDDSWLNFDGTLLGNITIFPHQFINLRSNNSATIIVPYVNAVPMDSMPRHNNWSLVIIPICPLESDGQTPVPITISISPMCAEFSGARAKSQ

SEQ ID NO: 35 - protein, 904 aa, synthetic construct
MKAILVVLLYTFATANAGPSDMYVHVGNLIYRNLHLFNSEMHDSILISYSSDLVIYRTNTTGDDYIPTCDCTEATYYCKHKNRYFPIKVTSHDWYEIQESEYYPKHIQYNLLIGEGPCEPGDCGGKLLCKHGVIGMITAGGDGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKVKYLSELKKLNLLEKQIEHLRAADSATQEKIKCEIDTLHDLSCKFLPLYASEAKRIKVLHNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSLLTPPTITSLPAMNRRFFMDLDIIVCDKYKDAQGKLNVSAAFKPCDVDTKIGNARCCPFICGKAVMFKDRNTCRTYTLAQIYNQILEEDKRRRQVIDVMSAIFQGTQVSRQNVGTHSTQNSVSNGSSLNYFNINYFKDAASSGASKLEFSQDPSKFTDPVKDVLEKGIPTLQSPTVEACGYSDRIIQITRGDSTITSQDVANAVVGYGVWPHYLTPQDATAIDKPSRPDTSSNRFYTLESKMWTSDSKGWWWKLPDALKNMGIFGENMFYHFLGRSGYTVHVQCNASKFHQGTLIVVMIPEHQLASASTGNVTAGYNLTHPGEQGRDVGITRVEDLLKQPSDDSWLNFDGTLLGNITIFPHQFINLRSNNSATIIVPYVNAVPMDSMPRHNNWSLVIIPICPLESDGQTPVPITISISPMCAEFSGARAKSQ

SEQ ID NO: 36 - protein, 584 aa, synthetic construct
MKAILVVLLYTFATANAGPSDQCVHTKDAIYTCAHLTEPNSNTILLAITADLQVDSTDTPGPDFIPTCDCVQACYYAKHAQRYYPITVTPHDWYEIQESQYYPKHIQYNILIGEGPCEPGDCGGKLLCRHGVIGIITAGGDGHVAFTDLRPYACLSTHQGLVSDYVNQLGAAFGDGFSSNIKDHLTGLCTTVSDKITGKVIKWLVRVISALTIMIRNSSDTATVLATLALLGCHGSPWSFLKEKICQWLGIPRPPTRQGESWLKKFTECCNAAKGLEWVAQKIGKFIDWLKEKLIPTVQRKKETLDQCKKIGLYEEQTKGFSHSEAEAQQSLILEVAKLKRGLDDLAPLYASENKRVTIIQKELQRLSAYQKTHRHEPVCCLLRGPPGCGKSLVTSIIAHGLTNEANIYSLPPDPKHFDGYNQQTVVIMDDVGQNPDGKDLSMFCQMVSTTEFIVPMASIEDKGRAFTSQYVLASTNLDSLSPPTVTIPEAISRRFYLDADLQVTSKFKAHNGLLDVAKALQPCAKCPKPNHYKQCCPILCGQALVLRDRRTSANYPLLAVVEQLRMENNTRDKVKSNLRAIFQ

SEQ ID NO: 37 - protein, 584 aa, synthetic construct
MKAILVVLLYTFATANAGPSDMYVHTKEAIYKNAHLTSANEQTILIALTADLQVDAADHPGDDVIPDCDCTTGTYYCKSKDRYYPVEVVSHAWYPIEETCYYPKHIQYNILLGEGPCVPGDCGGKLLCKHGVIGIVTAGGENHVAFTDLRPYSNLAHTQGPISDYVTQLGNAFGTGFTQTLETNLRETCSGMFDAITSKTVKWVVRIISALTIVIRNSSDIPTILATMALLGCTGSPWQYLKSKLCNWLGVQKPPSKQSDSWLKKFTEWCNAAKGLEWIGYKISKFIDWLKEKLIPTVQRKKDTLLECKKLTLYEDQVRAFPQSPEAFQNELTTKLQILKKNLDDMCPLYAAENKRVTNMLRDIKTMTAYKKTHRTEPVCVLIHGGPGCGKSLATTVIARGLTDSGNVYSLPPSPKHFDGYCQQQVVMMDDLGQNPDGQDLAMFCQMVSTTDFIVPMAALEDKGKSFTSDFVLASTNLNQLSPPTVTIPEAITRRFFLDVDLKIMSGYRTHAGLLDTAKALQACPDCAKPPYYKQCCPLLCGKAVVLQNRRTSASLSLNMVVSQLREESNTRKRIHTNLNAIFQ

SEQ ID NO: 38 - protein, 348 aa, synthetic construct
MKAILVVLLYTFATANADTLGAQVSKQNVGSHESGISASSGSVIKYFNINYYKDSASSGLSKQDFSMDPEKFTKPIAETLTNPALMSPSIEACGFSDRLKQITIGNSTITTQDALNTVVAYGEWPQYLSDMDASAIDKPTHPETSTDRFYTLTSVIWDTTSKGWWWKIPDCLKEMGMFGQNMYHHALGRSGFIIHVQCNATKFHSGLLIVAVVPEHQLAYIGGTNVSVGYNHTHPGENGHTIGLNDQRGDRQPDEDPFFNCNGTLLGNLTIFPHQLINLRTNNSATIVVPYINCVPMDSMLRHNNLSLVIIPMVDLRFGTTGVTTLPITISIAPVKSEFSGARQSRTQ

SEQ ID NO: 39 - protein, 349 aa, synthetic construct
MKAILVVLLYTFATANADTLGAQVSRQTTGSHESAVNATNGGIIKYFNINYYRDSASSGLTKQDFSQDPSKFTQPLVDTLTNPALMSPSVEACGFSDRLKQITMGNSTITTQDALHTVLAYGEWPQYLSDLDATSVDKPTHPETSSDRFYTLSSVSWTNTSKGWWWKLPDALKDMGVFGQNLYYHAMGRAGYIIHTQCNATKFHSGALLVVLIPEHQLAYIGAEKVNIAYDLTHPGETGHVIGRNTSRGNNNPDEDPFFNCNGTLFGNLTIFPHQIINLRTNNSSTIITPYINCQPMDNMLKHNNLTLLIVPLVRLRFGTEASPTVSITVTIAPYKSEFSGAMETQKHQ

SEQ ID NO: 40 - protein, 350 aa, synthetic construct
MKAILVVLLYTFATANADTLGAQVSKQNVGSHESGISASSGSVIKYFNINYYKDSASSGLSKQDFSMDPEKFTKPLADVMTNPALMSPSIEACGFSDRLKQITIGSSTITTQDTLNTVVAYGEWPEYLRDTDASAVDKPTHPETSTDRFYTLTSVIWNGSSKGWWWKIPDCLKDMGMFGQNMYHHALGRSGYIFHIQCNATKFHSGLLLVAIVPEHQLAYVGGTYANVGYNHTHPGEGGHEIREPTGRDDKKPDEDPLFNCNGTLLGNLTIFPHQLINLRTNNSVTIVVPYINCVPMDSMLKHNNISLVIIPLVPLRSGSTQAPQTLPITISIAPDKSEFSGARQSNKTQ

SEQ ID NO: 41 - protein, 898 aa, synthetic construct
MGPSDMYVHTKEAIYKNAHLTSANEQTILIALTADLQVDAADHPGDDVIPDCDCTTGTYYCKSKDRYYPVEVVSHAWYPIEETCYYPKHIQYNILLGEGPCVPGDCGGKLLCKHGVIGIVTAGGENHVAFTDLRPYSNLAHTQGPISDYVTQLGNAFGTGFTQTLETNLRETCSGMFDAITSKTVKWVVRIISALTIVIRNSSDIPTILATMALLGCTGSPWQYLKSKLCNWLGVQKPPSKQSDSWLKKFTEWCNAAKGLEWIGYKISKFIDWLKEKLIPTVQRKKDTLLECKKLTLYEDQVRAFPQSPEAFQNELTTKLQILKKNLDDMCPLYAAENKRVTNMLRDIKTMTAYKKTHRTEPVCVLIHGGPGCGKSLATTVIARGLTDSGNVYSLPPSPKHFDGYCQQQVVMMDDLGQNPDGQDLAMFCQMVSTTDFIVPMAALEDKGKSFTSDFVLASTNLNQLSPPTVTIPEAITRRFFLDVDLKIMSGYRTHAGLLDTAKALQACPDCAKPPYYKQCCPLLCGKAVVLQNRRTSASLSLNMVVSQLREESNTRKRIHTNLNAIFQGAQVSKQNVGSHESGISASSGSVIKYFNINYYKDSASSGLSKQDFSMDPEKFTKPLADVMTNPALMSPSIEACGFSDRLKQITIGSSTITTQDTLNTVVAYGEWPEYLRDTDASAVDKPTHPETSTDRFYTLTSVIWNGSSKGWWWKIPDCLKDMGMFGQNMYHHALGRSGYIFHIQCNATKFHSGLLLVAIVPEHQLAYVGGTYANVGYNHTHPGEGGHEIREPTGRDDKKPDEDPLFNCNGTLLGNLTIFPHQLINLRTNNSVTIVVPYINCVPMDSMLKHNNISLVIIPLVPLRSGSTQAPQTLPITISIAPDKSEFSGARQSNKTQ

SEQ ID NO: 42 - protein, 914 aa, synthetic construct
MKAILVVLLYTFATANAGPSDMYVHTKEAIYKNAHLTSANEQTILIALTADLQVDAADHPGDDVIPDCDCTTGTYYCKSKDRYYPVEVVSHAWYPIEETCYYPKHIQYNILLGEGPCVPGDCGGKLLCKHGVIGIVTAGGENHVAFTDLRPYSNLAHTQGPISDYVTQLGNAFGTGFTQTLETNLRETCSGMFDAITSKTVKWVVRIISALTIVIRNSSDIPTILATMALLGCTGSPWQYLKSKLCNWLGVQKPPSKQSDSWLKKFTEWCNAAKGLEWIGYKISKFIDWLKEKLIPTVQRKKDTLLECKKLTLYEDQVRAFPQSPEAFQNELTTKLQILKKNLDDMCPLYAAENKRVTNMLRDIKTMTAYKKTHRTEPVCVLIHGGPGCGKSLATTVIARGLTDSGNVYSLPPSPKHFDGYCQQQVVMMDDLGQNPDGQDLAMFCQMVSTTDFIVPMAALEDKGKSFTSDFVLASTNLNQLSPPTVTIPEAITRRFFLDVDLKIMSGYRTHAGLLDTAKALQACPDCAKPPYYKQCCPLLCGKAVVLQNRRTSASLSLNMVVSQLREESNTRKRIHTNLNAIFQGAQVSKQNVGSHESGISASSGSVIKYFNINYYKDSASSGLSKQDFSMDPEKFTKPLADVMTNPALMSPSIEACGFSDRLKQITIGSSTITTQDTLNTVVAYGEWPEYLRDTDASAVDKPTHPETSTDRFYTLTSVIWNGSSKGWWWKIPDCLKDMGMFGQNMYHHALGRSGYIFHIQCNATKFHSGLLLVAIVPEHQLAYVGGTYANVGYNHTHPGEGGHEIREPTGRDDKKPDEDPLFNCNGTLLGNLTIFPHQLINLRTNNSVTIVVPYINCVPMDSMLKHNNISLVIIPLVPLRSGSTQAPQTLPITISIAPDKSEFSGARQSNKTQ

SEQ ID NO: 43 - protein, 896 aa, synthetic construct
MGPSDQCVHTKDAIYTCAHLTEPNSNTILLAITADLQVDSTDTPGPDFIPTCDCVQACYYAKHAQRYYPITVTPHDWYEIQESQYYPKHIQYNILIGEGPCEPGDCGGKLLCRHGVIGIITAGGDGHVAFTDLRPYACLSHQGLVSDYVNQLGAAFGDGFSSNIKDHLTGLCTTVSDKITGKVIKWLVRVISALTIMIRNSSDTATVLATLALLGCHGSPWSFLKEKICQWLGIPRPPTRQGESWLKKFTECCNAAKGLEWVAQKIGKFIDWLKEKLIPTVQRKKETLDQCKKIGLYEEQTKGFSHSEAEAQQSLILEVAKLKRGLDDLAPLYASENKRVTIIQKELQRLSAYQKTHRHEPVCCLLRGPPGCGKSLVTSIIAHGLTNEANIYSLPPDPKHFDGYNQQTVVIMDDVGQNPDGKDLSMFCQMVSTTEFIVPMASIEDKGRAFTSQYVLASTNLDSLSPPTVTIPEAISRRFYLDADLQVTSKFKAHNGLLDVAKALQPCAKCPKPNHYKQCCPILCGQALVLRDRRTSANYPLLAVVEQLRMENNTRDKVKSNLRAIFQGAQVSRQTTGSHESAVNATNGGIIKYFNINYYRDSASSGLTKQDFSQDPSKFTQPLVDTLTNPALMSPSVEACGFSDRLKQITMGNSTITTQDALHTVLAYGEWPQYLSDLDATSVDKPTHPETSSDRFYTLSSVSWTNTSKGWWWKLPDALKDMGVFGQNLYYHAMGRAGYIIHTQCNATKFHSGALLVVLIPEHQLAYIGAEKVNIAYDLTHPGETGHVIGRNTSRGNNNPDEDPFFNCNGTLFGNLTIFPHQIINLRTNNSSTIITPYINCQPMDNMLKHNNLTLLIVPLVRLRFGTEASPTVSITVTIAPYKSEFSGAMETQKHQ

SEQ ID NO: 44 - protein, 913 aa, synthetic construct
MKAILVVLLYTFATANAGPSDQCVHTKDAIYTCAHLTEPNSNTILLAITADLQVDSTDTPGPDFIPTCDCVQACYYAKHAQRYYPITVTPHDWYEIQESQYYPKHIQYNILIGEGPCEPGDCGGKLLCRHGVIGIITAGGDGHVAFTDLRPYACLSTHQGLVSDYVNQLGAAFGDGFSSNIKDHLTGLCTTVSDKITGKVIKWLVRVISALTIMIRNSSDTATVLATLALLGCHGSPWSFLKEKICQWLGIPRPPTRQGESWLKKFTECCNAAKGLEWVAQKIGKFIDWLKEKLIPTVQRKKETLDQCKKIGLYEEQTKGFSHSEAEAQQSLILEVAKLKRGLDDLAPLYASENKRVTIIQKELQRLSAYQKTHRHEPVCCLLRGPPGCGKSLVTSIIAHGLTNEANIYSLPPDPKHFDGYNQQTVVIMDDVGQNPDGKDLSMFCQMVSTTEFIVPMASIEDKGRAFTSQYVLASTNLDSLSPPTVTIPEAISRRFYLDADLQVTSKFKAHNGLLDVAKALQPCAKCPKPNHYKQCCPILCGQALVLRDRRTSANYPLLAVVEQLRMENNTRDKVKSNLRAIFQGAQVSRQTTGSHESAVNATNGGIIKYFNINYYRDSASSGLTKQDFSQDPSKFTQPLVDTLTNPALMSPSVEACGFSDRLKQITMGNSTITTQDALHTVLAYGEWPQYLSDLDATSVDKPTHPETSSDRFYTLSSVSWTNTSKGWWWKLPDALKDMGVFGQNLYYHAMGRAGYIIHTQCNATKFHSGALLVVLIPEHQLAYIGAEKVNIAYDLTHPGETGHVIGRNTSRGNNNPDEDPFFNCNGTLFGNLTIFPHQIINLRTNNSSTIITPYINCQPMDNMLKHNNLTLLIVPLVRLRFGTEASPTVSITVTIAPYKSEFSGAMETQKHQ

SEQ ID NO: 45 - protein, 2157 aa, Rhinovirus A
MGTQVSRQNVGTHSTQNSVSNGSSLNYFNINYFKDAASSGASKLEFSQDPSKFTDPVKDVLEKGIPTLQSPTVEACGYSDRIIQITRGDSTITSQDVANAVVGYGVWPHYLTPQDATAIDKPSRPDTSSNRFYTLESKMWTSDSKGWWWKLPDALKNMGIFGENMFYHFLGRSGYTVHVQCNASKFHQGTLIVVMIPEHQLASASTGNVTAGYNLTHPGEQGRDVGITRVEDLLKQPSDDSWLNFDGTLLGNITIFPHQFINLRSNNSATIIVPYVNAVPMDSMPRHNNWSLVIIPICPLESDGQTPVPITISISPMCAEFSGARAKSQGLPVMLTPGSGQFMTTDDFQSPSALPWFHPTKEISIPGQVTNLIELCQVDTLIPVNNTETNGVTNINMYTVTINREVNITPAKEIFAIKVDIASQPLATTLIGEIANYYTHWTGSIRFSFLFCGTANTTLKLLVAYTPPGIKKPENRKDAMLGTHVVWDVGLQSTISMVVPWISASHYRNTTPDKYSSAGYITCWYQTNLVVPPNTPTSAKMLCFVSGCKDFCLRMARDTSLHKQSAPITQNPVENYIDEVLNEVLVVPNIRESHGTTSNSAPALDAAETGHTSNVQPEDMVETRYVQTSQTRDEMSIESFLGRSGCIHMSKLVVNYDNYNTGENNISTWQINIKEMAQIRRKFEMFTYTRFDSEITLVPSIAARAGDIGHVVMQYMYVPPGAPIPKTREDFAWQSGTNASIFWQHGQTYPRFSLPFLSIASAYYMFYDGYDGDQTDSQYGAVVTNDMGSLCYRIVTGQHKHKIEVTTRIYHKAKHVKAWCPRPPRAVEYTHTHVTNYKIANHEVTSAVESRRTIVTVGPSDMYVHVGNLIYRNLHLFNSEMHDSILISYSSDLVIYRTNTTGDDYIPTCDCTEATYYCKHKNRYFPIKVTSHDWYEIQESEYYPKHIQYNLLIGEGPCEPGDCGGKLLCKHGVIGMITAGGDGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKVKYLSELKKLNLLEKQIEHLRAADSATQEKIKCEIDTLHDLSCKFLPLYASEAKRIKVLHNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSLLTPPTITSLPAMNRRFFMDLDIIVCDKYKDAQGKLNVSAAFKPCDVDTKIGNARCCPFICGKAVMFKDRNTCRTYTLAQIYNQILEEDKRRRQVIDVMSAIFQGPISLEGPPPAAISDLLQSVRTPEVIKYCEANKWIIPAECKVERDLNIANTIITIIANIISISGIIYVIYKLFCTLQGPYSGEPKPKTKMPERRVVAQGPEEEFGRSLIKHNSCVVTTQNGKFTGLGIYDRTLIIPTHADPGKEVQIDGIATKVEDSYDLFNKDGVKLEITVLKLKRNEKFKDIRKYIPENEDDYPDCNLALSANQPETTIINVGDVISYGNILLSGTQTARMLKYNYPTKSGYCGGILYKIGQVLGIHVGGNGRDGFAAMLLRSYFSETQGQIINSKPTDKCGLPSIHTPSKTKLQPSVFYDIFPGNKEPAVLSNKDPRLEVDFEKALFSKYKGNEHCVMNDHINVAISHYSAQLATLDINPQPISIEESVFGMDGLEALDLNTSAGFPYVTMGIKKRDLINKQTKDITKLKMALDKYGVDLPMVTFLKDELRKKEKISAGKTRVIEASSVNDTVLFRTTFGNLFSKFHLNPGIVTGSAVGCDPEVFWSKIPVMLDGDCIMAFDYTNYDGSIHPIWFQALKQVLTNLSFEASLIDRLCKSKHIFKNIYYEVEGGVPSGCSGTSIFNTMINNVIIRTLVLDAYKNIDLDKLKIIAYGDDVIFSYKYQLDMEAIANEGTKYGLTITPADKSTCFKQLDYSNVTFLKRGFKQDEKHQFLIHPTFPIEEIHESIRWTKNPSQMQEHVLSLCHLMWHNGRDVYKQFEKRIRSVGAGRALYIPPYDLLLHEWYEKF

SEQ ID NO: 46 - DNA, 1683 bp, synthetic construct
  source 1..1683: mol_type = other DNA
atgggcccttccgatatgtacgttcacgtggggaacctgatctatagaaatctgcacctctttaacagcgagatgcacgactccatcctgatcagctatagtagcgaccttgtgatctatcgcactaataccaccggagacgactacattccaacctgcgactgcactgaggccacctactactgcaagcataaaaatcgctacttccccatcaaggtcaccagccatgactggtatgagatccaggagtctgaatactatcctaagcacatccagtacaacctgctgatcggcgagggcccatgtgagcctggggattgtgggggcaagctgctgtgcaagcacggcgtgatcggaatgatcaccgccggcggggacggacacgtggctttcattgatctgaggcactttcactgtgctgaggagcagggcatcactgactacatccacatgctgggcgaagcctttgggtccggattcgtggatagcgtgaaggagcagatcaatgccatcaaccctatcaacaacatcagcaaaaagatcatcaaatggctgctgaggatcatttctgccatggttatcatcatcagaaactccagcgaccctcagacaatcatcgccaccctcactctgattggctgcagcgggtccccttggagattcctgaaggagaagttctgcaagtggacccagctgaactacatccacaaagagagcgacagttggctgaaaaaattcaccgaaatgtgtaacgccgcaaggggcctggaatggatcggcaataagatttctaagtttatcgactggatgaaatccatgctgcctcaggcccagctgaaggtgaaatacctgagcgaactgaagaagctgaacctgctggagaagcagattgagcacctgagagcagcagactccgccacacaggaaaaaatcaagtgcgagattgacactctgcatgacctgtcctgcaaattcctgcctctgtacgcttccgaggccaaaagaatcaaggtgctgcacaataagtgcaatgtggttatcaagcagaaaaagcggtgtgagccagtggccgtggttattcacggagagcctggcaccggcaagagcatgactactaattttctcgcccggatgatcaccaatgactctgacatctactccctgcccccagacccaaagtatttcgacggatatgaccagcagagcgtggttatcatggatgacatcatgcagaatccctccggcgacgatatgaccctgttttgtcagatggtttcctccgtgaccttcatccccccaatggccgatctgcctgacaagggcaagccttttgacagcaggttcgtgctgtgcagcacaaaccacagcctgctgaccccccctactattacctctctgcccgccatgaacaggagattctttatggacctggacatcatcgtgtgtgataaatataaggacgcccagggaaagctgaatgtcagcgccgctttcaagccctgcgacgtggacaccaagatcggcaacgctcggtgttgtcctttcatctgcggcaaagccgtgatgttcaaagacagaaatacatgtcgcacctacactctggcccagatctacaaccagattctggaggaggataagaggagacgccaggtcattgacgtcatgtccgccattttccagtga

SEQ ID NO: 47 - DNA, 1683 bp, synthetic construct
  source 1..1683: mol_type = other DNA
atggggccaagcgacatgtatgtgcatgtgggaaacctgatctacagaaacctgcacctctttaacagcgagatgcacgattccattctgatttcctattcctctgatctcgttatctaccggaccaacaccacaggagacgactatattccaacatgcgactgcacagaggccacttactactgtaagcacaaaaacagatacttccccatcaaggtgacttcccacgactggtacgagatccaggaatctgaatactaccctaagcatatccagtacaacctgctgattggagaagggccatgcgagccaggcgattgtggcggcaagctgctgtgtaaacacggcgtgatcggcatgattaccgccgggggcgacggccatgtggcattcatcgacctgaggcatttccattgtgcagaggagcagggcattaccgattacattcacatgctgggcgaggctttcggcagcggctttgtggacagcgtgaaggagcagattaacgcaattaaccctatcaataacatttccaagaagatcatcaagtggctgctgagaatcatctccgccatggttattatcatccgcaactctagcgacccccagaccattatcgcaacactgaccctgatcggatgttccggctccccttggcgctttctgaaggagaagttttgcaagtggacccagctgaactacattcacaaggagagcgactcttggctgaaaaagttcacagaaatgtgtaatgccgccagagggctggaatggatcggaaataagatctccaagttcatcgactggatgaagtccatgctgccacaggcccagctgaaagtcaaatacctgtctgagctgaaaaaactgaacctgctggagaagcagattgaacaccttcgggccgctgactctgccactcaggagaagatcaagtgcgaaattgacaccctgcacgacctgagctgcaaattcctgcctctgtatgcctccgaggccaagaggatcaaggtgctgcacaacaaatgcaatgtcgttatcaagcagaagaagcggtgcgagcccgtggccgtcgtgatccacggggagcctggaacaggaaagtctatgactactaacttcctcgcccggatgatcacaaacgacagcgacatctatagcctgccacccgaccctaaatatttcgatggatacgaccagcagtctgtcgtgatcatggatgacatcatgcagaatcctagcggggatgatatgacactgttctgccagatggtgagcagcgtgacattcatcccaccaatggctgacctgccagacaagggcaagcctttcgactccagattcgtgctgtgctccaccaaccactccctcctgacccctcccacaattacctccctgccagccatgaataggcgcttctttatggacctggatatcatcgtctgcgacaaatacaaggatgcccaggggaaactgaacgtgtccgccgcctttaagccttgtgacgtggacacaaagattgggaatgccagatgctgcccttttatttgcggaaaggccgtcatgttcaaggaccggaatacctgcaggacatacactctggctcagatctacaaccagattctggaggaggacaagaggagaaggcaggtcatcgacgtgatgtctgccatcttccagtga

SEQ ID NO: 48 - DNA, 990 bp, synthetic construct
  source 1..990: mol_type = other DNA
atgggaacccaggtgtctcgccagaatgtgggaacccactccactcagaacagcgtgagtaacgggagcagcctgaactattttaacatcaattattttaaggacgccgcttcctccggcgcaagcaagctggagttcagccaggacccctccaagtttaccgatccagtgaaagatgtcctggagaaaggcatccccaccctgcagagccccacagtggaggcctgtgggtatagcgacagaatcatccagatcactaggggcgactccaccattactagccaggatgttgccaatgccgtggtgggatatggggtgtggccacactatctcacaccacaggacgccaccgccatcgataaaccttccaggcctgacacttcctctaatcgcttttataccctggagagcaagatgtggaccagcgacagcaagggctggtggtggaagctgccagatgctctgaagaatatgggcattttcggagagaacatgttttatcacttcctggggcggtctggatacaccgtgcacgtgcagtgcaatgcttccaagtttcaccagggcactctgatcgtggttatgattcctgagcaccagctggcaagcgcatccaccggaaacgttaccgccggatacaatctgacacatcccggcgagcagggcagggatgtgggcattacccgggtggaagacctgctgaagcagcctagcgacgatagctggctgaattttgacggcacactgctgggaaacatcactattttcccccaccagtttatcaacctgagaagcaacaactccgctacaattatcgtgccctacgtgaatgccgttcctatggactccatgccacgccacaacaattggagcctggtcatcattccaatttgccctctggagagtgatggacagacacccgtgccaatcactatctccatttccccaatgtgtgccgagttttccggcgccagagccaagtcacagtga

SEQ ID NO: 49 - DNA, 990 bp, synthetic construct
  source 1..990: mol_type = other DNA
atgggcacccaggtgtctcggcagaatgtcggaacccactcaacacagaatagcgtgagcaacggcagcagcctgaactactttaatatcaattactttaaggacgctgccagcagcggagccagcaaactcgagttcagccaagacccaagcaagttcaccgaccccgtgaaagacgtcctggaaaagggaatccccaccctgcagagcccaacagtcgaggcttgtgggtattctgatagaatcatccagattaccagaggagacagtaccatcacatctcaggacgtggcaaatgcagtggtgggctacggagtgtggcctcactacctgaccccccaggatgccaccgccattgacaagccctcccgccctgacacttcttccaataggttttacaccctggagtctaagatgtggacatccgattccaagggatggtggtggaagctgcctgatgctctgaaaaacatgggcatcttcggggagaacatgttctatcactttctgggaaggtccggctatacagttcacgtgcagtgcaacgctagcaagttccatcagggcactctgatcgtggtcatgattcccgagcaccagctggcatctgcctccacagggaacgtgactgccggatataacctgacccaccctggagagcagggcagggacgtcggaatcaccagagtggaagacctgctcaaacagcccagcgacgactcttggctgaacttcgacggcacactgctggggaacattacaattttccctcaccagttcatcaacctgaggtctaacaacagcgccacaatcattgttccatatgtgaacgccgtgcccatggatagcatgccccggcacaacaactggagcctggttatcatccccatttgcccactggagagcgacggacagacacctgtgcccatcacaatctccatctctcccatgtgtgccgagttctctggggcacgcgctaaatcccagtga

SEQ ID NO: 50 - DNA, 1731 bp, synthetic construct
  source 1..1731: mol_type = other DNA
atgaaggccattctggtggtgctgctgtataccttcgctacagctaatgccggcccttccgatatgtacgttcacgtggggaacctgatctatagaaatctgcacctctttaacagcgagatgcacgactccatcctgatcagctatagtagcgaccttgtgatctatcgcactaataccaccggagacgactacattccaacctgcgactgcactgaggccacctactactgcaagcataaaaatcgctacttccccatcaaggtcaccagccatgactggtatgagatccaggagtctgaatactatcctaagcacatccagtacaacctgctgatcggcgagggcccatgtgagcctggggattgtgggggcaagctgctgtgcaagcacggcgtgatcggaatgatcaccgccggcggggacggacacgtggctttcattgatctgaggcactttcactgtgctgaggagcagggcatcactgactacatccacatgctgggcgaagcctttgggtccggattcgtggatagcgtgaaggagcagatcaatgccatcaaccctatcaacaacatcagcaaaaagatcatcaaatggctgctgaggatcatttctgccatggttatcatcatcagaaactccagcgaccctcagacaatcatcgccaccctcactctgattggctgcagcgggtccccttggagattcctgaaggagaagttctgcaagtggacccagctgaactacatccacaaagagagcgacagttggctgaaaaaattcaccgaaatgtgtaacgccgcaaggggcctggaatggatcggcaataagatttctaagtttatcgactggatgaaatccatgctgcctcaggcccagctgaaggtgaaatacctgagcgaactgaagaagctgaacctgctggagaagcagattgagcacctgagagcagcagactccgccacacaggaaaaaatcaagtgcgagattgacactctgcatgacctgtcctgcaaattcctgcctctgtacgcttccgaggccaaaagaatcaaggtgctgcacaataagtgcaatgtggttatcaagcagaaaaagcggtgtgagccagtggccgtggttattcacggagagcctggcaccggcaagagcatgactactaattttctcgcccggatgatcaccaatgactctgacatctactccctgcccccagacccaaagtatttcgacggatatgaccagcagagcgtggttatcatggatgacatcatgcagaatccctccggcgacgatatgaccctgttttgtcagatggtttcctccgtgaccttcatccccccaatggccgatctgcctgacaagggcaagccttttgacagcaggttcgtgctgtgcagcacaaaccacagcctgctgaccccccctactattacctctctgcccgccatgaacaggagattctttatggacctggacatcatcgtgtgtgataaatataaggacgcccagggaaagctgaatgtcagcgccgctttcaagccctgcgacgtggacaccaagatcggcaacgctcggtgttgtcctttcatctgcggcaaagccgtgatgttcaaagacagaaatacatgtcgcacctacactctggcccagatctacaaccagattctggaggaggataagaggagacgccaggtcattgacgtcatgtccgccattttccagtga

SEQ ID NO: 51 - DNA, 1731 bp, synthetic construct
  source 1..1731: mol_type = other DNA
atgaaggcaatcctcgtggtcctgctgtacaccttcgctacagccaacgccgggccaagcgacatgtatgtgcatgtgggaaacctgatctacagaaacctgcacctctttaacagcgagatgcacgattccattctgatttcctattcctctgatctcgttatctaccggaccaacaccacaggagacgactatattccaacatgcgactgcacagaggccacttactactgtaagcacaaaaacagatacttccccatcaaggtgacttcccacgactggtacgagatccaggaatctgaatactaccctaagcatatccagtacaacctgctgattggagaagggccatgcgagccaggcgattgtggcggcaagctgctgtgtaaacacggcgtgatcggcatgattaccgccgggggcgacggccatgtggcattcatcgacctgaggcatttccattgtgcagaggagcagggcattaccgattacattcacatgctgggcgaggctttcggcagcggctttgtggacagcgtgaaggagcagattaacgcaattaaccctatcaataacatttccaagaagatcatcaagtggctgctgagaatcatctccgccatggttattatcatccgcaactctagcgacccccagaccattatcgcaacactgaccctgatcggatgttccggctccccttggcgctttctgaaggagaagttttgcaagtggacccagctgaactacattcacaaggagagcgactcttggctgaaaaagttcacagaaatgtgtaatgccgccagagggctggaatggatcggaaataagatctccaagttcatcgactggatgaagtccatgctgccacaggcccagctgaaagtcaaatacctgtctgagctgaaaaaactgaacctgctggagaagcagattgaacaccttcgggccgctgactctgccactcaggagaagatcaagtgcgaaattgacaccctgcacgacctgagctgcaaattcctgcctctgtatgcctccgaggccaagaggatcaaggtgctgcacaacaaatgcaatgtcgttatcaagcagaagaagcggtgcgagcccgtggccgtcgtgatccacggggagcctggaacaggaaagtctatgactactaacttcctcgcccggatgatcacaaacgacagcgacatctatagcctgccacccgaccctaaatatttcgatggatacgaccagcagtctgtcgtgatcatggatgacatcatgcagaatcctagcggggatgatatgacactgttctgccagatggtgagcagcgtgacattcatcccaccaatggctgacctgccagacaagggcaagcctttcgactccagattcgtgctgtgctccaccaaccactccctcctgacccctcccacaattacctccctgccagccatgaataggcgcttctttatggacctggatatcatcgtctgcgacaaatacaaggatgcccaggggaaactgaacgtgtccgccgcctttaagccttgtgacgtggacacaaagattgggaatgccagatgctgcccttttatttgcggaaaggccgtcatgttcaaggaccggaatacctgcaggacatacactctggctcagatctacaaccagattctggaggaggacaagaggagaaggcaggtcatcgacgtgatgtctgccatcttccagtga

SEQ ID NO: 52 - DNA, 1047 bp, synthetic construct
  source 1..1047: mol_type = other DNA
atgaaggccattctggtggtgctgctgtataccttcgctacagctaatgccgatactctgggaacccaggtgtctcgccagaatgtgggaacccactccactcagaacagcgtgagtaacgggagcagcctgaactattttaacatcaattattttaaggacgccgcttcctccggcgcaagcaagctggagttcagccaggacccctccaagtttaccgatccagtgaaagatgtcctggagaaaggcatccccaccctgcagagccccacagtggaggcctgtgggtatagcgacagaatcatccagatcactaggggcgactccaccattactagccaggatgttgccaatgccgtggtgggatatggggtgtggccacactatctcacaccacaggacgccaccgccatcgataaaccttccaggcctgacacttcctctaatcgcttttataccctggagagcaagatgtggaccagcgacagcaagggctggtggtggaagctgccagatgctctgaagaatatgggcattttcggagagaacatgttttatcacttcctggggcggtctggatacaccgtgcacgtgcagtgcaatgcttccaagtttcaccagggcactctgatcgtggttatgattcctgagcaccagctggcaagcgcatccaccggaaacgttaccgccggatacaatctgacacatcccggcgagcagggcagggatgtgggcattacccgggtggaagacctgctgaagcagcctagcgacgatagctggctgaattttgacggcacactgctgggaaacatcactattttcccccaccagtttatcaacctgagaagcaacaactccgctacaattatcgtgccctacgtgaatgccgttcctatggactccatgccacgccacaacaattggagcctggtcatcattccaatttgccctctggagagtgatggacagacacccgtgccaatcactatctccatttccccaatgtgtgccgagttttccggcgccagagccaagtcacagtga

SEQ ID NO: 53 - DNA, 1047 bp, synthetic construct
  source 1..1047: mol_type = other DNA
atgaaggcaatcctcgtggtcctgctgtacaccttcgctacagccaacgccgatacactcggcacccaggtgtctcggcagaatgtcggaacccactcaacacagaatagcgtgagcaacggcagcagcctgaactactttaatatcaattactttaaggacgctgccagcagcggagccagcaaactcgagttcagccaagacccaagcaagttcaccgaccccgtgaaagacgtcctggaaaagggaatccccaccctgcagagcccaacagtcgaggcttgtgggtattctgatagaatcatccagattaccagaggagacagtaccatcacatctcaggacgtggcaaatgcagtggtgggctacggagtgtggcctcactacctgaccccccaggatgccaccgccattgacaagccctcccgccctgacacttcttccaataggttttacaccctggagtctaagatgtggacatccgattccaagggatggtggtggaagctgcctgatgctctgaaaaacatgggcatcttcggggagaacatgttctatcactttctgggaaggtccggctatacagttcacgtgcagtgcaacgctagcaagttccatcagggcactctgatcgtggtcatgattcccgagcaccagctggcatctgcctccacagggaacgtgactgccggatataacctgacccaccctggagagcagggcagggacgtcggaatcaccagagtggaagacctgctcaaacagcccagcgacgactcttggctgaacttcgacggcacactgctggggaacattacaattttccctcaccagttcatcaacctgaggtctaacaacagcgccacaatcattgttccatatgtgaacgccgtgcccatggatagcatgccccggcacaacaactggagcctggttatcatccccatttgcccactggagagcgacggacagacacctgtgcccatcacaatctccatctctcccatgtgtgccgagttctctggggcacgcgctaaatcccagtga

SEQ ID NO: 54 - DNA, 2667 bp, synthetic construct
  source 1..2667: mol_type = other DNA
atgggcccttccgatatgtacgttcacgtggggaacctgatctatagaaatctgcacctctttaacagcgagatgcacgactccatcctgatcagctatagtagcgaccttgtgatctatcgcactaataccaccggagacgactacattccaacctgcgactgcactgaggccacctactactgcaagcataaaaatcgctacttccccatcaaggtcaccagccatgactggtatgagatccaggagtctgaatactatcctaagcacatccagtacaacctgctgatcggcgagggcccatgtgagcctggggattgtgggggcaagctgctgtgcaagcacggcgtgatcggaatgatcaccgccggcggggacggacacgtggctttcattgatctgaggcactttcactgtgctgaggagcagggcatcactgactacatccacatgctgggcgaagcctttgggtccggattcgtggatagcgtgaaggagcagatcaatgccatcaaccctatcaacaacatcagcaaaaagatcatcaaatggctgctgaggatcatttctgccatggttatcatcatcagaaactccagcgaccctcagacaatcatcgccaccctcactctgattggctgcagcgggtccccttggagattcctgaaggagaagttctgcaagtggacccagctgaactacatccacaaagagagcgacagttggctgaaaaaattcaccgaaatgtgtaacgccgcaaggggcctggaatggatcggcaataagatttctaagtttatcgactggatgaaatccatgctgcctcaggcccagctgaaggtgaaatacctgagcgaactgaagaagctgaacctgctggagaagcagattgagcacctgagagcagcagactccgccacacaggaaaaaatcaagtgcgagattgacactctgcatgacctgtcctgcaaattcctgcctctgtacgcttccgaggccaaaagaatcaaggtgctgcacaataagtgcaatgtggttatcaagcagaaaaagcggtgtgagccagtggccgtggttattcacggagagcctggcaccggcaagagcatgactactaattttctcgcccggatgatcaccaatgactctgacatctactccctgcccccagacccaaagtatttcgacggatatgaccagcagagcgtggttatcatggatgacatcatgcagaatccctccggcgacgatatgaccctgttttgtcagatggtttcctccgtgaccttcatccccccaatggccgatctgcctgacaagggcaagccttttgacagcaggttcgtgctgtgcagcacaaaccacagcctgctgaccccccctactattacctctctgcccgccatgaacaggagattctttatggacctggacatcatcgtgtgtgataaatataaggacgcccagggaaagctgaatgtcagcgccgctttcaagccctgcgacgtggacaccaagatcggcaacgctcggtgttgtcctttcatctgcggcaaagccgtgatgttcaaagacagaaatacatgtcgcacctacactctggcccagatctacaaccagattctggaggaggataagaggagacgccaggtcattgacgtcatgtccgccattttccagggaacccaggtgtctcgccagaatgtgggaacccactccactcagaacagcgtgagtaacgggagcagcctgaactattttaacatcaattattttaaggacgccgcttcctccggcgcaagcaagctggagttcagccaggacccctccaagtttaccgatccagtgaaagatgtcctggagaaaggcatccccaccctgcagagccccacagtggaggcctgtgggtatagcgacagaatcatccagatcactaggggcgactccaccattactagccaggatgttgccaatgccgtggtgggatatggggtgtggccacactatctcacaccacaggacgccaccgccatcgataaaccttccaggcctgacacttcctctaatcgcttttataccctggagagcaagatgtggaccagcgacagcaagggctggtggtggaagctgccagatgctctgaagaatatgggcattttcggagagaacatgttttatcacttcctggggcggtctggatacaccgtgcacgtgcagtgcaatgcttccaagtttcaccagggcactctgatcgtggttatgattcctgagcaccagctggcaagcgcatccaccggaaacgttaccgccggatacaatctgacacatcccggcgagcagggcagggatgtgggcattacccgggtggaagacctgctgaagcagcctagcgacgatagctggctgaattttgacggcacactgctgggaaacatcactattttcccccaccagtttatcaacctgagaagcaacaactccgctacaattatcgtgccctacgtgaatgccgttcctatggactccatgccacgccacaacaattggagcctggtcatcattccaatttgccctctggagagtgatggacagacacccgtgccaatcactatctccatttccccaatgtgtgccgagttttccggcgccagagccaagtcacagtga

SEQ ID NO: 55 - DNA, 2667 bp, synthetic construct
  source 1..2667: mol_type = other DNA
atggggccaagcgacatgtatgtgcatgtgggaaacctgatctacagaaacctgcacctctttaacagcgagatgcacgattccattctgatttcctattcctctgatctcgttatctaccggaccaacaccacaggagacgactatattccaacatgcgactgcacagaggccacttactactgtaagcacaaaaacagatacttccccatcaaggtgacttcccacgactggtacgagatccaggaatctgaatactaccctaagcatatccagtacaacctgctgattggagaagggccatgcgagccaggcgattgtggcggcaagctgctgtgtaaacacggcgtgatcggcatgattaccgccgggggcgacggccatgtggcattcatcgacctgaggcatttccattgtgcagaggagcagggcattaccgattacattcacatgctgggcgaggctttcggcagcggctttgtggacagcgtgaaggagcagattaacgcaattaaccctatcaataacatttccaagaagatcatcaagtggctgctgagaatcatctccgccatggttattatcatccgcaactctagcgacccccagaccattatcgcaacactgaccctgatcggatgttccggctccccttggcgctttctgaaggagaagttttgcaagtggacccagctgaactacattcacaaggagagcgactcttggctgaaaaagttcacagaaatgtgtaatgccgccagagggctggaatggatcggaaataagatctccaagttcatcgactggatgaagtccatgctgccacaggcccagctgaaagtcaaatacctgtctgagctgaaaaaactgaacctgctggagaagcagattgaacaccttcgggccgctgactctgccactcaggagaagatcaagtgcgaaattgacaccctgcacgacctgagctgcaaattcctgcctctgtatgcctccgaggccaagaggatcaaggtgctgcacaacaaatgcaatgtcgttatcaagcagaagaagcggtgcgagcccgtggccgtcgtgatccacggggagcctggaacaggaaagtctatgactactaacttcctcgcccggatgatcacaaacgacagcgacatctatagcctgccacccgaccctaaatatttcgatggatacgaccagcagtctgtcgtgatcatggatgacatcatgcagaatcctagcggggatgatatgacactgttctgccagatggtgagcagcgtgacattcatcccaccaatggctgacctgccagacaagggcaagcctttcgactccagattcgtgctgtgctccaccaaccactccctcctgacccctcccacaattacctccctgccagccatgaataggcgcttctttatggacctggatatcatcgtctgcgacaaatacaaggatgcccaggggaaactgaacgtgtccgccgcctttaagccttgtgacgtggacacaaagattgggaatgccagatgctgcccttttatttgcggaaaggccgtcatgttcaaggaccggaatacctgcaggacatacactctggctcagatctacaaccagattctggaggaggacaagaggagaaggcaggtcatcgacgtgatgtctgccatcttccagggcacccaggtgtctcggcagaatgtcggaacccactcaacacagaatagcgtgagcaacggcagcagcctgaactactttaatatcaattactttaaggacgctgccagcagcggagccagcaaactcgagttcagccaagacccaagcaagttcaccgaccccgtgaaagacgtcctggaaaagggaatccccaccctgcagagcccaacagtcgaggcttgtgggtattctgatagaatcatccagattaccagaggagacagtaccatcacatctcaggacgtggcaaatgcagtggtgggctacggagtgtggcctcactacctgaccccccaggatgccaccgccattgacaagccctcccgccctgacacttcttccaataggttttacaccctggagtctaagatgtggacatccgattccaagggatggtggtggaagctgcctgatgctctgaaaaacatgggcatcttcggggagaacatgttctatcactttctgggaaggtccggctatacagttcacgtgcagtgcaacgctagcaagttccatcagggcactctgatcgtggtcatgattcccgagcaccagctggcatctgcctccacagggaacgtgactgccggatataacctgacccaccctggagagcagggcagggacgtcggaatcaccagagtggaagacctgctcaaacagcccagcgacgactcttggctgaacttcgacggcacactgctggggaacattacaattttccctcaccagttcatcaacctgaggtctaacaacagcgccacaatcattgttccatatgtgaacgccgtgcccatggatagcatgccccggcacaacaactggagcctggttatcatccccatttgcccactggagagcgacggacagacacctgtgcccatcacaatctccatctctcccatgtgtgccgagttctctggggcacgcgctaaatcccagtga

SEQ ID NO: 56 - DNA, 2667 bp, synthetic construct
  source 1..2667: mol_type = other DNA
atgggaacccaggtgtctcgccagaatgtgggaacccactccactcagaacagcgtgagtaacgggagcagcctgaactattttaacatcaattattttaaggacgccgcttcctccggcgcaagcaagctggagttcagccaggacccctccaagtttaccgatccagtgaaagatgtcctggagaaaggcatccccaccctgcagagccccacagtggaggcctgtgggtatagcgacagaatcatccagatcactaggggcgactccaccattactagccaggatgttgccaatgccgtggtgggatatggggtgtggccacactatctcacaccacaggacgccaccgccatcgataaaccttccaggcctgacacttcctctaatcgcttttataccctggagagcaagatgtggaccagcgacagcaagggctggtggtggaagctgccagatgctctgaagaatatgggcattttcggagagaacatgttttatcacttcctggggcggtctggatacaccgtgcacgtgcagtgcaatgcttccaagtttcaccagggcactctgatcgtggttatgattcctgagcaccagctggcaagcgcatccaccggaaacgttaccgccggatacaatctgacacatcccggcgagcagggcagggatgtgggcattacccgggtggaagacctgctgaagcagcctagcgacgatagctggctgaattttgacggcacactgctgggaaacatcactattttcccccaccagtttatcaacctgagaagcaacaactccgctacaattatcgtgccctacgtgaatgccgttcctatggactccatgccacgccacaacaattggagcctggtcatcattccaatttgccctctggagagtgatggacagacacccgtgccaatcactatctccatttccccaatgtgtgccgagttttccggcgccagagccaagtcacagggcccttccgatatgtacgttcacgtggggaacctgatctatagaaatctgcacctctttaacagcgagatgcacgactccatcctgatcagctatagtagcgaccttgtgatctatcgcactaataccaccggagacgactacattccaacctgcgactgcactgaggccacctactactgcaagcataaaaatcgctacttccccatcaaggtcaccagccatgactggtatgagatccaggagtctgaatactatcctaagcacatccagtacaacctgctgatcggcgagggcccatgtgagcctggggattgtgggggcaagctgctgtgcaagcacggcgtgatcggaatgatcaccgccggcggggacggacacgtggctttcattgatctgaggcactttcactgtgctgaggagcagggcatcactgactacatccacatgctgggcgaagcctttgggtccggattcgtggatagcgtgaaggagcagatcaatgccatcaaccctatcaacaacatcagcaaaaagatcatcaaatggctgctgaggatcatttctgccatggttatcatcatcagaaactccagcgaccctcagacaatcatcgccaccctcactctgattggctgcagcgggtccccttggagattcctgaaggagaagttctgcaagtggacccagctgaactacatccacaaagagagcgacagttggctgaaaaaattcaccgaaatgtgtaacgccgcaaggggcctggaatggatcggcaataagatttctaagtttatcgactggatgaaatccatgctgcctcaggcccagctgaaggtgaaatacctgagcgaactgaagaagctgaacctgctggagaagcagattgagcacctgagagcagcagactccgccacacaggaaaaaatcaagtgcgagattgacactctgcatgacctgtcctgcaaattcctgcctctgtacgcttccgaggccaaaagaatcaaggtgctgcacaataagtgcaatgtggttatcaagcagaaaaagcggtgtgagccagtggccgtggttattcacggagagcctggcaccggcaagagcatgactactaattttctcgcccggatgatcaccaatgactctgacatctactccctgcccccagacccaaagtatttcgacggatatgaccagcagagcgtggttatcatggatgacatcatgcagaatccctccggcgacgatatgaccctgttttgtcagatggtttcctccgtgaccttcatccccccaatggccgatctgcctgacaagggcaagccttttgacagcaggttcgtgctgtgcagcacaaaccacagcctgctgaccccccctactattacctctctgcccgccatgaacaggagattctttatggacctggacatcatcgtgtgtgataaatataaggacgcccagggaaagctgaatgtcagcgccgctttcaagccctgcgacgtggacaccaagatcggcaacgctcggtgttgtcctttcatctgcggcaaagccgtgatgttcaaagacagaaatacatgtcgcacctacactctggcccagatctacaaccagattctggaggaggataagaggagacgccaggtcattgacgtcatgtccgccattttccagtga

SEQ ID NO: 57 - DNA, 2667 bp, synthetic construct
  source 1..2667: mol_type = other DNA
atgggcacccaggtgtctcggcagaatgtcggaacccactcaacacagaatagcgtgagcaacggcagcagcctgaactactttaatatcaattactttaaggacgctgccagcagcggagccagcaaactcgagttcagccaagacccaagcaagttcaccgaccccgtgaaagacgtcctggaaaagggaatccccaccctgcagagcccaacagtcgaggcttgtgggtattctgatagaatcatccagattaccagaggagacagtaccatcacatctcaggacgtggcaaatgcagtggtgggctacggagtgtggcctcactacctgaccccccaggatgccaccgccattgacaagccctcccgccctgacacttcttccaataggttttacaccctggagtctaagatgtggacatccgattccaagggatggtggtggaagctgcctgatgctctgaaaaacatgggcatcttcggggagaacatgttctatcactttctgggaaggtccggctatacagttcacgtgcagtgcaacgctagcaagttccatcagggcactctgatcgtggtcatgattcccgagcaccagctggcatctgcctccacagggaacgtgactgccggatataacctgacccaccctggagagcagggcagggacgtcggaatcaccagagtggaagacctgctcaaacagcccagcgacgactcttggctgaacttcgacggcacactgctggggaacattacaattttccctcaccagttcatcaacctgaggtctaacaacagcgccacaatcattgttccatatgtgaacgccgtgcccatggatagcatgccccggcacaacaactggagcctggttatcatccccatttgcccactggagagcgacggacagacacctgtgcccatcacaatctccatctctcccatgtgtgccgagttctctggggcacgcgctaaatcccaggggccaagcgacatgtatgtgcatgtgggaaacctgatctacagaaacctgcacctctttaacagcgagatgcacgattccattctgatttcctattcctctgatctcgttatctaccggaccaacaccacaggagacgactatattccaacatgcgactgcacagaggccacttactactgtaagcacaaaaacagatacttccccatcaaggtgacttcccacgactggtacgagatccaggaatctgaatactaccctaagcatatccagtacaacctgctgattggagaagggccatgcgagccaggcgattgtggcggcaagctgctgtgtaaacacggcgtgatcggcatgattaccgccgggggcgacggccatgtggcattcatcgacctgaggcatttccattgtgcagaggagcagggcattaccgattacattcacatgctgggcgaggctttcggcagcggctttgtggacagcgtgaaggagcagattaacgcaattaaccctatcaataacatttccaagaagatcatcaagtggctgctgagaatcatctccgccatggttattatcatccgcaactctagcgacccccagaccattatcgcaacactgaccctgatcggatgttccggctccccttggcgctttctgaaggagaagttttgcaagtggacccagctgaactacattcacaaggagagcgactcttggctgaaaaagttcacagaaatgtgtaatgccgccagagggctggaatggatcggaaataagatctccaagttcatcgactggatgaagtccatgctgccacaggcccagctgaaagtcaaatacctgtctgagctgaaaaaactgaacctgctggagaagcagattgaacaccttcgggccgctgactctgccactcaggagaagatcaagtgcgaaattgacaccctgcacgacctgagctgcaaattcctgcctctgtatgcctccgaggccaagaggatcaaggtgctgcacaacaaatgcaatgtcgttatcaagcagaagaagcggtgcgagcccgtggccgtcgtgatccacggggagcctggaacaggaaagtctatgactactaacttcctcgcccggatgatcacaaacgacagcgacatctatagcctgccacccgaccctaaatatttcgatggatacgaccagcagtctgtcgtgatcatggatgacatcatgcagaatcctagcggggatgatatgacactgttctgccagatggtgagcagcgtgacattcatcccaccaatggctgacctgccagacaagggcaagcctttcgactccagattcgtgctgtgctccaccaaccactccctcctgacccctcccacaattacctccctgccagccatgaataggcgcttctttatggacctggatatcatcgtctgcgacaaatacaaggatgcccaggggaaactgaacgtgtccgccgcctttaagccttgtgacgtggacacaaagattgggaatgccagatgctgcccttttatttgcggaaaggccgtcatgttcaaggaccggaatacctgcaggacatacactctggctcagatctacaaccagattctggaggaggacaagaggagaaggcaggtcatcgacgtgatgtctgccatcttccagtga

SEQ ID NO: 58 - DNA, 2715 bp, synthetic construct
  source 1..2715: mol_type = other DNA
atgaaggccattctggtggtgctgctgtataccttcgctacagctaatgccggcccttccgatatgtacgttcacgtggggaacctgatctatagaaatctgcacctctttaacagcgagatgcacgactccatcctgatcagctatagtagcgaccttgtgatctatcgcactaataccaccggagacgactacattccaacctgcgactgcactgaggccacctactactgcaagcataaaaatcgctacttccccatcaaggtcaccagccatgactggtatgagatccaggagtctgaatactatcctaagcacatccagtacaacctgctgatcggcgagggcccatgtgagcctggggattgtgggggcaagctgctgtgcaagcacggcgtgatcggaatgatcaccgccggcggggacggacacgtggctttcattgatctgaggcactttcactgtgctgaggagcagggcatcactgactacatccacatgctgggcgaagcctttgggtccggattcgtggatagcgtgaaggagcagatcaatgccatcaaccctatcaacaacatcagcaaaaagatcatcaaatggctgctgaggatcatttctgccatggttatcatcatcagaaactccagcgaccctcagacaatcatcgccaccctcactctgattggctgcagcgggtccccttggagattcctgaaggagaagttctgcaagtggacccagctgaactacatccacaaagagagcgacagttggctgaaaaaattcaccgaaatgtgtaacgccgcaaggggcctggaatggatcggcaataagatttctaagtttatcgactggatgaaatccatgctgcctcaggcccagctgaaggtgaaatacctgagcgaactgaagaagctgaacctgctggagaagcagattgagcacctgagagcagcagactccgccacacaggaaaaaatcaagtgcgagattgacactctgcatgacctgtcctgcaaattcctgcctctgtacgcttccgaggccaaaagaatcaaggtgctgcacaataagtgcaatgtggttatcaagcagaaaaagcggtgtgagccagtggccgtggttattcacggagagcctggcaccggcaagagcatgactactaattttctcgcccggatgatcaccaatgactctgacatctactccctgcccccagacccaaagtatttcgacggatatgaccagcagagcgtggttatcatggatgacatcatgcagaatccctccggcgacgatatgaccctgttttgtcagatggtttcctccgtgaccttcatccccccaatggccgatctgcctgacaagggcaagccttttgacagcaggttcgtgctgtgcagcacaaaccacagcctgctgaccccccctactattacctctctgcccgccatgaacaggagattctttatggacctggacatcatcgtgtgtgataaatataaggacgcccagggaaagctgaatgtcagcgccgctttcaagccctgcgacgtggacaccaagatcggcaacgctcggtgttgtcctttcatctgcggcaaagccgtgatgttcaaagacagaaatacatgtcgcacctacactctggcccagatctacaaccagattctggaggaggataagaggagacgccaggtcattgacgtcatgtccgccattttccagggaacccaggtgtctcgccagaatgtgggaacccactccactcagaacagcgtgagtaacgggagcagcctgaactattttaacatcaattattttaaggacgccgcttcctccggcgcaagcaagctggagttcagccaggacccctccaagtttaccgatccagtgaaagatgtcctggagaaaggcatccccaccctgcagagccccacagtggaggcctgtgggtatagcgacagaatcatccagatcactaggggcgactccaccattactagccaggatgttgccaatgccgtggtgggatatggggtgtggccacactatctcacaccacaggacgccaccgccatcgataaaccttccaggcctgacacttcctctaatcgcttttataccctggagagcaagatgtggaccagcgacagcaagggctggtggtggaagctgccagatgctctgaagaatatgggcattttcggagagaacatgttttatcacttcctggggcggtctggatacaccgtgcacgtgcagtgcaatgcttccaagtttcaccagggcactctgatcgtggttatgattcctgagcaccagctggcaagcgcatccaccggaaacgttaccgccggatacaatctgacacatcccggcgagcagggcagggatgtgggcattacccgggtggaagacctgctgaagcagcctagcgacgatagctggctgaattttgacggcacactgctgggaaacatcactattttcccccaccagtttatcaacctgagaagcaacaactccgctacaattatcgtgccctacgtgaatgccgttcctatggactccatgccacgccacaacaattggagcctggtcatcattccaatttgccctctggagagtgatggacagacacccgtgccaatcactatctccatttccccaatgtgtgccgagttttccggcgccagagccaagtcacagtga

SEQ ID NO: 59 - DNA, 2715 bp, synthetic construct
  source 1..2715: mol_type = other DNA
atgaaggcaatcctcgtggtcctgctgtacaccttcgctacagccaacgccgggccaagcgacatgtatgtgcatgtgggaaacctgatctacagaaacctgcacctctttaacagcgagatgcacgattccattctgatttcctattcctctgatctcgttatctaccggaccaacaccacaggagacgactatattccaacatgcgactgcacagaggccacttactactgtaagcacaaaaacagatacttccccatcaaggtgacttcccacgactggtacgagatccaggaatctgaatactaccctaagcatatccagtacaacctgctgattggagaagggccatgcgagccaggcgattgtggcggcaagctgctgtgtaaacacggcgtgatcggcatgattaccgccgggggcgacggccatgtggcattcatcgacctgaggcatttccattgtgcagaggagcagggcattaccgattacattcacatgctgggcgaggctttcggcagcggctttgtggacagcgtgaaggagcagattaacgcaattaaccctatcaataacatttccaagaagatcatcaagtggctgctgagaatcatctccgccatggttattatcatccgcaactctagcgacccccagaccattatcgcaacactgaccctgatcggatgttccggctccccttggcgctttctgaaggagaagttttgcaagtggacccagctgaactacattcacaaggagagcgactcttggctgaaaaagttcacagaaatgtgtaatgccgccagagggctggaatggatcggaaataagatctccaagttcatcgactggatgaagtccatgctgccacaggcccagctgaaagtcaaatacctgtctgagctgaaaaaactgaacctgctggagaagcagattgaacaccttcgggccgctgactctgccactcaggagaagatcaagtgcgaaattgacaccctgcacgacctgagctgcaaattcctgcctctgtatgcctccgaggccaagaggatcaaggtgctgcacaacaaatgcaatgtcgttatcaagcagaagaagcggtgcgagcccgtggccgtcgtgatccacggggagcctggaacaggaaagtctatgactactaacttcctcgcccggatgatcacaaacgacagcgacatctatagcctgccacccgaccctaaatatttcgatggatacgaccagcagtctgtcgtgatcatggatgacatcatgcagaatcctagcggggatgatatgacactgttctgccagatggtgagcagcgtgacattcatcccaccaatggctgacctgccagacaagggcaagcctttcgactccagattcgtgctgtgctccaccaaccactccctcctgacccctcccacaattacctccctgccagccatgaataggcgcttctttatggacctggatatcatcgtctgcgacaaatacaaggatgcccaggggaaactgaacgtgtccgccgcctttaagccttgtgacgtggacacaaagattgggaatgccagatgctgcccttttatttgcggaaaggccgtcatgttcaaggaccggaatacctgcaggacatacactctggctcagatctacaaccagattctggaggaggacaagaggagaaggcaggtcatcgacgtgatgtctgccatcttccagggcacccaggtgtctcggcagaatgtcggaacccactcaacacagaatagcgtgagcaacggcagcagcctgaactactttaatatcaattactttaaggacgctgccagcagcggagccagcaaactcgagttcagccaagacccaagcaagttcaccgaccccgtgaaagacgtcctggaaaagggaatccccaccctgcagagcccaacagtcgaggcttgtgggtattctgatagaatcatccagattaccagaggagacagtaccatcacatctcaggacgtggcaaatgcagtggtgggctacggagtgtggcctcactacctgaccccccaggatgccaccgccattgacaagccctcccgccctgacacttcttccaataggttttacaccctggagtctaagatgtggacatccgattccaagggatggtggtggaagctgcctgatgctctgaaaaacatgggcatcttcggggagaacatgttctatcactttctgggaaggtccggctatacagttcacgtgcagtgcaacgctagcaagttccatcagggcactctgatcgtggtcatgattcccgagcaccagctggcatctgcctccacagggaacgtgactgccggatataacctgacccaccctggagagcagggcagggacgtcggaatcaccagagtggaagacctgctcaaacagcccagcgacgactcttggctgaacttcgacggcacactgctggggaacattacaattttccctcaccagttcatcaacctgaggtctaacaacagcgccacaatcattgttccatatgtgaacgccgtgcccatggatagcatgccccggcacaacaactggagcctggttatcatccccatttgcccactggagagcgacggacagacacctgtgcccatcacaatctccatctctcccatgtgtgccgagttctctggggcacgcgctaaatcccagtga

SEQ ID NO: 60 - DNA, 2724 bp, synthetic construct
  source 1..2724: mol_type = other DNA
atgaaggccattctggtggtgctgctgtataccttcgctacagctaatgccgatactctgggaacccaggtgtctcgccagaatgtgggaacccactccactcagaacagcgtgagtaacgggagcagcctgaactattttaacatcaattattttaaggacgccgcttcctccggcgcaagcaagctggagttcagccaggacccctccaagtttaccgatccagtgaaagatgtcctggagaaaggcatccccaccctgcagagccccacagtggaggcctgtgggtatagcgacagaatcatccagatcactaggggcgactccaccattactagccaggatgttgccaatgccgtggtgggatatggggtgtggccacactatctcacaccacaggacgccaccgccatcgataaaccttccaggcctgacacttcctctaatcgcttttataccctggagagcaagatgtggaccagcgacagcaagggctggtggtggaagctgccagatgctctgaagaatatgggcattttcggagagaacatgttttatcacttcctggggcggtctggatacaccgtgcacgtgcagtgcaatgcttccaagtttcaccagggcactctgatcgtggttatgattcctgagcaccagctggcaagcgcatccaccggaaacgttaccgccggatacaatctgacacatcccggcgagcagggcagggatgtgggcattacccgggtggaagacctgctgaagcagcctagcgacgatagctggctgaattttgacggcacactgctgggaaacatcactattttcccccaccagtttatcaacctgagaagcaacaactccgctacaattatcgtgccctacgtgaatgccgttcctatggactccatgccacgccacaacaattggagcctggtcatcattccaatttgccctctggagagtgatggacagacacccgtgccaatcactatctccatttccccaatgtgtgccgagttttccggcgccagagccaagtcacagggcccttccgatatgtacgttcacgtggggaacctgatctatagaaatctgcacctctttaacagcgagatgcacgactccatcctgatcagctatagtagcgaccttgtgatctatcgcactaataccaccggagacgactacattccaacctgcgactgcactgaggccacctactactgcaagcataaaaatcgctacttccccatcaaggtcaccagccatgactggtatgagatccaggagtctgaatactatcctaagcacatccagtacaacctgctgatcggcgagggcccatgtgagcctggggattgtgggggcaagctgctgtgcaagcacggcgtgatcggaatgatcaccgccggcggggacggacacgtggctttcattgatctgaggcactttcactgtgctgaggagcagggcatcactgactacatccacatgctgggcgaagcctttgggtccggattcgtggatagcgtgaaggagcagatcaatgccatcaaccctatcaacaacatcagcaaaaagatcatcaaatggctgctgaggatcatttctgccatggttatcatcatcagaaactccagcgaccctcagacaatcatcgccaccctcactctgattggctgcagcgggtccccttggagattcctgaaggagaagttctgcaagtggacccagctgaactacatccacaaagagagcgacagttggctgaaaaaattcaccgaaatgtgtaacgccgcaaggggcctggaatggatcggcaataagatttctaagtttatcgactggatgaaatccatgctgcctcaggcccagctgaaggtgaaatacctgagcgaactgaagaagctgaacctgctggagaagcagattgagcacctgagagcagcagactccgccacacaggaaaaaatcaagtgcgagattgacactctgcatgacctgtcctgcaaattcctgcctctgtacgcttccgaggccaaaagaatcaaggtgctgcacaataagtgcaatgtggttatcaagcagaaaaagcggtgtgagccagtggccgtggttattcacggagagcctggcaccggcaagagcatgactactaattttctcgcccggatgatcaccaatgactctgacatctactccctgcccccagacccaaagtatttcgacggatatgaccagcagagcgtggttatcatggatgacatcatgcagaatccctccggcgacgatatgaccctgttttgtcagatggtttcctccgtgaccttcatccccccaatggccgatctgcctgacaagggcaagccttttgacagcaggttcgtgctgtgcagcacaaaccacagcctgctgaccccccctactattacctctctgcccgccatgaacaggagattctttatggacctggacatcatcgtgtgtgataaatataaggacgcccagggaaagctgaatgtcagcgccgctttcaagccctgcgacgtggacaccaagatcggcaacgctcggtgttgtcctttcatctgcggcaaagccgtgatgttcaaagacagaaatacatgtcgcacctacactctggcccagatctacaaccagattctggaggaggataagaggagacgccaggtcattgacgtcatgtccgccattttccagtga

SEQ ID NO: 61 - DNA, 2724 bp, synthetic construct
  source 1..2724: mol_type = other DNA
atgaaggcaatcctcgtggtcctgctgtacaccttcgctacagccaacgccgatacactcggcacccaggtgtctcggcagaatgtcggaacccactcaacacagaatagcgtgagcaacggcagcagcctgaactactttaatatcaattactttaaggacgctgccagcagcggagccagcaaactcgagttcagccaagacccaagcaagttcaccgaccccgtgaaagacgtcctggaaaagggaatccccaccctgcagagcccaacagtcgaggcttgtgggtattctgatagaatcatccagattaccagaggagacagtaccatcacatctcaggacgtggcaaatgcagtggtgggctacggagtgtggcctcactacctgaccccccaggatgccaccgccattgacaagccctcccgccctgacacttcttccaataggttttacaccctggagtctaagatgtggacatccgattccaagggatggtggtggaagctgcctgatgctctgaaaaacatgggcatcttcggggagaacatgttctatcactttctgggaaggtccggctatacagttcacgtgcagtgcaacgctagcaagttccatcagggcactctgatcgtggtcatgattcccgagcaccagctggcatctgcctccacagggaacgtgactgccggatataacctgacccaccctggagagcagggcagggacgtcggaatcaccagagtggaagacctgctcaaacagcccagcgacgactcttggctgaacttcgacggcacactgctggggaacattacaattttccctcaccagttcatcaacctgaggtctaacaacagcgccacaatcattgttccatatgtgaacgccgtgcccatggatagcatgccccggcacaacaactggagcctggttatcatccccatttgcccactggagagcgacggacagacacctgtgcccatcacaatctccatctctcccatgtgtgccgagttctctggggcacgcgctaaatcccaggggccaagcgacatgtatgtgcatgtgggaaacctgatctacagaaacctgcacctctttaacagcgagatgcacgattccattctgatttcctattcctctgatctcgttatctaccggaccaacaccacaggagacgactatattccaacatgcgactgcacagaggccacttactactgtaagcacaaaaacagatacttccccatcaaggtgacttcccacgactggtacgagatccaggaatctgaatactaccctaagcatatccagtacaacctgctgattggagaagggccatgcgagccaggcgattgtggcggcaagctgctgtgtaaacacggcgtgatcggcatgattaccgccgggggcgacggccatgtggcattcatcgacctgaggcatttccattgtgcagaggagcagggcattaccgattacattcacatgctgggcgaggctttcggcagcggctttgtggacagcgtgaaggagcagattaacgcaattaaccctatcaataacatttccaagaagatcatcaagtggctgctgagaatcatctccgccatggttattatcatccgcaactctagcgacccccagaccattatcgcaacactgaccctgatcggatgttccggctccccttggcgctttctgaaggagaagttttgcaagtggacccagctgaactacattcacaaggagagcgactcttggctgaaaaagttcacagaaatgtgtaatgccgccagagggctggaatggatcggaaataagatctccaagttcatcgactggatgaagtccatgctgccacaggcccagctgaaagtcaaatacctgtctgagctgaaaaaactgaacctgctggagaagcagattgaacaccttcgggccgctgactctgccactcaggagaagatcaagtgcgaaattgacaccctgcacgacctgagctgcaaattcctgcctctgtatgcctccgaggccaagaggatcaaggtgctgcacaacaaatgcaatgtcgttatcaagcagaagaagcggtgcgagcccgtggccgtcgtgatccacggggagcctggaacaggaaagtctatgactactaacttcctcgcccggatgatcacaaacgacagcgacatctatagcctgccacccgaccctaaatatttcgatggatacgaccagcagtctgtcgtgatcatggatgacatcatgcagaatcctagcggggatgatatgacactgttctgccagatggtgagcagcgtgacattcatcccaccaatggctgacctgccagacaagggcaagcctttcgactccagattcgtgctgtgctccaccaaccactccctcctgacccctcccacaattacctccctgccagccatgaataggcgcttctttatggacctggatatcatcgtctgcgacaaatacaaggatgcccaggggaaactgaacgtgtccgccgcctttaagccttgtgacgtggacacaaagattgggaatgccagatgctgcccttttatttgcggaaaggccgtcatgttcaaggaccggaatacctgcaggacatacactctggctcagatctacaaccagattctggaggaggacaagaggagaaggcaggtcatcgacgtgatgtctgccatcttccagtga

SEQ ID NO: 62 - DNA, 2667 bp, synthetic construct
  source 1..2667: mol_type = other DNA
atgggcccttccgatatgtacgttcacgtggggaacctgatctatagaaatctgcacctctttaacagcgagatgcacgactccatcctgatcagctatagtagcgaccttgtgatctatcgcactaataccaccggagacgactacattccaacctgcgactgcactgaggccacctactactgcaagcataaaaatcgctacttccccatcaaggtcaccagccatgactggtatgagatccaggagtctgaatactatcctaagcacatccagtacaacctgctgatcggcgagggcccatgtgagcctggggattgtgggggcaagctgctgtgcaagcacggcgtgatcggaatgatcaccgccggcggggacggacacgtggctttcattgatctgaggcactttcactgtgctgaggagcagggcatcactgactacatccacatgctgggcgaagcctttgggtccggattcgtggatagcgtgaaggagcagatcaatgccatcaaccctatcaacaacatcagcaaaaagatcatcaaatggctgctgaggatcatttctgccatggttatcatcatcagaaactccagcgaccctcagacaatcatcgccaccctcactctgattggctgcagcgggtccccttggagattcctgaaggagaagttctgcaagtggacccagctgaactacatccacaaagagagcgacagttggctgaaaaaattcaccgaaatgtgtaacgccgcaaggggcctggaatggatcggcaataagatttctaagtttatcgactggatgaaatccatgctgcctcaggcccagctgaaggtgaaatacctgagcgaactgaagaagctgaacctgctggagaagcagattgagcacctgagagcagcagactccgccacacaggaaaaaatcaagtgcgagattgacactctgcatgacctgtcctgcaaattcctgcctctgtacgcttccgaggccaaaagaatcaaggtgctgcacaataagtgcaatgtggttatcaagcagaaaaagcggtgtgagccagtggccgtggttattcacggagagcctggcaccggcaagagcatgactactaattttctcgcccggatgatcaccaatgactctgacatctactccctgcccccagacccaaagtatttcgacggatatgaccagcagagcgtggttatcatggatgacatcatgcagaatccctccggcgacgatatgaccctgttttgtcagatggtttcctccgtgaccttcatccccccaatggccgatctgcctgacaagggcaagccttttgacagcaggttcgtgctgtgcagcacaaaccacagcctgctgaccccccctactattacctctctgcccgccatgaacaggagattctttatggacctggacatcatcgtgtgtgataaatataaggacgcccagggaaagctgaatgtcagcgccgctttcaagccctgcgacgtggacaccaagatcggcaacgctcggtgttgtcctttcatctgcggcaaagccgtgatgttcaaagacagaaatacatgtcgcacctacactctggcccagatctacaaccagattctggaggaggataagaggagacgccaggtcattgacgtcatgtccgccattttccagggcacccaggtgtctcggcagaatgtcggaacccactcaacacagaatagcgtgagcaacggcagcagcctgaactactttaatatcaattactttaaggacgctgccagcagcggagccagcaaactcgagttcagccaagacccaagcaagttcaccgaccccgtgaaagacgtcctggaaaagggaatccccaccctgcagagcccaacagtcgaggcttgtgggtattctgatagaatcatccagattaccagaggagacagtaccatcacatctcaggacgtggcaaatgcagtggtgggctacggagtgtggcctcactacctgaccccccaggatgccaccgccattgacaagccctcccgccctgacacttcttccaataggttttacaccctggagtctaagatgtggacatccgattccaagggatggtggtggaagctgcctgatgctctgaaaaacatgggcatcttcggggagaacatgttctatcactttctgggaaggtccggctatacagttcacgtgcagtgcaacgctagcaagttccatcagggcactctgatcgtggtcatgattcccgagcaccagctggcatctgcctccacagggaacgtgactgccggatataacctgacccaccctggagagcagggcagggacgtcggaatcaccagagtggaagacctgctcaaacagcccagcgacgactcttggctgaacttcgacggcacactgctggggaacattacaattttccctcaccagttcatcaacctgaggtctaacaacagcgccacaatcattgttccatatgtgaacgccgtgcccatggatagcatgccccggcacaacaactggagcctggttatcatccccatttgcccactggagagcgacggacagacacctgtgcccatcacaatctccatctctcccatgtgtgccgagttctctggggcacgcgctaaatcccagtga

SEQ ID NO: 63 - DNA, 2667 bp, synthetic construct
  source 1..2667: mol_type = other DNA
atggggccaagcgacatgtatgtgcatgtgggaaacctgatctacagaaacctgcacctctttaacagcgagatgcacgattccattctgatttcctattcctctgatctcgttatctaccggaccaacaccacaggagacgactatattccaacatgcgactgcacagaggccacttactactgtaagcacaaaaacagatacttccccatcaaggtgacttcccacgactggtacgagatccaggaatctgaatactaccctaagcatatccagtacaacctgctgattggagaagggccatgcgagccaggcgattgtggcggcaagctgctgtgtaaacacggcgtgatcggcatgattaccgccgggggcgacggccatgtggcattcatcgacctgaggcatttccattgtgcagaggagcagggcattaccgattacattcacatgctgggcgaggctttcggcagcggctttgtggacagcgtgaaggagcagattaacgcaattaaccctatcaataacatttccaagaagatcatcaagtggctgctgagaatcatctccgccatggttattatcatccgcaactctagcgacccccagaccattatcgcaacactgaccctgatcggatgttccggctccccttggcgctttctgaaggagaagttttgcaagtggacccagctgaactacattcacaaggagagcgactcttggctgaaaaagttcacagaaatgtgtaatgccgccagagggctggaatggatcggaaataagatctccaagttcatcgactggatgaagtccatgctgccacaggcccagctgaaagtcaaatacctgtctgagctgaaaaaactgaacctgctggagaagcagattgaacaccttcgggccgctgactctgccactcaggagaagatcaagtgcgaaattgacaccctgcacgacctgagctgcaaattcctgcctctgtatgcctccgaggccaagaggatcaaggtgctgcacaacaaatgcaatgtcgttatcaagcagaagaagcggtgcgagcccgtggccgtcgtgatccacggggagcctggaacaggaaagtctatgactactaacttcctcgcccggatgatcacaaacgacagcgacatctatagcctgccacccgaccctaaatatttcgatggatacgaccagcagtctgtcgtgatcatggatgacatcatgcagaatcctagcggggatgatatgacactgttctgccagatggtgagcagcgtgacattcatcccaccaatggctgacctgccagacaagggcaagcctttcgactccagattcgtgctgtgctccaccaaccactccctcctgacccctcccacaattacctccctgccagccatgaataggcgcttctttatggacctggatatcatcgtctgcgacaaatacaaggatgcccaggggaaactgaacgtgtccgccgcctttaagccttgtgacgtggacacaaagattgggaatgccagatgctgcccttttatttgcggaaaggccgtcatgttcaaggaccggaatacctgcaggacatacactctggctcagatctacaaccagattctggaggaggacaagaggagaaggcaggtcatcgacgtgatgtctgccatcttccagggaacccaggtgtctcgccagaatgtgggaacccactccactcagaacagcgtgagtaacgggagcagcctgaactattttaacatcaattattttaaggacgccgcttcctccggcgcaagcaagctggagttcagccaggacccctccaagtttaccgatccagtgaaagatgtcctggagaaaggcatccccaccctgcagagccccacagtggaggcctgtgggtatagcgacagaatcatccagatcactaggggcgactccaccattactagccaggatgttgccaatgccgtggtgggatatggggtgtggccacactatctcacaccacaggacgccaccgccatcgataaaccttccaggcctgacacttcctctaatcgcttttataccctggagagcaagatgtggaccagcgacagcaagggctggtggtggaagctgccagatgctctgaagaatatgggcattttcggagagaacatgttttatcacttcctggggcggtctggatacaccgtgcacgtgcagtgcaatgcttccaagtttcaccagggcactctgatcgtggttatgattcctgagcaccagctggcaagcgcatccaccggaaacgttaccgccggatacaatctgacacatcccggcgagcagggcagggatgtgggcattacccgggtggaagacctgctgaagcagcctagcgacgatagctggctgaattttgacggcacactgctgggaaacatcactattttcccccaccagtttatcaacctgagaagcaacaactccgctacaattatcgtgccctacgtgaatgccgttcctatggactccatgccacgccacaacaattggagcctggtcatcattccaatttgccctctggagagtgatggacagacacccgtgccaatcactatctccatttccccaatgtgtgccgagttttccggcgccagagccaagtcacagtga

SEQ ID NO: 64 - DNA, 2667 bp, synthetic construct
  source 1..2667: mol_type = other DNA
atgggcacccaggtgtctcggcagaatgtcggaacccactcaacacagaatagcgtgagcaacggcagcagcctgaactactttaatatcaattactttaaggacgctgccagcagcggagccagcaaactcgagttcagccaagacccaagcaagttcaccgaccccgtgaaagacgtcctggaaaagggaatccccaccctgcagagcccaacagtcgaggcttgtgggtattctgatagaatcatccagattaccagaggagacagtaccatcacatctcaggacgtggcaaatgcagtggtgggctacggagtgtggcctcactacctgaccccccaggatgccaccgccattgacaagccctcccgccctgacacttcttccaataggttttacaccctggagtctaagatgtggacatccgattccaagggatggtggtggaagctgcctgatgctctgaaaaacatgggcatcttcggggagaacatgttctatcactttctgggaaggtccggctatacagttcacgtgcagtgcaacgctagcaagttccatcagggcactctgatcgtggtcatgattcccgagcaccagctggcatctgcctccacagggaacgtgactgccggatataacctgacccaccctggagagcagggcagggacgtcggaatcaccagagtggaagacctgctcaaacagcccagcgacgactcttggctgaacttcgacggcacactgctggggaacattacaattttccctcaccagttcatcaacctgaggtctaacaacagcgccacaatcattgttccatatgtgaacgccgtgcccatggatagcatgccccggcacaacaactggagcctggttatcatccccatttgcccactggagagcgacggacagacacctgtgcccatcacaatctccatctctcccatgtgtgccgagttctctggggcacgcgctaaatcccagggcccttccgatatgtacgttcacgtggggaacctgatctatagaaatctgcacctctttaacagcgagatgcacgactccatcctgatcagctatagtagcgaccttgtgatctatcgcactaataccaccggagacgactacattccaacctgcgactgcactgaggccacctactactgcaagcataaaaatcgctacttccccatcaaggtcaccagccatgactggtatgagatccaggagtctgaatactatcctaagcacatccagtacaacctgctgatcggcgagggcccatgtgagcctggggattgtgggggcaagctgctgtgcaagcacggcgtgatcggaatgatcaccgccggcggggacggacacgtggctttcattgatctgaggcactttcactgtgctgaggagcagggcatcactgactacatccacatgctgggcgaagcctttgggtccggattcgtggatagcgtgaaggagcagatcaatgccatcaaccctatcaacaacatcagcaaaaagatcatcaaatggctgctgaggatcatttctgccatggttatcatcatcagaaactccagcgaccctcagacaatcatcgccaccctcactctgattggctgcagcgggtccccttggagattcctgaaggagaagttctgcaagtggacccagctgaactacatccacaaagagagcgacagttggctgaaaaaattcaccgaaatgtgtaacgccgcaaggggcctggaatggatcggcaataagatttctaagtttatcgactggatgaaatccatgctgcctcaggcccagctgaaggtgaaatacctgagcgaactgaagaagctgaacctgctggagaagcagattgagcacctgagagcagcagactccgccacacaggaaaaaatcaagtgcgagattgacactctgcatgacctgtcctgcaaattcctgcctctgtacgcttccgaggccaaaagaatcaaggtgctgcacaataagtgcaatgtggttatcaagcagaaaaagcggtgtgagccagtggccgtggttattcacggagagcctggcaccggcaagagcatgactactaattttctcgcccggatgatcaccaatgactctgacatctactccctgcccccagacccaaagtatttcgacggatatgaccagcagagcgtggttatcatggatgacatcatgcagaatccctccggcgacgatatgaccctgttttgtcagatggtttcctccgtgaccttcatccccccaatggccgatctgcctgacaagggcaagccttttgacagcaggttcgtgctgtgcagcacaaaccacagcctgctgaccccccctactattacctctctgcccgccatgaacaggagattctttatggacctggacatcatcgtgtgtgataaatataaggacgcccagggaaagctgaatgtcagcgccgctttcaagccctgcgacgtggacaccaagatcggcaacgctcggtgttgtcctttcatctgcggcaaagccgtgatgttcaaagacagaaatacatgtcgcacctacactctggcccagatctacaaccagattctggaggaggataagaggagacgccaggtcattgacgtcatgtccgccattttccagtga

SEQ ID NO: 65 - DNA, 2667 bp, synthetic construct
  source 1..2667: mol_type = other DNA
atgggaacccaggtgtctcgccagaatgtgggaacccactccactcagaacagcgtgagtaacgggagcagcctgaactattttaacatcaattattttaaggacgccgcttcctccggcgcaagcaagctggagttcagccaggacccctccaagtttaccgatccagtgaaagatgtcctggagaaaggcatccccaccctgcagagccccacagtggaggcctgtgggtatagcgacagaatcatccagatcactaggggcgactccaccattactagccaggatgttgccaatgccgtggtgggatatggggtgtggccacactatctcacaccacaggacgccaccgccatcgataaaccttccaggcctgacacttcctctaatcgcttttataccctggagagcaagatgtggaccagcgacagcaagggctggtggtggaagctgccagatgctctgaagaatatgggcattttcggagagaacatgttttatcacttcctggggcggtctggatacaccgtgcacgtgcagtgcaatgcttccaagtttcaccagggcactctgatcgtggttatgattcctgagcaccagctggcaagcgcatccaccggaaacgttaccgccggatacaatctgacacatcccggcgagcagggcagggatgtgggcattacccgggtggaagacctgctgaagcagcctagcgacgatagctggctgaattttgacggcacactgctgggaaacatcactattttcccccaccagtttatcaacctgagaagcaacaactccgctacaattatcgtgccctacgtgaatgccgttcctatggactccatgccacgccacaacaattggagcctggtcatcattccaatttgccctctggagagtgatggacagacacccgtgccaatcactatctccatttccccaatgtgtgccgagttttccggcgccagagccaagtcacaggggccaagcgacatgtatgtgcatgtgggaaacctgatctacagaaacctgcacctctttaacagcgagatgcacgattccattctgatttcctattcctctgatctcgttatctaccggaccaacaccacaggagacgactatattccaacatgcgactgcacagaggccacttactactgtaagcacaaaaacagatacttccccatcaaggtgacttcccacgactggtacgagatccaggaatctgaatactaccctaagcatatccagtacaacctgctgattggagaagggccatgcgagccaggcgattgtggcggcaagctgctgtgtaaacacggcgtgatcggcatgattaccgccgggggcgacggccatgtggcattcatcgacctgaggcatttccattgtgcagaggagcagggcattaccgattacattcacatgctgggcgaggctttcggcagcggctttgtggacagcgtgaaggagcagattaacgcaattaaccctatcaataacatttccaagaagatcatcaagtggctgctgagaatcatctccgccatggttattatcatccgcaactctagcgacccccagaccattatcgcaacactgaccctgatcggatgttccggctccccttggcgctttctgaaggagaagttttgcaagtggacccagctgaactacattcacaaggagagcgactcttggctgaaaaagttcacagaaatgtgtaatgccgccagagggctggaatggatcggaaataagatctccaagttcatcgactggatgaagtccatgctgccacaggcccagctgaaagtcaaatacctgtctgagctgaaaaaactgaacctgctggagaagcagattgaacaccttcgggccgctgactctgccactcaggagaagatcaagtgcgaaattgacaccctgcacgacctgagctgcaaattcctgcctctgtatgcctccgaggccaagaggatcaaggtgctgcacaacaaatgcaatgtcgttatcaagcagaagaagcggtgcgagcccgtggccgtcgtgatccacggggagcctggaacaggaaagtctatgactactaacttcctcgcccggatgatcacaaacgacagcgacatctatagcctgccacccgaccctaaatatttcgatggatacgaccagcagtctgtcgtgatcatggatgacatcatgcagaatcctagcggggatgatatgacactgttctgccagatggtgagcagcgtgacattcatcccaccaatggctgacctgccagacaagggcaagcctttcgactccagattcgtgctgtgctccaccaaccactccctcctgacccctcccacaattacctccctgccagccatgaataggcgcttctttatggacctggatatcatcgtctgcgacaaatacaaggatgcccaggggaaactgaacgtgtccgccgcctttaagccttgtgacgtggacacaaagattgggaatgccagatgctgcccttttatttgcggaaaggccgtcatgttcaaggaccggaatacctgcaggacatacactctggctcagatctacaaccagattctggaggaggacaagaggagaaggcaggtcatcgacgtgatgtctgccatcttccagtga

SEQ ID NO: 66 - protein, 888 aa, synthetic construct
MGTQVSRQNVGTHSTQNSVSNGSSLNYFNINYFKDAASSGASKLEFSQDPSKFTDPVKDVLEKGIPTLQSPTVEACGYSDRIIQITRGDSTITSQDVANAVVGYGVWPHYLTPQDATAIDKPSRPDTSSNRFYTLESKMWTSDSKGWWWKLPDALKNMGIFGENMFYHFLGRSGYTVHVQCNASKFHQGTLIVVMIPEHQLASASTGNVTAGYNLTHPGEQGRDVGITRVEDLLKQPSDDSWLNFDGTLLGNITIFPHQFINLRSNNSATIIVPYVNAVPMDSMPRHNNWSLVIIPICPLESDGQTPVPITISISPMCAEFSGARAKSQGPSDMYVHVGNLIYRNLHLFNSEMHDSILISYSSDLVIYRTNTTGDDYIPTCDCTEATYYCKHKNRYFPIKVTSHDWYEIQESEYYPKHIQYNLLIGEGPCEPGDCGGKLLCKHGVIGMITAGGDGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKVKYLSELKKLNLLEKQIEHLRAADSATQEKIKCEIDTLHDLSCKFLPLYASEAKRIKVLHNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSLLTPPTITSLPAMNRRFFMDLDIIVCDKYKDAQGKLNVSAAFKPCDVDTKIGNARCCPFICGKAVMFKDRNTCRTYTLAQIYNQILEEDKRRRQVIDVMSAIFQ

SEQ ID NO: 67 - protein, 2151 aa, Rhinovirus A
MGAQVSRQNVGTHSTQNSVSNGSSLNYFNINYFKDAASSGASKLEFSQDPSKFTDPVKDVLEKGIPTLQSPTVEACGYSDRIIQITRGDSTITSQDVANAVVAYGVWPHYLTPQDATAIDKPSRPDTSSNRFYTLESKTWTGTSKGWWWKLPDALKDMGIFGENMFYHFLGRSGYTVHVQCNASKFHQGTLIVAMIPEHQLASATTGNARAGYKLTHPGEVGRNVDPITRDNPTKQPSDDSWLNFDGTLLGNITIYPHQLINLRSNNSATIIVPYVNAVPMDSMLRHNNWSLVIIPICPLENSDQVPNTVPITISISPMCAEFSGARAKSQGLPVMITPGSGQFLTTDDFQSPCALPWFHPTKEIFIPGQVRNLIELCQVDTLIPINSIEANIKSTNMYTVTLTRETDLAKEIFAIKVDVASHPLATTLIGEIASYYTHWTGSLRFSFLFCGTACTTLKLLIAYTPPGIRKPENRKEAMLGTHVVWDVGLQSTVSMVVPWVSASHFRNTTPDKYSLAGYITCWYQTNLVVPPNTPTTAKMLCFVSGCKDFCLRMARDTNLHLQSAPIEQNPVENYVDEILNEVLVVPNIKESKPSTSNSAPALDAAETGHTSNVQPEDMVETRYVQTSQTRDETSIESFLGRSGCIHMSKLTVDYDNYDDKNFFKWQINLQEMAQIRRKFEMFTYTRFDSEITIVPCIAAKGDDIGHVVMQYMYIPPGAPVPTKRNDYTWQSGTNASVFWQHGQTYPRFSLPFLSIASAYYMFYDGYDGDQYDSIYGSVVTNDMGTLCYRIVTDTHNHKIEITTRIYHKAKHVKVWCPRPPRAVEYTHTHVTNYKHHEREVKTAIEPRRGIKTVGPSDMYVHVGNLIYRNLHLFNSEMHDSILVSYSSDLVIYRTNTIGDDYIPTCDCTEATYYCKHKNRYYPIKVTSHDWYEIQESEYYPKHIQYNLLIGEGPCEPGDCGGKLLCKHGVIGIITAGGEGHVAFIDLRHFHCADEQGITDYIHMLGEAFGSGFVDSVKEQINAINPINSISKKIIKWLLRIISAMVIIIRNSSDPQTIIATLTLIGCSGSPWRFLKEKFCKWTQLNYIHKESDSWLKKFTEMCNAARGLEWIGNKISKFIEWMKSMLPQAQLKIKYLSELKKLNLLEKQIEHLRVADSNTQEKIKCEIDTLHDLSCKFLPLYASEAKRIKVLYNKCNVIIKQKKRCEPVAVVIHGEPGTGKSMTTNFLARMITNDSDIYSLPPDPKYFDGYDQQSVVIMDDIMQNPSGEDMTLFCQMVSSVTFIPPMADLPDKGKPFDSRFVLCSTNHGMLAPPTITSLPAMNRRFFLDLDIIVNDKYKDSQGKLNVSAAFQPCDVNAKIGNAKCCPFICGKAVMFKDRNTCTSYTLAQVYNNILDEDKRRRQVVDVMSAIFQGPITLEGPPPAAINDLLKSVRTPEVIKYCEDNNWIIPAECKIERDLNIANTIITIMANIISIAGIIYVIYKLFCTLQGPYSGEPIKKTKVPERRVVAQGPEEEFGRSLIKHNSCVVTTQNGKFTGLGIYDRVIIIPTHADPGKEVQIDGINTTVEDSYDLYNKDGVKLEITVLKLKRNEKFKDIRKYLPENEDDYPDCSLALSANQPETTIINVGDVVSYGNILLSGNQTARMLKYNYPTKSGYCGGILYKIGQVLGIHVGGNGRDGFAAMLLRSYFSETQGQIVTTKSTFDCNLPSIHTPSKTKLQPSIFHDVFPGSKEPAVLSEHDPRLEVNFKEALFSKYKGNNQCELNDHIKVAVSHYSAQLTTLDICPNPISLEDSVFGIEGLEALDLNTSAGFPYVTMGIKKRDLINKQTKDITKLKMAMDKYGVDLPMVTFLKDELRKKEKIAAGKTRVIEASSVNDTVLFRTTFGNLFSKFHLNPGIVTGSAVGCDPETFWSKIPVMLDGECIMAFDYTNYDGSIHPIWFQALKEVLSNLGFECGLVDRLCKSRHIFKNMYYEVEGGIPSGCSGTSIFNTMINNIIIRTLVLDAYKNIDLDKLKIIAYGDDVIFSYKYQLDMEAIAKEGMKYGLTITPADKSSEFKQLDYSNVTFLKRGFRQDEKHHFLIHPTFPIEEIQESIRWTKKPSQMQEHVLSLCHLMWHNGRDTYKQFEQKIRSVSAGRALYIPPYDLLLHEWY

SEQ ID NO: 68 - protein, 2151 aa, Rhinovirus A; identical to SEQ ID NO: 67

SEQ ID NO: 69 - protein, 330 aa, Rhinovirus A
MGAQVSRQNVGTHSTQNSVSNGSSLNYFNINYFKDAASSGASKLEFSQDPSKFTDPVKDVLEKGIPTLQSPTVEACGYSDRIIQITRGDSTITSQDVANAVVGYGVWPHYLTPQDATAIDKPSRPDTSSNRFYTLESQTWSGDSKGWWWKLPDALKDMGIFGENMFYHFLGRSGYTVHVQCNASKFHQGTLLVVMIPEHQLATAAGGSVSAGYNYTHPGEKGRDVGKEGREANVRQPSDDNWLNFDGTLLGNLTIFPHQFINLRSNNSATIIVPYVNAVPMDSMLRHNNWSLVIIPICRLNSMGGEPTVPITISISPMCAEFSGARAKNQ

SEQ ID NO: 70 - protein, 560 aa, Rhinovirus A
MGPSDMYVHVGNLIYRNLHLFNSEMHDSILVSYSSDLVIYRTNTIGDDYIPTCDCTEATYYCKHKNRYYPIKVTSHDWYEIQESEYYPKHIQYNLLIGEGPCEPGDCGGKLLCKHGVIGIITAGGEGHVAFIDLRHFHCADEQGITDYIHMLGEAFGSGFVDSVKEQINAINPINSISKKIIKWLLRIISAMVIIIRNSSDPQTIIATLTLIGCSGSPWRFLKEKFCKWTQLNYIHKESDSWLKKFTEMCNAARGLEWIGNKISKFIEWMKSMLPQAQLKIKYLSELKKLNLLEKQIEHLRVADSNTQEKIKCEIDTLHDLSCKFLPLYASEAKRIKVLYNKCNVIIKQKKRCEPVAVVIHGEPGTGKSMTTNFLARMITNDSDIYSLPPDPKYFDGYDQQSVVIMDDIMQNPSGEDMTLFCQMVSSVTFIPPMADLPDKGKPFDSRFVLCSTNHGMLAPPTITSLPAMNRRFFLDLDIIVNDKYKDSQGKLNVSAAFQPCDVNAKIGNAKCCPFICGKAVMFKDRNTCTSYTLAQVYNNILDEDKRRRQVVDVMSAIFQ

SEQ ID NO: 71 - DNA, 51 bp, Influenza A
  source 1..51: mol_type = genomic DNA
atgaaggccattctggtggtgctgctgtataccttcgctacagctaatgcc

SEQ ID NO: 72 - DNA, 51 bp, Influenza A
  source 1..51: mol_type = genomic DNA
atgaaggcaatcctcgtggtcctgctgtacaccttcgctacagccaacgcc

SEQ ID NO: 73 - protein, 8 aa, synthetic construct
DYKDDDDK

SEQ ID NO: 74 - protein, 5 aa, synthetic construct
GGGGS

SEQ ID NO: 75 - DNA, 24 bp, synthetic construct
  source 1..24: mol_type = other DNA
gactacaaggacgatgacgataag

SEQ ID NO: 76 - DNA, 15 bp, synthetic construct
  source 1..15: mol_type = other DNA
ggaggcggaggcagc

SEQ ID NO: 77 - protein, 560 aa, synthetic construct
MGPSDMYVHVGNLIYRNLHLFNSEMHDSILISYSSDLVIYRTNTTGDDYIPTCDCTEATYYCKHKNRYFPIKVTSHDWYEIQESEYYPKHIQYNLLIGEGPCEPGDAGGKLLCKHGVIGMITAGGDGHVAFIDLRHFHCAEEQGITDYIHMLGEAFGSGFVDSVKEQINAINPINNISKKIIKWLLRIISAMVIIIRNSSDPQTIIATLTLIGCSGSPWRFLKEKFCKWTQLNYIHKESDSWLKKFTEMCNAARGLEWIGNKISKFIDWMKSMLPQAQLKVKYLSELKKLNLLEKQIEHLRAADSATQEKIKCEIDTLHDLSCKFLPLYASEAKRIKVLHNKCNVVIKQKKRCEPVAVVIHGEPGTGKSMTTNFLARMITNDSDIYSLPPDPKYFDGYDQQSVVIMDDIMQNPSGDDMTLFCQMVSSVTFIPPMADLPDKGKPFDSRFVLCSTNHSLLTPPTITSLPAMNRRFFMDLDIIVCDKYKDAQGKLNVSAAFKPCDVDTKIGNARCCPFICGKAVMFKDRNTCRTYTLAQIYNQILEEDKRRRQVIDVMSAIFQ

SEQ ID NO: 78 - protein, 567 aa, synthetic construct
GPSDQCVHTKDAIYTCAHLTEPNSNTILLAITADLQVDSTDTPGPDFIPTCDCVQACYYAKHAQRYYPITVTPHDWYEIQESQYYPKHIQYNILIGEGPCEPGDAGGKLLCRHGVIGIITAGGDGHVAFTDLRPYACLSTHQGLVSDYVNQLGAAFGDGFSSNIKDHLTGLCTTVSDKITGKVIKWLVRVISALTIMIRNSSDTATVLATLALLGCHGSPWSFLKEKICQWLGIPRPPTRQGESWLKKFTECCNAAKGLEWVAQKIGKFIDWLKEKLIPTVQRKKETLDQCKKIGLYEEQTKGFSHSEAEAQQSLILEVAKLKRGLDDLAPLYASENKRVTIIQKELQRLSAYQKTHRHEPVCCLLRGPPGCGKSLVTSIIAHGLTNEANIYSLPPDPKHFDGYNQQTVVIMDDVGQNPDGKDLSMFCQMVSTTEFIVPMASIEDKGRAFTSQYVLASTNLDSLSPPTVTIPEAISRRFYLDADLQVTSKFKAHNGLLDVAKALQPCAKCPKPNHYKQCCPILCGQALVLRDRRTSANYPLLAVVEQLRMENNTRDKVKSNLRAIFQ

SEQ ID NO: 79 - protein, 567 aa, synthetic construct
GPSDMYVHTKEAIYKNAHLTSANEQTILIALTADLQVDAADHPGDDVIPDCDCTTGTYYCKSKDRYYPVEVVSHAWYPIEETCYYPKHIQYNILLGEGPCVPGDAGGKLLCKHGVIGIVTAGGENHVAFTDLRPYSNLAHTQGPISDYVTQLGNAFGTGFTQTLETNLRETCSGMFDAITSKTVKWVVRIISALTIVIRNSSDIPTILATMALLGCTGSPWQYLKSKLCNWLGVQKPPSKQSDSWLKKFTEWCNAAKGLEWIGYKISKFIDWLKEKLIPTVQRKKDTLLECKKLTLYEDQVRAFPQSPEAFQNELTTKLQILKKNLDDMCPLYAAENKRVTNMLRDIKTMTAYKKTHRTEPVCVLIHGGPGCGKSLATTVIARGLTDSGNVYSLPPSPKHFDGYCQQQVVMMDDLGQNPDGQDLAMFCQMVSTTDFIVPMAALEDKGKSFTSDFVLASTNLNQLSPPTVTIPEAITRRFFLDVDLKIMSGYRTHAGLLDTAKALQACPDCAKPPYYKQCCPLLCGKAVVLQNRRTSASLSLNMVVSQLREESNTRKRIHTNLNAIFQ

SEQ ID NO: 80 - protein, 562 aa, Rhinovirus A
MGPSEMFVHTTNLMYRNYHLTPEQELDSAIQVVYTADLVIHRTNDKGDDYIPDCNCTDCCYYCAHKNRYIPVKVRYYNYYTIQESEYYPKHIQYDILLGEGPSEPGDCGGKLLCKHGVIGMVTAGGDNHVAFIDLRKYRITEAEEQGITDYVKSLGDAFGVGFVEQIKEQVSNINPLNKISAKVIKWLIRVISALVIAVRSQGDLATLSATLVLLGCSDSPWRFLKQKVCQWLDLRYVHKESDGWIKKFTEMCNAARGLEWIGCKISKFIDWLKSMLPQAQNKIKFLQFTKQLQLKEKQIDGLPYATIKQQEEYLQEMEEMLDISNKLLPLYPRENKMIKDLLKQAKNMTVASKRIEPVAVMFHGDPGSGKSICTNILARMITNPSDIYSLPPDPKYFDGYHQQTVVIMDDVMQNPNGEDMSTFCQMVSSVNYVVPMADLPDKGTLFSSDYVFCSTNQHILTPPTISTLPALNRRLFLDLTIKVNPKYLESGKLNLDCALKACDQEQKIGNARCCPLICGKAVSFVNRNNNEELSLSRVYNQIVHEHNRRLNVSKHMEAIFQ

SEQ ID NO: 81 - protein, 562 aa, synthetic construct
MGPSEMFVHTTNLMYRNYHLTPEQELDSAIQVVYTADLVIHRTNDKGDDYIPDCNCTDCCYYCAHKNRYIPVKVRYYNYYTIQESEYYPKHIQYDILLGEGPSEPGDAGGKLLCKHGVIGMVTAGGDNHVAFIDLRKYRITEAEEQGITDYVKSLGDAFGVGFVEQIKEQVSNINPLNKISAKVIKWLIRVISALVIAVRSQGDLATLSATLVLLGCSDSPWRFLKQKVCQWLDLRYVHKESDGWIKKFTEMCNAARGLEWIGCKISKFIDWLKSMLPQAQNKIKFLQFTKQLQLKEKQIDGLPYATIKQQEEYLQEMEEMLDISNKLLPLYPRENKMIKDLLKQAKNMTVASKRIEPVAVMFHGDPGSGKSICTNILARMITNPSDIYSLPPDPKYFDGYHQQTVVIMDDVMQNPNGEDMSTFCQMVSSVNYVVPMADLPDKGTLFSSDYVFCSTNQHILTPPTISTLPALNRRLFLDLTIKVNPKYLESGKLNLDCALKACDQEQKIGNARCCPLICGKAVSFVNRNNNEELSLSRVYNQIVHEHNRRLNVSKHMEAIFQ

SEQ ID NO: 82 - protein, 578 aa, synthetic construct
MKAILVVLLYTFATANAGPSEMFVHTTNLMYRNYHLTPEQELDSAIQVVYTADLVIHRTNDKGDDYIPDCNCTDCCYYCAHKNRYIPVKVRYYNYYTIQESEYYPKHIQYDILLGEGPSEPGDCGGKLLCKHGVIGMVTAGGDNHVAFIDLRKYRITEAEEQGITDYVKSLGDAFGVGFVEQIKEQVSNINPLNKISAKVIKWLIRVISALVIAVRSQGDLATLSATLVLLGCSDSPWRFLKQKVCQWLDLRYVHKESDGWIKKFTEMCNAARGLEWIGCKISKFIDWLKSMLPQAQNKIKFLQFTKQLQLKEKQIDGLPYATIKQQEEYLQEMEEMLDISNKLLPLYPRENKMIKDLLKQAKNMTVASKRIEPVAVMFHGDPGSGKSICTNILARMITNPSDIYSLPPDPKYFDGYHQQTVVIMDDVMQNPNGEDMSTFCQMVSSVNYVVPMADLPDKGTLFSSDYVFCSTNQHILTPPTISTLPALNRRLFLDLTIKVNPKYLESGKLNLDCALKACDQEQKIGNARCCPLICGKAVSFVNRNNNEELSLSRVYNQIVHEHNRRLNVSKHMEAIFQ

SEQ ID NO: 83 - protein, 331 aa, Rhinovirus C
MGAQVSRQSVGSHETMIHAGTGAVVKYFNVNYYKDAASSGLTKQDFSQDPSKFTQPVADVLTNPALMSPSVEACGFSDRLKQITIGNSTITTQDAVNTIVAYGEWPSYLSDLDATSVDKPTHPETSSDRFYTLKSVEWQSSSEGWWWKLPDCLKDMGIFGQNMFHHAMGRSGYIIHTQCNATKFHSGCLLVAVVPEHQLAYIGADARVSYEHTHPGEKGHVIGSNSDRNNHQPDESAFFNCNGTLLGNLTIFPHQLINLRTNNSSTIVVPYINCTPMDSMLRHNNVSLVVIPICPLRPPNPGQQTLPITISIAPIKSEFSGARQAIKATAQ

SEQ ID NO: 84 - protein, 330 aa, Rhinovirus C
MGAQVSKQSVGAHETMVHAGSGAVVKYFNINYYKDAASSGLTKQDFSQDPSKFTQPVADLLTNPALMSPSVEACGYSDRLKQITIGSSTITTQDSVNTIVAYGEWPSYLSDLDASSVDKPTHPETSADRFYTLDSVRWGGSSKGWWWKLPDCLKNMGIFGQNMYYHAMGRSGYIIHTQCNATKFHSGCLLVAVVPEHQLAYIGGTNAQVSYKHTHPGESGHEIGLNTSRGDNRPDEDPFFNCNGTLLGNLTIFPHQLINLRTNNSSTIVVPYINCTPMDSMLRHNNVSLVIIPICPLRASSGAPTTLPITVSIAPDRSEFSGARQSATRQ

SEQ ID NO: 85 - protein, 331 aa, Rhinovirus C; identical to SEQ ID NO: 1

SEQ ID NO: 86 - protein, 330 aa, Rhinovirus C; identical to SEQ ID NO: 2

SEQ ID NO: 87 - protein, 332 aa, Rhinovirus C
MGAQVSKQSNGTHENIVSASNGAVVKYFNINYYKDSASSGLSRQDFSQDPSKFTQPLVDTLTNPALMSPTVEACGFSDRLKQITIGNSTITTQDSLNSVLAYGEWPTYLSDIDATSVDKPTHPETSADRFYTLRSVNWGATSKGWWWKFPDALSEMGVFGQNMYYHAMGRSGYIIHTQCNATKFHSGAIIVAVVPEHQLAYIGGTKANVSYGHTHPGENGHEIRGPDSGHDRGNNNPDEDPLFNCNGTLLGNITIFPHQIINLRTNNSSTIIVPYINCTPMDSMLKHNNVSLVIIPIVPLRVNGTGPTTIPITVSVAPYKSEFSGAMEAQRQ

SEQ ID NO: 88 - protein, 329 aa, Rhinovirus C
MGAQVTKQKVGSHDNTIAAQSGSVVKYFNINYYKDAASSGLSKQDFSQDPSKFTQPLADVLTNPALMSPSIEACGYSDRLKQITIGDSTITTQDTLNSVVAYGEWPEYLSDIDASSIDKPTHPETSADRFYTLDSVDWSENSTGWWWKLPDCLRDMGLFGQNMYYHAMGRTGYIVHVQCNATKFHSGCLMVAAIPEHQLAYIGGGNANVKYKHTHPGDRGHTMRPSDVRGDNNPDEDPFYLCNGTLFGNIQVYPHQMINLRTNNSATLIIPYINCLPMDSMLRHNNISLVIIPLVRLKTGATGSTTLPITITIAPDKSEFSGPMQNQKQ

(19) United States
(12) Patent Application Publication
(10) Pub. No.: US 2026/0124291 A1
(43) Pub. Date: May 7, 2026

(54) RHINOVIRUS MRNA VACCINE

(71) Applicant: Sanofi Pasteur, Lyon Cedex (FR)

(72) Inventors: Catherine BERRY, Lyon Cedex (FR); Cyril CHAVAGNAC, Lyon Cedex (FR); Nicholas CLARK, Waltham, MA (US); Yves GIRERD-CHAMBAZ, Lyon Cedex (FR); Valerie LECOUTURIER, Lyon Cedex (FR); Nathalie MANTEL, Lyon Cedex (FR); Vincent PAVOT, Lyon Cedex (FR); Khang Anh TRAN, Cambridge, MA (US)

(21) Appl. No.: 19/242,040

(22) Filed: Jun. 18, 2025

Related U.S. Application Data
(63) Continuation of application No. PCT/EP2023/087041, filed on Dec. 20, 2023.

(30) Foreign Application Priority Data
Dec. 20, 2022 (EP) 22315341.2
Aug. 22, 2023 (EP) 23306405.4

(51) Int. Cl. A61K 39/125; A61K 39/00; A61P 31/04; C07K 14/005; G01N 30/10

(52) U.S. Cl. CPC A61K 39/125; A61P 31/04; C07K 14/005; G01N 30/10; A61K 2039/53; A61K 2039/572; A61K 2039/70; C12N 2770/32722; C12N 2770/32734

(57) ABSTRACT

The present invention provides a method for identifying the amino acid sequence of a naturally occurring polyprotein from a group A or C rhinovirus that can be used as an immunogen capable of eliciting an immune response against rhinoviruses from multiple serotypes within the same group. The invention also provides immunogenic compositions that comprise at least one mRNA comprising a non-naturally occurring optimized nucleic acid encoding a polyprotein identified by this method.

### RELATED APPLICATIONS

This application is a continuation of International Application PCT/EP2023/087041 filed 20 Dec. 2025, which claims priority from European patent application no. 22315341.2, filed Dec. 20, 2022, and European patent application no. 23306405.4, filed Aug. 22, 2023; the contents of which are herein incorporated by reference in their entirety.

### SEQUENCE LISTING

The present specification makes reference to a Sequence Listing submitted electronically as a .xml file named “pat22129_sequence_listing” on 20 Dec. 2023. The .xml file was generated on 18 Dec. 2023 and is 147,456 bytes in size. The entire contents of the sequence listing are herein incorporated by reference.

### FIELD OF THE INVENTION

The present invention relates to a messenger RNA (mRNA)-based rhinovirus vaccine. The vaccine is specifically designed to elicit an immune response that is effective against multiple rhinovirus serotypes of the same group, in particular rhinovirus group A or group C. The selected immunogens encoded by the mRNA are naturally occurring rhinovirus polyproteins that comprise highly conserved and likely T-cell epitope-rich regions.

### BACKGROUND OF THE INVENTION

Rhinoviruses are small, non-enveloped, positive-stranded RNA viruses that belong to the Picornaviridae family. They are characterized into three groups, A, B, and C, for which 81, 33, and 56 serotypes, respectively, have been described to date. The large number of serotypes accounts for the huge genetic and antigenic variability observed among rhinoviruses.

The rhinovirus genome encodes for a single polyprotein comprising both structural and non-structural proteins. The polyprotein is cleaved in a protease-dependent manner to produce the precursor proteins P1, P2, and P3. These are in turn further cleaved into four structural (capsid) proteins, VP1, VP2, VP3, and VP4, and seven non-structural proteins, 2A, 2B, 2C, 3A, 3B, 3C, and 3D, respectively. VP2 and VP4 result from the cleavage of intermediate polyprotein, VP0. FIG. 1 provides a schematic illustration of the domain structure of the rhinovirus mRNA.

Human rhinoviruses (HRV) are the principal cause of the common cold, accounting for two thirds of cases annually. Transmission occurs through direct contact with respiratory secretions and is associated with upper and lower respiratory tract infections. There is currently no approved antiviral therapy for the prevention or treatment of rhinovirus infection. Human challenge studies have indicated that pre-challenge antibodies reduce viral load and disease expression (Barclay et al., Epidemiol Infect. 1989 December; 103(3): 659-669; Alper et al., Clin Infect Dis. 1998 July; 27(1): 119-128; Touabi et al., Viruses. 2021 February; 13(3): 360). Symptom severity has also been reported to inversely correlate with T helper type 1 (T_{H}1) and interferon-gamma (IFNγ) responses to experimental rhinovirus infection (Parry et al., J Allergy Clin Immunol. 2000 April; 105(4): 692-698; Gem et al., Am J Respir Crit Care Med. 2000 December; 162(6): 2226-2231; Message et al., Proc Natl Acad Sci USA. 2008 September; 105(36): 13562-13567).

In healthy individuals, an effective T_{H}1 response is induced, which may minimize viral infection, thereby typically avoiding severe illness. In contrast, individuals that suffer from respiratory conditions, such as chronic obstructive pulmonary disease (COPD) and asthma, may experience viral-related exacerbations as a consequence of HRV infection due to an increased likelihood of initiating a T helper type 2 (T_{H}2) response. The induction of a T_{H}2 response, combined with a delayed IFN response, may contribute to asthma exacerbation through mucus hypersecretions and allergic inflammation.

The absence of cross-protection from natural infection represents a clinical challenge highlighting the need for a suitable vaccine strategy. Although rhinovirus infections are typically mild in healthy individuals, repeat infections causing severe symptoms of the common cold can place a considerable economic burden on society in terms of lost working days. Various approaches to rhinovirus vaccine development exist. One approach is the induction of broadly neutralizing antibodies (Katpally et al., J Virol. 2009 July; 83(14): 7040-7048). Clinical data supports the notion that T-cells can protect against development of symptomatic respiratory disease when antibody-mediated protection against rhinovirus infection is circumvented, e.g., because a patient's immune system has not previously been exposed to a particular serotype (Parry et al., J Allergy Clin Immunol. 2000 April; 105(4): 692-698; Gem et al., Am J Respir Crit Care Med. 2000 December; 162(6): 2226-2231; Message et al., Proc Natl Acad Sci USA. 2008 September; 105(36): 13562-13567).

Prior research indicates that the structural protein VP4 comprises conserved T-cell epitopes across rhinovirus A and C subtypes, and it has been proposed to target these conserved T-cell epitopes through a peptide-based approach (Gomez-Perozanz et al., Cells. 2021 September; 10(9); 2284). Unlike more established vaccine technologies, the effectiveness of peptide-based approaches requires further pre-clinical and clinical validation.

Accordingly, a need remains for a vaccine against rhinovirus infection, particularly for patients suffering from a respiratory condition, such as COPD and/or asthma, that is effective in preventing or reducing the complications associated with a rhinovirus infection.

### SUMMARY OF THE INVENTION

The invention is based on the identification of conserved regions within the complete rhinovirus polyprotein of rhinovirus group A and rhinovirus group C strains that may be suitable as immunogens to elicit an immune response against multiple rhinovirus serotypes in the same group. Such regions can be used for the identification of T-cell epitope rich regions to confirm that they provide a wide coverage of MHC-I and MHC-II alleles to elicit a T-cell response in the majority of the human population.

The inventors used a computational approach to identify conserved regions in the amino acid sequence of complete rhinovirus A and C polyproteins. This approach involved categorizing rhinovirus sequences from group A and group C into phylogenetic clusters and identifying sequences that could cover at least two of these clusters to ensure that a selected immunogen would be capable of eliciting an immune response against multiple rhinovirus serotypes. Without wishing to be bound by any particular theory, the inventors believe that a naturally occurring rhinovirus polyprotein having an average identity (and optionally a median identity) of at least 80% to the amino acid sequences of rhinoviruses from at least two phylogenetic clusters is suitable to provide broad protection.

The inventors analyzed publicly available amino acid sequences encoding all or at least a portion of the complete rhinovirus polyprotein. The inventors concentrated on sequences of at least 800 amino acids because this is the approximate length of the full VP capsid region of the polyprotein. The VP proteins are exposed on the virus surface and therefore should represent most if not all differences among various rhinovirus serotypes.

In particular, the invention relates to a method for identifying a rhinovirus polyprotein for use as an immunogen that is capable of eliciting an immune response against rhinoviruses from multiple serotypes within a group, said method comprising the following steps: (a) retrieving a plurality of amino acid sequences from a database comprising amino acid sequences from naturally occurring rhinovirus isolates; (b) removing from the plurality of amino acid sequences retrieved in step (a) amino acid sequences shorter than 800 amino acids; (c) assigning the amino acid sequences remaining after step (b) into different phylogenetic clusters; (d) aligning the amino acid sequences to determine a consensus amino acid sequence for a complete rhinovirus polyprotein for one or more phylogenetic clusters identified in step (c); (e) aligning the consensus amino acid sequence obtained in step (c) with complete polyproteins of naturally occurring rhinovirus isolates; and (f) selecting a rhinovirus polyprotein as an immunogen that has an average identity (and optionally a median identity) of at least 80% (e.g., at least 85%, at least 90% or at least 95%) to the corresponding amino acid sequences of rhinoviruses from at least two phylogenetic clusters identified in step (c).

In some embodiments, the rhinovirus polyprotein selected in step (f) is a VP0 polyprotein. In some embodiments, the rhinovirus polyprotein selected in step (f) is a P2 polyprotein. In some embodiments, the group is rhinovirus group A. In some embodiments, the group is rhinovirus group C.

In some embodiments, the one or more phylogenetic clusters comprise at least 5 different serotypes. In some embodiments, the one or more phylogenetic clusters comprises at least 10, 15, 20 or 25 different serotypes.

In some embodiments, the plurality of amino acid sequences retrieved in step (a) is greater than 400 (e.g., 500, 600, 700 or 800).

In some embodiments, determining the consensus sequence in step (d) comprises selecting the most frequent amino acid at each position. In some embodiments, determining the consensus sequence in step (d) comprises creating a gap when the sum of amino acids for a given position is lower than 50% of the number of retrieved sequences. In some embodiments, determining the consensus sequence in step (d) comprises selecting the most frequent amino acid when the sum of amino acids for a given position is equal to or greater than 50% of the number of retrieved sequences.

Without wishing to be bound by any particular theory, the inventors believe that an immunogen selected in step (f) of the method can effectively induce a desired immune response, including, e.g., an effective T-cell response, when the immunogen is administered to a subject (e.g., a subject in need of immunization) in form of at least one messenger RNA (mRNA) encoding the immunogen. Accordingly, in some embodiments, the method further comprises a step of generating an optimized nucleic acid sequence encoding the rhinovirus polyprotein selected in step (f).

Furthermore, the invention relates to immunogenic compositions (e.g., vaccines) that include a non-naturally occurring mRNA encoding the immunogen identified by the method of the invention and an optional carrier (e.g., a lipid nanoparticle encapsulating the mRNA) or an adjuvant. The inventors are of the view that an immunogenic composition comprising a non-naturally occurring mRNA encoding a naturally occurring rhinovirus protein or polyprotein is more effective in eliciting an immune response against multiple rhinovirus serotypes of the same group than other types of vaccines. In some embodiments, to achieve efficient transcription and expression of the mRNA, the nucleic acid encoding the immunogen is optimized (e.g., expression-optimized) to produce a non-naturally occurring optimized nucleic acid sequence. Sometimes, the rhinovirus-derived polyprotein itself may interfere with efficient expression. Accordingly, to ensure efficient expression of the mRNA-encoded immunogen, one or more amino acid substitutions may be introduced into the naturally occurring amino acid sequence of the polyprotein that has been selected as the immunogen. In an embodiment, a single amino acid substitution is introduced into the naturally occurring amino acid sequence of the polyprotein that has been selected as the immunogen.

Accordingly, the invention also provides an immunogenic composition (e.g., a vaccine) comprising at least one messenger RNA (mRNA) comprising a non-naturally occurring optimized nucleic acid sequence encoding a polyprotein from a group A or C rhinovirus, wherein said polyprotein has an amino acid sequence that (a) has an average identity (and optionally a median identity) of at least 80% (e.g., at least 85%, at least 90% or at least 95%) to the amino acid sequences of corresponding polyproteins from at least two phylogenetic clusters of rhinoviruses of the same group; and (b) is naturally occurring aside from one or more optional amino acid substitutions (e.g., 1, 2, 3, 4, 5, 6, 7, 8, 9, 10 or more amino acid substitutions). In an embodiment, said polyprotein has an amino acid sequence that (a) has an average identity (and optionally a median identity) of at least 80% (e.g., at least 85%, at least 90% or at least 95%) to the amino acid sequences of corresponding polyproteins from at least two phylogenetic clusters of rhinoviruses of the same group; and (b) is naturally occurring aside from an optional single amino acid substitution. The amino acid sequence of the polyprotein can be obtained, or is obtainable, by the methods for identifying a rhinovirus polyprotein for use as an immunogen described here.

In some embodiments, the amino acid sequence of the polyprotein has an average identity (and optionally a median identity) of at least 80% (e.g., at least 85%, at least 90% or at least 95%) to the amino acid sequences of corresponding polyproteins from at least three, e.g., four, phylogenetic clusters of rhinoviruses of the same group.

In some embodiments, the polyprotein is a VP0 polyprotein comprising proteins VP2 and VP4. In some embodiments, the VP0 polyprotein is from a group C rhinovirus. In some embodiments, the group C rhinovirus is of serotype 11, 17 or 34. In some embodiments, the polyprotein has an amino acid sequence that is at least 80%, 85%, 90%, 95%, or 99% identical, or identical, to the amino acid sequence of the rhinovirus C serotype 11 VP0 polyprotein set forth in SEQ ID NO: 1. In some embodiments, the polyprotein has an amino acid sequence that is at least 80%, 85%, 90%, 95%, or 99% identical, or identical, to the amino acid sequence of the rhinovirus C serotype 17 VP0 polyprotein set forth in SEQ ID NO: 2. In some embodiments, the polyprotein has an amino acid sequence that is at least 80%, 85%, 90%, 95%, or 99% identical, or identical, to the amino acid sequence of the rhinovirus C serotype 34 VP0 polyprotein set forth in SEQ ID NO: 3.

In some embodiments, the VP0 polyprotein is from a group A rhinovirus. In some embodiments, the group A rhinovirus is of serotype 21 or 90. In some embodiments, the polyprotein has an amino acid sequence that is at least 80%, 85%, 90%, 95%, or 99% identical, or identical, to the amino acid sequence of the rhinovirus A serotype 21 VP0 polyprotein set forth in SEQ ID NO: 4. In some embodiments, the polyprotein has an amino acid sequence that is at least 80%, 85%, 90%, 95%, or 99% identical, or identical, to the amino acid sequence of the rhinovirus A serotype 90 VP0 polyprotein set forth in SEQ ID NO: 5.

In some embodiments, the polyprotein is a P2 polyprotein comprising proteins 2A, 2B and 2C. In some embodiments, the single amino acid substitution is in the 2A protein and reduces or abolishes the proteolytic activity of the P2 polyprotein. In some embodiments, the single amino acid substitution is C>A substitution or C>S substitution in the catalytic triad of the active site of the 2A protein.

In some embodiments, the P2 polyprotein is from a group A rhinovirus. In some embodiments, the group A rhinovirus is of serotype 21 or 57. In some embodiments, the polyprotein has an amino acid sequence that is at least 80%, 85%, 90%, 95%, or 99% identical, or aside from the optional single amino acid substitution identical, to the amino acid sequence of the rhinovirus A serotype 21 P2 polyprotein set forth in SEQ ID NO: 6. In some embodiments, the polyprotein has an amino acid sequence that is at least 80%, 85%, 90%, 95%, or 99% identical, or aside from the optional single amino acid substitution identical, to the amino acid sequence of the rhinovirus A serotype 57 P2 polyprotein set forth in SEQ ID NO: 7.

In some embodiments, the P2 polyprotein is from a group C rhinovirus. In some embodiments, the group C rhinovirus is of serotype 11 or 17. In some embodiments, the polyprotein has an amino acid sequence that is at least 80%, 85%, 90%, 95%, or 99% identical, or aside from the optional single amino acid substitution identical, to the amino acid sequence of the rhinovirus C serotype 11 P2 polyprotein set forth in SEQ ID NO: 8. In some embodiments, the polyprotein has an amino acid sequence that is at least 80%, 85%, 90%, 95%, or 99% identical, or aside from the optional single amino acid substitution identical, to the amino acid sequence of the rhinovirus C serotype 17 P2 polyprotein set forth in SEQ ID NO: 9.

In some embodiments, the immunogenic composition (e.g., vaccine) further comprises a second non-naturally occurring optimized nucleic acid sequence encoding a further polyprotein from a group A or C rhinovirus, wherein said further polyprotein is different from said polyprotein. In some embodiments, the first and second nucleic acid sequences are part of the same mRNA. In some embodiments, the mRNA encodes a fusion protein comprising the polyprotein and the further polyprotein. In some embodiments, the first and second nucleic acid sequences are encoded by separate non-naturally occurring mRNAs.

Combining multiple different polyproteins can be advantageous to extend the protection provided by the immunogenic composition, e.g., to elicit an immune response against multiple rhinovirus that are phylogenetically more distant to each other (e.g., multiple serotypes within the same group) or against multiple rhinoviruses from different groups (e.g., group A and group C). Accordingly, the invention also provides an immunogenic composition comprising at least one messenger RNA (mRNA) comprising (i) a first non-naturally occurring optimized nucleic acid sequence encoding a first polyprotein from a group A or C rhinovirus; and (ii) a second non-naturally occurring optimized nucleic acid sequence encoding a second polyprotein from a group A or C rhinovirus, wherein said second polyprotein is different from said first polyprotein and each of said first and second polyproteins has an amino acid sequence that (a) has an average identity (and optionally a median identity) of at least 80% (e.g., at least 85%, at least 90% or at least 95%) to the amino acid sequences of corresponding polyproteins from at least two phylogenetic clusters of rhinoviruses of the same group; and (b) is naturally occurring aside from one or more optional amino acid substitutions (e.g., 1, 2, 3, 4, 5, 6, 7, 8, 9, 10 or more amino acid substitutions). In an embodiment, said polyprotein has an amino acid sequence that (a) has an average identity (and optionally a median identity) of at least 80% (e.g., at least 85%, at least 90% or at least 95%) to the amino acid sequences of corresponding polyproteins from at least two phylogenetic clusters of rhinoviruses of the same group; and (b) is naturally occurring aside from an optional single amino acid substitution. In some embodiments, the amino acid sequence of each of said first and second polyproteins has an average identity (and optionally a median identity) of at least 80% (e.g., at least 85%, at least 90% or at least 95%) to the amino acid sequences of corresponding polyproteins from at least three, e.g., four, phylogenetic clusters of rhinoviruses of the same group. The amino acid sequences of the first and second polyproteins can be obtained, or are obtainable, by the methods for identifying a rhinovirus polyprotein for use as an immunogen described here.

In some embodiments, the first polyprotein is a VP0 polyprotein comprising proteins VP2 and VP4. In some embodiments, VP0 polyprotein is from a group A rhinovirus. In some embodiments, the group A rhinovirus is of serotype 21 or 90. In some embodiments, the first polyprotein has an amino acid sequence that is at least 80%, 85%, 90%, 95%, or 99% identical, or identical, to the amino acid sequence of the rhinovirus A serotype 21 VP0 polyprotein set forth in SEQ ID NO: 4. In some embodiments, the first polyprotein has an amino acid sequence that is at least 80%, 85%, 90%, 95%, or 99% identical, or identical, to the amino acid sequence of the rhinovirus A serotype 90 VP0 polyprotein set forth in SEQ ID NO: 5.

In some embodiments, the second polyprotein is a P2 polyprotein comprising proteins 2A, 2B and 2C. In some embodiments, the single amino acid substitution is in the 2A protein and reduces or abolishes the proteolytic activity of the P2 polyprotein. In some embodiments, the single amino acid substitution is C>A substitution or C>S substitution in the catalytic triad of the active site of the 2A protein.

In some embodiments, the P2 polyprotein is from a group A rhinovirus. In some embodiments, the group A rhinovirus is of serotype 21 or 57. In some embodiments, the second polyprotein has an amino acid sequence that is at least 80%, 85%, 90%, 95%, or 99% identical, or aside from the optional single amino acid substitution identical, to the amino acid sequence of the rhinovirus A serotype 21 P2 polyprotein set forth in SEQ ID NO: 6. In some embodiments, the second polyprotein has an amino acid sequence that is at least 80%, 85%, 90%, 95%, or 99% identical, or aside from the optional single amino acid substitution identical, to the amino acid sequence of the rhinovirus A serotype 57 P2 polyprotein set forth in SEQ ID NO: 7.

The inventors demonstrate herein that T-cell epitope-rich regions in the VP0 polyprotein and the P2 polyprotein of a group A rhinovirus of serogroup 21 cover the vast majority (>97%) of MHC-I and MHC-II alleles present in the human population. Accordingly, in some embodiments, an immunogenic composition of the invention is capable of eliciting a T-cell response in at least 95% of the human population. In some embodiments, the immunogenic composition is capable of eliciting a T-cell response in at least 96%, at least 97%, at least 98%, or at least 99% of the human population. In some embodiments, the VP0 polyprotein and the P2 polyprotein comprise T-cell epitope-rich regions that cover at least 95% of the MHC class-I alleles in Table 4 and/or 95% of the MHC-II alleles in Table 5. In some embodiments, the T-cell epitope-rich regions cover at least 96%, at least 97%, at least 98%, or at least 99% of the MHC class-I alleles in Table 4 and/or at least 96%, at least 97%, at least 98%, or at least 99% of the MHC class-II alleles in Table 5.

The VP proteins from rhinoviruses of different groups can be quite distinct. For example, rhinoviruses of group A predominantly bind the intercellular adhesion molecule 1 (ICAM-1) receptor. A minority utilizes the low-density lipoprotein receptor (LDLR) for binding. In contrast, a specific variant of cadherin-related family member 3 (CDHR3) is the primary receptor for group C rhinoviruses. An immunogenic composition capable of inducing an immune response against multiple serogroups therefore may include a VP0 polyprotein from a group A rhinovirus and a VP0 polyprotein from a group C rhinovirus.

Accordingly, in some embodiments, the second polyprotein is a VP0 polyprotein from a group C rhinovirus. In some embodiments, the group C rhinovirus is of serotype 11, 17 or 34. In some embodiments, the second polyprotein has an amino acid sequence that is at least 80%, 85%, 90%, 95%, or 99% identical, or identical, to the amino acid sequence of the rhinovirus C serotype 11 VP0 polyprotein set forth in SEQ ID NO: 1. In some embodiments, the second polyprotein has an amino acid sequence that is at least 80%, 85%, 90%, 95%, or 99% identical, or identical, to the amino acid sequence of the rhinovirus C serotype 17 VP0 polyprotein set forth in SEQ ID NO: 2. In some embodiments, the second polyprotein has an amino acid sequence that is at least 80%, 85%, 90%, 95%, or 99% identical, or identical, to the amino acid sequence of the rhinovirus C serotype 34 VP0 polyprotein set forth in SEQ ID NO: 3.

Based on the inventors' analysis, inducing an immune response against multiple phylogenetically distant serogroups of a group of rhinoviruses may be improved by including one or more non-naturally occurring mRNA encoding multiple different VP0 polyproteins and/or P2 polyproteins from the same group.

Accordingly, in some embodiments, the first polyprotein is a VP0 polyprotein and the second polyprotein is a VP0 polyprotein, wherein the two phylogenetic clusters referred to in option (a) are different for the first and second polyproteins. In some embodiments, the first and second polyproteins are from rhinovirus C. In some embodiments, the first polyprotein has an amino acid sequence that is at least 80%, 85%, 90%, 95%, or 99% identical, or identical, to the amino acid sequence of the rhinovirus C serotype 11 VP0 polyprotein set forth in SEQ ID NO: 1 and the second polyprotein has an amino acid sequence that is at least 80%, 85%, 90%, 95%, or 99% identical, or identical, to the amino acid sequence of the rhinovirus C serotype 17 VP0 polyprotein set forth in SEQ ID NO: 2.

In some embodiments, the first polyprotein is a P2 polyprotein and the second polyprotein is a P2 polyprotein, wherein the two phylogenetic clusters referred to in option (a) are different for the first and second polyproteins. In some embodiments, the first and second polyproteins are from rhinovirus C. In some embodiments, the first polyprotein has an amino acid sequence that is at least 80%, 85%, 90%, 95%, or 99% identical, or aside from the optional single amino acid substitution identical, to the amino acid sequence of the rhinovirus C serotype 11 P2 polyprotein set forth in SEQ ID NO: 8 and the second polyprotein has an amino acid sequence that is at least 80%, 85%, 90%, 95%, or 99% identical, or aside from the optional single amino acid substitution identical, to the amino acid sequence of the rhinovirus C serotype 17 P2 polyprotein set forth in SEQ ID NO: 9.

In some embodiments, the first and second nucleic acid sequences are part of the same mRNA. In some embodiments, the mRNA encodes a fusion protein comprising the first polyprotein and the second polyprotein.

In some embodiments, the first and second nucleic acid sequences are encoded by separate non-naturally occurring mRNAs.

In some embodiments, the immunogenic composition further comprises a third non-naturally occurring optimized nucleic acid sequence encoding a third polyprotein from a group A or C rhinovirus, wherein said third polyprotein is different from said first and second polyproteins.

Accordingly, the invention also provides an immunogenic composition comprising at least one messenger RNA (mRNA) (e.g., one, two or three mRNAs) comprising (i) a first non-naturally occurring optimized nucleic acid sequence encoding a first polyprotein from a group A or C rhinovirus; (ii) a second non-naturally occurring optimized nucleic acid sequence encoding a second polyprotein from a group A or C rhinovirus; and (iii) a third non-naturally occurring optimized nucleic acid sequence encoding a third polyprotein from a group A or C rhinovirus; wherein said first, second and third polyproteins are different from each other and each of said first, second and third polyproteins has an amino acid sequence that (a) has an average identity (and optionally a median identity) of at least 80%, 85%, 90%, 95%, or 99% to the amino acid sequences of corresponding polyproteins from at least two phylogenetic clusters of rhinoviruses of the same group; and (b) is naturally occurring aside from one or more optional amino acid substitutions (e.g., 1, 2, 3, 4, 5, 6, 7, 8, 9, 10 or more amino acid substitutions). In an embodiment, said polyprotein has an amino acid sequence that (a) has an average identity (and optionally a median identity) of at least 80% (e.g., at least 85%, at least 90% or at least 95%) to the amino acid sequences of corresponding polyproteins from at least two phylogenetic clusters of rhinoviruses of the same group; and (b) is naturally occurring aside from an optional single amino acid substitution. The amino acid sequences of the first, second and third polyproteins can be obtained, or are obtainable, by the methods for identifying a rhinovirus polyprotein for use as an immunogen described herein.

For example, in some embodiments, it may be desirable to combine polyproteins from a group A rhinovirus (e.g., a VP0 polyprotein and a P2 polyprotein) with a rhinovirus C polyprotein (e.g., a VP0 polyprotein), or a group A rhinovirus polyprotein (e.g., a VP0 polyprotein) with rhinovirus C polyproteins (e.g., two VP0 polyproteins), to elicit an immune response against as many group A and group C rhinoviruses as possible, while minimizing the number of mRNAs comprised in the immunogenic composition.

Accordingly, in some embodiments, the first polyprotein is a VP0 polyprotein comprising proteins VP2 and VP4. In some embodiments, VP0 polyprotein is from a group A rhinovirus. In some embodiments, group A rhinovirus of serotype 21 or 90. In some embodiments, the first polyprotein has an amino acid sequence that is at least 80%, 85%, 90%, 95%, or 99% identical, or identical, to the amino acid sequence of the rhinovirus A serotype 21 VP0 polyprotein set forth in SEQ ID NO: 4. In some embodiments, the first polyprotein has an amino acid sequence that is at least 80%, 85%, 90%, 95%, or 99% identical, or identical, to the amino acid sequence of the rhinovirus A serotype 90 VP0 polyprotein set forth in SEQ ID NO: 5.

In some embodiments, the second polyprotein is a P2 polyprotein comprising proteins 2A, 2B and 2C. In some embodiments, the single amino acid substitution is in the 2A protein and reduces or abolishes the proteolytic activity of the P2 polyprotein. In some embodiments, the single amino acid substitution is C>A substitution or C>S in the catalytic triad of the active site of the 2A protein.

In some embodiments, the P2 polyprotein is from a group A rhinovirus. In some embodiments, the group A rhinovirus is of serotype 21 or 57. In some embodiments, the second polyprotein has an amino acid sequence that is at least 80%, 85%, 90%, 95%, or 99% identical, or aside from the optional single amino acid substitution identical, to the amino acid sequence of the rhinovirus A serotype 21 P2 polyprotein set forth in SEQ ID NO: 6. In some embodiments, the second polyprotein has an amino acid sequence that is at least 80%, 85%, 90%, 95%, or 99% identical, or aside from the optional single amino acid substitution identical, to the amino acid sequence of the rhinovirus A serotype 57 P2 polyprotein set forth in SEQ ID NO: 7.

In some embodiments, the third polyprotein is a VP0 polyprotein from a group C rhinovirus. In some embodiments, the group C rhinovirus is of serotype 11, 17 or 34. In some embodiments, the third polyprotein has an amino acid sequence that is at least 80%, 85%, 90%, 95%, or 99% identical, or identical, to the amino acid sequence of the rhinovirus C serotype 11 VP0 polyprotein set forth in SEQ ID NO: 1. In some embodiments, the third polyprotein has an amino acid sequence that is at least 80%, 85%, 90%, 95%, or 99% identical, or identical, to the amino acid sequence of the rhinovirus C serotype 17 VP0 polyprotein set forth in SEQ ID NO: 2. In some embodiments, the third polyprotein has an amino acid sequence that is at least 80%, 85%, 90%, 95%, or 99% identical, or identical, to the amino acid sequence of the rhinovirus C serotype 34 VP0 polyprotein set forth in SEQ ID NO: 3.

In some embodiments, the at least one mRNA encodes a fusion protein comprising the first polyprotein (e.g., a rhinovirus A VP0 polyprotein) and the second polyprotein (e.g., a rhinovirus A P2 polyprotein) and optionally the third polyprotein, (e.g., a rhinovirus C VP0 polyprotein).

In some embodiments, the first, second and third nucleic acid sequences are encoded by separate non-naturally occurring mRNAs.

In some embodiments, the immunogenic composition is capable of eliciting a T-cell response in at least 95% of the human population. In some embodiments, the immunogenic composition is capable of eliciting a T-cell response in at least 96%, at least 97%, at least 98%, or at least 99% of the human population. In some embodiments, the VP0 polyprotein and the P2 polyprotein in the immunogenic composition comprise T-cell epitope-rich regions that cover at least 95% of the MHC class-I alleles in Table 4 and/or 95% of the MHC-II alleles in Table 5. In some embodiments, the T-cell epitope-rich regions cover at least 96%, at least 97%, at least 98%, or at least 99% of the MHC class-I alleles in Table 4 and/or at least 96%, at least 97%, at least 98%, or at least 99% of the MHC class-II alleles in Table 5.

In some embodiments, the first polyprotein is a VP0 polyprotein from a group A rhinovirus. In some embodiments, the group A rhinovirus is of serotype 21 or 90. In some embodiments, the first polyprotein has an amino acid sequence that is at least 80% identical, or identical, to the amino acid sequence of the rhinovirus A serotype 21 VP0 polyprotein set forth in SEQ ID NO: 4. In some embodiments, the first polyprotein has an amino acid sequence that is at least 80% identical, or identical, to the amino acid sequence of the rhinovirus A serotype 90 VP0 polyprotein set forth in SEQ ID NO: 5

In some embodiments, the second polyprotein is a first VP0 polyprotein from a group C rhinovirus. In some embodiments, the third polyprotein is a second VP0 polyprotein from a group C rhinovirus that is different from the second polyprotein. In some embodiments, the second polyprotein has an amino acid sequence that is at least 80% identical, or identical, to the amino acid sequence of the rhinovirus C serotype 11 VP0 polyprotein set forth in SEQ ID NO: 1. In some embodiments, the second polyprotein has an amino acid sequence that is at least 80% identical, or identical, to the amino acid sequence of the rhinovirus C serotype 17 VP0 polyprotein set forth in SEQ ID NO: 2.

In some embodiments, the at least one mRNA encodes a fusion protein comprising the first polyprotein (e.g., a rhinovirus A VP0 polyprotein), the second polyprotein (e.g., a first rhinovirus C VP0 polyprotein), and the third polyprotein (e.g., a second rhinovirus C VP0 polyprotein). In some embodiments, the first, second and third nucleic acid sequences are encoded by separate non-naturally occurring mRNAs.

In some embodiments, the immunogenic composition is capable of eliciting a T-cell response in at least 95% of the human population. In some embodiments, the immunogenic composition is capable of eliciting a T-cell response in at least 96%, at least 97%, at least 98%, or at least 99% of the human population.

The first, second and (optional) third nucleic acid sequences are typically optimized to (a) improve the yield of full-length mRNAs during in vitro synthesis, and/or (b) to maximize expression of the encoded polypeptide after delivery of the mRNA to a target cell in vivo.

In some embodiments, the mRNA(s) comprise(s) a 5′ untranslated region. In one embodiment, the 5′ untranslated region comprises the nucleotide sequence of SEQ ID NO: 10. In some embodiments, the mRNA(s) comprise(s) a 3′ untranslated region. In one embodiment, the 3′ untranslated region comprises the nucleotide sequence of SEQ ID Nos: 11, 12 or 13. In some embodiments, the mRNA(s) comprise(s) a 5′ cap. In some embodiments, the mRNA comprises a 5′ cap and a 3′ tail.

In some embodiments, the mRNA(s) comprise(s) a polyadenylation (polyA) sequence. In specific embodiments, the mRNA(s) comprise(s) a polyA sequence comprising at least 90 nucleotides. In specific embodiments, the mRNA(s) comprise(s) a polyA sequence comprising about 200 nucleotides. In some embodiments, the mRNA(s) comprise(s) N-1-methylpseudouridine in place of uridine.

In some embodiments, the immunogenic composition further comprises a plurality of lipid nanoparticles (LNPs) encapsulating the mRNA(s). In some embodiments, the lipid component of the LNPs comprises or consists of a cationic lipid, a non-cationic lipid, a PEG-modified lipid, and optionally a sterol-based lipid. In some embodiments, the cationic lipid is selected from cKK-E12, cKK-E10, HGT5000, HGT5001, ICE, HGT4001, HGT4002, HGT4003, TLT-01D-DMA, TLT-04D-DMA, TLT-08D-DMA, TLT-10D-DMA, OF-Deg-Lin, OF-02, GL-TES-SA-DMP-E18-2, GL-TES-SA-DME-E18-2, SY-3-E14-DMAPr, TL1-10D-DMA, HEP-E3-E10, HEP-E4-E10, RL3-DMA-07D, RL2-DMP-07D, cHse-E-3-E10, cHse-E-3-E12, cDD-TE-4-E12, SI-4-E14-DMAPr, TL-1-12D-DMA, SY-010, SY-011, 4-hydroxybutyl)azanediyl)bis(hexane-6,1-diyl)bis(2-hexyldecanoate (ALC-0315) and heptadecan-9-yl 8-((2-hydroxyethyl)(6-oxo-6-(undecyloxy)hexyl)amino)octanoate (SM-102). In some embodiments, the non-cationic lipid selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DEPE 1,2-dierucoyl-sn-glycero-3-phosphoethanolamine, DOPC (1,2-dioleyl-sn-glycero-3-phosphotidylcholine), DPPE (1,2-dipalmitoyl-sn-glycero-3-phosphoethanolamine), DMPE (1,2-dimyristoyl-sn-glycero-3-phosphoethanolamine), and DOPG (1,2-dioleoyl-sn-glycero-3-phospho-(1′-rac-glycerol)). In some embodiments, the PEG-modified lipid is selected from DMG-PEG-2K and 2-[(polyethylene glycol)-2000]-N,N-ditetradecylacetamide (ALC-0159). In some embodiments, the sterol-based lipid is cholesterol.

In specific embodiments, the cationic lipid is selected from cKK E10 and ALC-0315. In specific embodiments, the PEGylated lipid is selected from DMG-PEG2K or ALC-0159. In specific embodiments, the non-cationic lipid is selected from DOPE or DSPC.

In some embodiments, the invention also relates to a vaccine comprising an immunogenic composition disclosed herein and a pharmaceutically acceptable carrier or excipient.

The invention also relates to a method for eliciting an immune response in a subject, the method comprising administering an effective amount of an immunogenic composition or vaccine composition of the invention to the subject.

The invention also relates to a method of reducing or preventing one or more symptoms associated with a rhinovirus infection in a subject, the method comprising administering an effective amount of an immunogenic composition or vaccine composition of the invention to the subject.

The invention also relates to a method of reducing the severity of or preventing a rhinovirus infection in a subject, the method comprising administering an effective amount of an immunogenic composition or vaccine composition of the invention to the subject.

In some embodiments, administering the immunogenic composition or vaccine composition boosts a pre-existing rhinovirus T-cell response or re-orients the pre-existing rhinovirus T-cell response towards a T_{H}1 response.

In some embodiments, the subject suffers from asthma or chronic obstructive pulmonary disease (COPD). In some embodiments, administration of the immunogenic composition or vaccine composition reduces or prevents exacerbating symptoms associated with rhinovirus infection in a subject with asthma or COPD.

In some embodiments, administering the immunogenic composition or vaccine composition induces intracellular antibodies against the one or more non-structural polypeptides encoded by the one or more mRNAs.

In some embodiments, the subject is 40 years of age or older. In some embodiments, the subject is 65 years of age or older.

In some embodiments, the immunogenic composition or vaccine composition is administered intramuscularly.

In some embodiments, the immunogenic composition or vaccine composition is administered once to the subject.

In some embodiments, the immunogenic composition or vaccine composition is administered more than once to the subject. In some embodiments, the immunogenic composition or vaccine composition is administered at least twice. In some embodiments, the second or any subsequent administration occurs about one year or more after the first administration.

In some embodiments, the immunogenic composition or vaccine composition is administered once a year. In some embodiments, the immunogenic composition is administered twice five years apart.

In some embodiments, administration of the immunogenic composition or vaccine composition provides immunity against a rhinovirus infection caused by a group A strain, a group B strain, and/or group C strain. In some embodiments, immunity is provided against multiple serotypes of the same group. In some embodiments, immunity is provided against multiple serotypes of different groups.

### BRIEF DESCRIPTION OF THE DRAWINGS

Embodiments of the invention will be described, by way of example, with reference to the following drawings, in which:

FIG. 1 is a schematic illustration of the domain structure of a rhinovirus RNA encoding a polyprotein, comprising structural (capsid) proteins P1 and non-structural proteins P2 and P3. The P1 polyprotein is further processed into VP0, VP3 and VP1, and VP0 is further cleaved into VP4 and VP2. The P2 polyprotein is further processed into P2A, P2B and P2C. The P3 polyprotein is further processed into P3A, P3B, P3C and P3D. The positive-sense RNA encoding the polyprotein is flanked by a 5′-UTR and a poly(A) (AAAAn) tail, as indicated.

FIG. 2 is a schematic illustration of sequence conservation across a rhinovirus polyprotein. Peaks identify the percentage sequence conservation across the rhinovirus A polyprotein. Thin-lined boxes identify regions of more than 50 contiguous amino acids with at least 80% sequence conservation and identify conserved regions by the residue numbers within the consensus sequence of the rhinovirus A polyprotein. For each residue, the percentage of sequence conservation was calculated using a sliding 15 amino acid sequence window, centered on the position designated in the figure. The thick boxes designate the P2 non-structural protein and the VP0 structural protein, which were identified as comprising higher sequence conservation, comprising regions with at least 95% sequence conservation, as indicated by the dashed line. Sequence conservation was assessed using a 15 amino acid sliding window.

FIGS. 3A-3F illustrate the phylogenetic clustering of rhinovirus A serotypes based on the amino acid sequences of complete rhinovirus A polyproteins (FIGS. 3A and 3B), VP0 polyproteins (FIGS. 3C and 3D), or P2 polyproteins (FIGS. 3E and 3F), respectively. Sequences shorter than 800 amino acids in length or sequences comprising X stretches longer than 10 were excluded from the analysis. Independent of which polyprotein was selected for the analysis, multiple phylogenetic clusters were identified and are shown as outlined areas shaded in grey. Each cluster is assigned a number. The phylogenetic analyses based on the amino acid sequences of complete rhinovirus A polyproteins and P2 polyproteins resulted in nearly identical clusters 1-4, as indicated. For the VP0 polyproteins, only three clusters were identified. Cluster 1 was very similar to cluster 1 of the complete polyproteins and P2 polyproteins. The other two clusters predominantly included serotypes of clusters 2 and 3 or clusters 3 and 4 of the complete polyproteins and P2 polyproteins respectively, as indicated. The in silico consensus sequence identified for each polyprotein is shown at the center (marked by the white arrowhead) from which the phylogenetic clusters branch off. In FIG. 3A, the branches of the phylogenetic tree including the naturally occurring polyproteins that were identified as the best and second-best matches for the consensus sequence polyprotein (GenBank IDs FJ445121.1 and JN562727.1, respectively) are signified by black arrowheads. In FIG. 3C, the branches of the phylogenetic tree including the naturally occurring VP0 polyproteins that were identified as the best and second-best match for the consensus sequence VP0 polyprotein (GenBank IDs FJ445121.1 and FJ445167.1, respectively) are signified by black arrowheads. In FIG. 3E, the branches of the phylogenetic tree including the naturally occurring P2 polyproteins that were identified as the best and second-best matches for the consensus sequence P2 polyprotein (GenBank IDs FJ445121.1 and KY369874.1, respectively) are signified by black arrowheads. Branches of the trees more distant to the consensus sequences are indicated by areas with a dashed outline. FIGS. 3B, 3D and 3F show a schematic representation of the phylogenetic trees of FIGS. 3A, 3C and 3E, respectively. The number of serotypes is shown for the outlined areas shaded in grey, representing the phylogenetic clusters, and the areas with a dashed outline. FIGS. 3B, 3D and 3F also include boxes listing all serotypes within each cluster/area, as indicated. The best and second-best matches for the consensus sequence polyproteins are signified by black arrowheads.

FIG. 4, panel A, is a schematic illustration of the structural and non-structural polypeptides encoded by rhinovirus A polyproteins. FIG. 4, panel B, illustrates the locations of published T-cell epitopes.

FIG. 5 is an illustration of the location of human MHC class-I and MHC class-II epitopes along the VP0 polyprotein of rhinovirus A serotype 21 (GenBank ID FJ445121.1), corresponding to SEQ ID NO: 4. The amino acid sequence of the VP4 polypeptide is shown in black and the amino acid sequence of the VP2 polypeptide is shown in grey, with the location of the MHC class-I and MHC class-II epitopes indicated schematically below the amino acid sequence.

FIG. 6, panels A and B illustrate predicted human MHC class-I and MHC class-II T-cell epitopes across the length of the complete rhinovirus A polyprotein. FIG. 6, panel A, is a schematic illustration of the structural and non-structural polypeptides encoded by the rhinovirus A polyprotein. FIG. 6, panel B, illustrates predicted MHC class-I epitopes with a percentile rank <1%, indicating the likely locations of such epitopes within the polyprotein. FIG. 6, panel C, illustrates predicted MHC class-II epitopes with a percentile rank <3%, indicating the likely locations of such epitopes within the polyprotein.

FIG. 7, panels A-D map predicted T-cell epitopes on the P2 polyprotein of rhinovirus A serotype 21 (GenBank ID FJ445121.1). FIG. 7, panel A, provides a heat map of sequence conservation based on a previously generated consensus sequence of the rhinovirus A polyprotein. The darker regions illustrate higher sequence conservation relative to the lighter regions. FIG. 7, panel B, indicates the location of six published MHC class-I epitopes. FIG. 7, panels C and D show the percentile rank for predicted MHC class-I and class-II epitopes, respectively, indicating the likely locations of such epitopes within the polyprotein. Regions with a high degree of sequence conservation around residues 80-120, residues 250-280 and residues 380-450 are indicated by thin-lined black boxes.

FIG. 8, panels A-D illustrate predicted T-cell epitopes on the VP0 polyprotein of rhinovirus A serotype 21 (GenBank ID FJ445121.1). FIG. 8, panel A, provides a heat map of sequence conservation based on a previously generated consensus sequence of the rhinovirus A polyprotein. The darker regions illustrate higher sequence conservation relative to the lighter regions. FIG. 8, panel B, indicates the locations of three published MHC class-II epitopes and six published MHC class-I epitopes. FIG. 8, panels C and D show the percentile rank for predicted MHC class-I and class-II epitopes, respectively, indicating the likely locations of such epitopes within the polyprotein. Regions with a high degree of sequence conservation around residues 1-100, residues 150-200 and residues 229-299 are indicated by thin-lined black boxes.

FIG. 9 illustrates the expression of FLAG-tagged rhinovirus polyproteins in HeLa cells after transfection of mRNAs encoding them. Mock-transfected cells were included as a control. Each mRNA-encoded protein was FLAG-tagged. Cell lysates were probed using an anti-FLAG tag antibody (MAB8529) and visualized by Western blot. The first lane is the molecular weight ladder. The molecular weight of each band is indicated on the left-hand side of the figure. The identity of the protein encoded by the mRNA used for transfection is indicated at the bottom of the figure: a VP0 polyprotein in lanes 2 and 3, a P2 polyprotein in lanes 4 and 5, a P2-VP0 fusion protein in lanes 6 and 7, a VP0 polyprotein with a signal secretion sequence in lanes 8 and 9, a P2 polyprotein with a signal secretion sequence in lanes 10 and 11, and a P2-VP0 fusion protein with a secretion signal sequence in lanes 12 and 13. Detection of the mRNA-encoded protein at the expected molecular weight is indicated by white dashed-line boxes. Non-specific bands can be observed at ˜125 kDa and 50 kDa. Lysates of cells transfected with mRNAs encoding the P2 polyprotein, or a fusion protein comprising the P2 polyprotein, showed much lower levels of expression.

FIGS. 10A-10F illustrate the phylogenetic clustering of rhinovirus C serotypes based on the amino acid sequences of complete rhinovirus C polyproteins (FIGS. 10A and 10B), VP0 polyproteins (FIGS. 10C and 10D), or P2 polyproteins (FIGS. 10E and 10F), respectively. Sequences shorter than 800 amino acids in length or sequences comprising X stretches longer than 10 were excluded from the analysis. Independent of whether the complete polyprotein or the VP0 polyprotein was selected for the analysis, four phylogenetic clusters were identified, which are labelled as 1a, 1b, 2a, and 2b and comprise 13, 16, 5, and 9 serotypes, respectively. They are indicated by the outlined areas shaded in grey. The in silico consensus sequence identified for each polyprotein is shown at the center (marked by the white arrowhead) from which the phylogenetic clusters branch off. In FIG. 10A, the branches of the phylogenetic tree including the naturally occurring polyproteins that were identified as the best matches for the polyproteins of clusters 1a and 1b (GenBank ID: MZ153245.1) and 2a and 2b (GenBank ID: MZ268692.1), respectively, are signified by black arrowheads. In FIG. 10C, the branch of the phylogenetic tree including the naturally occurring VP0 polyprotein that was identified as the best match for the polyproteins of all four clusters (GenBank ID: MZ322913.1) is signified by a black arrowhead. Additionally, the branches of the phylogenetic tree including the naturally occurring VP0 polyproteins that were identified as the best matches for the polyproteins of clusters 1a and 1b (GenBank ID: MZ153277.1) and 2a and 2b (GenBank ID: MZ268689.1), respectively, are signified by arrowheads. In FIG. 10E, the branches of the phylogenetic tree including the naturally occurring P2 polyproteins that were identified as the best matches for the polyproteins of clusters 1a and 1b (GenBank ID: MZ153245.1) and 2a and 2b (GenBank ID: OK254863.1), respectively, are signified by black arrowheads. FIGS. 10B, 10D and 10F show a schematic representation of the phylogenetic trees of FIGS. 10A, 10C and 10E, respectively. The number of serotypes is shown for the outlined areas shaded in grey, representing the phylogenetic clusters, and the areas with a dashed outline. FIGS. 10B, 10D and 10F also include boxes listing all serotypes within each cluster/area, as indicated. The best matches for the consensus sequence polyprotein from each cluster are signified by black arrowheads.

FIGS. 11A and 11B illustrate that an immunogenic composition of the invention comprising an mRNA encoding a naturally occurring rhinovirus VP0 polyprotein, the precursor of VP4 and VP2 capsid proteins, is effective in eliciting an effective T-cell response in vivo against corresponding polyproteins from other phylogenetic clusters of rhinoviruses of the same group. C57BL/6 mice immunized twice 3 weeks apart with (i) a lipid nanoparticle (LNP) encapsulating an mRNA encoding the VP0 polyprotein of a rhinovirus A serotype A21, (ii) a recombinant VP0 polyprotein from rhinovirus A serotype A16 formulated with the T_{H}1 adjuvant SPA09, or (iii) an empty LNP, as indicated by dark grey, light grey and white boxes, respectively, at the bottom of the graph next to the mouse icon. Two weeks after immunizations, spleens were harvested and splenocytes were stimulated in vitro with an overlapping peptide library covering the full length VP0 polyprotein of the rhinovirus A serotype 21, or corresponding peptide libraries from VP0 polyproteins of rhinovirus A serotype 1b and 8, respectively, as indicated. FIG. 11A illustrates the percentages of specific polyfunctional IFN-γ, IL-2 and TNF-α-positive CD4+ T-cells after peptide stimulation (specific frequency among parental CD4 T cell population, after medium background subtraction). The percentages of polyfunctional CD4+ T-cells after stimulation with either of the serotype 21 and 1b VP0 peptides were significantly higher than after stimulation with the serotype 8 VP0 peptides (p<0.001; labelled as **). FIG. 11B illustrates the percentages of specific polyfunctional IFN-γ, IL-2 and TNF-α-positive CD8+ T-cells after peptide stimulation (specific frequency among CD8 parental CD8 T population, after medium background subtraction). No statistically significant differences were observed between the three peptide treatment groups (indicated by “n.s.”). In FIGS. 11A and 11B, the percentages of polyfunctional CD4+ or CD8+ T-cells for each animal per experimental group (n=6) are represented by filled circles (serotype 21 VP0 peptide), squares (serotype 1b VP0 peptide) and triangles (serotype 8 VP0 peptide), respectively. Brackets indicate the experimental groups compared for statistical analysis.

FIGS. 12A, 12B and 12C illustrate that immunization with immunogenic compositions of the invention comprising an mRNA encoding a naturally occurring rhinovirus polyprotein induces specific cross-reactive polyfunctional CD4+ (T_{H}1) and CD8+ T-cells. The T-cell response after immunization was assessed by intracellular cytokine staining (ICS). FIG. 12A and FIG. 12B show the induction of specific polyfunctional IFN-γ, IL-2 and TNF-α-positive CD3+CD4+ and CD3+CD8+ cells, respectively. FIG. 12C illustrates a T_{H}1-type response as indicated by the absence of IL-5-positive CD3+CD4+ cells. Mice were immunized twice 3 weeks apart (n=6 mice per group) with lipid nanoparticles (LNPs) encapsulating mRNAs encoding a VP0 polyprotein (VP0), a VP0 polyprotein with an HA secretion signal (HA-SS VP0), a P2 polyprotein (P2), or a P2 polyprotein with an HA secretion signal (HA-SS P2). The mRNA-encoded polyproteins were from rhinovirus A serotype 21. Immunizations with empty LNPs served as a negative control. Immunizations with adjuvanted recombinant VP0 polyprotein of rhinovirus A serotype 16 (adj. rec. protein VP0 RV-16) served as a positive control. The percentage of specific polyfunctional IFN-γ, IL-2 and TNF-α-positive CD3+CD4+ and CD3+CD8+ cells and the percentage of IL-5-positive CD3+CD4+ cells was assessed after in vitro stimulation of splenocytes with overlapping peptide libraries representative of the VP0 or P2 polyproteins of rhinovirus A serotype A21 (VP0 A21 or P2 A21), rhinovirus A serotype 1b (VP0 A1b or P2 A1b) and rhinovirus A serotype A8 (VP0 A8 or P2 A8), respectively, as indicated in the figure. Data are plotted as individual values of specific percentage among parental population, after medium background subtraction (VP0 A21—filled circles; VP0 A1b—filled squares; VP0 A8—filled triangles) and bars represent the mean+95% confidence interval (CI). Statistical significance is represented by * (p<0.1), ** (p<0.01) and *** (p<0.001). Brackets indicate the experimental groups compared for statistical analysis. The data in FIGS. 12A, 12B and 12C indicate that immunogenic compositions of the invention elicit a T_{H}1-oriented immune response effective against multiple group A rhinoviruses representing different serotypes and phylogenetic clusters.

FIGS. 13A and 13B illustrate that immunization with an immunogenic composition of the invention comprising an mRNA encoding a P2 polyprotein with a single amino acid substitution in the 2A protein which abolishes its proteolytic activity is more effective in inducing a robust cross-reactive polyfunctional CD4+ (T_{H}1) T-cell response. FIG. 13A and FIG. 13B show the induction of specific polyfunctional IFN-γ, IL-2 and TNF-α-positive CD3+CD4+ and CD3+CD8+ cells, respectively. Mice were immunized twice 3 weeks apart (n=6 mice per group) with lipid nanoparticles (LNPs) encapsulating 2 μg mRNA encoding a wild-type rhinovirus A serotype 21 P2 polyprotein (Wild-type P2) or a corresponding P2 polyprotein with a single amino acid substitution in the 2A protein which abolished its proteolytic activity (Mutated P2). Immunizations with empty LNPs served as a negative control. The percentage of specific polyfunctional IFN-γ, IL-2 and TNF-α-positive CD3+CD4+ cells (FIG. 13A) and CD3+CD8+ cells (FIG. 13B) was assessed after in vitro stimulation of splenocytes with overlapping peptide libraries derived from the P2 polyproteins of rhinovirus A serotype 21 (P2 A21), rhinovirus A serotype 1b (P2 1b) and rhinovirus A serotype 8 (P2 8), respectively, as indicated in the figure. Data are plotted as individual values of percentage of specific polyfunctional CD4 or CD8 T cells among parental population, after medium background subtraction (P2 A21—filled circles; P2 1b—filled triangles; P2 8—filled diamonds) and bars represent the mean+95% confidence interval (CI). No statistical difference was observed between the wild type P2 and mutated P2 in CD4+ or CD8+ T cells.

FIG. 14 illustrates the number of immunogen-specific IFN-γ-secreting cells after immunization with immunogenic compositions of the invention, as assessed by ELISPOT assay. Mice were immunized twice 3 weeks apart (n=6 mice per group) with lipid nanoparticles (LNPs) encapsulating mRNAs encoding a VP0 polyprotein (VP0), a VP0 polyprotein with an HA secretion signal (HA-SS VP0), a P2-VP0 fusion protein (Fusion P2-VP0), a P2-VP0 fusion protein with an HA secretion signal (Fusion P2-VP0 HA-SS), a P2 polyprotein (P2), and a P2 polyprotein with an HA secretion signal (HA-SS P2). The mRNA-encoded polyproteins were from rhinovirus A serotype 21. Immunizations with empty LNPs served as a negative control. Immunizations with adjuvanted recombinant VP0 polyprotein of rhinovirus A serotype 16 (adj. rec. protein VP0 RV-16) served as a positive control. The figure shows the number of spot-forming cells per 10⁶ splenocytes on a log₁₀ scale after in vitro stimulation of splenocytes with overlapping peptide libraries representative of the VP0 or P2 polyproteins of rhinovirus A serotype 21 (VP0 A21 or P2 A21). Data are plotted as individual values (filled circles) and bars represent the mean+95% confidence interval (CI). Statistical significance is represented by * (p<0.1), ** (p<0.01) and *** (p<0.001). Brackets indicate the experimental groups compared for statistical analysis.

FIGS. 15A and 15B illustrate the dose-dependent induction of anti-VP2 IgG antibodies and anti-VP4 IgG antibodies, respectively, after immunization with immunogenic compositions of the invention, as assessed by ELISA assay. As indicated in the figure, two immunizations 3 weeks apart of mice (n=6 per group) was performed at an mRNA dose of either 0.2 μg or 2 μg. The mRNAs were encapsulated in lipid nanoparticles (LNPs) and encoded a VP0 polyprotein (VP0) and a VP0 polyprotein with an HA secretion signal (HA-SS VP0), respectively, as indicated in the figure. The mRNA-encoded VP0 polyprotein was from rhinovirus A serotype 21. Immunization with empty LNPs served as a negative control. Immunization with adjuvanted recombinant VP0 polyprotein of rhinovirus A serotype 16 (adj. rec. protein VP0 RV-16) served as a positive control. The assay threshold (limit of detection) is indicated by a dashed line. At the 2 μg-dose, immunization with HA-SS-VP0-encoding mRNA induced antibody titers that were equivalent to those induced by the adjuvanted protein-based vaccine that served as a positive control. Data are plotted as individual values (filled circles) and bars represent mean+95% confidence interval (CI). Antibody titers were calculated as the reciprocal dilution given an optical density (OD) of 1. Statistical significance is represented by * (p<0.1), ** (p<0.01) and *** (p<0.001). Brackets indicate the experimental groups compared for statistical analysis.

FIGS. 16A and 16B illustrate that immunization with immunogenic compositions of the invention indues IgG antibodies that can bind multiple group A rhinoviruses representing different serotypes and phylogenetic clusters after. IgG antibodies binding whole virus was assessed by ELISA assay. FIG. 16A and FIG. 16B show anti-virion IgG titers against group A rhinovirus serotypes 21 and 1b, respectively. Mice were immunized twice 3 weeks apart (n=6 per group) with an mRNA dose of either 0.2 μg or 2 μg. The mRNAs were encapsulated in lipid nanoparticles (LNPs) and encoded a VP0 polyprotein (VP0) and a VP0 polyprotein with an HA secretion signal (HA-SS VP0), respectively, as indicated in the figure. The mRNA-encoded VP0 polyprotein was from rhinovirus A serotype 21. Immunization with empty LNPs served as a negative control. Immunization with adjuvanted recombinant VP0 polyprotein of rhinovirus A serotype 16 (adj. rec. protein VP0 RV-16) served as a positive control. The assay threshold (limit of detection) is indicated by a dashed line. At the 2 μg-dose, immunization with the HA-SS-VP0-encoding mRNA induced antibody titers against both serotypes 21 and 1b virions that were equivalent to the titers induced by the adjuvanted protein-based vaccine. Data are plotted as individual values (filled circles) and bars represent mean+95% confidence interval (CI). Antibody titers were calculated as the reciprocal dilution given an optical density (OD) of 1. Statistical significance is represented by * (p<0.1), ** (p<0.01) and *** (p<0.001). Brackets indicate the experimental groups compared for statistical analysis.

FIGS. 17A-17F illustrate the CD4 T_{H}1 responses in human Peripheral Blood Mononuclear Cells (PBMCs) after in vitro stimulation with overlapping peptide libraries representative of VP0 polyproteins of various rhinovirus A serotypes. PBMCs isolated from healthy human volunteers were stimulated with overlapping peptide libraries representative of the VP0 polyproteins of rhinovirus A serotypes A21 (VP0 A21), 1b (VP0 A1b), and 8 (VP0 A8), respectively, as indicated in the figure. Cell culture medium was used as a negative control. FIGS. 17A-17F illustrate the percentage of human CD4+ T cells secreting IFN-γ, IL-2, TNF-α, MIP-11, IL-4, and IL-17A, respectively, after peptide stimulation, as determined by intracellular cytokine staining (ICS). Data are plotted as individual values (empty circles for negative control group; filled circles for experimental group), and lines indicate matched samples. Statistical significance is represented by * (p<0.1), ** (p<0.01) and *** (p<0.001). Brackets indicate the experimental groups compared for statistical analysis. Cumulatively, the data indicate that rhinovirus natural infection elicits significant CD4+ T_{H}1 responses against VP0 from group A in healthy humans.

FIGS. 18A-18D illustrate the low or rare CD8+ T cell responses in human PBMCs after in vitro stimulation with overlapping peptide libraries representative of VP0 polyproteins of various rhinovirus A serotypes. PBMCs isolated from healthy human volunteers were stimulated with overlapping peptide libraries representative of the VP0 polyprotein of rhinovirus A serotype 21 (VP0 A21), rhinovirus A serotype 1b (VP0 Alb) and rhinovirus A serotype 8 (VP0 A8), respectively, as indicated in the figure. Cell culture medium was used as a negative control. FIGS. 18A-18D illustrate the percentage of human CD8+ T-cells secreting IFN-γ, IL-2, TNF-α, and MIP-1, respectively, after peptide stimulation, as determined by intracellular cytokine staining (ICS). Data are plotted as individual values (empty circles for negative control group; filled circles for experimental group), and lines indicate matched samples. Statistical significance is represented by * (p<0.1), ** (p<0.01) and *** (p<0.001). Brackets indicate the experimental groups compared for statistical analysis. The data in FIGS. 18A-18D indicate low or rare VP0-specific CD8+ T cell responses against group A in healthy humans.

FIGS. 19A-19F illustrate CD4+ T_{H}1 responses in human PBMCs after in vitro stimulation with overlapping peptide libraries representative of various VP0 polyproteins of rhinovirus C serotypes. PBMCs isolated from healthy human volunteers were stimulated with overlapping peptide libraries representative of the VP0 polyprotein of rhinovirus C serotype 34 (VP0 C34), 11 (VP0 C11), 07 (VP0 C07), 01 (VP0 C01), 17 (VP0 C17), 41 (VP0 C41), and 53 (VP0 C53), respectively, as indicated in the figure. Cell culture medium was used as a negative control. FIGS. 19A-19F illustrate the percentage of CD4+ T-cells secreting IFN-γ, IL-2, TNF-α, MIP-1β, IL-4, and IL-17A, respectively, after peptide stimulation, as determined by intracellular cytokine staining (ICS). Data are plotted as individual values (empty circles for negative control group; filled circles for experimental group), and lines indicate matched samples. Statistical significance is represented by * (p<0.1), ** (p<0.01) and *** (p<0.001). Brackets indicate the experimental groups compared for statistical analysis. Cumulatively, the data in FIGS. 19A-19F indicate that rhinovirus natural infection elicits significant CD4+ T_{H}1 responses against VP0 from group C in healthy humans.

FIGS. 20A-20D illustrate the absence of CD8+ T cell response in human PBMCs after in vitro stimulation with overlapping peptide libraries representative of VP0 polyproteins of various rhinovirus C serotypes. PBMCs isolated from healthy human volunteers were stimulated with overlapping peptide libraries representative of the VP0 polyprotein of rhinovirus C serotype 34 (VP0 C34), 11 (VP0 C11), 07 (VP0 C07), 01 (VP0 C01), 17 (VP0 C17), 41 (VP0 C41), and 53 (VP0 C53), respectively, as indicated in the figure. Cell culture medium was used as a negative control. FIGS. 20A-20D illustrate the percentage of CD8+ T-cells secreting IFN-γ, IL-2, TNF-α, and MIP-1p, respectively, after peptide stimulation, as determined by intracellular cytokine staining (ICS). Data are plotted as individual values (empty circles for negative control group; filled circles for experimental group), and lines indicate matched samples. Statistical significance is represented by * (p<0.1), ** (p<0.01) and *** (p<0.001). Brackets indicate the experimental groups compared for statistical analysis. The data in FIGS. 20A-20D indicate no statistically significant VP0-specific CD8+ T cell responses against group C in healthy humans.

FIGS. 21A and 21B illustrate that immunization with immunogenic compositions of the invention comprising an mRNA encoding a naturally occurring rhinovirus C VP0 polyprotein is effective in eliciting antigen-specific CD4+ and CD8+ T-cells. C57BL/6 mice were immunized twice 3 weeks apart with (i) a lipid nanoparticle (LNP) encapsulating an mRNA encoding the VP0 polyprotein, or (ii) an empty LNP (negative control), as indicated by dark grey and light grey boxes, respectively, at the bottom of the graphs next to the mouse icon. Spleens were harvested from immunized mice 2 weeks after the last injection. The induction of antigen-specific CD4+ and CD8+ T-cells was assessed by intracellular cytokine staining (ICS) after in vitro stimulation of splenocytes with overlapping peptide libraries covering full-length VP0 polyproteins of rhinovirus C serotype 34 (VP0 C34), 11 (VP0 C11), 07 (VP0 C07), 01 (VP0 C01), 17 (VP0 C17), 41 (VP0 C41), and 53 (VP0 C53), respectively, as indicated in the figures. CD4+ and CD8+ cells were identified as antigen-specific T-cells if they stained positively for any combination of IFN-γ, IL-2, and/or TNF-α, i.e., including single, double, or triple positive staining. Data are shown as antigen specific CD4+ or CD8+ T cell among parental population, after medium background subtraction. Data are plotted as individual values (filled circles), and bars represent the mean+95% confidence interval (CI). Negative control data are only shown for T-cells from mock-treated mice stimulated with a pool of peptides derived from the VP0 polyprotein of rhinovirus C serotype 34. Statistical significance relative to the corresponding negative control (i.e., T-cells from mock-treated mice stimulated with relevant overlapping peptide libraries) is represented by * (p<0.1), ** (p<0.01), *** (p<0.001) and **** (p<0.0001).

FIGS. 22A and 22B illustrate that immunization with an immunogenic composition of the invention comprising an mRNA encoding a naturally occurring rhinovirus C VP0 polyprotein induces cross-reactive polyfunctional CD4+ and CD8+ T cells. Spleens were harvested from C57BL/6 mice immunized with (i) a lipid nanoparticle (LNP) encapsulating an mRNA encoding the VP0 polyprotein, or (ii) an empty LNP (negative control), as indicated by dark grey and light grey boxes, respectively, at the bottom of the graphs next to the mouse icon. Using intracellular cytokine staining (ICS), the percentage of IFN-γ, IL-2 and TNF-α-positive CD3+CD4+ and CD3+CD8+ cells was determined after in vitro stimulation with an overlapping peptide libraries covering full-length VP0 polyproteins of rhinovirus C serotype 34 (VP0 C34), 11 (VP0 C11), 07 (VP0 C07), 01 (VP0 C01), 17 (VP0 C17), 41 (VP0 C41), and 53 (VP0 C53), respectively, as indicated in the figures. Data are shown as antigen specific CD4+ or CD8+ T cell among parental population, after medium background subtraction. Data are plotted as individual values (filled circles), and bars represent the mean+95% confidence interval (CI). Negative control data are only shown for T-cells from mock-treated mice stimulated with a pool of peptides derived from the VP0 polyprotein of rhinovirus C serotype 34. Statistical significance relative to the corresponding negative control (i.e., T-cells from mock-treated mice stimulated with relevant overlapping peptide library) is represented by * (p<0.1), ** (p<0.01), *** (p<0.001) and **** (p<0.0001).

FIG. 23 illustrates that immunization with an immunogenic composition of the invention comprising an mRNA encoding a naturally occurring rhinovirus C VP0 polyprotein induces a T_{H}1-directed CD4+ T cell response. C57BL/6 mice were immunized twice 3 weeks apart with (i) a lipid nanoparticle (LNP) encapsulating an mRNA encoding the VP0 polyprotein, or (ii) an empty LNP (negative control), as indicated by dark grey and light grey boxes, respectively, at the bottom of the graph next to the mouse icon. Using intracellular cytokine staining (ICS), only a small percentage of CD4+ cells producing the T_{H}2 cytokine IL-5 was detected after in vitro stimulation with an overlapping peptide libraries covering full-length VP0 polyproteins of rhinovirus C serotype 34 (VP0 C34), 11 (VP0 C11), 07 (VP0 C07), 01 (VP0 C01), 17 (VP0 C17), 41 (VP0 C41), and 53 (VP0 C53), respectively, as indicated in the figure. Data are shown as IL-5 secreting specific CD4+ T-cells among parental population, after medium background subtraction. Data are plotted as individual values (filled circles), and bars represent the mean+95% confidence interval (CI). Negative control data are only shown for T-cells from mock-treated mice stimulated with a pool of peptides derived from the VP0 polyprotein of rhinovirus C serotype 34. Statistical significance relative to the corresponding negative control (i.e., T-cells from mock-treated mice stimulated with peptides derived from corresponding peptide pools) is represented by * (p<0.1), ** (p<0.01), *** (p<0.001) and **** (p<0.0001).

### DEFINITIONS

In order for the present invention to be more readily understood, certain terms are first defined below. Additional definitions for the following terms and other terms are set forth throughout the specification.

As used in this specification and the appended claims, the singular forms “a”, “an” and “the” include plural referents unless the context clearly dictates otherwise.

Unless specifically stated or obvious from context, as used herein, the term “or” is understood to be inclusive and covers both “or” and “and”.

As used herein, the term “mRNA” refers to a polyribonucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, in vitro transcribed, or chemically synthesized. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. A typical mRNA comprises a 5′ cap, a 5′ untranslated region (5′ UTR), a protein-coding region, a 3′ untranslated region (3′ UTR), and a 3′ tail. In some embodiments, the tail structure is a poly(C) tail. More typically, the tail structure is a poly(A) tail.

The term “naturally occurring” as used herein to describe a rhinovirus polypeptide, protein or polyprotein refers to the amino acid sequence of the polypeptide, protein or polyproteins being present in a rhinovirus isolate. In some embodiments, the rhinovirus polypeptides, proteins or polyproteins disclosed herein include a single amino acid substitution relative to the naturally occurring amino acid sequence of a rhinovirus isolate in order to render the protein more suitable for use in the immunogenic compositions of the invention. For example, the inventors found that expression of a P2 polyprotein of the invention can result in eIF4g cleavage, which can lead to global translational repression. Accordingly, a P2 polyprotein encoded by an mRNA of the invention typically is modified to produce a 2A protein with reduced or no proteolytic activity, e.g., by substituting the cysteine of the 2A protein, which acts as the nucleophile in the catalytic triad, with a serine or alanine. Without wishing to be bound by any particular theory, the inventors believe that the immunogen or antigen function of such a modified polypeptide, protein or polyprotein is essentially identical to the natural occurring version.

As used herein the term “sequence-optimized” is used to describe a nucleotide sequence that is modified relative to a naturally occurring or wild-type nucleic acid. Such modifications may include, e.g., codon optimization as well as the use of 5′ UTRs and 3′ UTRs which are not normally associated with the naturally occurring or wild-type nucleic acid. As used herein, the terms “codon optimization” and “codon-optimized” refer to modifications of the codon composition of a naturally occurring or wild-type nucleic acid encoding a peptide, polypeptide or protein that do not alter its amino acid sequence, thereby improving protein expression of said nucleic acid. In the context of the present invention, “codon optimization” may also refer to the process by which one or more optimized nucleotide sequences are arrived at by using filters to remove less than optimal nucleotide sequences from a list of nucleotide sequences, such as filtering by guanine-cytosine (GC) content, codon adaptation index (CAI), presence of destabilizing nucleic acid sequences or motifs, and/or presence of pause sites and/or terminator signals.

As used herein, the term “template DNA” (or “DNA template”) relates to a DNA molecule comprising a nucleic acid sequence encoding an mRNA transcript to be synthesized by in vitro transcription. The template DNA is used as template for in vitro transcription in order to produce the mRNA transcript encoded by the template DNA. The template DNA comprises all elements necessary for in vitro transcription, particularly a promoter element for binding of a DNA-dependent RNA polymerase, such as, e.g., T3, T7 and SP6 RNA polymerases, which is operably linked to the DNA sequence encoding a desired mRNA transcript. Furthermore, the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

As used herein, the term “localization sequence” relates to an amino acid sequence which facilitates transcytosis of a linked polypeptide across an epithelium. The localization sequence may be linked to the carboxy-terminus (C-terminus) of a polypeptide. The localization sequence may be linked to the polypeptide by a linker sequence. The localization sequence may also be exogenous to the polypeptide. For example, the localization sequence may facilitate transport of the linked polypeptide across a layer of airway epithelial cells such that polypeptides including one of these sequences may be more effectively delivered to the airway or lung lumen.

As used herein, the term “adjuvant” refers to a substance or combination of substances that may be used to enhance an immune response to an antigen.

As used herein, the term “immunogen” or “immunogenic” refers to a compound, composition, or substance which is capable, under appropriate conditions, of stimulating an immune response, such as the production of antibodies, a T-cell response, or both, in a subject, including compositions that are injected or absorbed into an animal. As used herein, the term “immunogenic composition” refers to a composition that generates an immune response that may or may not be a protective immune response. As used herein, “immunize” means to induce in a subject a protective immune response against an infectious disease (e.g., a rhinovirus infection).

As used herein, the term “vaccine composition” or “vaccine” refers to a composition that generates a protective immune response in a subject. As used herein, a “protective immune response” refers to an immune response that protects a subject from infection (prevents infection or prevents the development of disease associated with infection) or reduces the symptoms of infection (for instance an infection by a rhinovirus). Vaccines may elicit both prophylactic (preventative) and therapeutic responses.

As used herein, the term “subject” refers to a mammal, such as a human or other animal. Typically, a subject is a human. The subject can be male or female and can be any suitable age, including infant, juvenile, adolescent, adult, and geriatric subjects.

All technical and scientific terms used herein have the same meaning as commonly understood by one of ordinary skill in the art to which this application belongs and as commonly used in the art to which this application belongs. The publications and other reference materials referenced herein to describe the background of the invention and to provide additional detail regarding its practice are hereby incorporated by reference.

### GENERAL REMARKS

Various embodiments of the methods, processes, steps, functions, and/or operations described herein may be implemented by or using a computer, a processor, or the like. For example, a computer or the like may be programmed to perform some or all of the methods, processes, steps, functions, and/or operations described herein. The computer may be controlled by a software program(s) comprised of program instructions in source code, object code, executable code, or the like. The software may be embodied on a computer readable medium, which may include RAM (random access memory), ROM (read-only memory), EPROM (erasable, programmable ROM), EEPROM (electrically erasable, programmable ROM), and magnetic disks, optical disks, solid-state disks, or the like.

### DETAILED DESCRIPTION OF THE INVENTION

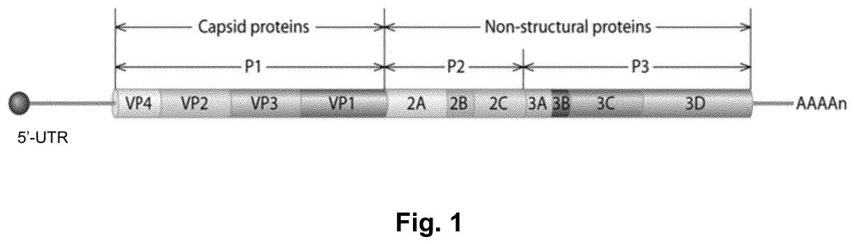

The rhinovirus mRNA genome encodes a polyprotein comprising both structural and non-structural rhinovirus polypeptides (FIG. 1). The inventors aligned 539 amino acid sequences from various rhinovirus A serotypes and 463 amino acid sequences from various rhinovirus C serotypes to identify regions of high sequence conservation. They identified regions of high sequence conservation in the structural polypeptides VP4 and VP2, which derive from a naturally occurring precursor, a VP0 polyprotein. The inventors also identified regions of high sequence conservation in the non-structural polypeptides P2A, P2B and P2C, which derive form a naturally occurring precursor, a P2 polyprotein.

The inventors are of the view that an immunogenic composition comprising a non-naturally occurring mRNA encoding a naturally occurring rhinovirus polyprotein is more effective in eliciting an immune response against multiple rhinovirus serotypes of the same group than other types of vaccines.

Accordingly, the present invention provides anti-rhinovirus vaccines (in particular mRNA-based vaccines) comprising one or more non-naturally occurring mRNAs encoding one or more naturally occurring rhinovirus polyproteins that are highly conserved among multiple rhinoviruses of the same subgroup. The vaccines disclosed here are designed to be capable of eliciting an effective immune response, including, e.g., an effective T-cell response, against multiple rhinovirus serotypes of either the same, or multiple, rhinovirus groups.

The inventors have discovered that structural polypeptides located within the precursor VP0 polyprotein of the rhinovirus polyprotein (namely VP4 and VP2) and non-structural polypeptides located within the precursor P2 polyprotein (namely 2A, 2B and 2C) are particularly plentiful in conserved T-cell epitope-rich regions. Including these polyproteins, or mRNAs encoding them, in vaccination approaches is expected to yield a particularly potent T-cell response against multiple serotypes of group A and group C rhinoviruses. A combination of VP0 and P2 polyproteins, e.g., as a fusion protein (or an mRNA encoding it) is expected to elicit a T-cell response in at least 95% of the human population.

Moreover, the inventors' discovery opens up an opportunity of combining mRNAs encoding VP0 polyproteins and/or P2 polyproteins from group A and group C rhinoviruses to provide combination vaccines that are capable of eliciting an effective immune response against multiple serotypes from each group.

### Identifying Naturally Occurring Rhinovirus Polyproteins Suitable as Immunogens

Without wishing to be bound by any particular theory, the inventors are of the view that an immunogenic composition comprising a non-naturally occurring mRNA encoding a naturally occurring rhinovirus protein or polyprotein is more effective in eliciting an immune response against multiple rhinovirus serotypes of the same group than other types of vaccines, in particular those that generated only a limited selection of rhinovirus-derived T-cell epitope containing peptides or polypeptides. In part, this view is based on the discovery that predicted T-cell epitopes may only elicit a T-cell response in carriers of particular HLA alleles. Therefore, in order to achieve broad coverage in a large portion of the population, an immunogenic composition ideally induces the expression of a plurality of T-cell epitopes to ensure that a T-cell response is elicited in the vast majority of recipients.

Moreover, a naturally occurring rhinovirus protein or polyprotein is expected to be processed by immune cells in a manner that mirrors the natural infection process. As the antigen design approach disclosed herein is based on the in silico prediction of T-cell epitopes, using a naturally occurring protein reduces the risk inherent to such an approach. In particular, some of the predicted T-cell epitopes may not be generated by immune cells in vivo, whereas the use of naturally occurring protein (or an mRNA encoding the same) guarantees that a plurality of T-cell epitopes will be generated after administration of the immunogenic composition.

Accordingly, the invention provides one or more non-naturally occurring mRNAs encoding one or more non-structural rhinovirus polypeptides comprising one or more T-cell epitope-rich regions and/or one or more structural rhinovirus polypeptides comprising one or more T-cell epitope-rich regions in the context of a naturally occurring rhinovirus polyprotein in which they have been identified.

In a specific embodiment, an immunogenic composition of the invention (e.g., a vaccine) comprises a non-naturally occurring mRNA encoding a non-structural rhinovirus protein or polyprotein, which is, e.g., naturally occurring aside from one or more optional amino acid substitutions (e.g., 1, 2, 3, 4, 5, 6, 7, 8, 9, 10 or more amino acid substitutions), that may be introduced, e.g., to improve expression (e.g., through provision of a non-native signal sequence and/or removal of proteolytic activity). In some embodiments, the non-structural rhinovirus protein is or comprises one of P2A, P2B and P2C. In one specific embodiment, the non-structural rhinovirus protein is a P2 polyprotein. In some embodiments, an immunogenic composition of the invention (e.g., a vaccine) comprises an mRNA encoding multiple non-structural rhinovirus proteins or polyproteins (e.g., multiple P2 polyproteins from rhinovirus A and/or C).

In other particular embodiments, non-naturally occurring mRNAs encoding the one or more structural rhinovirus polypeptides comprising one or more T-cell epitope-rich regions described herein are provided as a rhinovirus protein or polyprotein, typically a naturally occurring rhinovirus protein or polyprotein. In a specific embodiment, an immunogenic composition of the invention (e.g., a vaccine) comprises a non-naturally occurring mRNA encoding a structural rhinovirus protein or polyprotein, which is, e.g., naturally occurring aside from one or more optional amino acid substitutions (e.g., 1, 2, 3, 4, 5, 6, 7, 8, 9, 10 or more amino acid substitutions), that may be introduced, e.g., to improve expression (e.g., through provision of a non-native signal sequence and/or removal of proteolytic activity). In some embodiments, the structural rhinovirus protein is or comprises one of VP2 and VP4. In one specific embodiment, the structural rhinovirus protein is a VP0 polyprotein. In some embodiments, an immunogenic composition of the invention (e.g., a vaccine) comprises an mRNA encoding multiple structural rhinovirus proteins or polyproteins (e.g., multiple VP0 polyproteins from rhinovirus A and/or C).

In some embodiments, it is convenient to provide an mRNA encoding a non-naturally occurring fusion protein of two or more naturally occurring rhinovirus proteins or polyproteins, e.g., a fusion protein comprising one or more naturally occurring structural rhinovirus proteins (or polyproteins) and one or more non-structural rhinovirus proteins (or polyproteins). For example, the naturally occurring structural protein may be selected from VP2 and VP4. The non-naturally occurring non-structural protein may be selected from P2A, P2B and P2C. The one or more structural proteins and the one or more non-structural proteins may be arranged in any order in the fusion protein. More typically, the one or more structural proteins and the one or more non-structural proteins are arranged in a similar manner as in a naturally occurring polyprotein. For example, P2A, P2B and P2C may be arranged as in a naturally occurring P2 polyprotein. Similarly, VP4 and VP2 may be arranged as in a naturally occurring VP0 polyprotein. The VP0 polyprotein and the P2 polyprotein may be arranged in either of the two possible combinations. In one embodiment, the fusion protein is VP0-P2 (i.e., VP0 is in the N-terminal position). In another embodiment, the fusion protein is P2-VP0 (i.e., P2 is in the N-terminal position).

In some embodiments, a fusion protein comprises multiple naturally occurring structural or non-structural rhinovirus proteins (or polyproteins), e.g., multiple VP0 polyproteins or multiple P2 polyproteins from different serotypes of rhinovirus A and/or C.

Accurately predicting T-cell epitope-rich regions across multiple rhinovirus serotypes can be difficult. Therefore, the bioinformatics approach of the inventors—at least in part—focuses on the identification of regions in the rhinovirus polyprotein that are highly conserved across multiple rhinovirus polyproteins from different serotypes. In particular, the invention provides a method of identifying a rhinovirus polyprotein for use as an immunogen that is capable of eliciting an immune response against rhinoviruses from multiple serotypes within a group. A method in accordance with the invention comprises the following steps: (a) retrieving a plurality of amino acid sequences from a database comprising amino acid sequences from naturally occurring rhinovirus isolates; (b) removing from the plurality of amino acid sequences retrieved in step (a) amino acid sequences shorter than 800 amino acids; (c) assigning the amino acid sequences remaining after step (b) into different phylogenetic clusters; (d) aligning the amino acid sequences to determine a consensus amino acid sequence for a complete rhinovirus polyprotein for one or more phylogenetic clusters identified in step (c); (e) aligning the consensus amino acid sequence obtained in step (c) with complete polyproteins of naturally occurring rhinovirus isolates; and (f) selecting a rhinovirus polyprotein as an immunogen that has an average identity (and optionally a median identity) of at least 80% (e.g., at least 85%, at least 90%, or at least 95%) to the corresponding amino acid sequences of rhinoviruses from at least two phylogenetic clusters identified in step (c). A suitable algorithm for performing the alignments in, e.g., step (d) is MAFFT (Katoh & Toh, Bioinformatics 2010; 26(15):1899-900 which is incorporated herewith by reference).

The inventors used this computational approach to identify conserved regions in the amino acid sequence of complete rhinovirus A and C polyproteins. The inventors believe that a naturally occurring rhinovirus polyprotein having an average identity (and optionally a median identity) of at least 80% (e.g., at least 85%, at least 90% or at least 95%) to the amino acid sequences of rhinoviruses from at least two phylogenetic clusters is capable of eliciting an immune response against substantially all serotypes in the at least two phylogenetic clusters, e.g., by inducing an effective T-cell response. Highly conserved regions particularly suitable for implementing the invention have an average identity (and optionally a median identity) of at least 80% (e.g., at least 85%, at least 90% or at least 95%) to the amino acid sequences of rhinoviruses from multiple phylogenetic clusters.

In particular, the inventors identified naturally occurring VP0 polyproteins and P2 polyproteins that have an amino acid sequence with an average identity (and optionally a median identity) of at least 80% (e.g., at least 85%, at least 90%, or at least 95%) to the amino acid sequences of VP0 polyproteins or P2 polyproteins from at least two phylogenetic clusters of rhinovirus group A or C. More typically, these naturally occurring VP0 polyproteins and P2 polyproteins have an amino acid sequence with an average identity (and optionally a median identity) of at least 80% (e.g., at least 85%, at least 90%, or at least 95%) to the amino acid sequences of VP0 polyproteins or P2 polyproteins from at least three, e.g., four, phylogenetic clusters of rhinovirus group A or C.

For example, the exemplified naturally occurring rhinovirus A VP0 polyproteins have an amino acid sequence with an average identity (and optionally a median identity) of at least about 80% to the amino acid sequences of VP0 polyproteins from at least three phylogenetic clusters. The exemplified naturally occurring rhinovirus A P2 polyproteins have an amino acid sequence with an average identity (and optionally a median identity) of at least about 80% to the amino acid sequences of P2 polyproteins from at least four phylogenetic clusters. The exemplified naturally occurring rhinovirus C VP0 polyproteins have an amino acid sequence with an average identity (and optionally a median identity) of at least about 80% to the amino acid sequences of VP0 polyproteins from at least four phylogenetic clusters. The exemplified naturally occurring rhinovirus P2 polyproteins have an amino acid sequence with an average identity (and optionally a median identity) of at least about 80% to the amino acid sequences of P2 polyproteins from at least two phylogenetic clusters.

Phylogenetic clusters can be identified using a suitable algorithm, e.g., based on a maximum-likelihood method. Maximum-likelihood trees can be calculated for large alignments using an algorithm such as FastTree 2 (Price et al., PLoS One 2010; 5(3): e9490, which is incorporated herewith by reference). Another suitable algorithm is PhyML (Guindon et al. Nucleic Acids Res. 2005; 33(Web Server issue): W557-9, which is incorporated herewith by reference). Both FastTree and PhyML provide nearly identical trees and therefore can be used interchangeable to implement the computational methods described herein.

A phylogenetic cluster typically represents at least 5 different serogroups of rhinovirus group A or C. In some embodiments, a phylogenetic cluster represents at least 10 different serogroups of rhinovirus group A or C. In some embodiments, a phylogenetic cluster represents 3, 4, 5, 6, 7, 8, 9, 10, 11, 12, 13, 14, 15, 16, 17, 18, 19, 20, 21, 22, 23, 24, 25, 26, 27, 28, 29 or 30 different serogroups of rhinovirus group A or C.

For example, rhinovirus group A can be divided into four phylogenetic clusters (referred herein as clusters 1-4) based on the amino acid sequences of complete polyproteins. These four phylogenetic clusters represent 20, 5, 28 and 3 serotypes, respectively. Cluster 1 comprises serotypes 9, 13, 15, 19, 22, 32, 38, 41, 43, 57, 60, 61, 64, 67, 73, 74, 75, 82, 94 and 96. Cluster 2 comprises serotypes 2, 23, 30, 39 and 49. Cluster 3 comprises serotypes 10, 11, 18, 21, 24, 25, 29, 31, 33, 34, 40, 44, 47, 50, 54, 55, 56, 57, 59, 62, 63, 66, 76, 77, 85, 90, 98 and 100. Cluster 4 comprises serotypes 1, 16 and 81. Corresponding clusters may also be identified when the phylogenetic analysis is limited to amino acid sequences of the non-structural P2 polyproteins.

In some embodiments, the VP0 polyprotein, the P2 polyprotein, or both, of a human rhinovirus A serotype 21 strain (GenBank ID: FJ445121.1, strain name: ATCC VR-1131) is/are capable of eliciting an immune response (e.g., a protective immune response) against rhinovirus A serotypes of at least cluster 3. In other embodiments, the VP0 polyprotein, the P2 polyprotein, or both, of a human rhinovirus A serotype 21 strain (GenBank ID: FJ445121.1, strain name: ATCC VR-1131) is/are capable of eliciting an immune response (e.g., a protective immune response) against rhinovirus A serotypes of at least clusters 2 and 3. In further embodiments, the VP0 polyprotein, the P2 polyprotein, or both, of a human rhinovirus A serotype 21 strain (GenBank ID: FJ445121.1, strain name: ATCC VR-1131) is/are capable of eliciting an immune response (e.g., a protective immune response) against rhinovirus A serotypes of clusters 1-4.

Rhinovirus group C can be divided into two phylogenic clusters (referred herein as 1ab and 2ab) representing 29 and 14 serotypes, respectively, based on the amino acid sequences of complete polyproteins. Each of these clusters can be subdivided further into four phylogenetic clusters (referred herein as 1a, 1b, 2a and 2b) representing 13, 16, 5 and 9 serotypes, respectively. Cluster 1a comprises rhinovirus C serotypes 8, 12, 15, 16, 17, 23, 25, 28, 30, 31, 41, 42 and 56. Cluster 1b comprises rhinovirus C serotypes 2, 4, 9, 19, 26, 33, 35, 36, 40, 47, 48, 49, 50, 51, 53 and 55. Cluster 2a comprises rhinovirus C serotypes 5, 11, 34, 45 and 54. Cluster 2b comprises rhinovirus C serotypes 1, 3, 6, 7, 22, 32, 39, 43 and 57. These four clusters are also identified when the phylogenetic analysis is limited to amino acid sequences of the structural VP0 polyproteins. Corresponding clusters may also be identified when the phylogenetic analysis is limited to amino acid sequences of the non-structural P2 polyproteins.

In some embodiments, the VP0 polyprotein of a human rhinovirus C serotype 34 strain (GenBank ID: MZ322913.1, strain name: 7H8M5V) is capable of eliciting an immune response (e.g., a protective immune response) against rhinovirus C serotypes of clusters 1a, 1b, 2a and 2b. In some embodiments, the P2 polyprotein of a human rhinovirus C serotype 17 strain (GenBank ID: MZ153245.1, strain name: RvC17/USA/2021/RCC55) is capable of eliciting an immune response (e.g., a protective immune response) against rhinovirus C serotypes of at least clusters 1a and 1b. In some embodiments, the VP0 polyprotein of a human rhinovirus C serotype 17 strain (GenBank ID: MZ153277.1, strain name: RvC17/USA/2021/368038-4) is capable of eliciting an immune response (e.g., a protective immune response) against rhinovirus C serotypes of at least clusters 1a and 1b. In some embodiments, the P2 polyprotein of a human rhinovirus C serotype 11 strain (GenBank ID: OK254863.1, strain name:RvC11/USA/2021/L2PJH9) is capable of eliciting an immune response (e.g., a protective immune response) against rhinovirus C serotypes of at least clusters 2a and 2b. In some embodiments, the VP0 polyprotein of a human rhinovirus C serotype 11 strain (GenBank ID: MZ268689.1, isolate: 469843) is capable of eliciting an immune response (e.g., a protective immune response) against rhinovirus C serotypes of at least clusters 2a and 2b.

In some embodiments, the invention provides an immunogenic composition comprising at least one non-naturally occurring messenger RNA (mRNA) encoding a rhinovirus A VP0 polyprotein that has an amino acid sequence with an average identity (and optionally a median identity) of at least 80% (e.g., at least 85%, at least 90%, or at least 95%) to the amino acid sequences of VP0 polyproteins from at least two (e.g., at least three or at least four) phylogenetic clusters of rhinovirus A. In some embodiments, the invention provides an immunogenic composition comprising at least one non-naturally occurring messenger RNA (mRNA) encoding a rhinovirus A VP0 polyprotein that has an amino acid sequence with an average identity and a median identity of at least 80% (e.g., at least 85%, at least 90%, or at least 95%) to the amino acid sequences of VP0 polyproteins from at least two (e.g., at least three or at least four) phylogenetic clusters of rhinovirus A.

In some embodiments, the invention provides an immunogenic composition comprising at least one non-naturally occurring messenger RNA (mRNA) encoding a rhinovirus C VP0 polyprotein that has an amino acid sequence with an average identity (and optionally a median identity) of at least 80% (e.g., at least 85%, at least 90%, or at least 95%) to the amino acid sequences of VP0 polyproteins from at least two (e.g., at least three or at least four) phylogenetic clusters of rhinovirus C. In some embodiments, the invention provides an immunogenic composition comprising at least one non-naturally occurring messenger RNA (mRNA) encoding a rhinovirus C VP0 polyprotein that has an amino acid sequence with an average identity and a median identity of at least 80% (e.g., at least 85%, at least 90%, or at least 95%) to the amino acid sequences of VP0 polyproteins from at least two (e.g., at least three or at least four) phylogenetic clusters of rhinovirus C.

In some embodiments, the invention provides an immunogenic composition comprising at least one non-naturally occurring messenger RNA (mRNA) encoding a rhinovirus A P2 polyprotein that has an amino acid sequence with an average identity (and optionally a median identity) of at least 80% (e.g., at least 85%, at least 90%, or at least 95%) to the amino acid sequences of P2 polyproteins from at least two (e.g., at least three or at least four) phylogenetic clusters of rhinovirus A. In some embodiments, the invention provides an immunogenic composition comprising at least one non-naturally occurring messenger RNA (mRNA) encoding a rhinovirus A P2 polyprotein that has an amino acid sequence with an average identity and a median identity of at least 80% (e.g., at least 85%, at least 90% or at least 95%) to the amino acid sequences of P2 polyproteins from at least two (e.g., at least three or at least four) phylogenetic clusters of rhinovirus A.

In some embodiments, the invention provides an immunogenic composition comprising at least one non-naturally occurring messenger RNA (mRNA) encoding a rhinovirus C P2 polyprotein that has an amino acid sequence with an average identity (and optionally a median identity) of at least 80% (e.g., at least 85%, at least 90%, or at least 95%) to the amino acid sequences of P2 polyproteins from at least two (e.g., at least three or at least four) phylogenetic clusters of rhinovirus C. In some embodiments, the invention provides an immunogenic composition comprising at least one non-naturally occurring messenger RNA (mRNA) encoding a rhinovirus C P2 polyprotein that has an amino acid sequence with an average identity and a median identity of at least 80% (e.g., at least 85%, at least 90%, or at least 95%) to the amino acid sequences of P2 polyproteins from at least two (e.g., at least three or at least four) phylogenetic clusters of rhinovirus C.

Tables 2 and 3 provide exemplary amino acid sequences of naturally occurring rhinovirus A and C proteins and polyproteins that may be encoded by one or more mRNAs as described herein. The exemplary rhinovirus A sequences are derived from a human rhinovirus A serotype 21 strain (GenBank ID: FJ445121.1, strain name: ATCC VR-1131). In some embodiments, amino acid sequences derived from other naturally occurring rhinovirus A proteins or polyproteins may be used in practicing the invention. For example, human rhinovirus A serotype 24, 57 or 90 strains (e.g., GenBank IDs: JN562727.1, strain name: HRV-A24_p1025_sR2625_2009; KY369874.1, strain name: SC9723; and FJ445167.1, strain name: ATCC VR-1291; respectively) may be a suitable alternative to aforementioned human rhinovirus A serotype 21.

For example, as the circulating rhinovirus A strains naturally mutate, repeating the analysis provided in the Example 1 of the present application may yield a polyprotein from a different rhinovirus A strain that provides a better match to the polyproteins of circulating rhinovirus A strains. As discussed above, the polyprotein may be selected on the basis that it has an amino acid sequence with an average identity (and optionally a median identity) of at least 80% (e.g., at least 85%, at least 90% or at least 95%) to the amino acid sequences of the polyproteins from at least two (e.g., at least three or at least four) phylogenetic clusters of circulating rhinoviruses of group A.

Accordingly, in some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus A serotype 21 VP0 polyprotein as set forth in SEQ ID NO: 4, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus A serotype 21 VP0 polyprotein as set forth in SEQ ID NO: 4.

In some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus A serotype 90 VP0 polyprotein as set forth in SEQ ID NO: 5, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus A serotype 90 VP0 polyprotein as set forth in SEQ ID NO: 5.

In some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus A serotype 21 P2 polyprotein as set forth in SEQ ID NO: 6, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus A serotype 21 P2 polyprotein as set forth in SEQ ID NO: 6.

In some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus A serotype 57 P2 polyprotein as set forth in SEQ ID NO: 7, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus A serotype 57 P2 polyprotein as set forth in SEQ ID NO: 7.

The exemplary VP0 rhinovirus C sequences are derived from serotypes 11, 17 and 34 of human rhinovirus C (GenBank ID: MZ268689.1, isolate: 469843, GenBank ID: MZ153277.1, strain name: RvC17/USA/2021/368038 and GenBank ID: MZ322913.1, isolate: 7H8M5V, respectively). The exemplary P2 rhinovirus C sequences are derived from serotypes 11 and 17 of human rhinovirus C (GenBank ID: OK254863.1, strain name: RvC11/USA/2021/L2PJH9 and GenBank ID: MZ153245.1, strain name: RvC17/USA/2021/RCC55, respectively). In some embodiments, amino acid sequences derived from other naturally occurring rhinovirus C proteins or polyproteins may be used in practicing the invention. As the circulating rhinovirus C strains naturally mutate, repeating the analysis provided in Example 8 of the present application may yield a different rhinovirus C strain that provides a better match to the circulating rhinovirus C strains.

Accordingly, in some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus C serotype 34 VP0 polyprotein as set forth in SEQ ID NO: 3, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus C serotype 34 VP0 polyprotein as set forth in SEQ ID NO: 3.

In some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus C serotype 11 VP0 polyprotein as set forth in SEQ ID NO: 1, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus C serotype 11 VP0 polyprotein as set forth in SEQ ID NO: 1.

In some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus C serotype 17 VP0 polyprotein as set forth in SEQ ID NO: 2, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus C serotype 17 VP0 polyprotein as set forth in SEQ ID NO: 2.

In some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus C serotype 11 P2 polyprotein as set forth in SEQ ID NO: 8, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus C serotype 11 P2 polyprotein as set forth in SEQ ID NO: 8.

In some embodiments, the invention provides a non-naturally occurring nucleic acid (e.g., an mRNA or DNA template for producing such as mRNA by in vitro transcription) comprising an optimized nucleotide sequence that encodes an amino acid sequence that is at least 80% identical to the amino acid sequence of the rhinovirus C serotype 17 P2 polyprotein as set forth in SEQ ID NO: 9, as long as it meets stringent selection criteria of the method for identifying a rhinovirus polyprotein for use as an immunogen disclosed herein. In some embodiments, the non-naturally occurring nucleic acid comprises an optimized nucleotide sequence that encodes an amino acid sequence that is at least 85%, at least 90%, at least 95%, at least 99% or 100% identical to the amino acid sequence of the rhinovirus C serotype 17 P2 polyprotein as set forth in SEQ ID NO: 9.

### Secretion Signal Sequence

In some embodiments of the invention, the rhinovirus polypeptides, proteins and polyproteins described herein are operationally linked to a non-native secretion signal sequence from a different virus. Such sequences have been found to increase secretion from mammalian cells into the surrounding extracellular space. Typically, the secretion signal sequence is derived from a virus capable of infecting human cells. Without wishing to be bound by any particular theory, the inventors hypothesize that fusing the rhinovirus polypeptides, proteins and polyproteins described herein to such a non-native viral secretion signal sequence promotes immunogenicity and therefore increases efficacy of the immunogenic compositions described herein.

In some embodiments, the secretion signal sequence for use with the invention is derived from an influenza secretion signal sequence, a SARS CoV-2 secretion signal sequence, a varicella-zoster virus (VZV) secretion signal sequence, a measles secretion signal sequence, a rubella secretion signal sequence, a mumps secretion signal sequence, an Ebola secretion signal sequence, and a smallpox secretion signal sequence.

In specific embodiments, the secretion signal sequence for use with the invention is selected from an influenza hemagglutinin (HA) secretion signal sequence, a SARS CoV-2 spike protein secretion signal sequence, a VZV gB secretion signal sequence, a VZV gE secretion signal sequence, a VZV gI secretion signal sequence, a VZV gK secretion signal sequence, a measles F-protein secretion signal sequence, a rubella E1 protein secretion signal sequence, a rubella E2 protein secretion signal sequence, a mumps F-protein secretion signal sequence, an Ebola GP protein secretion signal sequence, and a smallpox 6 kDa IC protein secretion signal sequence. These secretion signal sequences are derived from viruses which have been previously administered to humans as part of an immunogenic composition and therefore are considered safe for use in humans.

In some embodiments, the secretion signal sequence is selected from Table 1.

TABLE 1
Exemplary secretion signal sequences

| Secretion signal | | | Amino acid sequence |
|---|---|---|---|
| name | Organism | Strain | |
| HA (H1N1) | Influenza virus | A/New Caledonia/20/1999 | MKAKLLVLLCTFTATYA |
| | | | (SEQ ID NO: 14) |
| HA (H1N1pdm) | Influenza virus | A/California/7/2009 | MKAILVVLLYTFATANA |
| | | | (SEQ ID NO: 15) |
| HA (H3N2) | Influenza virus | A/Moscow/10/1999 | MKTIIALSYILCLVFA |
| | | | (SEQ ID NO: 16) |
| HA B | Influenza virus | B/Phuket/3073/2013 | MKAIIVLLMVVTSNA |
| | | | (SEQ ID NO: 17) |
| Spike | SARS CoV-2 | Wuhan-1 | MFVFLVLLPLVS |
| | | | (SEQ ID NO: 18) |
| Spike | SARS CoV-2 | Wuhan-1 (long version) | MFLLTTKRTMFVFLVLLPLV |
| | | | S |
| | | | (SEQ ID NO: 19) |
| gB | VZV | Oka strain | MSPCGYYSKWRNRDRPEYR |
| | | | RNLRFRRFFSSIHPNAAAGSG |
| | | | FNGPGVFITSVTGVWLCFLCI |
| | | | FSMFVTAVVS |
| | | | (SEQ ID NO: 20) |
| gE | VZV | Oka strain | MGTVNKPVVGVLMGFGIIT |
| | | | GTLRITNPVRA |
| | | | (SEQ ID NO: 21) |
| gI | VZV | Oka strain | MFLIQCLISAVIFYIQVTNA |
| | | | (SEQ ID NO: 22) |
| gK | VZV | Oka strain | MQALGIKTEHFIIMCLLSGH |
| | | | A |
| | | | (SEQ ID NO: 23) |
| F | Measles | Edmonston- | MGLKVNVSAIFMAVLLTLQ |
| | | Zagreb strain | TPTG |
| | | | (SEQ ID NO: 24) |
| E1 | Rubella | RA27/3 strain | MGAAAALTAVVLQGYNPPA |
| | | | YG |
| | | | (SEQ ID NO: 25) |
| E2 | Rubella | RA27/3 strain | MGAPQAFLAGLLLAAVAVG |
| | | | TARA |
| | | | (SEQ ID NO: 26) |
| F | Mumps | Miyahara strain | MKVFLVTCLGFAVFSSSVC |
| | | | (SEQ ID NO: 27) |
| GP | Ebola | Mayinga-76 strain | MGVTGILQLPRDRFKRTSFF |
| | | | LWVIILFQRTFS |
| | | | (SEQ ID NO: 28) |
| 6 kDa IC | Smallpox | Germany 91-3 strain | MRSLIIFLLFPSIIYS |
| | | | (SEQ ID NO: 29) |

In particular embodiments, the secretion signal sequences for use with the rhinovirus polypeptides, proteins and polyproteins described herein comprises an HA secretion signal sequence from influenza A or influenza B. In one specific embodiment, the secretion signal sequence is derived from the HA secretion signal sequence of an influenza A virus (e.g., an H1N1 subtype such as A/California/7/2009).

Without wishing to be bound by any particular theory, the inventors hypothesize that fusing a non-structural rhinovirus polypeptide (such as P2), which is not normally secreted, to a secretion signal sequence further enhances the immune response (e.g., the T-cell response) to this polypeptide.

### Exemplary Rhinovirus A Polyproteins

The inventors found that the amino acid sequence of the polyprotein of a human rhinovirus A serotype 21 strain (GenBank ID: FJ445121.1, strain name: ATCC VR-1131) is most similar to the consensus sequence created from 539 individual rhinovirus A polyprotein sequences. Without wishing to be bound by any particular theory, the inventors hypothesize that the polyprotein sequence of this particular rhinovirus A serotype can be used to construct immunogenic compositions that are effective at eliciting an immune response against multiple rhinovirus A serotypes.

Accordingly, in specific embodiments, an immunogenic composition of the invention comprises a non-naturally occurring mRNA encoding a rhinovirus A P2 polyprotein having the amino acid sequence of SEQ ID NO: 6, or a non-proteolytic version thereof. In another specific embodiment, an immunogenic composition of the invention comprises a non-naturally occurring mRNA encoding a rhinovirus A VP0 polyprotein having the amino acid sequence of SEQ ID NO: 4.

In some embodiments, the rhinovirus A VP0 polyprotein or the rhinovirus A P2 polyprotein are operationally linked to a non-native secretion signal sequence from an influenza A virus to increase secretion of the mRNA encoded protein(s). Accordingly, in specific embodiments, an immunogenic composition of the invention comprises a non-naturally occurring mRNA encoding a rhinovirus A P2 polyprotein having the amino acid sequence of SEQ ID NO: 30, or a non-proteolytic version thereof. In another specific embodiment, an immunogenic composition of the invention comprises a non-naturally occurring mRNA encoding a rhinovirus A P2 polyprotein having the amino acid sequence of SEQ ID NO: 31, or a non-proteolytic version thereof. In another specific embodiment, an immunogenic composition of the invention comprises a non-naturally occurring mRNA encoding a rhinovirus A VP0 polyprotein having the amino acid sequence of SEQ ID NO: 32. In another specific embodiment, an immunogenic composition of the invention comprises a non-naturally occurring mRNA encoding a rhinovirus VP0 polyprotein having the amino acid sequence of SEQ ID NO: 33.

Together, the P2 polyprotein and VP0 polyprotein derived from the rhinovirus A serotype 21 strain are predicted to cover 97-99% of all human MHC-I and MHC-II alleles worldwide. Therefore, in a further specific embodiment, an immunogenic composition of the invention comprises a non-naturally occurring mRNA encoding a rhinovirus A P2 polyprotein having the amino acid sequence of SEQ ID NOs: 6 or 30, or a non-proteolytic version thereof, and a non-naturally occurring mRNA encoding a rhinovirus A VP0 polyprotein having the amino acid sequence of SEQ ID NOs: 4 or 32. In particular embodiments, an immunogenic composition of the invention comprises a non-naturally occurring mRNA encoding a rhinovirus A P2 polyprotein having the amino acid sequence of SEQ ID NO: 30, or a non-proteolytic version thereof, and a non-naturally occurring mRNA encoding a rhinovirus A VP0 polyprotein having the amino acid sequence of SEQ ID NO: 32. In another particular embodiment, an immunogenic composition of the invention comprises a non-naturally occurring mRNA encoding a rhinovirus A P2 polyprotein having the amino acid sequence of SEQ ID NO: 6, or a non-proteolytic version thereof, and a non-naturally occurring mRNA encoding a rhinovirus A VP0 polyprotein having the amino acid sequence of SEQ ID NO: 4. In some embodiments, an immunogenic composition of the invention comprises a non-naturally occurring mRNA encoding a rhinovirus A P2 polyprotein having the amino acid sequence of SEQ ID NO: 30, or a non-proteolytic version thereof, and a non-naturally occurring mRNA encoding a rhinovirus A VP0 polyprotein having the amino acid sequence of SEQ ID NO: 4. In other embodiments, an immunogenic composition of the invention comprises a non-naturally occurring mRNA encoding a rhinovirus A P2 polyprotein having the amino acid sequence of SEQ ID NO: 6, or a non-proteolytic version thereof, and a non-naturally occurring mRNA encoding a rhinovirus A VP0 polyprotein having the amino acid sequence of SEQ ID NO: 32.

Even broader protection can be achieved by further including an mRNA encoding a polyprotein from a phylogenetically distant serogroup in an immunogenic composition of the invention. Rhinovirus A serotype 8 (GenBank ID: FJ445113.1, strain name: ATCC VR-1118) is from a phylogenetically distant serogroup relative to rhinovirus A serotype 8. Inclusion of a polyprotein P2 of rhinovirus A serotype 8 may be beneficial to expand the effectiveness of an immunogenic composition of the invention. Accordingly, in specific embodiments, an immunogenic composition of the invention comprises a non-naturally occurring mRNA encoding a rhinovirus A P2 polyprotein having the amino acid sequence of SEQ ID NO: 80, or a non-proteolytic version thereof (e.g., having the amino acid sequence of SEQ ID NO: 81). In some embodiments, the rhinovirus A P2 polyprotein is operationally linked to a non-native secretion signal sequence from an influenza A virus to increase secretion of the mRNA encoded protein. Therefore, in some embodiments, an immunogenic composition of the invention comprises a non-naturally occurring mRNA encoding a rhinovirus A P2 polyprotein having the amino acid sequence of SEQ ID NO: 82, or a non-proteolytic version thereof.

For example, in order to provide immunity against infection caused by multiple rhinovirus A serotypes the immunogenic composition may include one or more non-naturally occurring mRNA(s) encoding one or more naturally occurring polyproteins of rhinovirus A serotype 21 and one or more non-naturally occurring mRNA(s) encoding one or more naturally occurring polyproteins of rhinovirus A serotype 8.

In some embodiments, an immunogenic composition of the invention comprises a first non-naturally occurring mRNA encoding a rhinovirus A serotype 21 VP0 polyprotein having the amino acid sequence of SEQ ID NO: 4, a second non-naturally occurring mRNA encoding a rhinovirus A serotype 21 P2 polyprotein having the amino acid sequence of SEQ ID NO: 6, or a non-proteolytic version thereof (e.g., SEQ ID NO: 77), and a third non-naturally occurring mRNA encoding a rhinovirus A serotype 8 P2 polyprotein having the amino acid sequence of SEQ ID NO: 80, or a non-proteolytic version thereof (e.g., SEQ ID NO: 81).

In some embodiments, an immunogenic composition of the invention comprises a first non-naturally occurring mRNA encoding the rhinovirus A serotype 21 VP0 polyprotein having the amino acid sequence of SEQ ID NO: 32, a second non-naturally occurring mRNA encoding the rhinovirus A serotype 21 P2 polyprotein having the amino acid sequence of SEQ ID NO: 6, or a non-proteolytic version thereof (e.g., SEQ ID NO: 77), and a third non-naturally occurring mRNA encoding the rhinovirus A serotype 8 P2 polyprotein having the amino acid sequence of SEQ ID NO: 80, or a non-proteolytic version thereof (e.g., SEQ ID NO: 81).

In some embodiments, an immunogenic composition of the invention comprises a first non-naturally occurring mRNA encoding the rhinovirus A serotype 21 VP0 polyprotein having the amino acid sequence of SEQ ID NO: 32, a second non-naturally occurring mRNA encoding the rhinovirus A serotype 21 P2 polyprotein having the amino acid sequence of SEQ ID NO: 30, or a non-proteolytic version thereof, and a third non-naturally occurring mRNA encoding the rhinovirus A serotype 8 P2 polyprotein having the amino acid sequence of SEQ ID NO: 82, or a non-proteolytic version thereof.

In Table 2, rhinovirus A P2 polypeptides are in bold and rhinovirus A VP0 polypeptides are underlined. Secretion signal sequences are shown in italics. The VP0 polyprotein comprising the influenza virus A-derived secretion signal sequence comprises the sequence DTL between the secretion signal and the VP0 polyprotein. DTL corresponds to the first three amino acids at the N-terminus of the mature influenza HA polypeptide, from which the exemplary secretion signal was derived.

TABLE 2
Exemplary amino acid sequences encoding native
rhinovirus A proteins or polyproteins

| | | SEQ | |
|---|---|---|---|
| | | ID | |
| Serotype | Name | NO | Sequence |
| A21 | P2 | 6 | MGPSDMYVHVGNLIYRNLHLFNSEMHDSILISYSSDLVIY |
| | | | RTNTTGDDYIPTCDCTEATYYCKHKNRYFPIKVTSHDWYE |
| | | | IQESEYYPKHIQYNLLIGEGPCEPGDCGGKLLCKHGVIGM |
| | | | ITAGGDGHVAFIDLRHFHCAEEQGITDYIHMLGEAFGSGF |
| | | | VDSVKEQINAINPINNISKKIIKWLLRIISAMVIIIRNSS |
| | | | DPQTIIATLTLIGCSGSPWRFLKEKFCKWTQLNYIHKESD |
| | | | SWLKKFTEMCNAARGLEWIGNKISKFIDWMKSMLPQAQLK |
| | | | VKYLSELKKLNLLEKQIEHLRAADSATQEKIKCEIDTLHD |
| | | | LSCKFLPLYASEAKRIKVLHNKCNVVIKQKKRCEPVAVVI |
| | | | HGEPGTGKSMTTNFLARMITNDSDIYSLPPDPKYFDGYDQ |
| | | | QSVVIMDDIMQNPSGDDMTLFCQMVSSVTFIPPMADLPDK |
| | | | GKPFDSRFVLCSTNHSLLTPPTITSLPAMNRRFFMDLDII |
| | | | VCDKYKDAQGKLNVSAAFKPCDVDTKIGNARCCPFICGKA |
| | | | VMFKDRNTCRTYTLAQIYNQILEEDKRRRQVIDVMSAIFQ |
| A57 | P2 | 7 | MGPSDMYVHVGNLIYRNLHLFNSDMHDSILVSYSSDLVIY |
| | | | RTNTTGDDYIPTCDCTEATYYCRHKNRYYPIKVTSHDWYE |
| | | | IQESEYYPKHIQYNLLIGEGPCEPGDCGGKLLCKHGVIGI |
| | | | ITAGGEGHVAFIDLRHFHCAEEQGITDYIHMLGEAFGSGF |
| | | | VDSVKEQINAINPINNISKKIIKWLLRIISAMVIIIRNSS |
| | | | DPQTIIATLTLIGCSGSPWRFLKEKFCKWTQLNYIHKESD |
| | | | SWLKKFTEMCNAARGLEWIGNKISKFIDWMKSMLPQAQLK |
| | | | IKYLSELKKLNLLEKQIEHLRVADSATQEKIKCEIDTLHD |
| | | | LSCKFLPLYASEAKRIKVLYNKCNVVIKQKKRCEPVAVVI |
| | | | HGEPGTGKSMTTNFLARMITNDSDIYSLPPDPKYFDGYDQ |
| | | | QSVVIMDDIMQNPSGDDMTLFCQMVSSVTFIPPMADLPDK |
| | | | GKPFDSRFVLCSTNHSMLAPPTITSLPAMNRRFFMDLDII |
| | | | VNDKYKDSQGKLNVSAAFKPCDVDTKIGNARCCPFICGKA |
| | | | VTFKDRNTCHTFSLAQVYNQILDEDKRRRQVIDVMSAIFQ |
| A21 | VPO | 4 | MGTQVSRQNVGTHSTQNSVSNGSSLNYFNINYFKDAASSG |
| | | | ASKLEFSQDPSKFTDPVKDVLEKGIPTLQSPTVEACGYSD |
| | | | RIIQITRGDSTITSQDVANAVVGYGVWPHYLTPQDATAID |
| | | | KPSRPDTSSNRFYTLESKMWTSDSKGWWWKLPDALKNMGI |
| | | | FGENMFYHFLGRSGYTVHVQCNASKFHQGTLIVVMIPEHQ |
| | | | LASASTGNVTAGYNLTHPGEQGRDVGITRVEDLLKQPSDD |
| | | | SWLNFDGTLLGNITIFPHQFINLRSNNSATIIVPYVNAVP |
| | | | MDSMPRHNNWSLVIIPICPLESDGQTPVPITISISPMCAE |
| | | | FSGARAKSQ |
| A90 | VPO | 5 | MGAQVSRQNVGTHSTQNSVSNGSSLNYFNINYFKDAASSG |
| | | | ASKLEFSQDPSKFTDPVKDVLEKGIPTLQSPSVEACGYSD |
| | | | RIIQITRGDSTITSQDVANAVVAYGVWPHYLTPQDATAID |
| | | | KPSRPDTSSNRFYTLESKTWTGSSKGWWWKLPDALKGMGI |
| | | | FGENMFYHFLGRSGYTVHVQCNASKFHQGTLIVAMIPEHQ |
| | | | LASALAGNVTAGYNLTHPGEGGRTVDQTNRENQSKQPSDD |
| | | | SWLNFDGTLLGNITIFPHQFINLRSNNSATIIVPYVNAVP |
| | | | MDSMLRHNNWSLVIIPICPLESMQVPNTVPITISISPMCA |
| | | | EFSGARARSQ |
| A21 | P2 | 30 | MKAILVVLLYTFATANAGPSDMYVHVGNLIYRNLHLFNSE |
| | comprising a | | MHDSILISYSSDLVIYRTNTTGDDYIPTCDCTEATYYCKH |
| | secretion | | KNRYFPIKVTSHDWYEIQESEYYPKHIQYNLLIGEGPCEP |
| | signal | | GDCGGKLLCKHGVIGMITAGGDGHVAFIDLRHFHCAEEQG |
| | sequence | | ITDYIHMLGEAFGSGFVDSVKEQINAINPINNISKKIIKW |
| | | | LLRIISAMVIIIRNSSDPQTIIATLTLIGCSGSPWRFLKE |
| | | | KFCKWTQLNYIHKESDSWLKKFTEMCNAARGLEWIGNKIS |
| | | | KFIDWMKSMLPQAQLKVKYLSELKKLNLLEKQIEHLRAAD |
| | | | SATQEKIKCEIDTLHDLSCKFLPLYASEAKRIKVLHNKCN |
| | | | VVIKQKKRCEPVAVVIHGEPGTGKSMTTNFLARMITNDSD |
| | | | IYSLPPDPKYFDGYDQQSVVIMDDIMQNPSGDDMTLFCQM |
| | | | VSSVTFIPPMADLPDKGKPFDSRFVLCSTNHSLLTPPTIT |
| | | | SLPAMNRRFFMDLDIIVCDKYKDAQGKLNVSAAFKPCDVD |
| | | | TKIGNARCCPFICGKAVMFKDRNTCRTYTLAQIYNQILEE |
| | | | DKRRRQVIDVMSAIFQ |
| A57 | P2 | 31 | MKAILVVLLYTFATANAGPSDMYVHVGNLIYRNLHLFNSD |
| | comprising a | | MHDSILVSYSSDLVIYRTNTTGDDYIPTCDCTEATYYCRH |
| | signal | | KNRYYPIKVTSHDWYEIQESEYYPKHIQYNLLIGEGPCEP |
| | secretion | | GDCGGKLLCKHGVIGIITAGGEGHVAFIDLRHFHCAEEQG |
| | sequence | | ITDYIHMLGEAFGSGFVDSVKEQINAINPINNISKKIIKW |
| | | | LLRIISAMVIIIRNSSDPQTIIATLTLIGCSGSPWRFLKE |
| | | | KFCKWTQLNYIHKESDSWLKKFTEMCNAARGLEWIGNKIS |
| | | | KFIDWMKSMLPQAQLKIKYLSELKKLNLLEKQIEHLRVAD |
| | | | SATQEKIKCEIDTLHDLSCKFLPLYASEAKRIKVLYNKCN |
| | | | VVIKQKKRCEPVAVVIHGEPGTGKSMTTNFLARMITNDSD |
| | | | IYSLPPDPKYFDGYDQQSVVIMDDIMQNPSGDDMTLFCQM |
| | | | VSSVTFIPPMADLPDKGKPFDSRFVLCSTNHSMLAPPTIT |
| | | | SLPAMNRRFFMDLDIIVNDKYKDSQGKLNVSAAFKPCDVD |
| | | | TKIGNARCCPFICGKAVTFKDRNTCHTFSLAQVYNQILDE |
| | | | DKRRRQVIDVMSAIFQ |
| A21 | VPO | 32 | MKAILVVLLYTFATANADTLGTQVSRQNVGTHSTQNSVSN |
| | comprising a | | GSSLNYFNINYFKDAASSGASKLEFSQDPSKFTDPVKDVL |
| | secretion | | EKGIPTLQSPTVEACGYSDRIIQITRGDSTITSQDVANAV |
| | signal | | VGYGVWPHYLTPQDATAIDKPSRPDTSSNRFYTLESKMWT |
| | sequence | | SDSKGWWWKLPDALKNMGIFGENMFYHFLGRSGYTVHVQC |
| | | | NASKFHQGTLIVVMIPEHQLASASTGNVTAGYNLTHPGEQ |
| | | | GRDVGITRVEDLLKQPSDDSWLNFDGTLLGNITIFPHQFI |
| | | | NLRSNNSATIIVPYVNAVPMDSMPRHNNWSLVIIPICPLE |
| | | | SDGQTPVPITISISPMCAEFSGARAKSQ |
| A90 | VPO | 33 | MKAILVVLLYTFATANADTLGAQVSRQNVGTHSTQNSVSN |
| | comprising a | | GSSLNYFNINYFKDAASSGASKLEFSQDPSKFTDPVKDVL |
| | secretion | | EKGIPTLQSPSVEACGYSDRIIQITRGDSTITSQDVANAV |
| | signal | | VAYGVWPHYLTPQDATAIDKPSRPDTSSNRFYTLESKTWT |
| | sequence | | GSSKGWWWKLPDALKGMGIFGENMFYHFLGRSGYTVHVQC |
| | | | NASKFHQGTLIVAMIPEHQLASALAGNVTAGYNLTHPGEG |
| | | | GRTVDQTNRENQSKQPSDDSWLNFDGTLLGNITIFPHQFI |
| | | | NLRSNNSATIIVPYVNAVPMDSMLRHNNWSLVIIPICPLE |
| | | | SMQVPNTVPITISISPMCAEFSGARARSQ |
| A8 | P2 | 80 | MGPSEMFVHTTNLMYRNYHLTPEQELDSAIQVVYTADLVI |
| | | | HRTNDKGDDYIPDCNCTDCCYYCAHKNRYIPVKVRYYNYY |
| | | | TIQESEYYPKHIQYDILLGEGPSEPGDCGGKLLCKHGVIG |
| | | | MVTAGGDNHVAFIDLRKYRITEAEEQGITDYVKSLGDAFG |
| | | | VGFVEQIKEQVSNINPLNKISAKVIKWLIRVISALVIAVR |
| | | | SQGDLATLSATLVLLGCSDSPWRFLKQKVCQWLDLRYVHK |
| | | | ESDGWIKKFTEMCNAARGLEWIGCKISKFIDWLKSMLPQA |
| | | | QNKIKFLQFTKQLQLKEKQIDGLPYATIKQQEEYLQEMEE |
| | | | MLDISNKLLPLYPRENKMIKDLLKQAKNMTVASKRIEPVA |
| | | | VMFHGDPGSGKSICTNILARMITNPSDIYSLPPDPKYFDG |
| | | | YHQQTVVIMDDVMQNPNGEDMSTFCQMVSSVNYVVPMADL |
| | | | PDKGTLFSSDYVFCSTNQHILTPPTISTLPALNRRLFLDL |
| | | | TIKVNPKYLESGKLNLDCALKACDQEQKIGNARCCPLICG |
| | | | KAVSFVNRNNNEELSLSRVYNQIVHEHNRRLNVSKHMEAI |
| | | | FQ |
| A8 | P2 | 82 | MKAILVVLLYTFATANAGPSEMFVHTTNLMYRNYHLTPEQ |
| | comprising a | | ELDSAIQVVYTADLVIHRTNDKYNYYTIQESEYYPKHIQY |
| | secretion | | DILLGEGPSEPGDGDDYIPDCNCTDCCYYCAHKNRYIPVK |
| | signal | | VRYCGGKLLCKHGVIGMVTAGGDNHVAFIDLRKYRITEAE |
| | sequence | | EQGITDYVKSLGDAFGVGFVEQIKEQVSNINPLNKISAKV |
| | | | IKWLIRVISALVIAVRSQGDLATLSATLVLLGCSDSPWRF |
| | | | LKQKVCQWLDLRYVHKESDGWIKKFTEMCNAARGLEWIGC |
| | | | KISKFIDWLKSMLPQAQNKIKFLQFTKQLQLKEKQIDGLP |
| | | | YATIKQQEEYLQEMEEMLDISNKLLPLYPRENKMIKDLLK |
| | | | QAKNMTVASKRIEPVAVMFHGDPGSGKSICTNILARMITN |
| | | | PSDIYSLPPDPKYFDGYHQQTVVIMDDVMQNPNGEDMSTF |
| | | | CQMVSSVNYVVPMADLPDKGTLFSSDYVFCSTNQHILTPP |
| | | | TISTLPALNRRLFLDLTIKVNPKYLESGKLNLDCALKACD |
| | | | QEQKIGNARCCPLICGKAVSFVNRNNNEELSLSRVYNQIV |
| | | | HEHNRRLNVSKHMEAIFQ |

The active site of P2A in rhinovirus is highly conserved. In rhinovirus A, the active site consists of a catalytic triad of a cysteine (C) residue, e.g., at position 106 (Cys106), a histidine (H) residue at position 18 (His18) and an aspartic acid (D) residue at position 35 (Asp35), although the exact residue number can vary from serotype to serotype. For example, in rhinovirus A serotype 21, the catalytic triad is formed by Cys106, His18 and Asp35. In rhinovirus A serotype 8, the catalytic triad is formed by Cys107, His18 and Asp36. A non-proteolytic version of P2A can be generated by mutating one or more of these sites, e. g., Cys106 or Cys107, respectively.

In some embodiments, an immunogenic composition of the invention comprises a non-naturally occurring mRNA encoding a fusion protein comprising a rhinovirus A P2 polyprotein having the amino acid sequence of SEQ ID NO: 6, or a non-proteolytic version thereof (e.g., SEQ ID NO: 77), and a rhinovirus A VP0 polyprotein having the amino acid sequence of SEQ ID NO: 4. In some embodiments, the fusion is P2-VP0 (i.e., P2 is at the N-terminus). In alternative embodiments, the fusion is VP0-P2 (i.e., VP0 is at the N-terminus). In either arrangement, the rhinovirus A P2 polyprotein may be a non-proteolytic version. Providing both the P2 polyprotein and the VP0 polyprotein in one mRNA is convenient as it simplifies production of the immunogenic composition. For example, only a single mRNA needs to be manufactured by in vitro transcription. Similarly, where the mRNA is encapsulated in a lipid nanoparticle, only a single mRNA needs to be encapsulated during manufacturing.

In some embodiments, the fusion protein may comprise a secretion signal sequence as shown for the amino acids set forth in SEQ ID NOs: 30 and 32. Typically, the secretion signal sequence is located at the N-terminus of the fusion protein. Accordingly, in some embodiments, the fusion protein comprises the rhinovirus A VP0 polyprotein having the amino acid sequence of SEQ ID NO: 4 and the rhinovirus A P2 polyprotein having the amino acid sequence of SEQ ID NO: 6, or a non-proteolytic version thereof (e.g., SEQ ID NO: 77). In other embodiments, the rhinovirus A P2 polyprotein having the amino acid sequence of SEQ ID NO: 6, or a non-proteolytic version thereof (e.g., SEQ ID NO: 77) and the rhinovirus A VP0 polyprotein having the amino acid sequence of SEQ ID NO: 4.

In a particular embodiment, an immunogenic composition of the invention comprises a non-naturally occurring mRNA encoding a P2-VP0 fusion protein having the amino acid sequence of SEQ ID NO: 34. In another particular embodiment, an immunogenic composition of the invention comprises a non-naturally occurring mRNA encoding a P2-VP0 fusion protein with an N-terminal influenza virus A-derived secretion signal sequence having the amino acid sequence of SEQ ID NO: 35. In some embodiments, the exemplified fusion protein comprises a non-proteolytic version of P2.

In some embodiments, an immunogenic composition of the invention comprises a first non-naturally occurring mRNA encoding a fusion protein comprising a rhinovirus A serotype 21 P2 polyprotein having the amino acid sequence of SEQ ID NO: 6, or a non-proteolytic version thereof (e.g., SEQ ID NO: 77), and a rhinovirus A serotype 21 VP0 polyprotein having the amino acid sequence of SEQ ID NO: 4, and a second non-naturally occurring mRNA encoding a rhinovirus A serotype 8 P2 polyprotein having the amino acid sequence of SEQ ID NO: 80, or a non-proteolytic version thereof (e.g., SEQ ID NO: 81). In some embodiments, the fusion is P2-VP0 (i.e., P2 is at the N-terminus). In alternative embodiments, the fusion is VP0-P2 (i.e., VP0 is at the N-terminus).

In some embodiments, the fusion protein may comprise a secretion signal sequence as shown for the amino acids set forth in SEQ ID NOs: 30 and 32. Accordingly, in some embodiments, the immunogenic composition comprises a first non-naturally occurring mRNA encoding a fusion protein comprising a rhinovirus A serotype 21 P2 polyprotein having the amino acid sequence of SEQ ID NO: 30, or a non-proteolytic version thereof, and a rhinovirus A serotype 21 VP0 polyprotein having the amino acid sequence of SEQ ID NO: 4, and a second non-naturally occurring mRNA encoding a rhinovirus A serotype 8 P2 polyprotein having the amino acid sequence of SEQ ID NO: 80, or a non-proteolytic version thereof (e.g., SEQ ID NO: 81). In alternative embodiments, the immunogenic composition comprises a first non-naturally occurring mRNA encoding a fusion protein comprising a rhinovirus A serotype 21 VP0 polyprotein having the amino acid sequence of SEQ ID NO: 32, and a rhinovirus A serotype 21 P2 polyprotein having the amino acid sequence of SEQ ID NO: 6, or a non-proteolytic version thereof (e.g., SEQ ID NO: 77), and a second non-naturally occurring mRNA encoding a rhinovirus A serotype 8 P2 polyprotein having the amino acid sequence of SEQ ID NO: 80, or a non-proteolytic version thereof (e.g., SEQ ID NO: 81).

In a particular embodiment, an immunogenic composition of the invention comprises a first non-naturally occurring mRNA encoding a P2-VP0 fusion protein having the amino acid sequence of SEQ ID NO: 34, and a second non-naturally occurring mRNA encoding a rhinovirus A serotype 8 P2 polyprotein having the amino acid sequence of SEQ ID NO: 80, or a non-proteolytic version thereof (e.g., SEQ ID NO: 81). In some embodiments, the exemplified fusion protein comprises a non-proteolytic version of P2. In alternative embodiments, an immunogenic composition of the invention comprises a first non-naturally occurring mRNA encoding a P2-VP0 fusion protein having the amino acid sequence of SEQ ID NO: 35, and a second non-naturally occurring mRNA encoding a rhinovirus A serotype 8 P2 polyprotein having the amino acid sequence of SEQ ID NO: 80, or a non-proteolytic version thereof (e.g., SEQ ID NO: 81). In some embodiments, the exemplified fusion protein comprises a non-proteolytic version of P2.

### Exemplary Rhinovirus C Polyproteins

The inventors found that the amino acid sequence of the VP0 polyprotein of a human rhinovirus C serotype 34 having the amino acid sequence set forth in SEQ ID NO: 3, is most similar to a single VP0 consensus sequence created from 463 individual rhinovirus C polyprotein sequences. Without wishing to be bound by any particular theory, the inventors hypothesize that the VP0 polyprotein of this particular rhinovirus C serotype (or an mRNA encoding the same) can be used to construct immunogenic compositions that are effective at eliciting an immune response against multiple rhinovirus C serotypes.

Accordingly, in specific embodiments, an immunogenic composition of the invention comprises a non-naturally occurring mRNA encoding a rhinovirus C VP0 polyprotein having the amino acid sequence of SEQ ID NO: 3.

The inventors' analysis revealed that the group C rhinoviruses can be divided into two large phylogenetic clusters 1ab and 2ab, which each can be split further into two phylogenetic clusters 1a and 1b and 2a and 2b, respectively. For each of the two larger phylogenetic clusters, separate consensus sequences were determined in silico. The inventors also found that the amino acid sequence of the VP0 polyprotein of a human rhinovirus C serotype 17 having the amino acid sequence set forth in SEQ ID NO: 2, is most similar to the VP0 consensus sequence determined for the phylogenetic cluster 1ab. The amino acid sequence of the VP0 polyprotein of a human rhinovirus C serotype 11 having the amino acid sequence set forth in SEQ ID NO: 1, is most similar to the VP0 consensus sequence determined for the phylogenetic cluster 2ab. Without wishing to be bound by any particular theory, the inventors hypothesize that the VP0 polyproteins of these two rhinovirus C serotypes can also be used to construct immunogenic compositions that are effective at eliciting an immune response against multiple rhinovirus C serotypes.

Accordingly, in specific embodiments, an immunogenic composition of the invention comprises one or more non-naturally occurring mRNAs encoding a rhinovirus C VP0 polyprotein having the amino acid sequences of SEQ ID NO: 2 and a rhinovirus C VP0 polyprotein having the amino acid sequences of SEQ ID NO: 1.

In view of the phylogenetic divergence of the group C rhinoviruses, non-structural rhinovirus C polypeptide sequences may be used to construct immunogenic compositions capable of eliciting an effective immune response against as many rhinovirus C serotypes as possible. The inventors found that the amino acid sequence of the P2 polyprotein of a human rhinovirus C serotype 17 having the amino acid sequence set forth in SEQ ID NO: 9, is most similar to the P2 consensus sequence determined for the phylogenetic cluster 1ab. The amino acid sequence of the P2 polyprotein of a human rhinovirus C serotype 11 having the amino acid sequence set forth in SEQ ID NO: 8, is most similar to the P2 consensus sequence determined for the phylogenetic cluster 2ab.

Accordingly, in specific embodiments, an immunogenic composition of the invention comprises one or more non-naturally occurring mRNAs encoding a rhinovirus C P2 polyprotein having the amino acid sequence of SEQ ID NO: 9, or a non-proteolytic version thereof and a rhinovirus C P2 polyprotein having the amino acid sequence of SEQ ID NO: 8, or a non-proteolytic version thereof.

It can be expected that, together, the identified VP0 polyproteins and P2 polyproteins may be even more effective in eliciting a comprehensive immune response against group C rhinoviruses. Therefore, in one embodiment, an immunogenic composition of the invention comprises a non-naturally occurring mRNA encoding a VP0 polyprotein having the amino acid sequence of SEQ ID NO: 3, a non-naturally occurring mRNA encoding a P2 polyprotein having the amino acid sequence of SEQ ID NO: 9, or a non-proteolytic version thereof and a non-naturally occurring mRNA encoding a P2 polyprotein having the amino acid sequence of SEQ ID NO: 8, or a non-proteolytic version thereof. In another embodiment, an immunogenic composition of the invention comprises a non-naturally occurring mRNA encoding a rhinovirus C VP0 polyprotein having the amino acid sequences of SEQ ID NO: 2, a non-naturally occurring mRNA encoding a rhinovirus C VP0 polyprotein having the amino acid sequences of 1, a non-naturally occurring mRNA encoding a rhinovirus C P2 polyprotein having the amino acid sequences of SEQ ID NO: 9, or a non-proteolytic version thereof and a non-naturally occurring mRNA encoding a rhinovirus C P2 polyprotein having the amino acid sequences of 8, or a non-proteolytic version thereof.

In some embodiments, the rhinovirus C VP0 polyprotein and/or the rhinovirus C P2 polyprotein are operationally linked to a non-native secretion signal sequence from an influenza A virus to increase secretion of the mRNA encoded protein(s). Accordingly, in specific embodiments, an immunogenic composition of the invention comprises a non-naturally occurring mRNA encoding a rhinovirus C P2 polyprotein having the amino acid sequence of SEQ ID NO: 36, or a non-proteolytic version thereof. In another specific embodiment, an immunogenic composition of the invention comprises a non-naturally occurring mRNA encoding a rhinovirus C P2 polyprotein having the amino acid sequence of SEQ ID NO: 37. In another specific embodiment, an immunogenic composition of the invention comprises a non-naturally occurring mRNA encoding a rhinovirus C VP0 polyprotein having the amino acid sequence of SEQ ID NO: 38. In another specific embodiment, an immunogenic composition of the invention comprises a non-naturally occurring mRNA encoding a rhinovirus C VP0 polyprotein having the amino acid sequence of SEQ ID NO: 39. In another specific embodiment, an immunogenic composition of the invention comprises a non-naturally occurring mRNA encoding a rhinovirus C VP0 polyprotein having the amino acid sequence of SEQ ID NO: 40.

Therefore, in a further specific embodiment, an immunogenic composition of the invention comprises a non-naturally occurring mRNA encoding a rhinovirus C P2 polyprotein having the amino acid sequence of SEQ ID NOs: 8, 9, 36 or 37, or a non-proteolytic version thereof and a non-naturally occurring mRNA encoding a rhinovirus C VP0 polyprotein having the amino acid sequence of SEQ ID NOs: 1, 2, 3, 38, 39 or 40.

In Table 3, rhinovirus C VP0 polypeptides are underlined and rhinovirus C P2 polypeptides are in bold. Secretion signal sequences are shown in italics. The VP0 polyprotein comprising the influenza virus A-derived secretion signal sequence comprises the sequence DTL between the secretion signal and the VP0 polyprotein. DTL corresponds to the first three amino acids at the N-terminus of the mature influenza HA polypeptide, from which the exemplary secretion signal was derived.

TABLE 3
Exemplary amino acid sequences encoding native
rhinovirus C proteins or polyproteins

| | | SEQ | |
|---|---|---|---|
| | | ID | |
| Serotype | Name | NO | Sequence |
| C34 | VPO | 3 | MGAQVSKQNVGSHESGISASSGSVIKYFNI |
| | | | NYYKDSASSGLSKQDESMDPEKFTKPIAET |
| | | | LTNPALMSPSIEACGFSDRLKQITIGNSTI |
| | | | TTQDALNTVVAYGEWPQYLSDMDASAIDKP |
| | | | THPETSTDRFYTLTSVIWDTTSKGWWWKIP |
| | | | DCLKEMGMFGQNMYHHALGRSGFIIHVQCN |
| | | | ATKFHSGLLIVAVVPEHQLAYIGGTNVSVG |
| | | | YNHTHPGENGHTIGLNDQRGDRQPDEDPFF |
| | | | NCNGTLLGNLTIFPHQLINLRTNNSATIVV |
| | | | PYINCVPMDSMLRHNNLSLVIIPMVDLRFG |
| | | | TTGVTTLPITISIAPVKSEFSGARQSRTQ |
| C17 | VPO | 2 | MGAQVSRQTTGSHESAVNATNGGIIKYFNI |
| | | | NYYRDSASSGLTKQDFSQDPSKFTQPLVDT |
| | | | LTNPALMSPSVEACGFSDRLKQITMGNSTI |
| | | | TTQDALHTVLAYGEWPQYLSDLDATSVDKP |
| | | | THPETSSDRFYTLSSVSWTNTSKGWWWKLP |
| | | | DALKDMGVFGQNLYYHAMGRAGYIIHTQCN |
| | | | ATKFHSGALLVVLIPEHQLAYIGAEKVNIA |
| | | | YDLTHPGETGHVIGRNTSRGNNNPDEDPFF |
| | | | NCNGTLFGNLTIFPHQIINLRTNNSSTIIT |
| | | | PYINCQPMDNMLKHNNLTLLIVPLVRLRFG |
| | | | TEASPTVSITVTIAPYKSEFSGAMETQKHQ |
| C11 | VPO | 1 | MGAQVSKQNVGSHESGISASSGSVIKYFNI |
| | | | NYYKDSASSGLSKQDFSMDPEKFTKPLADV |
| | | | MTNPALMSPSIEACGESDRLKQITIGSSTI |
| | | | TTQDTLNTVVAYGEWPEYLRDTDASAVDKP |
| | | | THPETSTDRFYTLTSVIWNGSSKGWWWKIP |
| | | | DCLKDMGMFGQNMYHHALGRSGYIFHIQCN |
| | | | ATKFHSGLLLVAIVPEHQLAYVGGTYANVG |
| | | | YNHTHPGEGGHEIREPTGRDDKKPDEDPLF |
| | | | NCNGTLLGNLTIFPHQLINLRTNNSVTIVV |
| | | | PYINCVPMDSMLKHNNISLVIIPLVPLRSG |
| | | | STQAPQTLPITISIAPDKSEFSGARQSNKT |
| | | | Q |
| C17 | P2 | 9 | MGPSDQCVHTKDAIYTCAHLTEPNSNTILL |
| | | | AITADLQVDSTDTPGPDFIPTCDCVQACYY |
| | | | AKHAQRYYPITVTPHDWYEIQESQYYPKHI |
| | | | QYNILIGEGPCEPGDCGGKLLCRHGVIGII |
| | | | TAGGDGHVAFTDLRPYACLSHQGLVSDYVN |
| | | | QLGAAFGDGFSSNIKDHLTGLCTTVSDKIT |
| | | | GKVIKWLVRVISALTIMIRNSSDTATVLAT |
| | | | LALLGCHGSPWSFLKEKICQWLGIPRPPTR |
| | | | QGESWLKKFTECCNAAKGLEWVAQKIGKFI |
| | | | DWLKEKLIPTVQRKKETLDQCKKIGLYEEQ |
| | | | TKGFSHSEAEAQQSLILEVAKLKRGLDDLA |
| | | | PLYASENKRVTIIQKELQRLSAYQKTHRHE |
| | | | PVCCLLRGPPGCGKSLVTSIIAHGLTNEAN |
| | | | IYSLPPDPKHFDGYNQQTVVIMDDVGQNPD |
| | | | GKDLSMFCQMVSTTEFIVPMASIEDKGRAF |
| | | | TSQYVLASTNLDSLSPPTVTIPEAISRRFY |
| | | | LDADLQVTSKFKAHNGLLDVAKALQPCAKC |
| | | | PKPNHYKQCCPILCGQALVLRDRRTSANYP |
| | | | LLAVVEQLRMENNTRDKVKSNLRAIFQ |
| C11 | P2 | 8 | MGPSDMYVHTKEAIYKNAHLTSANEQTILI |
| | | | ALTADLQVDAADHPGDDVIPDCDCTTGTYY |
| | | | CKSKDRYYPVEVVSHAWYPIEETCYYPKHI |
| | | | QYNILLGEGPCVPGDCGGKLLCKHGVIGIV |
| | | | TAGGENHVAFTDLRPYSNLAHTQGPISDYV |
| | | | TQLGNAFGTGFTQTLETNLRETCSGMFDAI |
| | | | TSKTVKWVVRIISALTIVIRNSSDIPTILA |
| | | | TMALLGCTGSPWQYLKSKLCNWLGVQKPPS |
| | | | KQSDSWLKKFTEWCNAAKGLEWIGYKISKF |
| | | | IDWLKEKLIPTVQRKKDTLLECKKLTLYED |
| | | | QVRAFPQSPEAFQNELTTKLQILKKNLDDM |
| | | | CPLYAAENKRVTNMLRDIKTMTAYKKTHRT |
| | | | EPVCVLIHGGPGCGKSLATTVIARGLTDSG |
| | | | NVYSLPPSPKHFDGYCQQQVVMMDDLGQNP |
| | | | DGQDLAMFCQMVSTTDFIVPMAALEDKGKS |
| | | | FTSDFVLASTNLNQLSPPTVTIPEAITRRF |
| | | | FLDVDLKIMSGYRTHAGLLDTAKALQACPD |
| | | | CAKPPYYKQCCPLLCGKAVVLQNRRTSASL |
| | | | SLNMVVSQLREESNTRKRIHTNLNAIFQ |
| C34 | VPO | 38 | MKAILVVLLYTFATANADTLGAQVSKQNVG |
| | comprising a | | SHESGISASSGSVIKYFNINYYKDSASSGL |
| | secretion | | SKQDESMDPEKFTKPIAETLTNPALMSPSI |
| | signal | | EACGFSDRLKQITIGNSTITTQDALNTVVA |
| | sequence | | YGEWPQYLSDMDASAIDKPTHPETSTDRFY |
| | | | TLTSVIWDTTSKGWWWKIPDCLKEMGMFGQ |
| | | | NMYHHALGRSGFIIHVQCNATKFHSGLLIV |
| | | | AVVPEHQLAYIGGTNVSVGYNHTHPGENGH |
| | | | TIGLNDQRGDRQPDEDPFFNCNGTLLGNLT |
| | | | IFPHQLINLRTNNSATIVVPYINCVPMDSM |
| | | | LRHNNLSLVIIPMVDLRFGTTGVTTLPITI |
| | | | SIAPVKSEFSGARQSRTQ |
| C17 | VPO | 39 | MKAILVVLLYTFATANADTLGAQVSRQTTG |
| | comprising | | SHESAVNATNGGIIKYFNINYYRDSASSGL |
| | a secretion | | TKQDESQDPSKFTQPLVDTLTNPALMSPSV |
| | signal | | EACGESDRLKQITMGNSTITTQDALHTVLA |
| | sequence | | YGEWPQYLSDLDATSVDKPTHPETSSDRFY |
| | | | TLSSVSWTNTSKGWWWKLPDALKDMGVFGQ |
| | | | NLYYHAMGRAGYIIHTQCNATKFHSGALLV |
| | | | VLIPEHQLAYIGAEKVNIAYDLTHPGETGH |
| | | | VIGRNTSRGNNNPDEDPFFNCNGTLFGNLT |
| | | | IFPHQIINLRTNNSSTIITPYINCQPMDNM |
| | | | LKHNNLTLLIVPLVRLRFGTEASPTVSITV |
| | | | TIAPYKSEFSGAMETQKHQ |
| C11 | VPO comprising | 40 | MKAILVVLLYTFATANADTLGAQVSKQNVG |
| | a secretion | | SHESGISASSGSVIKYFNINYYKDSASSGL |
| | signal | | SKQDFSMDPEKFTKPLADVMTNPALMSPSI |
| | sequence | | EACGESDRLKQITIGSSTITTQDTLNTVVA |
| | | | YGEWPEYLRDTDASAVDKPTHPETSTDRFY |
| | | | TLTSVIWNGSSKGWWWKIPDCLKDMGMFGQ |
| | | | NMYHHALGRSGYIFHIQCNATKFHSGLLLV |
| | | | AIVPEHQLAYVGGTYANVGYNHTHPGEGGH |
| | | | EIREPTGRDDKKPDEDPLFNCNGTLLGNLT |
| | | | IFPHQLINLRTNNSVTIVVPYINCVPMDSM |
| | | | LKHNNISLVIIPLVPLRSGSTQAPQTLPIT |
| | | | ISIAPDKSEFSGARQSNKTQ |
| C17 | P2 comprising | 36 | MKAILVVLLYTFATANAGPSDQCVHTKDAI |
| | a secretion | | YTCAHLTEPNSNTILLAITADLQVDSTDTP |
| | signal | | GPDFIPTCDCVQACYYAKHAQRYYPITVTP |
| | sequence | | HDWYEIQESQYYPKHIQYNILIGEGPCEPG |
| | | | DCGGKLLCRHGVIGIITAGGDGHVAFTDLR |
| | | | PYACLSTHQGLVSDYVNQLGAAFGDGFSSN |
| | | | IKDHLTGLCTTVSDKITGKVIKWLVRVISA |
| | | | LTIMIRNSSDTATVLATLALLGCHGSPWSF |
| | | | LKEKICQWLGIPRPPTRQGESWLKKFTECC |
| | | | NAAKGLEWVAQKIGKFIDWLKEKLIPTVQR |
| | | | KKETLDQCKKIGLYEEQTKGFSHSEAEAQQ |
| | | | SLILEVAKLKRGLDDLAPLYASENKRVTII |
| | | | QKELQRLSAYQKTHRHEPVCCLLRGPPGCG |
| | | | KSLVTSIIAHGLTNEANIYSLPPDPKHFDG |
| | | | YNQQTVVIMDDVGQNPDGKDLSMFCQMVST |
| | | | TEFIVPMASIEDKGRAFTSQYVLASTNLDS |
| | | | LSPPTVTIPEAISRRFYLDADLQVTSKFKA |
| | | | HNGLLDVAKALQPCAKCPKPNHYKQCCPIL |
| | | | CGQALVLRDRRTSANYPLLAVVEQLRMENN |
| | | | TRDKVKSNLRAIFQ |
| C11 | P2 comprising | 37 | MKAILVVLLYTFATANAGPSDMYVHTKEAI |
| | a secretion | | YKNAHLTSANEQTILIALTADLQVDAADHP |
| | signal | | GDDVIPDCDCTTGTYYCKSKDRYYPVEVVS |
| | sequence | | HAWYPIEETCYYPKHIQYNILLGEGPCVPG |
| | | | DCGGKLLCKHGVIGIVTAGGENHVAFTDLR |
| | | | PYSNLAHTQGPISDYVTQLGNAFGTGFTQT |
| | | | LETNLRETCSGMFDAITSKTVKWVVRIISA |
| | | | LTIVIRNSSDIPTILATMALLGCTGSPWQY |
| | | | LKSKLCNWLGVQKPPSKQSDSWLKKFTEWC |
| | | | NAAKGLEWIGYKISKFIDWLKEKLIPTVQR |
| | | | KKDTLLECKKLTLYEDQVRAFPQSPEAFQN |
| | | | ELTTKLQILKKNLDDMCPLYAAENKRVTNM |
| | | | LRDIKTMTAYKKTHRTEPVCVLIHGGPGCG |
| | | | KSLATTVIARGLTDSGNVYSLPPSPKHFDG |
| | | | YCQQQVVMMDDLGQNPDGQDLAMFCQMVST |
| | | | TDFIVPMAALEDKGKSFTSDFVLASTNLNQ |
| | | | LSPPTVTIPEAITRRFFLDVDLKIMSGYRT |
| | | | HAGLLDTAKALQACPDCAKPPYYKQCCPLL |
| | | | CGKAVVLQNRRTSASLSLNMVVSQLREESN |
| | | | TRKRIHTNLNAIFQ |

The active site of P2A in rhinovirus is highly conserved. In rhinovirus C, the active site consists of a catalytic triad of a cysteine (C) residue at position 105 (Cys105), a histidine (H) residue at position 18 (His18) and an aspartic acid (D) residue at position 34 (Asp34). A non-proteolytic version of P2A can be generated by mutating one or more of these sites, e.g., Cys105.

In some embodiments, an immunogenic composition of the invention comprises a non-naturally occurring mRNA encoding a fusion protein comprising a P2 polyprotein having the amino acid sequence of SEQ ID NOs: 8 or 9, or a non-proteolytic version thereof and a VP0 polyprotein having the amino acid sequence of SEQ ID NOs: 1, 2 or 3. In some embodiments, the fusion is P2-VP0 (i.e. P2 is at the N-terminus). In alternative embodiments, the fusion is VP0-P2 (i.e. VP0 is at the N-terminus). In either arrangement, the rhinovirus A P2 polyprotein may be a non-proteolytic version. Providing both the P2 polyprotein and the VP0 polyprotein in one mRNA is convenient as it simplifies production of the immunogenic composition. For example, only a single mRNA needs to be manufactured by in vitro transcription. Similarly, where the mRNA is encapsulated in a lipid nanoparticle, only a single mRNA needs to be encapsulated during manufacturing.

In some embodiments, the fusion protein may comprise a secretion signal sequence as shown for the amino acids set forth in SEQ ID NOs: 36, 37, 38, 39 and 40. Typically, the secretion signal sequence is located at the N-terminus of the fusion protein. Accordingly, in some embodiments, the fusion protein comprises the rhinovirus C VP0 polyprotein having the amino acid sequence of SEQ ID NOs: 38, 39 or 40 and the rhinovirus C P2 polyprotein having the amino acid sequence of SEQ ID NOs: 36 or 37, or a non-proteolytic version thereof. In other embodiments, the fusion protein comprises the rhinovirus C P2 polyprotein having the amino acid sequence of SEQ ID NOs: 36 or 37, or a non-proteolytic version thereof and the rhinovirus C VP0 polyprotein having the amino acid sequence of SEQ ID NOs: 38, 39 or 40.

In a particular embodiment, an immunogenic composition of the invention comprises a non-naturally occurring mRNA encoding a P2-VP0 fusion protein having the amino acid sequence of SEQ ID NO: 41. In another particular embodiment, an immunogenic composition of the invention comprises an mRNA encoding a P2-VP0 fusion protein with an N-terminal influenza virus A-derived secretion signal sequence having the amino acid sequence of SEQ ID NO: 42). In some embodiments, the exemplified fusion protein comprises a non-proteolytic version of P2. In a particular embodiment, an immunogenic composition of the invention comprises a non-naturally occurring mRNA encoding a P2-VP0 fusion protein having the amino acid sequence of SEQ ID NO: 43. In another particular embodiment, an immunogenic composition of the invention comprises a non-naturally occurring mRNA encoding a P2-VP0 fusion protein with an N-terminal influenza virus A-derived secretion signal sequence having the amino acid sequence of SEQ ID NO: 44.

### Coverage

In order to provide immunity against infection caused by multiple rhinovirus serotypes, the immunogenic composition may include one or more non-naturally occurring mRNAs encoding a first naturally occurring rhinovirus polyprotein and one or more non-naturally occurring mRNAs encoding a second naturally occurring rhinovirus polyprotein.

For example, the first naturally occurring rhinovirus polyprotein may be a structural rhinovirus polyprotein (e.g., VP0), and the second naturally occurring rhinovirus polyprotein may be a non-structural rhinovirus polyprotein (e.g., P2). In some embodiments, the structural rhinovirus polyprotein and the non-structural rhinovirus polyprotein are from the same group of rhinoviruses (e.g., group A or group C). In some embodiments, the structural rhinovirus polyprotein and the non-structural rhinovirus polyprotein are from the same rhinoviruses (i.e., the same strain). In some embodiments, the structural rhinovirus polyprotein and the non-structural rhinovirus polyprotein are from the different rhinoviruses (i.e., different strains). In some embodiments, the structural rhinovirus polyprotein and the non-structural rhinovirus polyprotein are from different groups of rhinoviruses (e.g., group A and group C).

In some embodiments, the first naturally occurring rhinovirus polyprotein is a structural rhinovirus polyprotein (e.g., VP0) from a first rhinovirus, and the second naturally occurring rhinovirus polyprotein is a structural rhinovirus polyprotein (e.g., VP0) from a second rhinovirus. The first and second rhinoviruses may be from the same group of rhinoviruses (e.g., group A or group C). For example, first structural polyprotein (e.g., VP0) may be from a first rhinovirus A, and the second structural polyprotein (e.g., VP0) may be from a second rhinovirus A. In some embodiments, first structural polyprotein (e.g., VP0) may be from a first rhinovirus C, and the second structural polyprotein (e.g., VP0) may be from a second rhinovirus C. In some embodiments, the first and second rhinoviruses are from different groups of rhinoviruses (e.g., group A and group C). In some embodiments, the first naturally occurring rhinovirus polyprotein is a non-structural rhinovirus polyprotein (e.g., P2) from a first rhinovirus, and the second naturally occurring rhinovirus polyprotein is a non-structural rhinovirus polyprotein (e.g., P2) from a second rhinovirus. The first and second rhinoviruses may be from the same group of rhinoviruses (e.g., group A or group C). For example, first non-structural polyprotein (e.g., P2) may be from a first rhinovirus A, and the second non-structural polyprotein (e.g., P2) may be from a second rhinovirus A. In some embodiments, first non-structural polyprotein (e.g., P2) may be from a first rhinovirus C, and the second non-structural polyprotein (e.g., P2) may be from a second rhinovirus C. In some embodiments, the first and second rhinoviruses are from different groups of rhinoviruses (e.g., group A and group C).

In some embodiments, an immunogenic composition of the invention comprises a first non-naturally occurring mRNA encoding a rhinovirus A VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus A, and a second non-naturally occurring mRNA encoding a rhinovirus C VP0 polyprotein, wherein the rhinovirus C VP0 polyprotein has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C.

In some embodiments, an immunogenic composition of the invention comprises a non-naturally occurring mRNA comprising a first nucleic acid sequence encoding a rhinovirus A VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus A, and a second nucleic acid sequence encoding a rhinovirus C VP0 polyprotein, wherein the rhinovirus C VP0 polyprotein has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C.

In some embodiments, an immunogenic composition of the invention comprises a first non-naturally occurring mRNA encoding a first rhinovirus C VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C, and a second non-naturally occurring mRNA encoding a second rhinovirus C VP0 polyprotein, wherein the second rhinovirus C VP0 polyprotein is different from the first rhinovirus C VP0 polyprotein and has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C, wherein the two phylogenetic clusters comprising VP0 polyproteins with amino acid sequences having an average identity (and optionally a median identity) of at least 80% (e.g., at least 85%, at least 90%, or at least 95%) to the amino acid sequence of the second rhinovirus C VP0 polyprotein are different from the two phylogenetic clusters comprising VP0 polyproteins with amino acid sequences having an average identity (and optionally a median identity) of at least 80% (e.g., at least 85%, at least 90%, or at least 95%) to the amino acid sequence of the first rhinovirus C VP0 polyprotein.

In some embodiments, an immunogenic composition of the invention comprises a non-naturally occurring mRNA comprising a first nucleic acid sequence encoding a first rhinovirus C VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C, and a second nucleic acid sequence encoding a second rhinovirus C VP0 polyprotein, wherein the second rhinovirus C VP0 polyprotein is different from the first rhinovirus C VP0 polyprotein and has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C, wherein the two phylogenetic clusters comprising VP0 polyproteins with amino acid sequences having an average identity (and optionally a median identity) of at least 80% (e.g., at least 85%, at least 90%, or at least 95%) to the amino acid sequence of the second rhinovirus C VP0 polyprotein are different from the two phylogenetic clusters comprising VP0 polyproteins with amino acid sequences having an average identity (and optionally a median identity) of at least 80% (e.g., at least 85%, at least 90%, or at least 95%) to the amino acid sequence of the first rhinovirus C VP0 polyprotein.

In some embodiments, an immunogenic composition of the invention comprises a first non-naturally occurring mRNA encoding a rhinovirus A VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus A, and a second non-naturally occurring mRNA encoding a rhinovirus A P2 polyprotein that has an amino acid sequence with an average identity (and optionally a median identity) of at least 80% (e.g., at least 85%, at least 90%, or at least 95%) to the amino acid sequences of P2 polyproteins from at least two phylogenetic clusters of rhinovirus A.

In some embodiments, an immunogenic composition of the invention comprises a non-naturally occurring mRNA comprising a first nucleic acid sequence encoding a rhinovirus A VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus A, and a second nucleic acid sequence encoding a rhinovirus A P2 polyprotein that has an amino acid sequence with an average identity (and optionally a median identity) of at least 80% (e.g., at least 85%, at least 90%, or at least 95%) to the amino acid sequences of P2 polyproteins from at least two phylogenetic clusters of rhinovirus A.

In some embodiments, an immunogenic composition of the invention comprises a first non-naturally occurring mRNA encoding a rhinovirus C VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C, and a second non-naturally occurring mRNA encoding a rhinovirus C P2 polyprotein that has an amino acid sequence with an average identity (and optionally a median identity) of at least 80% (e.g., at least 85%, at least 90%, or at least 95%) to the amino acid sequences of P2 polyproteins from at least two phylogenetic clusters of rhinovirus C.

In some embodiments, an immunogenic composition of the invention comprises a non-naturally occurring mRNA comprising a first nucleic acid sequence encoding a rhinovirus C VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C, and a second nucleic acid sequence encoding a rhinovirus C P2 polyprotein that has an amino acid sequence with an average identity (and optionally a median identity) of at least 80% (e.g., at least 85%, at least 90%, or at least 95%) to the amino acid sequences of P2 polyproteins from at least two phylogenetic clusters of rhinovirus C.

In some embodiments, an immunogenic composition of the invention comprises a first non-naturally occurring mRNA encoding a rhinovirus C VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C, a second non-naturally occurring mRNA encoding a first rhinovirus C P2 polyprotein that has an amino acid sequence with an average identity (and optionally a median identity) of at least 80% (e.g., at least 85%, at least 90%, or at least 95%) to the amino acid sequences of P2 polyproteins from at least two phylogenetic clusters of rhinovirus C, and a third non-naturally occurring mRNA encoding a second rhinovirus C P2 polyprotein that has an amino acid sequence with an average identity (and optionally a median identity) of at least 80% (e.g., at least 85%, at least 90%, or at least 95%) to the amino acid sequences of P2 polyproteins from at least two phylogenetic clusters of rhinovirus C, wherein the two phylogenetic clusters comprising P2 polyproteins with amino acid sequences having an average identity (and optionally a median identity) of at least 80% (e.g., at least 85%, at least 90%, or at least 95%) to the amino acid sequence of the second rhinovirus C P2 polyprotein are different from the two phylogenetic clusters comprising P2 polyproteins with amino acid sequences having an average identity (and optionally a median identity) of at least 80% (e.g., at least 85%, at least 90%, or at least 95%) sequences to the amino acid sequence of the first rhinovirus C P2 polyprotein.

In some embodiments, an immunogenic composition of the invention comprises a non-naturally occurring mRNA comprising a first nucleic acid sequence encoding a rhinovirus C VP0 polyprotein that has an amino acid sequence with an average identity (and optionally a median identity) of at least 80% (e.g., at least 85%, at least 90%, or at least 95%) to the amino acid sequences of VP0 polyproteins from at least two phylogenetic clusters of rhinovirus C, a second nucleic acid sequence encoding a first rhinovirus C P2 polyprotein that has an amino acid sequence with an average identity (and optionally a median identity) of at least 80% (e.g., at least 85%, at least 90%, or at least 95%) to the amino acid sequences of P2 polyproteins from at least two phylogenetic clusters of rhinovirus C, and a third nucleic acid sequence encoding a second rhinovirus C P2 polyprotein that has an amino acid sequence with an average identity (and optionally a median identity) of at least 80% (e.g., at least 85%, at least 90%, or at least 95%) to the amino acid sequences of P2 polyproteins from at least two phylogenetic clusters of rhinovirus C, wherein the two phylogenetic clusters comprising P2 polyproteins with amino acid sequences having an average identity (and optionally a median identity) of at least 80% (e.g., at least 85%, at least 90%, or at least 95%) to the amino acid sequence of the second rhinovirus C P2 polyprotein are different from the two phylogenetic clusters comprising P2 polyproteins with amino acid sequences having an average identity (and optionally a median identity) of at least 80% (e.g., at least 85%, at least 90%, or at least 95%) sequences to the amino acid sequence of the first rhinovirus C P2 polyprotein.

### Rhinovirus Polypeptides Comprising T-Cell Epitope-Rich Regions

Up to a third of all rhinovirus infections are asymptomatic, both in healthy subjects and in subjects suffering from asthma. Among subjects suffering from asthma, symptomatic infections can lead to exacerbations of asthma symptoms and are thought to cause 20 to 30% of asthma exacerbations. Previous research suggests that CD4+ and CD8+ T-cells capable of recognizing rhinovirus-associated antigens are present in the circulation of healthy subjects. These T-cells may be involved in immune surveillance and may rapidly induce an adaptive immune response after rhinovirus infection.

It has been hypothesized that the rapid adaptive immune responses results from an engagement of effector memory T-cells which are cross-reactive with T-cell epitopes conserved across multiple rhinoviruses. These memory T-cells were formed after a previous infection with a different rhinovirus strain.

The inventors demonstrate for the first time that the P2 polyprotein of rhinovirus A comprises conserved regions that are T-cell epitope-rich. Therefore, without wishing to be bound by any particular theory, the P2 polyprotein may be able to induce an immune response, and especially a T-cell response, that is broadly protective against infection by multiple rhinoviruses.

Specifically, the inventors found that non-structural rhinovirus polypeptides P2A, P2B and P2C, which make up the P2 polyprotein, comprise regions of high sequence conservation. Regions of high sequence conservation comprise one or more stretches of at least 30 contiguous amino acids with at least 80% sequence identity among at least 20 (e.g., at least 30, 40 or 50) rhinovirus serotypes. In some embodiments, regions of high sequence conservation comprise one or more stretches of at least 50 contiguous amino acids with at least 80% sequence identity among at least 20 (e.g., at least 30, 40 or 50) rhinovirus serotypes.

Based on the inventors' analysis, the P2 polyprotein of rhinovirus comprises regions with a high degree of sequence conservation across various serotypes, e.g., around residues 80-120, residues 250-280 and residues 380-450, respectively, of the rhinovirus A VP0 polyprotein. These conserved regions are also rich in T-cell epitopes. Therefore, without wishing to be bound by any particular theory, the rhinovirus A proteins P2A, P2B and P2C, or polypeptides derived therefrom comprising one or more of these conserved T-cell rich regions, may be particularly effective in inducing a T-cell response against multiple rhinovirus serotypes (especially multiple serotypes of rhinovirus A). For example, a non-structural rhinovirus polypeptide of the invention may comprise residues 80-120, residues 250-280 and residues 380-450 of a rhinovirus A P2 polyprotein (e.g., of the P2 polyprotein encoded by SEQ ID NO: 6).

Based on the inventors' analysis, the VP0 polyprotein of rhinovirus also comprises regions with a high degree of sequence conservation across various serotypes, e.g., around residues 1-200 and residues 220-300, respectively, of the rhinovirus A VP0 polyprotein. These conserved regions are also rich in T-cell epitopes. Therefore, without wishing to be bound by any particular theory, the VP0 polyprotein may also be able to induce an immune response, and especially a T-cell response, that is broadly protective against infection by multiple rhinoviruses.

Specifically, the structural rhinovirus polypeptides, in particular VP4 and the N-terminal 30 residues of VP2 of the rhinovirus A VP0 polyprotein, show especially high sequence conservation. Moreover, residues 1-100 of VP0 which comprises part of the structural capsid polypeptides, are also rich in T-cell epitopes. In addition, the inventors identified the region comprising residues 150-200 and, particularly, the region comprising residues 220-300, as rich in T-cell epitopes. Therefore, without wishing to be bound by any particular theory, rhinovirus proteins VP4 and VP2, or polypeptides derived therefrom comprising one or more of these T-cell rich regions, may be particularly effective in inducing a T-cell response against multiple rhinovirus serotypes (especially multiple serotypes of rhinovirus A). For example, a structural rhinovirus polypeptide of the invention may comprise residues 1-200 and residues 220-300 of a rhinovirus A VP0 polyprotein (e.g., of the VP0 polyprotein encoded by SEQ ID NO: 4).

The inventors used a computational approach to identify conserved regions of a complete rhinovirus polyprotein and to predict both MHC class-I and class-II T-cell epitopes. Using this approach, the inventors discovered several conserved regions comprising stretches of at least 30 contiguous amino acids that had at least 80% sequence identity among rhinoviruses belonging to the same group. In fact, in some instances, the conserved stretches with at least 80% sequence identity were longer (e.g., about 50 amino acids long or about 100 amino acids long). The inventors also identified stretches of at least 40 contiguous amino acids within these conserved regions that had 90% sequence identity among rhinovirus of the same group. Within some conserved regions, shorter stretches of at least 30 contiguous amino acids within these conserved regions had at least 95% sequence identity among rhinovirus of the same group.

Notably, the inventors found that some of the conserved T-cell epitope-rich regions were located in non-structural polypeptides, particularly within the precursor P2 polyprotein). To the inventors' knowledge, these non-structural polypeptides comprising one or more T-cell epitope-rich regions, especially, 2A, 2B and 2C provided as a polyprotein, have not been previously used in vaccination approaches against rhinovirus. Without wishing to be bound by any particular theory, the inventors believe that these non-structural polypeptides may be particularly effective in eliciting an immune response against multiple rhinovirus serotypes, and potentially rhinoviruses from different groups, because they are not exposed to the same evolutionary pressures as the structural polypeptides that form the rhinovirus capsid and that have likely resulted in the many known rhinovirus serotypes.

In addition, the inventors found that the structural polypeptides located within the VP0 region of the complete rhinovirus polyprotein (namely VP4 and VP2) are particularly plentiful in conserved T-cell epitope-rich regions. Accordingly, including these structural polypeptides in immunogenic compositions is expected to yield a particularly potent T-cell response against multiple rhinovirus serotypes. Indeed, the inventors' computational analysis suggests that a combination of P2 and VP0 polyproteins can elicit a T-cell response in at least 95% of the human population.

Without wishing to be bound by any particular theory, the inventors believe that one or more mRNAs encoding one or more non-structural rhinovirus polypeptides comprising one or more T-cell epitope-rich regions, either alone or in combination with one or more mRNAs encoding one or more structural rhinovirus polypeptides comprising one or more T-cell epitope-rich regions, provide particularly effective immunogenic compositions because the one or more mRNAs are expressed within cells and therefore can mimic virus-infected cells in a subject in vivo. Accordingly, in particular embodiments, the invention is specifically directed to an immunogenic composition comprising one or more mRNAs encoding one or more non-structural rhinovirus polypeptides comprising one or more T-cell epitope-rich regions. In typical embodiments, the one or more T-cell epitope-rich region(s) of the one or more non-structural rhinovirus polypeptides comprise(s) class-I T-cell epitopes and/or class-II T-cell epitopes.

In specific embodiments, the one or more non-naturally occurring mRNAs encoding the one or more non-structural rhinovirus polypeptides encodes a first naturally occurring rhinovirus protein or polyprotein. In particular embodiments, the first naturally occurring rhinovirus protein or polyprotein comprises at least one of the rhinovirus proteins 2A, 2B and 2C. In some embodiments, the first naturally occurring rhinovirus protein or polyprotein is a polyprotein comprising rhinovirus proteins 2A, 2B and 2C.

In some embodiments, the one or more T-cell epitopes of one or more non-structural rhinovirus polypeptides are located in conserved regions comprising one or more stretches of at least 30 contiguous amino acids with at least 80% sequence identity to corresponding stretches of the rhinovirus A serotype 21 polyprotein as set forth in SEQ ID NO: 45. In some embodiments, the one or more T-cell epitopes are located in conserved regions comprising one or more stretches of at least 50 contiguous amino acids with at least 80% sequence identity to corresponding stretches of the rhinovirus A serotype 21 polyprotein as set forth in SEQ ID NO: 45. In particular embodiments, the one or more T-cell epitopes are located within one or more regions corresponding to residues 80-120, 250-280 and 380-450, respectively of the rhinovirus A serotype 21 P2 polyprotein as set forth in SEQ ID NO: 6.

In some embodiments, the first protein or polyprotein has or comprises an amino acid sequence that is at least 80% identical to the amino acid sequence of the rhinovirus A serotype 21 P2 polyprotein as set forth in SEQ ID NO: 6. In some embodiments, the first protein or polyprotein has or comprises an amino acid sequence that is at least 85% identical to the amino acid sequence of the rhinovirus A serotype 21 P2 polyprotein set forth in SEQ ID NO: 6. In some embodiments, the first protein or polyprotein has or comprises an amino acid sequence that is at least 90% identical to the amino acid sequence of the rhinovirus A serotype 21 P2 polyprotein set forth in SEQ ID NO: 6. In some embodiments, the first protein or polyprotein has or comprises an amino acid sequence that is at least 95% identical to the amino acid sequence of the rhinovirus A serotype 21 P2 polyprotein set forth in SEQ ID NO: 6. In some embodiments, the first protein or polyprotein has or comprises an amino acid sequence that is at least 98% identical to the amino acid sequence of the rhinovirus A serotype 21 P2 polyprotein set forth in SEQ ID NO: 6. In some embodiments, the first protein or polyprotein has or comprises an amino acid sequence that is at least 99% identical to the amino acid sequence of the rhinovirus A serotype 21 P2 polyprotein set forth in SEQ ID NO: 6. In some embodiments, the first protein or polyprotein has or comprises an amino acid sequence that is identical to the amino acid sequence of the rhinovirus A serotype 21 P2 polyprotein set forth in SEQ ID NO: 6.

Many T-cell epitopes are located within structural rhinovirus polypeptides. Including one or more mRNAs encoding one or more structural rhinovirus polypeptides comprising one or more T-cell epitope-rich regions may render an immunogenic composition more effective in inducing a broad T-cell response against rhinovirus. Accordingly, in some embodiments, the one or more mRNAs further encode(s) one or more structural rhinovirus polypeptides comprising one or more T-cell epitope-rich regions. In some embodiments, the one or more T-cell epitope-rich regions of the one or more non-structural rhinovirus polypeptides comprises class-I T-cell epitopes and/or class-II T-cell epitopes.

In some embodiments, an immunogenic composition of the invention is capable of inducing a T_{H}1-directed T-cell response (e.g., T_{H}1-directed CD4+ T-cell response). In some embodiments, an immunogenic composition of the invention induces polyreactive T-cells (e.g., CD4+ T cells expressing IFN-γ, IL-2 and TNF-α). In particular embodiments, the T-cell response is cross-reactive against multiple rhinoviruses of the same group (e.g., group A or group C). In some embodiments, the immunogenic composition of the invention is capable of eliciting an effective T-cell response in the absence of an adjuvant.

In specific embodiments, the one or more non-naturally occurring mRNAs encoding the one or more structural rhinovirus polypeptides encodes a second naturally occurring rhinovirus protein or polyprotein. In particular embodiments, the second naturally occurring rhinovirus protein or polyprotein comprises at least one of the rhinovirus proteins VP4 and VP2. In some embodiments, the second naturally occurring rhinovirus protein or polyprotein is a polyprotein comprising rhinovirus proteins VP4 and VP2.

In some embodiments, the one or more T-cell epitopes of the one or more structural rhinovirus polypeptides are located in conserved regions comprising one or more stretches of at least 30 contiguous amino acids with at least 80% sequence identity to corresponding stretches of the rhinovirus A serotype 21 polyprotein as set forth in SEQ ID NO: 45. In some embodiments, the one or more T-cell epitopes are located in conserved regions comprising one or more stretches of at least 50 contiguous amino acids with at least 80% sequence identity to corresponding stretches of the rhinovirus A serotype 21 polyprotein as set forth in SEQ ID NO: 45. In particular embodiments, the one or more T-cell epitope-rich regions are located within one or more regions corresponding to residues 1-100, 150-200 and 220-300, respectively, of the rhinovirus A serotype 21 polyprotein VP0 as set forth in SEQ ID NO: 4.

In some embodiments, the second protein or polyprotein has or comprises an amino acid sequence that is at least 80% identical to the amino acid sequence of the rhinovirus A serotype 21 VP0 polyprotein as set forth in SEQ ID NO: 4. In some embodiments, the second protein or polyprotein has or comprises an amino acid sequence that is at least 85% identical to the amino acid sequence of the rhinovirus A serotype 21 VP0 polyprotein as set forth in SEQ ID NO: 4. In some embodiments, the second protein or polyprotein has or comprises an amino acid sequence that is at least 90% identical to the amino acid sequence of the rhinovirus A serotype 21 VP0 polyprotein as set forth in SEQ ID NO: 4. In some embodiments, the second protein or polyprotein has or comprises an amino acid sequence that is at least 95% identical to the amino acid sequence of the rhinovirus A serotype 21 VP0 polyprotein as set forth in SEQ ID NO: 4. In some embodiments, the second protein or polyprotein has or comprises an amino acid sequence that is at least 98% identical to the amino acid sequence of the rhinovirus A serotype 21 VP0 polyprotein as set forth in SEQ ID NO: 4. In some embodiments, the second protein or polyprotein has or comprises an amino acid sequence that is at least 99% identical to the amino acid sequence of the rhinovirus A serotype 21 VP0 polyprotein as set forth in SEQ ID NO: 4. In some embodiments, the second protein or polyprotein has or comprises an amino acid sequence that is identical to the amino acid sequence of the rhinovirus A serotype 21 VP0 polyprotein as set forth in SEQ ID NO: 4.

In some embodiments, separate non-naturally occurring mRNA molecules encode the one or more non-structural polypeptides and the one or more structural polypeptides.

In some embodiments, the same mRNA molecule encodes the one or more non-structural polypeptides and the one or more structural polypeptides. In some embodiments, the mRNA molecule encoding the one or more non-structural polypeptides and the one or more structural polypeptides encodes a fusion protein. In some embodiments, the fusion protein comprises a naturally occurring polyprotein comprising the one or more non-structural polypeptides and a naturally occurring polyprotein comprising the one or more structural polypeptides. In some embodiments, the polyprotein comprising the one or more non-structural polypeptides comprises at least one of the rhinovirus proteins 2A, 2B and 2C. In some embodiments, the polyprotein comprising the one or more non-structural polypeptides comprises rhinovirus proteins 2A, 2B and 2C. In some embodiments, the polyprotein comprising the one or more structural polypeptides comprises rhinovirus proteins VP4 and VP2.

In some embodiments, the fusion protein has or comprises an amino acid sequence that is at least 80% identical to the amino acid sequence set forth in SEQ ID NO: 34. In some embodiments, the fusion protein has or comprises an amino acid sequence that is at least 85% identical to the amino acid sequence set forth in SEQ ID NO: 34. In some embodiments, the fusion protein has or comprises an amino acid sequence that is at least 90% identical to the amino acid sequence set forth in SEQ ID NO: 34. In some embodiments, the fusion protein has or comprises an amino acid sequence that is at least 95% identical to the amino acid sequence set forth in SEQ ID NO: 34. In some embodiments, the fusion protein has or comprises an amino acid sequence that is at least 98% identical to the amino acid sequence set forth in SEQ ID NO: 34. In some embodiments, the fusion protein has or comprises an amino acid sequence that is at least 99% identical to the amino acid sequence set forth in SEQ ID NO: 34. In some embodiments, the fusion protein has or comprises an amino acid sequence that is identical to the amino acid sequence set forth in SEQ ID NO: 34.

### MHC-I/MHC-II Allele Coverage

The polyproteins disclosed herein have been specifically selected because the plurality of T-cell epitope-rich regions they include provide a wide coverage of MHC-I and MHC-II alleles. Published T-cell epitope sequences identified for rhinovirus are publicly available at the IEDB website (http://www.iedb.org/). The IEDB website also indicates whether the epitope type is B-cell, T-cell MHC-I or T-cell MHC-II.

The IEDB alleles have been set up to cover 97% of worldwide human population MHC-I alleles and 99% of worldwide human population MHC-II alleles. MHC-I and MHC-II alleles resulting in coverage of 97% and 99% of the worldwide human population are provided in Tables 3 and 4, respectively.

TABLE 4
MHC-I allele distribution

| MHC-I allele | count |
|---|---|
| HLA-A*01:01 | 209 |
| HLA-A*02:01 | 106 |
| HLA-A*02:03 | 118 |
| HLA-A*02:06 | 133 |
| HLA-A*03:01 | 143 |
| HLA-A*11:01 | 137 |
| HLA-A*23:01 | 183 |
| HLA-A*24:02 | 194 |
| HLA-A*26:01 | 184 |
| HLA-A*30:01 | 123 |
| HLA-A*30:02 | 217 |
| HLA-A*31:01 | 93 |
| HLA-A*32:01 | 137 |
| HLA-A*33:01 | 97 |
| HLA-A*68:01 | 124 |
| HLA-A*68:02 | 145 |
| HLA-B*07:02 | 127 |
| HLA-B*08:01 | 136 |
| HLA-B*15:01 | 158 |
| HLA-B*35:01 | 188 |
| HLA-B*40:01 | 120 |
| HLA-B*44:02 | 146 |
| HLA-B*44:03 | 145 |
| HLA-B*51:01 | 153 |
| HLA-B*53:01 | 196 |
| HLA-B*57:01 | 162 |
| HLA-B*58:01 | 173 |

TABLE 5
MHC-II allele distribution

| MHC-II allele | count |
|---|---|
| HLA-DPA1*01:03/DPB1*02:01 | 159 |
| HLA-DPA1*01:03/DPB1*04:01 | 311 |
| HLA-DPA1*02:01/DPB1*01:01 | 61 |
| HLA-DPA1*02:01/DPB1*05:01 | 189 |
| HLA-DPA1*02:01/DPB1*14:01 | 164 |
| HLA-DPA1*03:01/DPB1*04:02 | 24 |
| HLA-DQA1*01:01/DQB1*05:01 | 252 |
| HLA-DQA1*01:02/DQB1*06:02 | 93 |
| HLA-DQA1*03:01/DQB1*03:02 | 14 |
| HLA-DQA1*04:01/DQB1*04:02 | 13 |
| HLA-DQA1*05:01/DQB1*02:01 | 150 |
| HLA-DQA1*05:01/DQB1*03:01 | 82 |
| HLA-DRB1*01:01 | 115 |
| HLA-DRB1*03:01 | 123 |
| HLA-DRB1*04:01 | 296 |
| HLA-DRB1*04:05 | 271 |
| HLA-DRB1*07:01 | 149 |
| HLA-DRB1*08:02 | 118 |
| HLA-DRB1*09:01 | 51 |
| HLA-DRB1*11:01 | 90 |
| HLA-DRB1*12:01 | 184 |
| HLA-DRB1*13:02 | 334 |
| HLA-DRB1*15:01 | 314 |
| HLA-DRB3*01:01 | 151 |
| HLA-DRB3*02:02 | 446 |
| HLA-DRB4*01:01 | 231 |
| HLA-DRB5*01:01 | 128 |

Without wishing to be bound by any particular theory, an immunogenic composition comprising T-cell epitopes for all MHC-I and/or MHC-II alleles identified in Tables 3 and 4 should provide coverage of the majority of the human population. Therefore, said composition should induce an immune response, and especially a T-cell response, that is broadly protective against infection by multiple rhinoviruses for the majority of the human population. As shown in the examples, immunogenic compositions disclosed herein comprise non-naturally occurring mRNAs encoding both structural and non-structural rhinovirus polypeptides comprising a plurality of T-cell epitope-rich regions that collectively can elicit a T-cell response in at least 95% (e.g., at least 96%, at least 97%, at least 98%, or at least 99%) of the human population. In particular, the structural and non-structural rhinovirus polypeptides encoded by these mRNA comprise T-cell epitope-rich regions that cover at least 95% (at least 96%, at least 97%, at least 98%, or at least 99%) of the MHC class-I alleles in Table 4 and at least 95% (at least 96%, at least 97%, at least 98%, or at least 99%) of the MHC-II alleles in Table 5.

In some embodiments, the one or more T-cell epitope-rich regions elicit a T-cell response in at least 95% of the human population. In some embodiments, the one or more T-cell epitope-rich regions elicit a T-cell response in at least 96% of the human population. In some embodiments, the one or more T-cell epitope-rich regions elicit a T-cell response in at least 97% of the human population. In some embodiments, the one or more T-cell epitope-rich regions elicit a T-cell response in at least 98% of the human population. In some embodiments, the one or more T-cell epitope-rich regions elicit a T-cell response in at least 99% of the human population.

In some embodiments, the one or more T-cell epitope-rich regions cover at least 95% of the MHC class-I alleles in Table 4 and/or 95% of the MHC-II alleles in Table 5. In some embodiments, the one or more T-cell epitope-rich regions cover at least 95% of the MHC class-I alleles in Table 4 and at least 95% of the MHC-II alleles in Table 5. In some embodiments, the one or more T-cell epitope-rich regions cover at least 96% of the MHC class-I alleles in Table 4 and/or 96% of the MHC-II alleles in Table 5. In some embodiments, the one or more T-cell epitope-rich regions cover at least 95% of the MHC class-I alleles in Table 4 and at least 96% of the MHC-II alleles in Table 5. In some embodiments, the one or more T-cell epitope-rich regions cover at least 97% of the MHC class-I alleles in Table 4 and/or 97% of the MHC-II alleles in Table 5. In some embodiments, the one or more T-cell epitope-rich regions cover at least 97% of the MHC class-I alleles in Table 4 and at least 97% of the MHC-II alleles in Table 5. In some embodiments, the one or more T-cell epitope-rich regions cover at least 98% of the MHC class-I alleles in Table 4 and/or 98% of the MHC-II alleles in Table 5. In some embodiments, the one or more T-cell epitope-rich regions cover at least 98% of the MHC class-I alleles in Table 4 and at least 98% of the MHC-II alleles in Table 5. In some embodiments, the one or more T-cell epitope-rich regions cover at least 99% of the MHC class-I alleles in Table 4 and/or 99% of the MHC-II alleles in Table 5. In some embodiments, the one or more T-cell epitope-rich regions cover at least 99% of the MHC class-I alleles in Table 4 and at least 99% of the MHC-II alleles in Table 5. Generation of Optimized Nucleotide Sequences

The present invention also provides sequence-optimized mRNAs that encode one or more non-structural rhinovirus polypeptides comprising one or more T-cell epitope-rich regions and/or one or more structural rhinovirus polypeptides comprising one or more T-cell epitope-rich regions. These mRNAs are modified relative to their naturally occurring counterparts to (a) improve the yield of full-length mRNAs during in vitro synthesis, and (b) to maximize expression of the encoded polypeptide after delivery of the mRNA to a target cell in vivo. Sequence motifs that favor rapid degradation of the mRNA in the target cell have also been removed.

A process for generating optimized nucleotide sequences may include first generating a list of codon-optimized sequences and then applying three filters to the list. Specifically, it applies a motif screen filter, guanine-cytosine (GC) content analysis filter, and codon adaptation index (CAI) analysis filter to produce an updated list of optimized nucleotide sequences. The updated list no longer includes nucleotide sequences containing features that are expected to interfere with effective transcription and/or translation of the encoded polypeptide.

### Codon Optimization

The genetic code has 64 possible codons. Each codon comprises a sequence of three nucleotides. The usage frequency for each codon in the protein-coding regions of the genome can be calculated by determining the number of instances that a specific codon appears within the protein-coding regions of the genome, and subsequently dividing the obtained value by the total number of codons that encode the same amino acid within protein-coding regions of the genome.

A codon usage table contains experimentally derived data regarding how often, for the particular biological source from which the table has been generated, each codon is used to encode a certain amino acid. This information is expressed, for each codon, as a percentage (0 to 100%), or fraction (0 to 1), of how often that codon is used to encode a certain amino acid relative to the total number of times a codon encodes that amino acid.

Codon usage tables are stored in publicly available databases, such as the Codon Usage Database (Nakamura et al. (2000) Nucleic Acids Research 28(1): 292; available online at https://www.kazusa.or.jp/codon/), and the High-performance Integrated Virtual Environment-Codon Usage Tables (HIVE-CUTs) database (Athey et al., (2017), BMC Bioinformatics 18(1): 391; available online at http://hive.biochemistry.gwu.edu/review/codon).

During the first step of codon optimization, codons are removed from a first codon usage table which reflects the frequency of each codon in a given organism (e.g., a mammal or human) if they are associated with a codon usage frequency which is less than a threshold frequency (e.g., 10%). The codon usage frequencies of the codons not removed in the first step are normalized to generate a normalized codon usage table. An optimized nucleotide sequence encoding an amino acid sequence of interest is generated by selecting a codon for each amino acid in the amino acid sequence based on the usage frequency of the one or more codons associated with a given amino acid in the normalized codon usage table. The probability of selecting a certain codon for a given amino acid is equal to the usage frequency associated with the codon associated with this amino acid in the normalized codon usage table.

The codon-optimized sequences of the invention are generated by a computer-implemented method for generating an optimized nucleotide sequence. The method comprises: (i) receiving an amino acid sequence, wherein the amino acid sequence encodes a peptide, polypeptide, or protein; (ii) receiving a first codon usage table, wherein the first codon usage table comprises a list of amino acids, wherein each amino acid in the table is associated with at least one codon and each codon is associated with a usage frequency; (iii) removing from the codon usage table any codons associated with a usage frequency which is less than a threshold frequency; (iv) generating a normalized codon usage table by normalizing the usage frequencies of the codons not removed in step (iii); and (v) generating an optimized nucleotide sequence encoding the amino acid sequence by selecting a codon for each amino acid in the amino acid sequence based on the usage frequency of the one or more codons associated with the amino acid in the normalized codon usage table. The threshold frequency can be in the range of 5%-30%, in particular 5%, 10%, 15%, 20%, 25%, or 30%. In the context of the present invention, the threshold frequency is typically 10%.

The step of generating a normalized codon usage table comprises: (a) distributing the usage frequency of each codon associated with a first amino acid and removed in step (iii) to the remaining codons associated with the first amino acid; and (b) repeating step (a) for each amino acid to produce a normalized codon usage table. In some embodiments, the usage frequency of the removed codons is distributed equally amongst the remaining codons. In some embodiments, the usage frequency of the removed codons is distributed amongst the remaining codons proportionally based on the usage frequency of each remaining codon. “Distributed” in this context may be defined as taking the combined magnitude of the usage frequencies of removed codons associated with a certain amino acid and apportioning some of this combined frequency to each of the remaining codons encoding the certain amino acid.

The step of selecting a codon for each amino acid comprises: (a) identifying, in the normalized codon usage table, the one or more codons associated with a first amino acid of the amino acid sequence; (b) selecting a codon associated with the first amino acid, wherein the probability of selecting a certain codon is equal to the usage frequency associated with the codon associated with the first amino acid in the normalized codon usage table; and (c) repeating steps (a) and (b) until a codon has been selected for each amino acid in the amino acid sequence.

The step of generating an optimized nucleotide sequence by selecting a codon for each amino acid in the amino acid sequence (step (v) in the above method) is performed n times to generate a list of optimized nucleotide sequences.

### Motif Screen

A motif screen filter is applied to the list of optimized nucleotide sequences. Optimized nucleotide sequences encoding any known negative cis-regulatory elements and negative repeat elements are removed from the list to generate an updated list.

For each optimized nucleotide sequence in the list, it is also determined whether it contains a termination signal. Any nucleotide sequence that contains one or more termination signals is removed from the list generating an updated list. In some embodiments, the termination signal has the following nucleotide sequence: 5′-X₁ATCTX₂TX₃-3′, wherein X₁, X₂ and X₃ are independently selected from A, C, T or G. In some embodiments, the termination signal has one of the following nucleotide sequences: TATCTGTT; and/or TTTTTT; and/or AAGCTT; and/or GAAGAGC; and/or TCTAGA. In a typical embodiment, the termination signal has the following nucleotide sequence: 5′-X₁AUCUX₂UX₃-3′, wherein X₁, X₂ and X₃ are independently selected from A, C, U or G. In a specific embodiment, the termination signal has one of the following nucleotide sequences: UAUCUGUU; and/or UUUUUU; and/or AAGCUU; and/or GAAGAGC; and/or UCUAGA.

### Guanine-Cytosine (GC) Content

The method further comprises determining a guanine-cytosine (GC) content of each of the optimized nucleotide sequences in the updated list of optimized nucleotide sequences. The GC content of a sequence is the percentage of bases in the nucleotide sequence that are guanine or cytosine. The list of optimized nucleotide sequences is further updated by removing any nucleotide sequence from the list, if its GC content falls outside a predetermined GC content range.

Determining a GC content of each of the optimized nucleotide sequences comprises, for each nucleotide sequence: determining a GC content of one or more additional portions of the nucleotide sequence, wherein the additional portions are non-overlapping with each other and with the first portion, and wherein updating the list of optimized sequences comprises: removing the nucleotide sequence if the GC content of any portion falls outside the predetermined GC content range, optionally wherein determining the GC content of the nucleotide sequence is halted when the GC content of any portion is determined to be outside the predetermined GC content range. In some embodiments, the first portion and/or the one or more additional portions of the nucleotide sequence comprise a predetermined number of nucleotides, optionally wherein the predetermined number of nucleotides is in the range of: 5 to 300 nucleotides, or 10 to 200 nucleotides, or 15 to 100 nucleotides, or 20 to 50 nucleotides. In the context of the present invention, the predetermined number of nucleotides is typically 30 nucleotides. The predetermined GC content range can be 15%-75%, or 40%-60%, or, 30%-70%. In the context of the present invention, the predetermined GC content range is typically 30%-70%.

A suitable GC content filter in the context of the invention may first analyze the first 30 nucleotides of the optimized nucleotide sequence, i.e., nucleotides 1 to 30 of the optimized nucleotide sequence. Analysis may comprise determining the number of nucleotides in the portion which are either G or C, and determining the GC content of the portion may comprise dividing the number of G or C nucleotides in the portion by the total number of nucleotides in the portion. The result of this analysis will provide a value describing the proportion of nucleotides in the portion that are G or C, and may be a percentage, for example 50%, or a decimal, for example 0.5. If the GC content of the first portion falls outside a predetermined GC content range, the optimized nucleotide sequence may be removed from the list of optimized nucleotide sequences.

If the GC content of the first portion falls inside the predetermined GC content range, the GC content filter may then analyze a second portion of the optimized nucleotide sequence. In this example, this may be the second 30 nucleotides, i.e., nucleotides 31 to 60, of the optimized nucleotide sequence. The portion analysis may be repeated for each portion until either: a portion is found having a GC content falling outside the predetermined GC content range, in which case the optimized nucleotide sequence may be removed from the list, or the whole optimized nucleotide sequence has been analyzed and no such portion has been found, in which case the GC content filter retains the optimized nucleotide sequence in the list and may move on to the next optimized nucleotide sequence in the list.

### Codon Adaptation Index (CAI)

The method further comprises determining a codon adaptation index of each of the optimized nucleotide sequences in the most recently updated list of optimized nucleotide sequences. The codon adaptation index of a sequence is a measure of codon usage bias and can be a value between 0 and 1. The most recently updated list of optimized nucleotide sequences is further updated by removing any nucleotide sequence if its codon adaptation index is less than or equal to a predetermined codon adaptation index threshold. The codon adaptation index threshold can be 0.7, or 0.75, or 0.8, or 0.85, or 0.9. The inventors have found that optimized nucleotide sequences with a codon adaptation index equal to or greater than 0.8 deliver very high protein yield. Therefore, in the context of the invention, the codon adaptation index threshold is typically 0.8.

A codon adaptation index may be calculated, for each optimized nucleotide sequence, in any way that would be apparent to a person skilled in the art, for example as described in “The codon adaptation index—a measure of directional synonymous codon usage bias, and its potential applications” (Sharp and Li, 1987. Nucleic Acids Research 15(3): p. 1281-1295); available online at: https://www.ncbi.nlm.nih.gov/pmc/articles/PMC340524/.

Implementing a codon adaptation index calculation may include a method according to, or similar to, the following. For each amino acid in a sequence, a weight of each codon in a sequence may be represented by a parameter termed relative adaptiveness (wᵢ). Relative adaptiveness may be computed from a reference sequence set, as the ratio between the observed frequency of the codon fᵢ and the frequency of the most frequent synonymous codon fⱼ for that amino acid. The codon adaptation index of a sequence may then be calculated as the geometric mean of the weight associated to each codon over the length of the sequence (measured in codons). The reference sequence set used to calculate codon adaptation index may be the same reference sequence set from which a codon usage table used with methods of the invention is derived.

### Exemplary Optimized Nucleotide Sequences

Exemplary optimized nucleotide sequences encoding a non-structural rhinovirus polyprotein and a structural rhinovirus polyprotein set out in Table 6 have been generated in accordance with the methods described herein.

In specific embodiments, an immunogenic composition of the invention comprises an mRNA comprising an optimized nucleotide sequence encoding a P2 polyprotein having the nucleic acid sequence of SEQ ID NOs: 46 or 47. In another specific embodiment, an immunogenic composition of the invention comprises an mRNA comprising an optimized nucleotide sequence encoding a VP0 polyprotein having the nucleic acid sequence of SEQ ID NOs: 48 or 49.

In some embodiments, the VP0 polyprotein or the P2 polyprotein are operationally linked to a non-native secretion signal sequence from an influenza A virus to increase secretion of the mRNA encoded protein(s). Accordingly, in specific embodiments, an immunogenic composition of the invention comprises an mRNA comprising an optimized nucleotide sequence encoding a P2 polyprotein having the nucleic acid sequence of SEQ ID NOs: 50 or 51. In another specific embodiment, an immunogenic composition of the invention comprises an mRNA comprising an optimized nucleotide sequence encoding a VP0 polyprotein having the nucleic acid sequence of SEQ ID NOs: 52 or 53.

Together, the P2 polyprotein and VP0 polyprotein derived from the rhinovirus A serotype 21 strain are predicted to provide T-cell epitopes that cover 97-99% of all human MHC-I and MHC-II alleles worldwide. Therefore, in a further specific embodiment, an immunogenic composition of the invention comprises an mRNA comprising an optimized nucleotide sequence encoding a P2 polyprotein having the nucleic acid sequence of SEQ ID NOs: 46, 47, 50, or 51 and an mRNA comprising an optimized nucleotide sequence encoding a VP0 polyprotein having the nucleic acid sequence of SEQ ID NOs: 48, 49, 52, or 53. In one particular embodiment, an immunogenic composition of the invention comprises an mRNA comprising an optimized nucleotide sequence encoding a P2 polyprotein having the nucleic acid sequence of SEQ ID NOs: 50 or 51 and an mRNA comprising an optimized nucleotide sequence encoding a VP0 polyprotein having the nucleic acid sequence of SEQ ID NOs: 52 or 53. In another particular embodiment, an immunogenic composition of the invention comprises an mRNA comprising an optimized nucleotide sequence encoding a P2 polyprotein having the nucleic acid sequence of SEQ ID NOs: 46 or 47 and an mRNA comprising an optimized nucleotide sequence encoding a VP0 polyprotein having the nucleic acid sequence of SEQ ID NOs: 48 or 49. In some embodiments, an immunogenic composition of the invention comprises an mRNA comprising an optimized nucleotide sequence encoding a P2 polyprotein having the nucleic acid sequence of SEQ ID NOs: 50 or 51 and an mRNA comprising an optimized nucleotide sequence encoding a VP0 polyprotein having the nucleic acid sequence of SEQ ID NOs: 48 or 49. In other embodiments, an immunogenic composition of the invention comprises an mRNA comprising an optimized nucleotide sequence encoding a P2 polyprotein having the nucleic acid sequence of SEQ ID NOs: 46 or 47 and an mRNA comprising an optimized nucleotide sequence encoding a VP0 polyprotein having the nucleic acid sequence of SEQ ID NOs: 52 or 53.

In Table 6, optimized nucleotide sequences encoding a VP0 polyprotein are underlined and optimized nucleotide sequence encoding a P2 polyprotein are shown in bold. Secretion signal sequences are shown in italics. The VP0 polyprotein comprising a secretion signal sequence comprises the nucleic acid sequence GATACTCTG, encoding the amino acids DTL, between the secretion signal sequence and the VP0 polyprotein.

TABLE 6
Exemplary optimized nucleotide sequences

| | SEQ ID | |
|---|---|---|
| Name | NO. | Sequence |
| P2 | 46 | ATGGGCCCTTCCGATATGTACGTTCACGTGGGGAACCTGA |
| | | TCTATAGAAATCTGCACCTCTTTAACAGCGAGATGCACGA |
| | | CTCCATCCTGATCAGCTATAGTAGCGACCTTGTGATCTAT |
| | | CGCACTAATACCACCGGAGACGACTACATTCCAACCTGCG |
| | | ACTGCACTGAGGCCACCTACTACTGCAAGCATAAAAATCG |
| | | CTACTTCCCCATCAAGGTCACCAGCCATGACTGGTATGAG |
| | | ATCCAGGAGTCTGAATACTATCCTAAGCACATCCAGTACA |
| | | ACCTGCTGATCGGCGAGGGCCCATGTGAGCCTGGGGATTG |
| | | TGGGGGCAAGCTGCTGTGCAAGCACGGCGTGATCGGAATG |
| | | ATCACCGCCGGCGGGGACGGACACGTGGCTTTCATTGATC |
| | | TGAGGCACTTTCACTGTGCTGAGGAGCAGGGCATCACTGA |
| | | CTACATCCACATGCTGGGCGAAGCCTTTGGGTCCGGATTC |
| | | GTGGATAGCGTGAAGGAGCAGATCAATGCCATCAACCCTA |
| | | TCAACAACATCAGCAAAAAGATCATCAAATGGCTGCTGAG |
| | | GATCATTTCTGCCATGGTTATCATCATCAGAAACTCCAGC |
| | | GACCCTCAGACAATCATCGCCACCCTCACTCTGATTGGCT |
| | | GCAGCGGGTCCCCTTGGAGATTCCTGAAGGAGAAGTTCTG |
| | | CAAGTGGACCCAGCTGAACTACATCCACAAAGAGAGCGAC |
| | | AGTTGGCTGAAAAAATTCACCGAAATGTGTAACGCCGCAA |
| | | GGGGCCTGGAATGGATCGGCAATAAGATTTCTAAGTTTAT |
| | | CGACTGGATGAAATCCATGCTGCCTCAGGCCCAGCTGAAG |
| | | GTGAAATACCTGAGCGAACTGAAGAAGCTGAACCTGCTGG |
| | | AGAAGCAGATTGAGCACCTGAGAGCAGCAGACTCCGCCAC |
| | | ACAGGAAAAAATCAAGTGCGAGATTGACACTCTGCATGAC |
| | | CTGTCCTGCAAATTCCTGCCTCTGTACGCTTCCGAGGCCA |
| | | AAAGAATCAAGGTGCTGCACAATAAGTGCAATGTGGTTAT |
| | | CAAGCAGAAAAAGCGGTGTGAGCCAGTGGCCGTGGTTATT |
| | | CACGGAGAGCCTGGCACCGGCAAGAGCATGACTACTAATT |
| | | TTCTCGCCCGGATGATCACCAATGACTCTGACATCTACTC |
| | | CCTGCCCCCAGACCCAAAGTATTTCGACGGATATGACCAG |
| | | CAGAGCGTGGTTATCATGGATGACATCATGCAGAATCCCT |
| | | CCGGCGACGATATGACCCTGTTTTGTCAGATGGTTTCCTC |
| | | CGTGACCTTCATCCCCCCAATGGCCGATCTGCCTGACAAG |
| | | GGCAAGCCTTTTGACAGCAGGTTCGTGCTGTGCAGCACAA |
| | | ACCACAGCCTGCTGACCCCCCCTACTATTACCTCTCTGCC |
| | | CGCCATGAACAGGAGATTCTTTATGGACCTGGACATCATC |
| | | GTGTGTGATAAATATAAGGACGCCCAGGGAAAGCTGAATG |
| | | TCAGCGCCGCTTTCAAGCCCTGCGACGTGGACACCAAGAT |
| | | CGGCAACGCTCGGTGTTGTCCTTTCATCTGCGGCAAAGCC |
| | | GTGATGTTCAAAGACAGAAATACATGTCGCACCTACACTC |
| | | TGGCCCAGATCTACAACCAGATTCTGGAGGAGGATAAGAG |
| | | GAGACGCCAGGTCATTGACGTCATGTCCGCCATTTTCCAG |
| | | TGA |
| P2 | 47 | ATGGGGCCAAGCGACATGTATGTGCATGTGGGAAACCTGA |
| | | TCTACAGAAACCTGCACCTCTTTAACAGCGAGATGCACGA |
| | | TTCCATTCTGATTTCCTATTCCTCTGATCTCGTTATCTAC |
| | | CGGACCAACACCACAGGAGACGACTATATTCCAACATGCG |
| | | ACTGCACAGAGGCCACTTACTACTGTAAGCACAAAAACAG |
| | | ATACTTCCCCATCAAGGTGACTTCCCACGACTGGTACGAG |
| | | ATCCAGGAATCTGAATACTACCCTAAGCATATCCAGTACA |
| | | ACCTGCTGATTGGAGAAGGGCCATGCGAGCCAGGCGATTG |
| | | TGGCGGCAAGCTGCTGTGTAAACACGGCGTGATCGGCATG |
| | | ATTACCGCCGGGGGCGACGGCCATGTGGCATTCATCGACC |
| | | TGAGGCATTTCCATTGTGCAGAGGAGCAGGGCATTACCGA |
| | | TTACATTCACATGCTGGGCGAGGCTTTCGGCAGCGGCTTT |
| | | GTGGACAGCGTGAAGGAGCAGATTAACGCAATTAACCCTA |
| | | TCAATAACATTTCCAAGAAGATCATCAAGTGGCTGCTGAG |
| | | AATCATCTCCGCCATGGTTATTATCATCCGCAACTCTAGC |
| | | GACCCCCAGACCATTATCGCAACACTGACCCTGATCGGAT |
| | | GTTCCGGCTCCCCTTGGCGCTTTCTGAAGGAGAAGTTTTG |
| | | CAAGTGGACCCAGCTGAACTACATTCACAAGGAGAGCGAC |
| | | TCTTGGCTGAAAAAGTTCACAGAAATGTGTAATGCCGCCA |
| | | GAGGGCTGGAATGGATCGGAAATAAGATCTCCAAGTTCAT |
| | | CGACTGGATGAAGTCCATGCTGCCACAGGCCCAGCTGAAA |
| | | GTCAAATACCTGTCTGAGCTGAAAAAACTGAACCTGCTGG |
| | | AGAAGCAGATTGAACACCTTCGGGCCGCTGACTCTGCCAC |
| | | TCAGGAGAAGATCAAGTGCGAAATTGACACCCTGCACGAC |
| | | CTGAGCTGCAAATTCCTGCCTCTGTATGCCTCCGAGGCCA |
| | | AGAGGATCAAGGTGCTGCACAACAAATGCAATGTCGTTAT |
| | | CAAGCAGAAGAAGCGGTGCGAGCCCGTGGCCGTCGTGATC |
| | | CACGGGGAGCCTGGAACAGGAAAGTCTATGACTACTAACT |
| | | TCCTCGCCCGGATGATCACAAACGACAGCGACATCTATAG |
| | | CCTGCCACCCGACCCTAAATATTTCGATGGATACGACCAG |
| | | CAGTCTGTCGTGATCATGGATGACATCATGCAGAATCCTA |
| | | GCGGGGATGATATGACACTGTTCTGCCAGATGGTGAGCAG |
| | | CGTGACATTCATCCCACCAATGGCTGACCTGCCAGACAAG |
| | | GGCAAGCCTTTCGACTCCAGATTCGTGCTGTGCTCCACCA |
| | | ACCACTCCCTCCTGACCCCTCCCACAATTACCTCCCTGCC |
| | | AGCCATGAATAGGCGCTTCTTTATGGACCTGGATATCATC |
| | | GTCTGCGACAAATACAAGGATGCCCAGGGGAAACTGAACG |
| | | TGTCCGCCGCCTTTAAGCCTTGTGACGTGGACACAAAGAT |
| | | TGGGAATGCCAGATGCTGCCCTTTTATTTGCGGAAAGGCC |
| | | GTCATGTTCAAGGACCGGAATACCTGCAGGACATACACTC |
| | | TGGCTCAGATCTACAACCAGATTCTGGAGGAGGACAAGAG |
| | | GAGAAGGCAGGTCATCGACGTGATGTCTGCCATCTTCCAG |
| | | TGA |
| VPO | 48 | ATGGGAACCCAGGTGTCTCGCCAGAATGTGGGAACCCACT |
| | | CCACTCAGAACAGCGTGAGTAACGGGAGCAGCCTGAACTA |
| | | TTTTAACATCAATTATTTTAAGGACGCCGCTTCCTCCGGC |
| | | GCAAGCAAGCTGGAGTTCAGCCAGGACCCCTCCAAGTTTA |
| | | CCGATCCAGTGAAAGATGTCCTGGAGAAAGGCATCCCCAC |
| | | CCTGCAGAGCCCCACAGTGGAGGCCTGTGGGTATAGCGAC |
| | | AGAATCATCCAGATCACTAGGGGCGACTCCACCATTACTA |
| | | GCCAGGATGTTGCCAATGCCGTGGTGGGATATGGGGTGTG |
| | | GCCACACTATCTCACACCACAGGACGCCACCGCCATCGAT |
| | | AAACCTTCCAGGCCTGACACTTCCTCTAATCGCTTTTATA |
| | | CCCTGGAGAGCAAGATGTGGACCAGCGACAGCAAGGGCTG |
| | | GTGGTGGAAGCTGCCAGATGCTCTGAAGAATATGGGCATT |
| | | TTCGGAGAGAACATGTTTTATCACTTCCTGGGGCGGTCTG |
| | | GATACACCGTGCACGTGCAGTGCAATGCTTCCAAGTTTCA |
| | | CCAGGGCACTCTGATCGTGGTTATGATTCCTGAGCACCAG |
| | | CTGGCAAGCGCATCCACCGGAAACGTTACCGCCGGATACA |
| | | ATCTGACACATCCCGGCGAGCAGGGCAGGGATGTGGGCAT |
| | | TACCCGGGTGGAAGACCTGCTGAAGCAGCCTAGCGACGAT |
| | | AGCTGGCTGAATTTTGACGGCACACTGCTGGGAAACATCA |
| | | CTATTTTCCCCCACCAGTTTATCAACCTGAGAAGCAACAA |
| | | CTCCGCTACAATTATCGTGCCCTACGTGAATGCCGTTCCT |
| | | ATGGACTCCATGCCACGCCACAACAATTGGAGCCTGGTCA |
| | | TCATTCCAATTTGCCCTCTGGAGAGTGATGGACAGACACC |
| | | CGTGCCAATCACTATCTCCATTTCCCCAATGTGTGCCGAG |
| | | TTTTCCGGCGCCAGAGCCAAGTCACAGTGA |
| VPO | 49 | ATGGGCACCCAGGTGTCTCGGCAGAATGTCGGAACCCACT |
| | | CAACACAGAATAGCGTGAGCAACGGCAGCAGCCTGAACTA |
| | | CTTTAATATCAATTACTTTAAGGACGCTGCCAGCAGCGGA |
| | | GCCAGCAAACTCGAGTTCAGCCAAGACCCAAGCAAGTTCA |
| | | CCGACCCCGTGAAAGACGTCCTGGAAAAGGGAATCCCCAC |
| | | CCTGCAGAGCCCAACAGTCGAGGCTTGTGGGTATTCTGAT |
| | | AGAATCATCCAGATTACCAGAGGAGACAGTACCATCACAT |
| | | CTCAGGACGTGGCAAATGCAGTGGTGGGCTACGGAGTGTG |
| | | GCCTCACTACCTGACCCCCCAGGATGCCACCGCCATTGAC |
| | | AAGCCCTCCCGCCCTGACACTTCTTCCAATAGGTTTTACA |
| | | CCCTGGAGTCTAAGATGTGGACATCCGATTCCAAGGGATG |
| | | GTGGTGGAAGCTGCCTGATGCTCTGAAAAACATGGGCATC |
| | | TTCGGGGAGAACATGTTCTATCACTTTCTGGGAAGGTCCG |
| | | GCTATACAGTTCACGTGCAGTGCAACGCTAGCAAGTTCCA |
| | | TCAGGGCACTCTGATCGTGGTCATGATTCCCGAGCACCAG |
| | | CTGGCATCTGCCTCCACAGGGAACGTGACTGCCGGATATA |
| | | ACCTGACCCACCCTGGAGAGCAGGGCAGGGACGTCGGAAT |
| | | CACCAGAGTGGAAGACCTGCTCAAACAGCCCAGCGACGAC |
| | | TCTTGGCTGAACTTCGACGGCACACTGCTGGGGAACATTA |
| | | CAATTTTCCCTCACCAGTTCATCAACCTGAGGTCTAACAA |
| | | CAGCGCCACAATCATTGTTCCATATGTGAACGCCGTGCCC |
| | | ATGGATAGCATGCCCCGGCACAACAACTGGAGCCTGGTTA |
| | | TCATCCCCATTTGCCCACTGGAGAGCGACGGACAGACACC |
| | | TGTGCCCATCACAATCTCCATCTCTCCCATGTGTGCCGAG |
| | | TTCTCTGGGGCACGCGCTAAATCCCAGTGA |
| P2 comprising a | 50 | ATGAAGGCCATTCTGGTGGTGCTGCTGTATACCTTCGCTA |
| secretion signal | | CAGCTAATGCCGGCCCTTCCGATATGTACGTTCACGTGGG |
| sequence | | GAACCTGATCTATAGAAATCTGCACCTCTTTAACAGCGAG |
| | | ATGCACGACTCCATCCTGATCAGCTATAGTAGCGACCTTG |
| | | TGATCTATCGCACTAATACCACCGGAGACGACTACATTCC |
| | | AACCTGCGACTGCACTGAGGCCACCTACTACTGCAAGCAT |
| | | AAAAATCGCTACTTCCCCATCAAGGTCACCAGCCATGACT |
| | | GGTATGAGATCCAGGAGTCTGAATACTATCCTAAGCACAT |
| | | CCAGTACAACCTGCTGATCGGCGAGGGCCCATGTGAGCCT |
| | | GGGGATTGTGGGGGCAAGCTGCTGTGCAAGCACGGCGTGA |
| | | TCGGAATGATCACCGCCGGCGGGGACGGACACGTGGCTTT |
| | | CATTGATCTGAGGCACTTTCACTGTGCTGAGGAGCAGGGC |
| | | ATCACTGACTACATCCACATGCTGGGCGAAGCCTTTGGGT |
| | | CCGGATTCGTGGATAGCGTGAAGGAGCAGATCAATGCCAT |
| | | CAACCCTATCAACAACATCAGCAAAAAGATCATCAAATGG |
| | | CTGCTGAGGATCATTTCTGCCATGGTTATCATCATCAGAA |
| | | ACTCCAGCGACCCTCAGACAATCATCGCCACCCTCACTCT |
| | | GATTGGCTGCAGCGGGTCCCCTTGGAGATTCCTGAAGGAG |
| | | AAGTTCTGCAAGTGGACCCAGCTGAACTACATCCACAAAG |
| | | AGAGCGACAGTTGGCTGAAAAAATTCACCGAAATGTGTAA |
| | | CGCCGCAAGGGGCCTGGAATGGATCGGCAATAAGATTTCT |
| | | AAGTTTATCGACTGGATGAAATCCATGCTGCCTCAGGCCC |
| | | AGCTGAAGGTGAAATACCTGAGCGAACTGAAGAAGCTGAA |
| | | CCTGCTGGAGAAGCAGATTGAGCACCTGAGAGCAGCAGAC |
| | | TCCGCCACACAGGAAAAAATCAAGTGCGAGATTGACACTC |
| | | TGCATGACCTGTCCTGCAAATTCCTGCCTCTGTACGCTTC |
| | | CGAGGCCAAAAGAATCAAGGTGCTGCACAATAAGTGCAAT |
| | | GTGGTTATCAAGCAGAAAAAGCGGTGTGAGCCAGTGGCCG |
| | | TGGTTATTCACGGAGAGCCTGGCACCGGCAAGAGCATGAC |
| | | TACTAATTTTCTCGCCCGGATGATCACCAATGACTCTGAC |
| | | ATCTACTCCCTGCCCCCAGACCCAAAGTATTTCGACGGAT |
| | | ATGACCAGCAGAGCGTGGTTATCATGGATGACATCATGCA |
| | | GAATCCCTCCGGCGACGATATGACCCTGTTTTGTCAGATG |
| | | GTTTCCTCCGTGACCTTCATCCCCCCAATGGCCGATCTGC |
| | | CTGACAAGGGCAAGCCTTTTGACAGCAGGTTCGTGCTGTG |
| | | CAGCACAAACCACAGCCTGCTGACCCCCCCTACTATTACC |
| | | TCTCTGCCCGCCATGAACAGGAGATTCTTTATGGACCTGG |
| | | ACATCATCGTGTGTGATAAATATAAGGACGCCCAGGGAAA |
| | | GCTGAATGTCAGCGCCGCTTTCAAGCCCTGCGACGTGGAC |
| | | ACCAAGATCGGCAACGCTCGGTGTTGTCCTTTCATCTGCG |
| | | GCAAAGCCGTGATGTTCAAAGACAGAAATACATGTCGCAC |
| | | CTACACTCTGGCCCAGATCTACAACCAGATTCTGGAGGAG |
| | | GATAAGAGGAGACGCCAGGTCATTGACGTCATGTCCGCCA |
| | | TTTTCCAGTGA |
| P2 comprising a | 51 | ATGAAGGCAATCCTCGTGGTCCTGCTGTACACCTTCGCTA |
| secretion signal | | CAGCCAACGCCGGGCCAAGCGACATGTATGTGCATGTGGG |
| sequence | | AAACCTGATCTACAGAAACCTGCACCTCTTTAACAGCGAG |
| | | ATGCACGATTCCATTCTGATTTCCTATTCCTCTGATCTCG |
| | | TTATCTACCGGACCAACACCACAGGAGACGACTATATTCC |
| | | AACATGCGACTGCACAGAGGCCACTTACTACTGTAAGCAC |
| | | AAAAACAGATACTTCCCCATCAAGGTGACTTCCCACGACT |
| | | GGTACGAGATCCAGGAATCTGAATACTACCCTAAGCATAT |
| | | CCAGTACAACCTGCTGATTGGAGAAGGGCCATGCGAGCCA |
| | | GGCGATTGTGGCGGCAAGCTGCTGTGTAAACACGGCGTGA |
| | | TCGGCATGATTACCGCCGGGGGCGACGGCCATGTGGCATT |
| | | CATCGACCTGAGGCATTTCCATTGTGCAGAGGAGCAGGGC |
| | | ATTACCGATTACATTCACATGCTGGGCGAGGCTTTCGGCA |
| | | GCGGCTTTGTGGACAGCGTGAAGGAGCAGATTAACGCAAT |
| | | TAACCCTATCAATAACATTTCCAAGAAGATCATCAAGTGG |
| | | CTGCTGAGAATCATCTCCGCCATGGTTATTATCATCCGCA |
| | | ACTCTAGCGACCCCCAGACCATTATCGCAACACTGACCCT |
| | | GATCGGATGTTCCGGCTCCCCTTGGCGCTTTCTGAAGGAG |
| | | AAGTTTTGCAAGTGGACCCAGCTGAACTACATTCACAAGG |
| | | AGAGCGACTCTTGGCTGAAAAAGTTCACAGAAATGTGTAA |
| | | TGCCGCCAGAGGGCTGGAATGGATCGGAAATAAGATCTCC |
| | | AAGTTCATCGACTGGATGAAGTCCATGCTGCCACAGGCCC |
| | | AGCTGAAAGTCAAATACCTGTCTGAGCTGAAAAAACTGAA |
| | | CCTGCTGGAGAAGCAGATTGAACACCTTCGGGCCGCTGAC |
| | | TCTGCCACTCAGGAGAAGATCAAGTGCGAAATTGACACCC |
| | | TGCACGACCTGAGCTGCAAATTCCTGCCTCTGTATGCCTC |
| | | CGAGGCCAAGAGGATCAAGGTGCTGCACAACAAATGCAAT |
| | | GTCGTTATCAAGCAGAAGAAGCGGTGCGAGCCCGTGGCCG |
| | | TCGTGATCCACGGGGAGCCTGGAACAGGAAAGTCTATGAC |
| | | TACTAACTTCCTCGCCCGGATGATCACAAACGACAGCGAC |
| | | ATCTATAGCCTGCCACCCGACCCTAAATATTTCGATGGAT |
| | | ACGACCAGCAGTCTGTCGTGATCATGGATGACATCATGCA |
| | | GAATCCTAGCGGGGATGATATGACACTGTTCTGCCAGATG |
| | | GTGAGCAGCGTGACATTCATCCCACCAATGGCTGACCTGC |
| | | CAGACAAGGGCAAGCCTTTCGACTCCAGATTCGTGCTGTG |
| | | CTCCACCAACCACTCCCTCCTGACCCCTCCCACAATTACC |
| | | TCCCTGCCAGCCATGAATAGGCGCTTCTTTATGGACCTGG |
| | | ATATCATCGTCTGCGACAAATACAAGGATGCCCAGGGGAA |
| | | ACTGAACGTGTCCGCCGCCTTTAAGCCTTGTGACGTGGAC |
| | | ACAAAGATTGGGAATGCCAGATGCTGCCCTTTTATTTGCG |
| | | GAAAGGCCGTCATGTTCAAGGACCGGAATACCTGCAGGAC |
| | | ATACACTCTGGCTCAGATCTACAACCAGATTCTGGAGGAG |
| | | GACAAGAGGAGAAGGCAGGTCATCGACGTGATGTCTGCCA |
| | | TCTTCCAGTGA |
| VPO comprising a | 52 | ATGAAGGCCATTCTGGTGGTGCTGCTGTATACCTTCGCTA |
| secretion | | CAGCTAATGCCGATACTCTGGGAACCCAGGTGTCTCGCCA |
| signal sequence | | GAATGTGGGAACCCACTCCACTCAGAACAGCGTGAGTAAC |
| | | GGGAGCAGCCTGAACTATTTTAACATCAATTATTTTAAGG |
| | | ACGCCGCTTCCTCCGGCGCAAGCAAGCTGGAGTTCAGCCA |
| | | GGACCCCTCCAAGTTTACCGATCCAGTGAAAGATGTCCTG |
| | | GAGAAAGGCATCCCCACCCTGCAGAGCCCCACAGTGGAGG |
| | | CCTGTGGGTATAGCGACAGAATCATCCAGATCACTAGGGG |
| | | CGACTCCACCATTACTAGCCAGGATGTTGCCAATGCCGTG |
| | | GTGGGATATGGGGTGTGGCCACACTATCTCACACCACAGG |
| | | ACGCCACCGCCATCGATAAACCTTCCAGGCCTGACACTTC |
| | | CTCTAATCGCTTTTATACCCTGGAGAGCAAGATGTGGACC |
| | | AGCGACAGCAAGGGCTGGTGGTGGAAGCTGCCAGATGCTC |
| | | TGAAGAATATGGGCATTTTCGGAGAGAACATGTTTTATCA |
| | | CTTCCTGGGGCGGTCTGGATACACCGTGCACGTGCAGTGC |
| | | AATGCTTCCAAGTTTCACCAGGGCACTCTGATCGTGGTTA |
| | | TGATTCCTGAGCACCAGCTGGCAAGCGCATCCACCGGAAA |
| | | CGTTACCGCCGGATACAATCTGACACATCCCGGCGAGCAG |
| | | GGCAGGGATGTGGGCATTACCCGGGTGGAAGACCTGCTGA |
| | | AGCAGCCTAGCGACGATAGCTGGCTGAATTTTGACGGCAC |
| | | ACTGCTGGGAAACATCACTATTTTCCCCCACCAGTTTATC |
| | | AACCTGAGAAGCAACAACTCCGCTACAATTATCGTGCCCT |
| | | ACGTGAATGCCGTTCCTATGGACTCCATGCCACGCCACAA |
| | | CAATTGGAGCCTGGTCATCATTCCAATTTGCCCTCTGGAG |
| | | AGTGATGGACAGACACCCGTGCCAATCACTATCTCCATTT |
| | | CCCCAATGTGTGCCGAGTTTTCCGGCGCCAGAGCCAAGTC |
| | | ACAGTGA |
| VPO comprising a | 53 | ATGAAGGCAATCCTCGTGGTCCTGCTGTACACCTTCGCTA |
| secretion signal | | CAGCCAACGCCGATACACTCGGCACCCAGGTGTCTCGGCA |
| sequence | | GAATGTCGGAACCCACTCAACACAGAATAGCGTGAGCAAC |
| | | GGCAGCAGCCTGAACTACTTTAATATCAATTACTTTAAGG |
| | | ACGCTGCCAGCAGCGGAGCCAGCAAACTCGAGTTCAGCCA |
| | | AGACCCAAGCAAGTTCACCGACCCCGTGAAAGACGTCCTG |
| | | GAAAAGGGAATCCCCACCCTGCAGAGCCCAACAGTCGAGG |
| | | CTTGTGGGTATTCTGATAGAATCATCCAGATTACCAGAGG |
| | | AGACAGTACCATCACATCTCAGGACGTGGCAAATGCAGTG |
| | | GTGGGCTACGGAGTGTGGCCTCACTACCTGACCCCCCAGG |
| | | ATGCCACCGCCATTGACAAGCCCTCCCGCCCTGACACTTC |
| | | TTCCAATAGGTTTTACACCCTGGAGTCTAAGATGTGGACA |
| | | TCCGATTCCAAGGGATGGTGGTGGAAGCTGCCTGATGCTC |
| | | TGAAAAACATGGGCATCTTCGGGGAGAACATGTTCTATCA |
| | | CTTTCTGGGAAGGTCCGGCTATACAGTTCACGTGCAGTGC |
| | | AACGCTAGCAAGTTCCATCAGGGCACTCTGATCGTGGTCA |
| | | TGATTCCCGAGCACCAGCTGGCATCTGCCTCCACAGGGAA |
| | | CGTGACTGCCGGATATAACCTGACCCACCCTGGAGAGCAG |
| | | GGCAGGGACGTCGGAATCACCAGAGTGGAAGACCTGCTCA |
| | | AACAGCCCAGCGACGACTCTTGGCTGAACTTCGACGGCAC |
| | | ACTGCTGGGGAACATTACAATTTTCCCTCACCAGTTCATC |
| | | AACCTGAGGTCTAACAACAGCGCCACAATCATTGTTCCAT |
| | | ATGTGAACGCCGTGCCCATGGATAGCATGCCCCGGCACAA |
| | | CAACTGGAGCCTGGTTATCATCCCCATTTGCCCACTGGAG |
| | | AGCGACGGACAGACACCTGTGCCCATCACAATCTCCATCT |
| | | CTCCCATGTGTGCCGAGTTCTCTGGGGCACGCGCTAAATC |
| | | CCAGTGA |

In some embodiments, an immunogenic composition of the invention comprises an mRNA comprising an optimized nucleotide sequence encoding a fusion protein comprising a P2 polyprotein and a VP0 polyprotein. Providing an mRNA comprising an optimized nucleotide sequence encoding the fusion protein is convenient as it simplifies production of the immunogenic composition. For example, only a single mRNA needs to be manufactured by in vitro transcription. Similarly, when the mRNA is encapsulated in a lipid nanoparticle, only a single mRNA needs to be encapsulated during manufacturing.

In some embodiments, the fusion protein is P2-VP0 (i.e., P2 is at the N-terminus). In some embodiments, the P2-VP0 fusion protein has the nucleic acid sequence of SEQ ID NOs: 54 or 55. In alternative embodiments, the fusion protein is VP0-P2 (i.e., VP0 is at the N-terminus). In some embodiments, the VP0-P2 fusion protein has the nucleic acid sequence of SEQ ID NOs: 56 or 57.

In some embodiments, the fusion protein is operationally linked to a non-native secretion signal sequence from an influenza A virus to increase secretion of the mRNA encoded protein(s). Typically, the secretion signal sequence is located at the N-terminus of the fusion protein. Accordingly, in some embodiments, the fusion protein comprises the P2 polyprotein having the amino acid sequence of SEQ ID NO: 30 at the N-terminus and the VP0 polyprotein having the amino acid sequence of SEQ ID NO: 4 at the C-terminus. Accordingly, in one specific embodiment, an immunogenic composition of the invention comprises an mRNA comprising an optimized nucleotide sequence encoding a fusion protein having the nucleic acid sequence of SEQ ID NOs: 58 or 59. In other embodiments, the fusion protein comprises the VP0 polyprotein having the amino acid sequence of SEQ ID NO: 32 at the N-terminus and the P2 polyprotein having the amino acid sequence of SEQ ID NO: 6 at the C-terminus. Accordingly, in one specific embodiment, an immunogenic composition of the invention comprises an mRNA comprising an optimized nucleotide sequence encoding a fusion protein having the nucleic acid sequence of SEQ ID NOs: 60 or 61.

### Additional Rhinovirus Nucleotide Sequences

Methods other than those described herein for optimizing nucleotide sequences for the use with mRNA therapy are known to the skilled person. These methods may result in diverging nucleotide sequences encoding the rhinovirus polypeptides, proteins or polyproteins of the invention. The present disclosure also encompasses such variant nucleotide sequences.

Accordingly, in some embodiments, an optimized nucleotide sequence for use in the immunogenic compositions of the invention comprises a nucleic acid sequence that is at least 80% identical to the nucleotide sequences of SEQ ID NOs: 46 or 47 and encodes an amino acid sequence as set forth in SEQ ID NO: 6. In some embodiments, an optimized nucleotide sequence for use in the immunogenic compositions of the invention comprises a nucleic acid sequence that is at least 85% identical to the nucleotide sequences of SEQ ID NOs: 46 or 47 and encodes an amino acid sequence as set forth in SEQ ID NO: 6. In some embodiments, an optimized nucleotide sequence for use in the immunogenic compositions of the invention comprises a nucleic acid sequence that is at least 90% identical to the nucleotide sequences of SEQ ID NOs: 46 or 47 and encodes an amino acid sequence as set forth in SEQ ID NO: 6. In some embodiments, an optimized nucleotide sequence for use in the immunogenic compositions of the invention comprises a nucleic acid sequence that is at least 95% identical to the nucleotide sequences of SEQ ID NOs: 46 or 47 and encodes an amino acid sequence as set forth in SEQ ID NO: 2. In some embodiments, an optimized nucleotide sequence for use in the immunogenic compositions of the invention comprises a nucleic acid sequence that is at least 99% identical to the nucleotide sequences of SEQ ID NOs: 46 or 47 and encodes an amino acid sequence as set forth in SEQ ID NO: 6.

In some embodiments, an optimized nucleotide sequence for use in the immunogenic compositions of the invention comprises a nucleic acid sequence that is at least 80% identical to the nucleotide sequences of SEQ ID NOs: 48 or 49 and encodes an amino acid sequence as set forth in SEQ ID NO: 4. In some embodiments, an optimized nucleotide sequence for use in the immunogenic compositions of the invention comprises a nucleic acid sequence is at least 85% identical to the nucleotide sequences of SEQ ID NOs: 48 or 49 and encodes an amino acid sequence as set forth in SEQ ID NO: 4. In some embodiments, an optimized nucleotide sequence for use in the immunogenic compositions of the invention comprises a nucleic acid sequence that is at least 90% identical to the nucleotide sequences of SEQ ID NOs: 48 or 49 and encodes an amino acid sequence as set forth in SEQ ID NO: 4. In some embodiments, an optimized nucleotide sequence for use in the immunogenic compositions of the invention comprises a nucleic acid sequence that is at least 99% identical to the nucleotide sequences of SEQ ID NOs: 48 or 49 and encodes an amino acid sequence as set forth in SEQ ID NO: 4.

In some embodiments, an optimized nucleotide sequence for use in the immunogenic compositions of the invention comprises a nucleic acid sequence that is at least 80% identical to the P2-VP0 fusion nucleotide sequences of SEQ ID NOs: 54, 55, 62, or 63 and encodes an amino acid sequence as set forth in SEQ ID NO: 34. In some embodiments, an optimized nucleotide sequence for use in the immunogenic compositions of the invention comprises a nucleic acid sequence that is at least 85% identical to the P2-VP0 fusion nucleotide sequences of SEQ ID NOs: 54, 55, 62, or 63 and encodes an amino acid sequence as set forth in SEQ ID NO: 34. In some embodiments, an optimized nucleotide sequence for use in the immunogenic compositions of the invention comprises a nucleic acid sequence that is at least 90% identical to the P2-VP0 fusion nucleotide sequences of SEQ ID NOs: 54, 55, 62, or 63 and encodes an amino acid sequence as set forth in SEQ ID NO: 34. In some embodiments, an optimized nucleotide sequence for use in the immunogenic compositions of the invention comprises a nucleic acid sequence that is at least 95% identical to the P2-VP0 fusion nucleotide sequences of SEQ ID NOs: 54, 55, 62, or 63 and encodes an amino acid sequence as set forth in SEQ ID NO: 34. In some embodiments, an optimized nucleotide sequence for use in the immunogenic compositions of the invention comprises a nucleic acid sequence that is at least 99% identical to the P2-VP0 fusion nucleotide sequences of SEQ ID NOs: 54, 55, 62, or 63 and encodes an amino acid sequence as set forth in SEQ ID NO: 34.

In some embodiments, an optimized nucleotide sequence for use in the immunogenic compositions of the invention comprises a nucleic acid sequence that is at least 80% identical to the VP0-P2 fusion nucleotide sequences of SEQ ID NOs: 56, 57, 64, or 65 and encodes an amino acid sequence as set forth in SEQ ID NO: 66. In some embodiments, an optimized nucleotide sequence for use in the immunogenic compositions of the invention comprises a nucleic acid sequence that is at least 85% identical to the VP0-P2 fusion nucleotide sequences of SEQ ID NOs: 56, 57, 64, or 65 and encodes an amino acid sequence as set forth in SEQ ID NO: 66. In some embodiments, an optimized nucleotide sequence for use in the immunogenic compositions of the invention comprises a nucleic acid sequence that is at least 90% identical to the VP0-P2 fusion nucleotide sequences of SEQ ID NOs: 56, 57, 64, or 65 and encodes an amino acid sequence as set forth in SEQ ID NO: 66. In some embodiments, an optimized nucleotide sequence for use in the immunogenic compositions of the invention comprises a nucleic acid sequence that is at least 95% identical to the VP0-P2 fusion nucleotide sequences of SEQ ID NOs: 56, 57, 64, or 65 and encodes an amino acid sequence as set forth in SEQ ID NO: 66. In some embodiments, an optimized nucleotide sequence for use in the immunogenic compositions of the invention comprises a nucleic acid sequence that is at least 99% identical to the VP0-P2 fusion nucleotide sequences of SEQ ID NOs: 56, 57, 64, or 65 and encodes an amino acid sequence as set forth in SEQ ID NO: 66.

mRNAs
Structural Elements of mRNAs

A typical mRNA in accordance with the invention comprises a 5′ cap, a 5′ untranslated region (5′ UTR), a protein-coding region, a 3′ untranslated region (3′ UTR), and a 3′ tail.

### 5′ Cap

In a specific embodiment, the mRNA of the invention comprises a 5′ cap with the following structure:

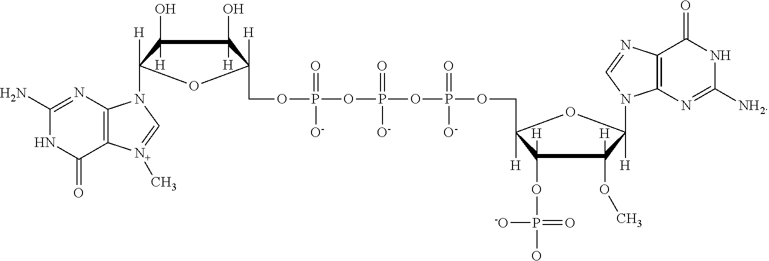

Typically, a 5′ cap and/or a 3′ tail may be added after mRNA synthesis. The presence of the cap is important in providing resistance to nucleases found in most eukaryotic cells. The presence of a “tail” serves to protect the mRNA from exonuclease degradation. Alternatively, the 5′ cap and/or a 3′ tail sequences are included in the DNA template sequences used in in vitro transcription reaction.

A 5′ cap may be added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

### 3′ Tail

In one specific embodiment, the tail structure of the mRNA comprises a poly(A) tail. In another specific embodiment, the tail structure of the mRNA comprises a poly(C) tail. In some embodiments, the tail structure comprises at least 50 adenosine or cytosine nucleotides. In a typical embodiment, the tail structure is approximately 100-500 nucleotides in length. For example, a tail structure (e.g., a poly(A) tail) of 100-250 nucleotides in length may be particularly useful in therapeutic uses of mRNA.

A poly(A) or poly(C) tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, respectively. In some embodiments, a tail structure includes combination of poly(A) and poly(C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

### 5′ UTRs and 3′ UTRs

In some embodiments, an mRNA comprising an optimized nucleotide sequence encoding a polypeptide comprising one more non-structural rhinovirus polyproteins or proteins and/or one or more structural rhinovirus polyproteins or proteins also contains 5′ and 3′ untranslated region (UTR) sequences. In some embodiments, the mRNA comprises a 5′ untranslated region (5′ UTR) different than the naturally occurring 5′ UTR in a naturally occurring mRNA encoding a rhinovirus polyprotein. In a specific embodiment, the 5′ UTR has the nucleotide sequence of SEQ ID NO: 10.

In some embodiments, the mRNA comprises a 3′ untranslated region (3′ UTR) different than the naturally occurring 3′ UTR in a naturally occurring mRNA encoding a rhinovirus polyprotein. In a specific embodiment, the 3′ UTR has the nucleotide sequence of SEQ ID NOs: 11, 12 or 13.

Exemplary 5′ and 3′ UTR sequences are shown in Table 7 below:

TABLE 7
Nucleotide sequences of exemplary 5′
untranslated region (5′ UTR) and 3′
untranslated region (3′ UTR)

| | SEQ | |
|---|---|---|
| | ID | |
| Name | NO. | Sequence |
| 5′ UTR | 10 | GGACAGAUCGCCUGGAGACGCCAUCCACG |
| sequence | | UCUGUUUGACCUCCAUAGAAGACACCGGG |
| | | ACCGAUCCAGCCUCCGCGGCCGGGAACGG |
| | | UGCAUUGGAACGCGGAUUCCCCGUGCCAA |
| | | GAGUGACUCACCGUCCUUGACACG |
| 3′ UTR | 11 | GGGUGGCAUCCCUGUGACCCCUCCCCAGU |
| sequence | | GCCUCUCCUGGCCCUGGAAGUUGCCACUC |
| | | CAGUGCCCACCAGCCUUGUCCUAAUAAAA |
| | | UUAAGUUGCAUC |
| 3′ UTR | 12 | GGGUGGCAUCCCUGUGACCCCUCCCCAGU |
| sequence | | GCCUCUCCUGGCCCUGGAAGUUGCCACUC |
| | | CAGUGCCCACCAGCCUUGUCCUAAUAAAA |
| | | UUAAGUUGCAUCAAAGCU |
| 3′ UTR | 13 | CGGGUGGCAUCCCUGUGACCCCUCCCCAG |
| sequence | | UGCCUCUCCUGGCCCUGGAAGUUGCCACU |
| | | CCAGUGCCCACCAGCCUUGUCCUAAUAAA |
| | | AUUAAGUUGCAUCAAGCU |

Typically, from 5′ to 3′, an mRNA in accordance with the invention comprises a 5′ cap as shown in paragraph [0258], a 5′ UTR as set for in SEQ ID NO: 10, an optimized nucleotides sequence of the invention, a 3′ UTR as set for in SEQ ID NOs: 11, 12 or 13 and a poly(A) tail of 100-250 nucleotides in length.

### Nucleotides

In some embodiments, the mRNA comprises or consists of naturally occurring nucleosides (or unmodified nucleosides; i.e., adenosine, guanosine, cytidine, and uridine). In some embodiments, the mRNA comprises one or more modified nucleosides, such as nucleoside analogs (e.g., adenosine analog, guanosine analog, cytidine analog, or uridine analog). The presence of one or more nucleoside analogs (e.g., N-1-methylpseudouridine) may render an mRNA more stable and/or less immunogenic than a control mRNA with the same sequence but containing only naturally occurring nucleosides.

Accordingly, in some embodiments, the mRNA comprises both unmodified nucleosides and modified nucleosides. In some embodiments, the one or more modified nucleosides is a nucleoside analog. In some embodiments, the one or more modified nucleosides comprises at least one modification selected from a modified sugar, and a modified nucleobase. In some embodiments, the mRNA comprises one or more modified internucleoside linkages.

In some embodiments, the one or more modified nucleosides is a nucleoside analog selected from the group consisting of: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine (e.g., N-1-methyl-pseudouridine), 2-thiouridine, and 2-thiocytidine.

For example, U.S. Pat. No. 8,278,036 and WO 2011/012316 include a discussion of 5-methyl-cytidine, pseudouridine, and 2-thio-uridine and their incorporation into mRNA. In some embodiments, the mRNA may be RNA wherein 25% of U residues are 2-thio-uridine and 25% of C residues are 5-methylcytidine. Teachings for the use of such modified RNA are disclosed in US Patent Publication US 2012/0195936 and international publication WO 2011/012316, both of which are hereby incorporated by reference in their entirety.

mRNAs comprising N-1-methylpseudouridine in place of uridine have been found particularly suitable for use in immunogenic compositions. Accordingly, in a specific embodiment, the mRNA comprises unmodified nucleosides (adenosine, guanosine, cytidine) and modified nucleosides (N-1-methylpseudouridine). In certain embodiments, every uridine in the mRNA is replaced by a pseudouridine, e.g., a methylpseudouridine, such as N-1-methylpseudouridine.

### In Vitro Transcription

mRNAs of the invention may be synthesized according to any of a variety of known methods. Various methods are described in published U.S. Application No. US 2018/0258423 and international patent publication WO 2018/157153, and can be used to practice the present invention, all of which are incorporated herein by reference. For example, mRNAs according to the present invention may be synthesized via in vitro transcription (IVT). Briefly, IVT is typically performed with a linear or circular DNA template or DNA vector containing a promoter, a pool of ribonucleotide triphosphates, a buffer system that may include DTT and magnesium ions, and an appropriate RNA polymerase (e.g., T3, T7, or SP6 RNA polymerase), DNase I, pyrophosphatase, and/or RNase inhibitor. The exact conditions will vary according to the specific application.

For the preparation of mRNA by IVT, a DNA template or DNA vector may be transcribed in vitro. A suitable DNA template or DNA vector typically has a promoter, for example a T3, T7 or SP6 promoter, for in vitro transcription, followed by desired nucleotide sequence for desired mRNA and a termination signal (terminator).

In one aspect, the invention provides a DNA vector encoding an mRNA comprising an optimized nucleotide sequence described herein. In some embodiments, the DNA vector further comprises a promoter and/or a terminator. In one embodiment, the promoter is a SP6 RNA polymerase promoter. In another embodiment, the promoter is a T7 RNA polymerase promoter. In some embodiments, the RNA polymerase promoter is operationally linked to the optimized nucleotide sequence. In some embodiments, the nucleic acid is linear or circular.

### Post-Synthesis Purification

Various methods may be used to purify mRNA after synthesis. In some embodiments, the mRNA is purified using Tangential Flow Filtration (TFF). Suitable purification methods include those described in published U.S. Application No. US 2016/0040154, published U.S. Application No. US 2015/0376220, published U.S. Application No. US 2018/0251755, published U.S. Application No. US 2018/0251754, published International Application No. WO 2020/097509 filed on Nov. 8, 2019, and published International Application No. WO 2020/232371 filed on May 15, 2020, all of which are incorporated by reference herein and may be used to practice the present invention. It may be advantageous to purify the mRNA of the invention which may be included in pharmaceutical compositions in some embodiments of the invention, as the purity requirements for mRNA products are more stringent for therapeutic applications.

In some embodiments, the mRNA is purified before capping and tailing. In some embodiments, the mRNA is purified after capping and tailing. In some embodiments, the mRNA is purified both before and after capping and tailing. In some embodiments, the mRNA is purified either before or after or both before and after capping and tailing, by centrifugation. In some embodiments, the mRNA is purified either before or after or both before and after capping and tailing, by filtration. In some embodiments, the mRNA is purified either before or after or both before and after capping and tailing, by Tangential Flow Filtration (TFF).

### Lipid Nanoparticles (LNPs)

A lipid nanoparticle (LNP) encapsulating an mRNA of the invention is also provided. In some embodiments, a lipid nanoparticle suitable for use with the present invention comprises one or more cationic lipids, one or more non-cationic lipids (e.g., DOPE and/or cholesterol), and one or more PEG-modified lipids (e.g., DMG-PEG2K).

A typical lipid nanoparticle for use with the invention is composed of four lipid components: a cationic lipid (e.g., a sterol-based cationic lipid), a non-cationic lipid (e.g., DOPE or DEPE), a cholesterol-based lipid (e.g., cholesterol) and a PEG-modified lipid (e.g., DMG-PEG2K). In a specific embodiment, the non-cationic lipid is DOPE. The molar ratio of cationic lipid to non-cationic lipid to cholesterol to PEG-modified lipid typically is between about 30-60:25-35:20-30:1-15, respectively. An exemplary LNP for use with the immunogenic compositions of the invention may be composed of a cationic lipid selected from cKK-E12, cKK-E10, OF-Deg-Lin and OF-02; a non-cationic lipid selected from DOPE and DEPE; a cholesterol-based lipid such as cholesterol; and a PEG-modified lipid such as DMG-PEG-2K.

In some embodiments, a lipid nanoparticle comprises no more than three distinct lipid components. An exemplary lipid nanoparticle is composed of three lipid components: a cationic lipid (e.g., a sterol-based cationic lipid), a non-cationic lipid (e.g., DOPE or DEPE) and a PEG-modified lipid (e.g., DMG-PEG2K). In a specific embodiment, the three distinct lipid components are HGT4002, DOPE and DMG-PEG2K. In an exemplary embodiment, HGT4002, DOPE and DMG-PEG2K are present in a molar ratio of approximately 60:35:5, respectively. Such LNPs may be particularly suitable for aerosol delivery of the mRNAs of the invention.

The lipid nanoparticles for use in the invention can be prepared by various techniques which are presently known in the art. Such methods are described, e.g., in published U.S. Application No. US 2011/0244026, published U.S. Application No. US 2016/0038432, published U.S. Application No. US 2018/0153822, published U.S. Application No. US 2018/0125989 and published International Application No. WO 2021/016430, filed Jul. 23, 2020, all of which are incorporated herein by reference.

### Lipid Nanoparticle Formulations

In some embodiments, the majority of LNPs in a composition of the invention, i.e., greater than about 50%, 55%, 60%, 65%, 70%, 75%, 80%, 85%, 90%, 95%, 96%, 97%, 98%, or 99% of the LNPs, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, the LNPs in a composition of the invention have a size of about 150 nm or less (e.g., about 145 nm or less, about 140 nm or less, about 135 nm or less, about 130 nm or less, about 125 nm or less, about 120 nm or less, about 115 nm or less, about 110 nm or less, about 105 nm or less, about 100 nm or less, about 95 nm or less, about 90 nm or less, about 85 nm or less, or about 80 nm or less).

In some embodiments, greater than about 70%, 75%, 80%, 85%, 90%, 95%, 96%, 97%, 98%, 99% of the LNPs in a composition provided by the present invention have a size ranging from about 40-90 nm (e.g., about 45-85 nm, about 50-80 nm, about 55-75 nm, about 60-70 nm). In some embodiments, the LNPs have a size ranging from about 40-90 nm (e.g., about 45-85 nm, about 50-80 nm, about 55-75 nm, about 60-70 nm). Compositions with LNPs having an average size of about 50-70 nm (e.g., 55-65 nm) may be particularly suitable for pulmonary delivery via nebulization.

In some embodiments, the dispersity, or measure of heterogeneity in size of molecules (PDI), of LNPs in a pharmaceutical composition provided by the present invention is less than about 0.5. In some embodiments, a LNP has a PDI of less than about 0.5. In some embodiments, a LNP has a PDI of less than about 0.4. In some embodiments, a LNP has a PDI of less than about 0.3. In some embodiments, a LNP has a PDI of less than about 0.28. In some embodiments, a LNP has a PDI of less than about 0.25. In some embodiments, a LNP has a PDI of less than about 0.23. In some embodiments, a LNP has a PDI of less than about 0.20. In some embodiments, a LNP has a PDI of less than about 0.18. In some embodiments, a LNP has a PDI of less than about 0.16. In some embodiments, a LNP has a PDI of less than about 0.14. In some embodiments, a LNP has a PDI of less than about 0.12. In some embodiments, a LNP has a PDI of less than about 0.10. In some embodiments, a LNP has a PDI of less than about 0.08.

In some embodiments, an LNP has an encapsulation efficiency of greater than about 80%. In some embodiments, an LNP has an encapsulation efficiency of greater than about 85%. In some embodiments, an LNP has an encapsulation efficiency of greater than about 90%. In some embodiments, an LNP has an encapsulation efficiency of greater than about 92%. In some embodiments, an LNP has an encapsulation efficiency of greater than about 95%. In some embodiments, an LNP has an encapsulation efficiency of greater than about 98%. In some embodiments, an LNP has an encapsulation efficiency of greater than about 99%. Typically, LNPs for use with compositions of the invention have an encapsulation efficiency of at least 90%-95%. Cationic Lipids

Various cationic lipids which are suitable for use in LNPs are known in the art. These include, for example, DOTAP (1,2-dioleyl-3-trimethylammonium propane), DODAP (1,2-dioleyl-3-dimethylammonium propane), DOTMA (N-[1-(2,3-dioleyloxy)propyl]-N,N,N-trimethylammonium chloride), DLinKC2DMA, DLin-KC2-DM, and C12-200. Exemplary cationic lipids suitable for use in the LNPs, compositions, pharmaceutical compositions and methods of the invention are described herein and include, for instance, the cationic lipids as described in International Patent Publication WO 2010/144740, which is incorporated herein by reference. In some embodiments, the LNPs, compositions, pharmaceutical compositions and methods of the present invention include a cationic lipid, (6Z,9Z,28Z,31Z)-heptatriaconta-6,9,28,31-tetraen-19-yl 4-(dimethylamino) butanoate, having a compound structure of:

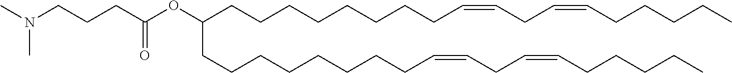 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the LNPs, compositions, pharmaceutical compositions and methods of the present invention include ionizable cationic lipids as described in International Patent Publication WO 2013/149140, which is incorporated herein by reference. In some embodiments, the LNPs, compositions, pharmaceutical compositions and methods of the present invention include a cationic lipid of one of the following formulas:

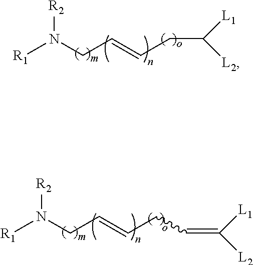

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In some embodiments, the LNPs, compositions, pharmaceutical compositions and methods of the present invention include the cationic lipid (15Z,18Z)-N,N-dimethyl-6-(9Z,12Z)-octadeca-9,12-dien-1-yl) tetracosa-15,18-dien-1-amine (“HGT5000”), having a compound structure of:

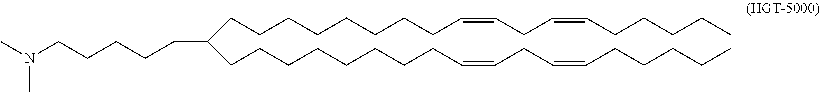 [SMILES: CCCCC/C=C\C/C=C\CCCCCCCCC(CCCCCCCC/C=C\C/C=C\CCCCC)CCCCCN(C)C]

and pharmaceutically acceptable salts thereof. In some embodiments, the LNPs, compositions, pharmaceutical compositions and methods of the present invention include the cationic lipid (15Z, 18Z)-N,N-dimethyl-6-((9Z,12Z)-octadeca-9,12-dien-1-yl) tetracosa-4,15,18-trien-1-amine (“HGT5001”), having a compound structure of:

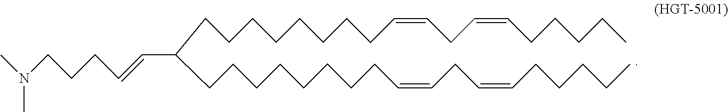 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In some embodiments, the LNPs, compositions, pharmaceutical compositions and methods of the present invention include the cationic lipid and (15Z,18Z)-N,N-dimethyl-6-((9Z,12Z)-octadeca-9,12-dien-1-yl) tetracosa-5,15,18-trien-1-amine (“HGT5002”), having a compound structure of:

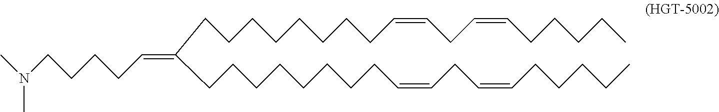 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the LNPs, compositions, pharmaceutical compositions and methods of the invention include cationic lipids described as aminoalcohol lipidoids in International Patent Publication WO 2010/053572, which is incorporated herein by reference. In some embodiments, the LNPs, compositions, pharmaceutical compositions and methods of the present invention include a cationic lipid having a compound structure of:

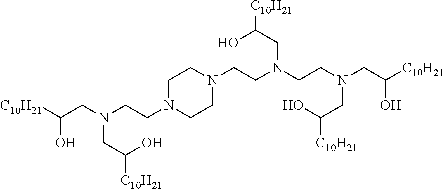

and pharmaceutically acceptable salts thereof.

Other suitable cationic lipids for use in the LNPs, compositions, pharmaceutical compositions and methods of the invention include the cationic lipids as described in International Patent Publication WO 2016/118725, which is incorporated herein by reference. In some embodiments, the LNPs, compositions, pharmaceutical compositions and methods of the present invention include a cationic lipid having a compound structure of:

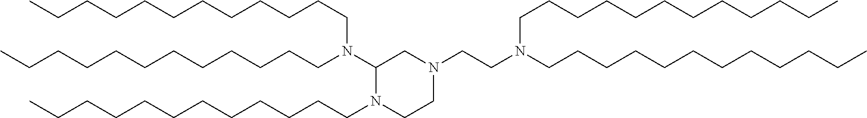 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the LNPs, compositions, pharmaceutical compositions and methods of the invention include the cationic lipids as described in International Patent Publication WO 2016/118724, which is incorporated herein by reference. In some embodiments, the LNPs, compositions, pharmaceutical compositions and methods of the present invention include a cationic lipid having a compound structure of:

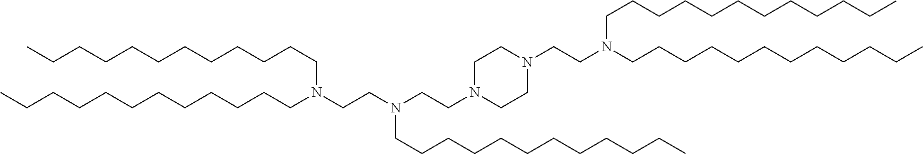 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the LNPs, compositions, pharmaceutical compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the LNPs, compositions, pharmaceutical compositions and methods of the invention include the cationic lipids as described in International Patent Publications WO 2013/063468 and WO 2016/205691, each of which are incorporated herein by reference. In some embodiments, the LNPs, compositions, pharmaceutical compositions and methods of the present invention include a cationic lipid of the following formula:

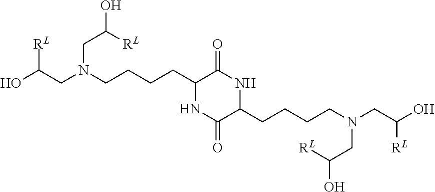

or pharmaceutically acceptable salts thereof, wherein each instance of R^{L} is independently optionally substituted C₆-C₄₀ alkenyl. In some embodiments, the LNPs, compositions, pharmaceutical compositions and methods of the present invention include a cationic lipid having a compound structure of:

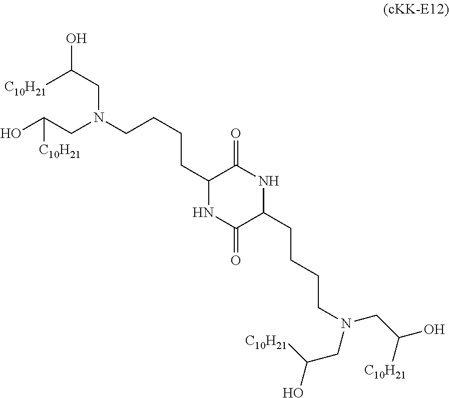

and pharmaceutically acceptable salts thereof. In some embodiments, the LNPs, compositions, pharmaceutical compositions and methods of the present invention include a cationic lipid having a compound structure of:

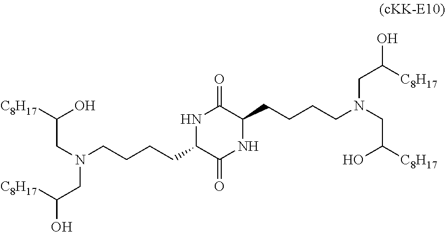

and pharmaceutically acceptable salts thereof.
In some embodiments, the LNPs, compositions, pharmaceutical compositions and methods of the present invention include a cationic lipid having a compound structure of:

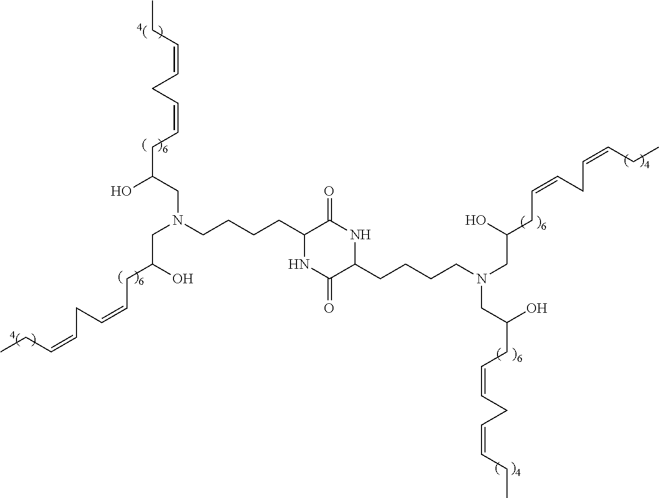

and pharmaceutically acceptable salts thereof. In some embodiments, the LNPs, compositions, pharmaceutical compositions and methods of the present invention include a cationic lipid having a compound structure of:

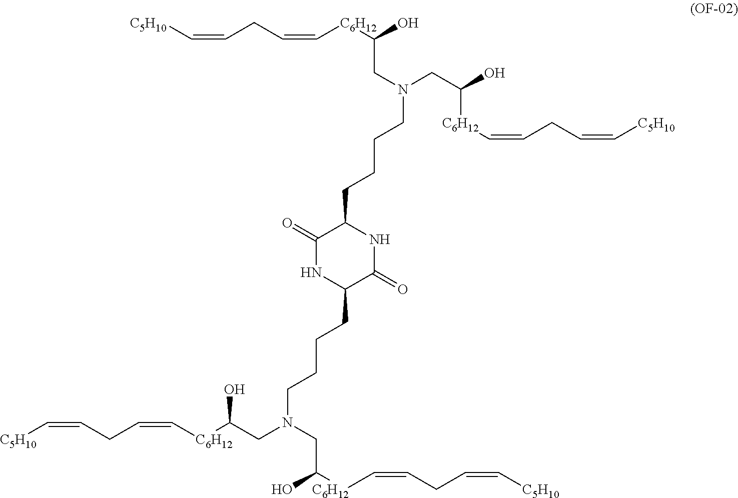

and pharmaceutically acceptable salts thereof. In some embodiments, the LNPs, compositions, pharmaceutical compositions and methods of the present invention include a cationic lipid having a compound structure of:

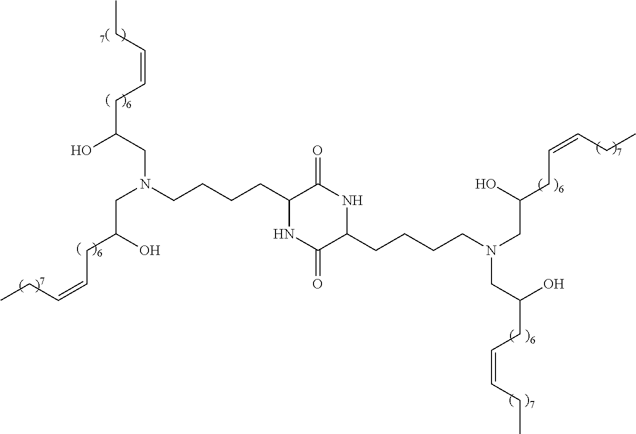

and pharmaceutically acceptable salts thereof. In some embodiments, the LNPs, compositions, pharmaceutical compositions and methods of the present invention include a cationic lipid having a compound structure of:

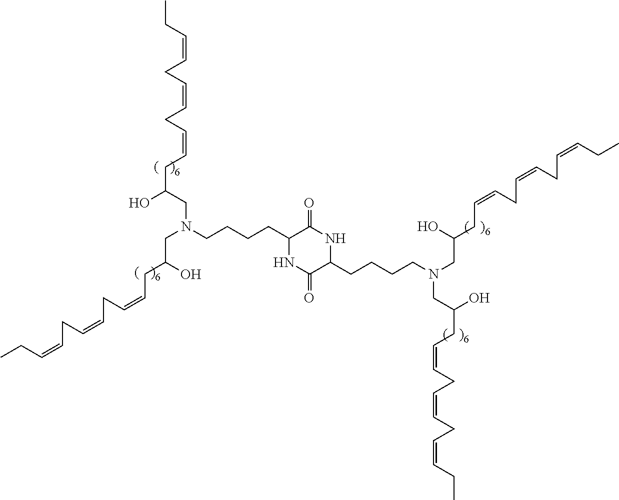

and pharmaceutically acceptable salts thereof.

Other suitable cationic lipids for use in the LNPs, compositions, pharmaceutical compositions and methods of the invention include the cationic lipids as described in International Patent Publication WO 2015/184256, which is incorporated herein by reference. In some embodiments, the LNPs, compositions, pharmaceutical compositions and methods of the present invention include a cationic lipid of the following formula:

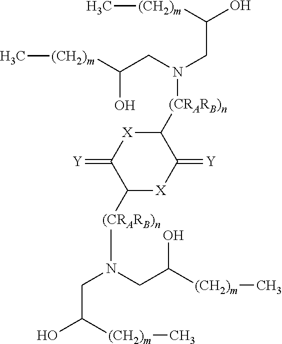

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In some embodiments, the LNPs, compositions, pharmaceutical compositions and methods of the present invention include a cationic lipid, “Target 23”, having a compound structure of:

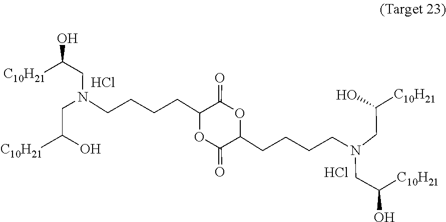

and pharmaceutically acceptable salts thereof.

Other suitable cationic lipids for use in the LNPs, compositions, pharmaceutical compositions and methods of the invention include the cationic lipids as described in International Patent Publication WO 2016/004202, which is incorporated herein by reference. In some embodiments, the LNPs, compositions, pharmaceutical compositions and methods of the present invention include a cationic lipid having the compound structure:

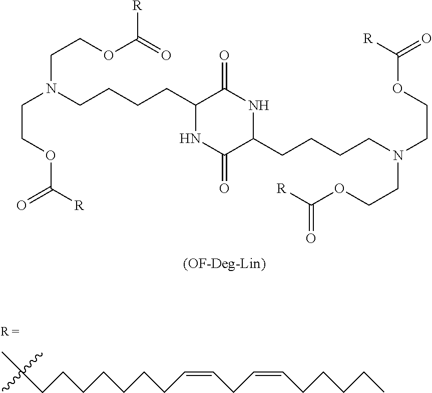

or a pharmaceutically acceptable salt thereof. In some embodiments, the LNPs, compositions, pharmaceutical compositions and methods of the present invention include a cationic lipid having the compound structure:

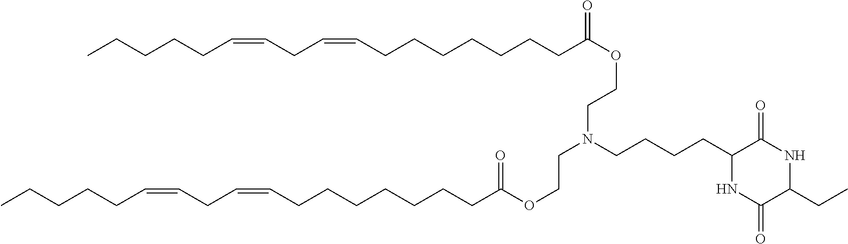 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the LNPs, compositions, pharmaceutical compositions and methods of the present invention include a cationic lipid having the compound structure:

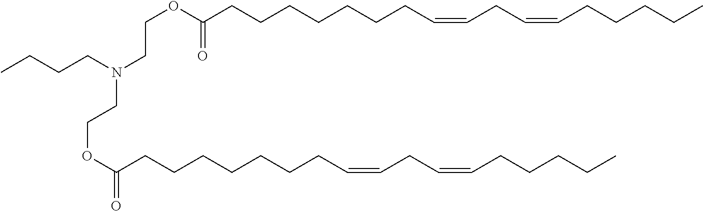 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the LNPs, compositions, pharmaceutical compositions and methods of the present invention include cationic lipids as described in International Patent Publication WO 2020/097384, which is incorporated herein by reference. In some embodiments, the LNPs, compositions, pharmaceutical compositions and methods of the present invention include a cationic lipid of the following formula:

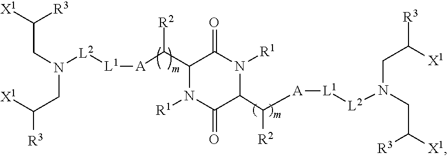

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the LNPs, compositions, pharmaceutical compositions and methods of the present invention include a cationic lipid of the following formula:

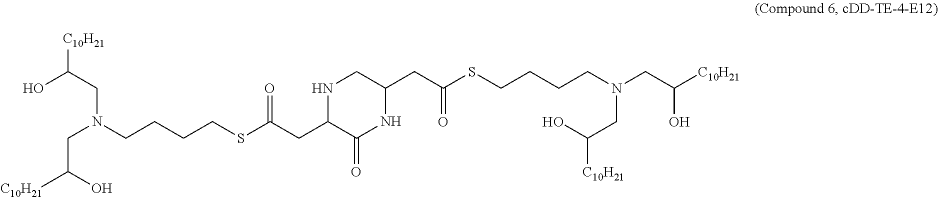

or a pharmaceutically acceptable salt thereof. In some embodiments, the LNPs, compositions, pharmaceutical compositions and methods of the present invention include a cationic lipid of the following formula:

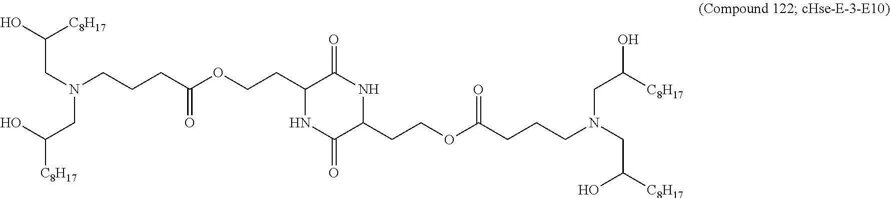

or a pharmaceutically acceptable salt thereof. In some embodiments, the LNPs, compositions, pharmaceutical compositions and methods of the present invention include a cationic lipid of the following formula:

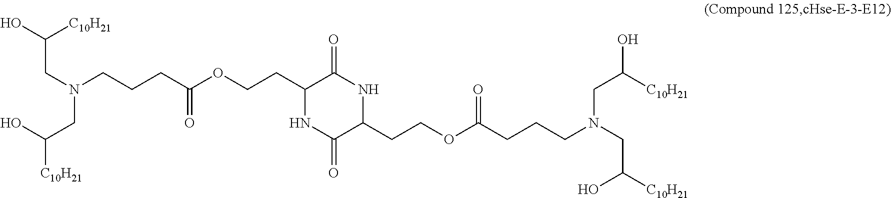

or a pharmaceutically acceptable salt thereof.

Other suitable cationic lipids for use in the pharmaceutical compositions and methods of the present invention include the cationic lipids as described in J. McClellan, M. C. King, Cell 2010, 141, 210-217 and in Whitehead et al., Nature Communications (2014) 5:4277, which is incorporated herein by reference. In some embodiments, the cationic lipids of the LNPs, compositions, pharmaceutical compositions and methods of the present invention include a cationic lipid having a compound structure of:

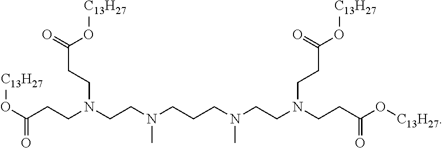

and pharmaceutically acceptable salts thereof.

Other suitable cationic lipids for use in the LNPs, compositions, pharmaceutical compositions and methods of the invention include the cationic lipids as described in International Patent Publication WO 2015/199952, which is incorporated herein by reference. In some embodiments, the LNPs, compositions, pharmaceutical compositions and methods of the present invention include a cationic lipid having the compound structure:

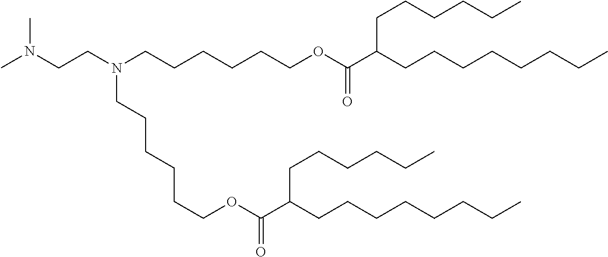 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

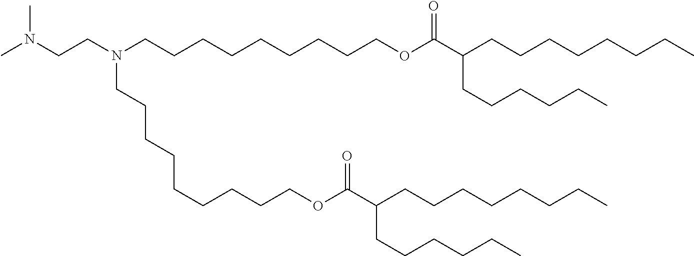 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

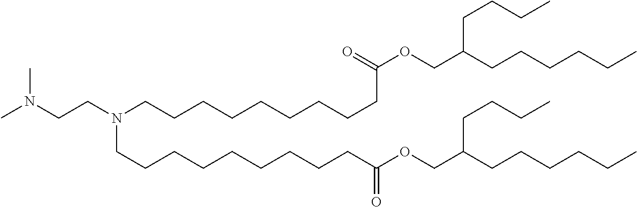 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the LNPs, compositions, pharmaceutical compositions and methods of the present invention include a cationic lipid having the compound structure:

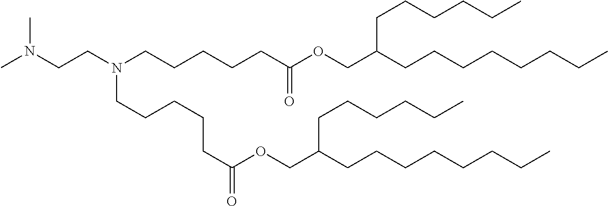 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

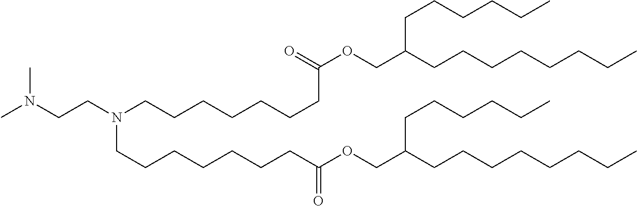 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

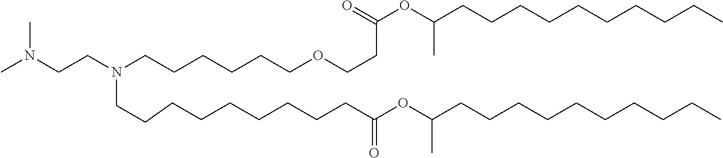 [SMILES: CCCCCCCCCCC(C)OC(=O)CCCCCCCCCN(CCCCCCOCCC(=O)OC(C)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

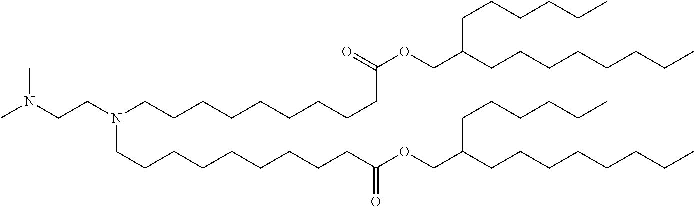 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

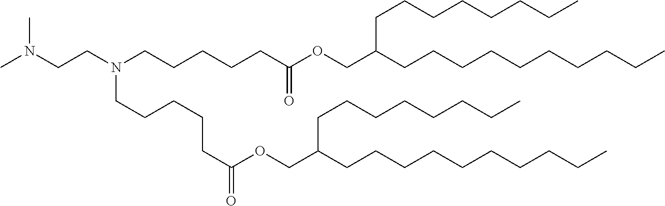 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

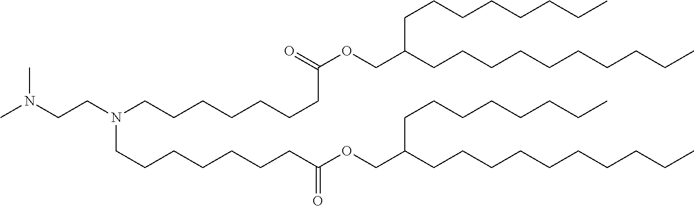 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

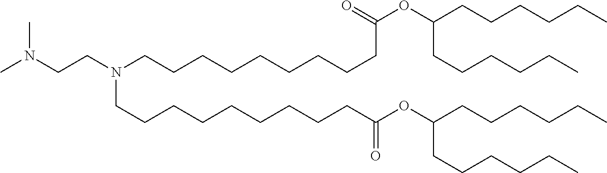 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the LNPs, compositions, pharmaceutical compositions and methods of the present invention include a cationic lipid having the compound structure:

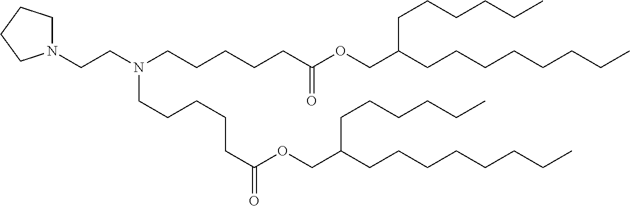 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the LNPs, compositions, pharmaceutical compositions and methods of the present invention include a cationic lipid having the compound structure:

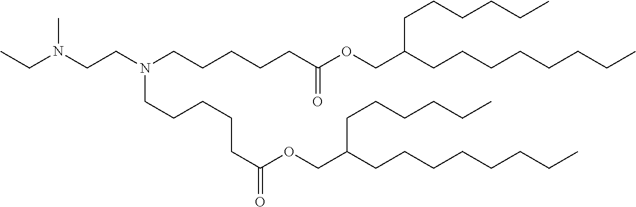 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the LNPs, compositions, pharmaceutical compositions and methods of the present invention include a cationic lipid having the compound structure:

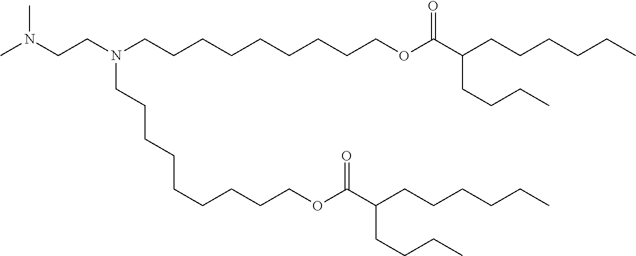 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the LNPs, compositions, pharmaceutical compositions and methods of the invention include the cationic lipids as described in International Patent Publication WO 2017/004143, which is incorporated herein by reference. In some embodiments, the LNPs, compositions, pharmaceutical compositions and methods of the present invention include a cationic lipid having the compound structure:

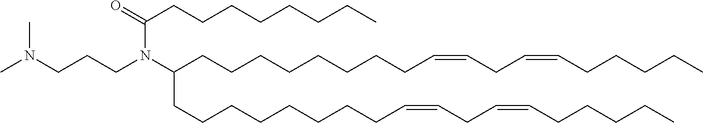 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the LNPs, compositions, pharmaceutical compositions and methods of the present invention include a cationic lipid having the compound structure:

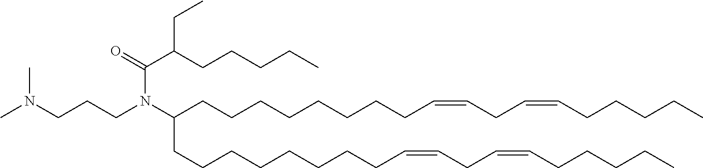 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the LNPs, compositions, pharmaceutical compositions and methods of the present invention include a cationic lipid having the compound structure:

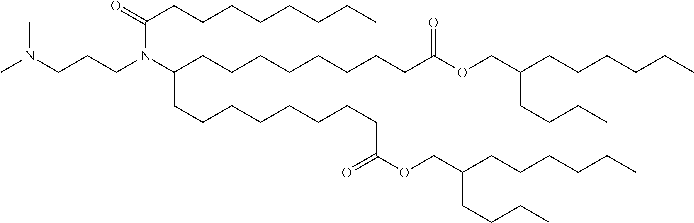 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

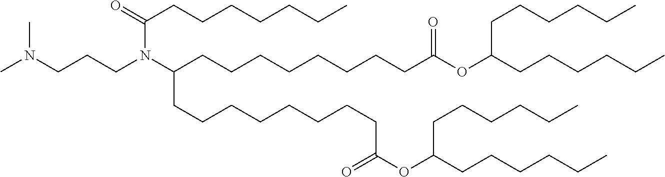 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the LNPs, compositions, pharmaceutical compositions and methods of the present invention include a cationic lipid having the compound structure:

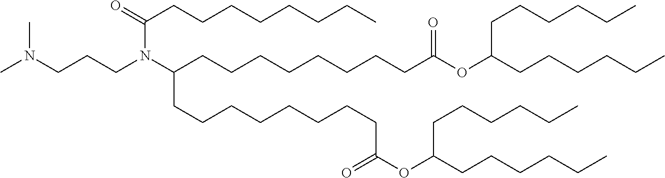 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the LNPs, compositions, pharmaceutical compositions and methods of the present invention include a cationic lipid having the compound structure:

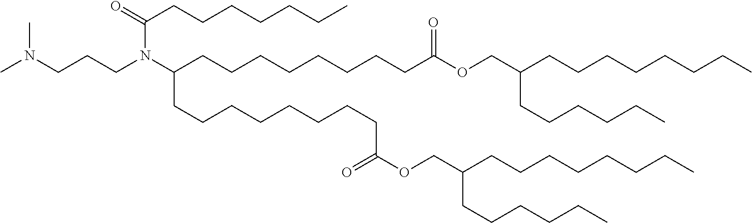 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the LNPs, compositions, pharmaceutical compositions and methods of the present invention include a cationic lipid having the compound structure:

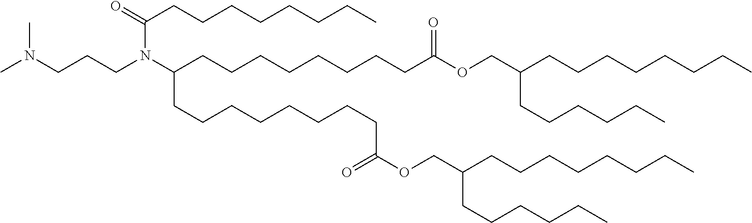 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the LNPs, compositions, pharmaceutical compositions and methods of the present invention include a cationic lipid having the compound structure:

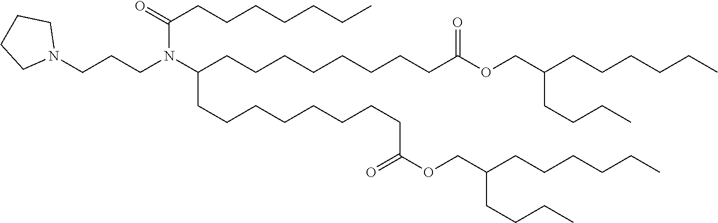 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

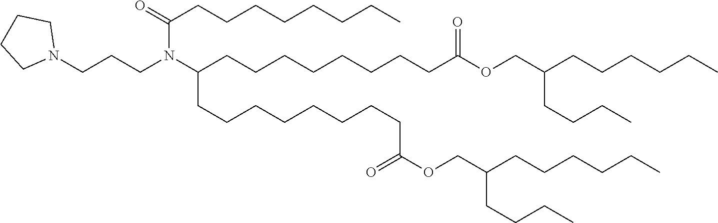 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

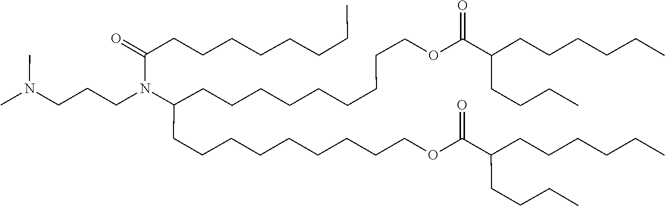 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the LNPs, compositions, pharmaceutical compositions and methods of the present invention include a cationic lipid having the compound structure:

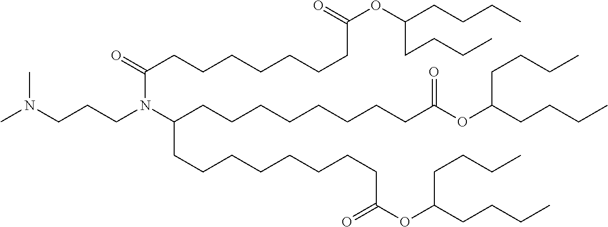 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the LNPs, compositions, pharmaceutical compositions and methods of the present invention include a cationic lipid having the compound structure:

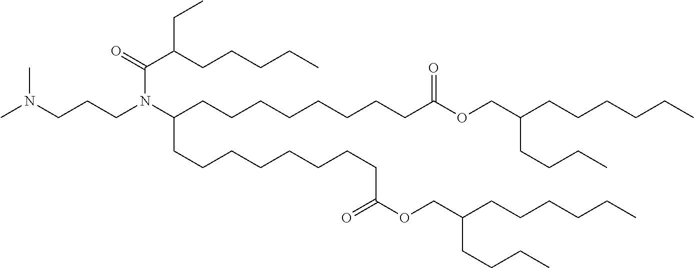 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the LNPs, compositions, pharmaceutical compositions and methods of the present invention include a cationic lipid having the compound structure:

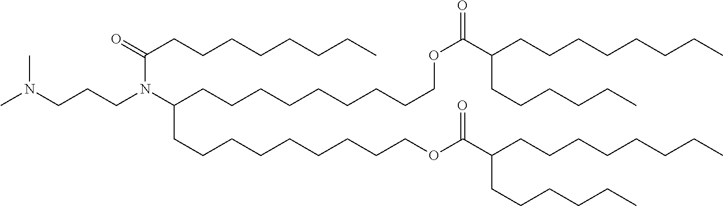 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the LNPs, compositions, pharmaceutical compositions and methods of the present invention include a cationic lipid having the compound structure;

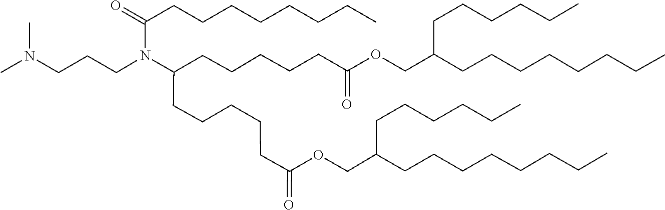 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the LNPs, compositions, pharmaceutical compositions and methods of the present invention include a cationic lipid having the compound structure:

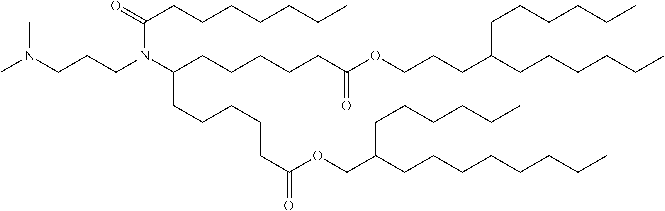 [SMILES: CCCCCCCCC(CCCCCC)COC(=O)CCCCCC(CCCCCC(=O)OCCCC(CCCCCC)CCCCCC)N(CCCN(C)C)C(=O)CCCCCCC]

and pharmaceutically acceptable salts thereof. In some embodiments, the LNPs, compositions, pharmaceutical compositions and methods of the present invention include a cationic lipid having the compound structure:

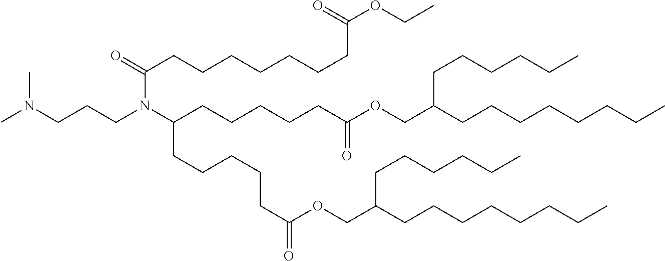 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the LNPs, compositions, pharmaceutical compositions and methods of the present invention include a cationic lipid having the compound structure:

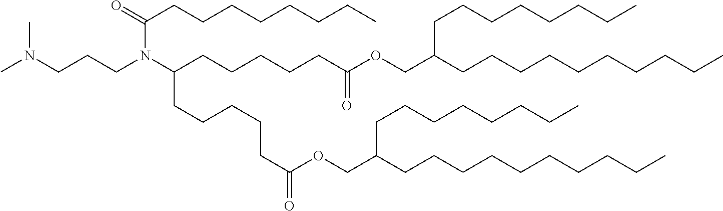 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the LNPs, compositions, pharmaceutical compositions and methods of the invention include the cationic lipids as described in International Patent Publication WO 2017/075531, which is incorporated herein by reference. In some embodiments, the LNPs, compositions, pharmaceutical compositions and methods of the present invention include a cationic lipid of the following formula:

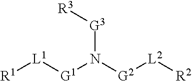

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₅ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵ C(═O)R⁴; R⁴ is C₁-C₁₂ alkyl; R⁵ is H or C₁-C₆ alkyl; and x is 0, 1 or 2.

Other suitable cationic lipids for use in the LNPs, compositions, pharmaceutical compositions and methods of the invention include the cationic lipids as described in International Patent Publication WO 2017/117528, which is incorporated herein by reference. In some embodiments, the LNPs, compositions, pharmaceutical compositions and methods of the present invention include a cationic lipid having the compound structure:

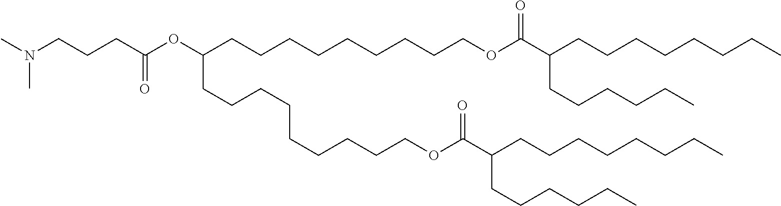 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the LNPs, compositions, pharmaceutical compositions and methods of the present invention include a cationic lipid having the compound structure:

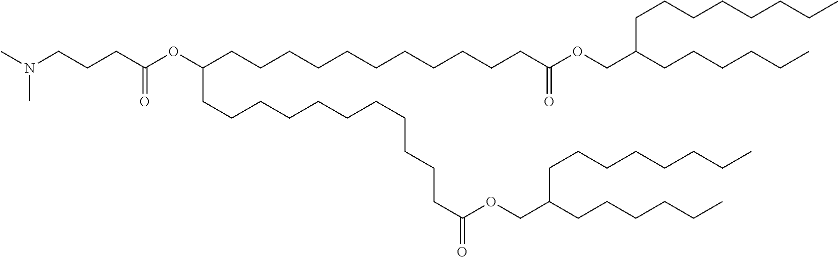 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the LNPs, compositions, pharmaceutical compositions and methods of the present invention include a cationic lipid having the compound structure:

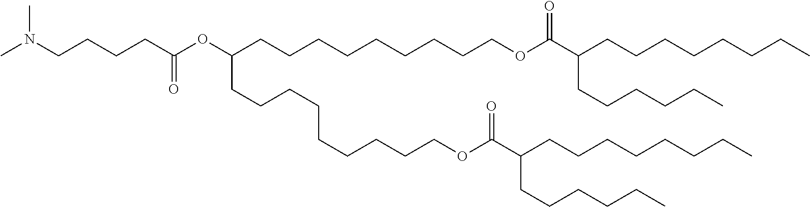 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the LNPs, compositions, pharmaceutical compositions and methods of the invention include the cationic lipids as described in International Patent Publication WO 2017/049245, which is incorporated herein by reference. In some embodiments, the cationic lipids of the LNPs, compositions, pharmaceutical compositions and methods of the present invention include a compound of one of the following formulas:

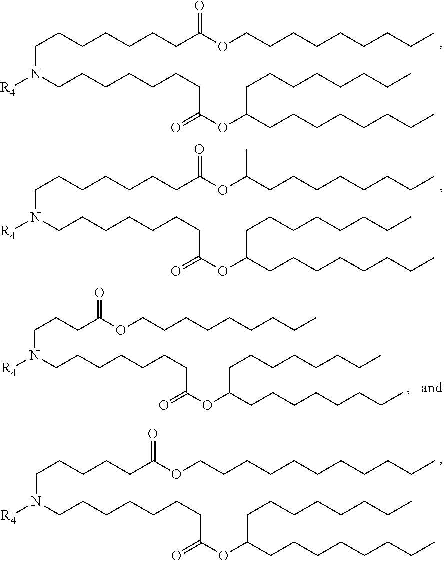

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In some embodiments, the LNPs, compositions, pharmaceutical compositions and methods of the present invention include a cationic lipid having a compound structure of:

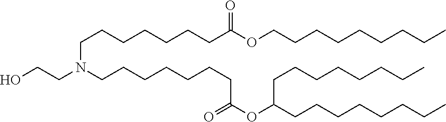 [SMILES: CCCCCCCCCOC(=O)CCCCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

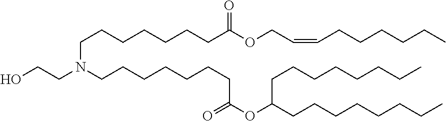 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

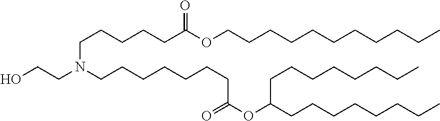 [SMILES: CCCCCCCCCCCOC(=O)CCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

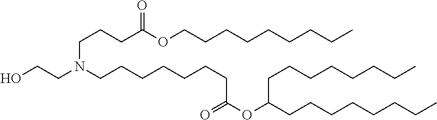 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the LNPs, compositions, pharmaceutical compositions and methods of the invention include the cationic lipids as described in International Patent Publication WO 2017/173054 and WO 2015/095340, each of which is incorporated herein by reference. In some embodiments, the LNPs, compositions, pharmaceutical compositions and methods of the present invention include a cationic lipid having a compound structure of:

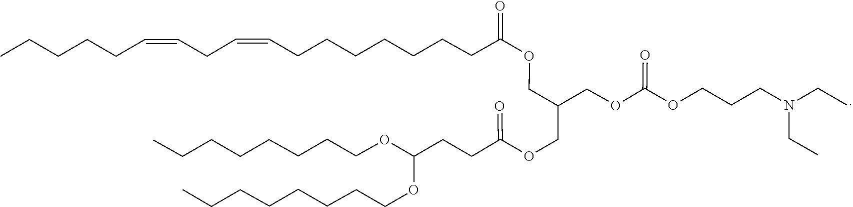 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In some embodiments, the LNPs, compositions, pharmaceutical compositions and methods of the present invention include a cationic lipid having a compound structure of:

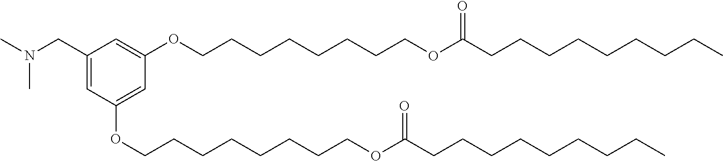 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In some embodiments, the LNPs, compositions, pharmaceutical compositions and methods of the present invention include a cationic lipid having a compound structure of:

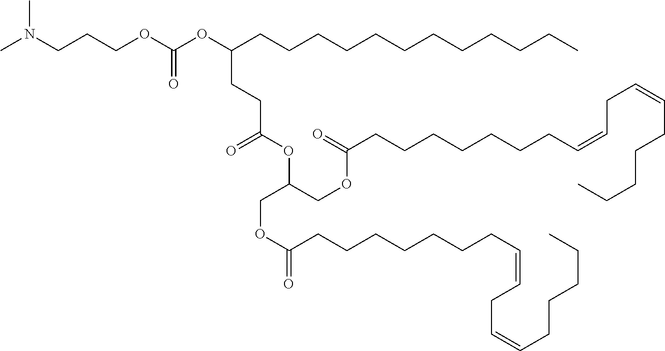 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In some embodiments, the LNPs, compositions, pharmaceutical compositions and methods of the present invention include a cationic lipid having a compound structure of:

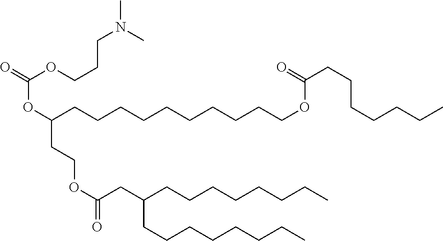 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the LNPs, compositions, pharmaceutical compositions and methods of the present invention include cationic lipids as described in published International Application No. WO 2022/066678, filed on Sep. 22, 2021, which is incorporated herein by reference. In some embodiments, the LNPs, compositions, pharmaceutical compositions and methods of the present invention include a cationic lipid having a compound structure of:

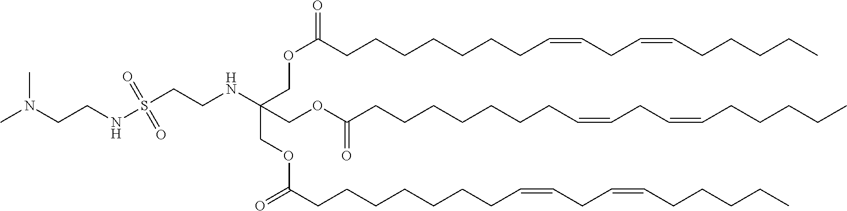 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

(GL-TES-SA-DME-E18-2) and pharmaceutically acceptable salts thereof.

In some embodiments, the LNPs, compositions, pharmaceutical compositions and methods of the present invention include a cationic lipid having a compound structure of:

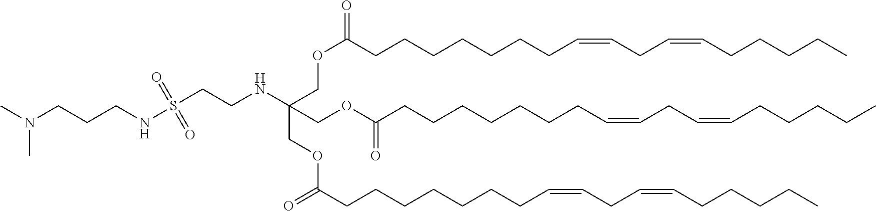 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

(GL-TES-SA-DMP-E18-2) and pharmaceutically acceptable salts thereof.

Other suitable cationic lipids for use in the LNPs, compositions, pharmaceutical compositions and methods of the present invention include cationic lipids as described in published International Application No. WO 2021/202694, filed on Mar. 31, 2021, which is incorporated herein by reference. In some embodiments, the LNPs, compositions, pharmaceutical compositions and methods of the present invention include a cationic lipid having a compound structure of:

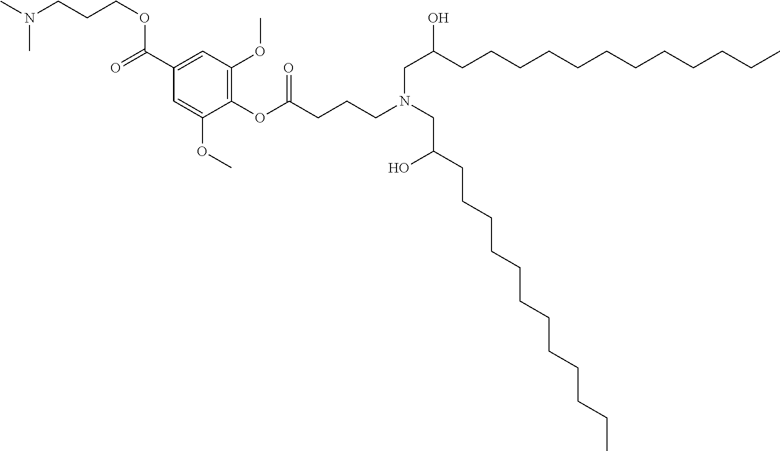 [SMILES: CCCCCCCCCCCCC(O)CN(CCCC(=O)Oc1c(OC)cc(C(=O)OCCCN(C)C)cc1OC)CC(O)CCCCCCCCCCCC]

(SY-3-E14-DMAPr) and pharmaceutically acceptable salts thereof.

Other suitable cationic lipids for use in the LNPs, compositions, pharmaceutical compositions and methods of the present invention include cationic lipids as described in published International Application No. WO 2022/066916, filed on Sep. 23, 2021, which is incorporated herein by reference. In some embodiments, the LNPs, compositions, pharmaceutical compositions and methods of the present invention include a cationic lipid having a compound structure of:

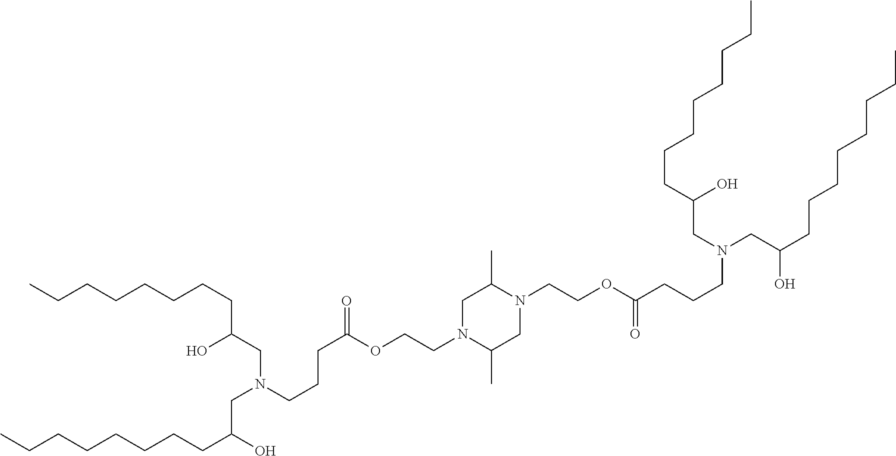 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

(HEP-E3-E10) and pharmaceutically acceptable salts thereof.

In some embodiments, the LNPs, compositions, pharmaceutical compositions and methods of the present invention include a cationic lipid having a compound structure of:

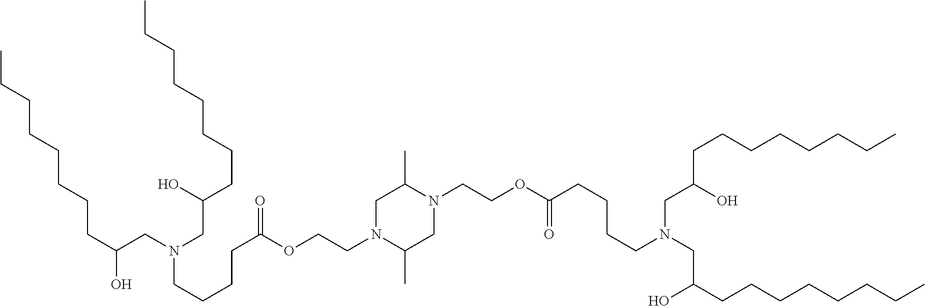 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

(HEP-E4-E10) and pharmaceutically acceptable salts thereof.

Other suitable cationic lipids for use in the LNPs, compositions, pharmaceutical compositions and methods of the present invention include cationic lipids as described in published International Application No. WO 2020/257716, filed on Jun. 19, 2020, which is incorporated herein by reference. In some embodiments, the LNPs, compositions, pharmaceutical compositions and methods of the present invention include a cationic lipid having a compound structure according to the following formula:

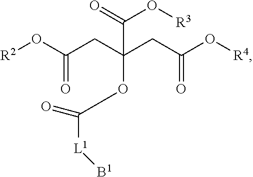

or a pharmaceutically acceptable salt thereof, wherein each of R², R³, and R⁴ is independently C₆-C₃₀ alkyl, C₆-C₃₀ alkenyl, or C₆-C₃₀ alkynyl; L¹ is C₁-C₃₀ alkylene; C₂-C₃₀ alkenylene; or C₂-C₃₀ alkynylene and B¹ is an ionizable nitrogen-containing group. In embodiments, L¹ is C₁-C₁₀ alkylene. In embodiments, L¹ is unsubstituted C₁-C₁₀ alkylene. In embodiments, L¹ is (CH₂)₂, (CH₂)₃, (CH₂)₄, or (CH₂)₅. In embodiments, L¹ is (CH₂), (CH₂)₆, (CH₂)₇, (CH₂)₈, (CH₂)₉, or (CH₂)₁₀. In embodiments, B¹ is independently NH₂, guanidine, amidine, a mono- or dialkylamine, 5- to 6-membered nitrogen-containing heterocycloalkyl, or 5- to 6-membered nitrogen-containing heteroaryl. In embodiments, B¹ is

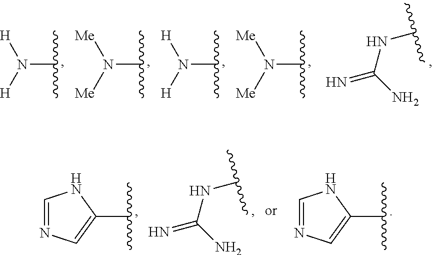

In embodiments, B¹ is

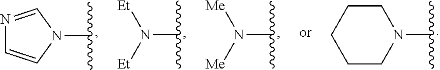

In embodiments, B¹ is

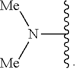 [SMILES: *N(C)C]

In embodiments, each of R², R³, and R⁴ is independently unsubstituted linear C₆-C₂₂ alkyl, unsubstituted linear C₆-C₂₂ alkenyl, unsubstituted linear C₆-C₂₂ alkynyl, unsubstituted branched C₆-C₂₂ alkyl, unsubstituted branched C₆-C₂₂ alkenyl, or unsubstituted branched C₆-C₂₂ alkynyl. In embodiments, each of R², R³, and R⁴ is unsubstituted C₆-C₂₂ alkyl. In embodiments, each of R², R³, and R⁴ is —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, —C₂₀H₄₁, —C₂₁H₄₃, —C₂₂H₄₅, —C₂₃H₄₇, —C₂₄H₄₉, or —C₂₅H₅₁. In embodiments, each of R², R³, and R⁴ is independently C₆-C₁₂ alkyl substituted by —O(CO)R⁵ or —C(O)OR⁵, wherein R⁵ is unsubstituted C₆-C₁₄ alkyl. In embodiments, each of R², R³, and R⁴ is unsubstituted C₆-C₂₂ alkenyl. In embodiments, each of R², R³, and R⁴ is —(CH₂)₄CH═CH₂, —(CH₂)₅CH═CH₂, —(CH₂)₆CH═CH₂, —(CH₂)₇CH═CH₂, —(CH₂)₈CH═CH₂, —(CH₂)₉CH═CH₂, —(CH₂)₁₀CH═CH₂, —(CH₂)₁₁CH═CH₂, —(CH₂)₁₂CH═CH₂, —(CH₂)₁₃CH═CH₂, —(CH₂)₁₄CH═CH₂, —(CH₂)₁₅CH═CH₂, —(CH₂)₁₆CH═CH₂, —(CH₂)₁₇CH═CH₂, —(CH₂)₁₈CH═CH₂, —(CH₂)₇CH═CH(CH₂)₃CH₃, —(CH₂)₇CH═CH(CH₂)₅CH₃, —(CH₂)₄CH═CH(CH₂)₈CH₃, —(CH₂)₇CH═CH(CH₂)₇CH₃, —(CH₂)₆CH═CHCH₂CH═CH(CH₂)₄CH₃, —(CH₂)₇CH═CHCH₂CH═CH(CH₂)₄CH₃, —(CH₂)₇CH═CHCH₂CH═CHCH₂CH═CHCH₂CH₃, —(CH₂)₃CH═CHCH₂CH═CHCH₂CH═CHCH₂CH═CH(CH₂)₄CH₃, —(CH₂)₃CH═CHCH₂CH═CHCH₂CH═CHCH₂CH═CHCH₂CH═CHCH₂CH₃, —(CH₂)₁₁CH═CH(CH₂)₇CH₃, or —(CH₂)₂CH═CHCH₂CH═CHCH₂CH═CHCH₂CH═CHCH₂CH═CHCH₂CH═CHCH₂CH₃.

In embodiments, said C₆-C₂₂ alkenyl is a monoalkenyl, a dienyl, or a trienyl. In embodiments, each of R², R³, and R⁴ is

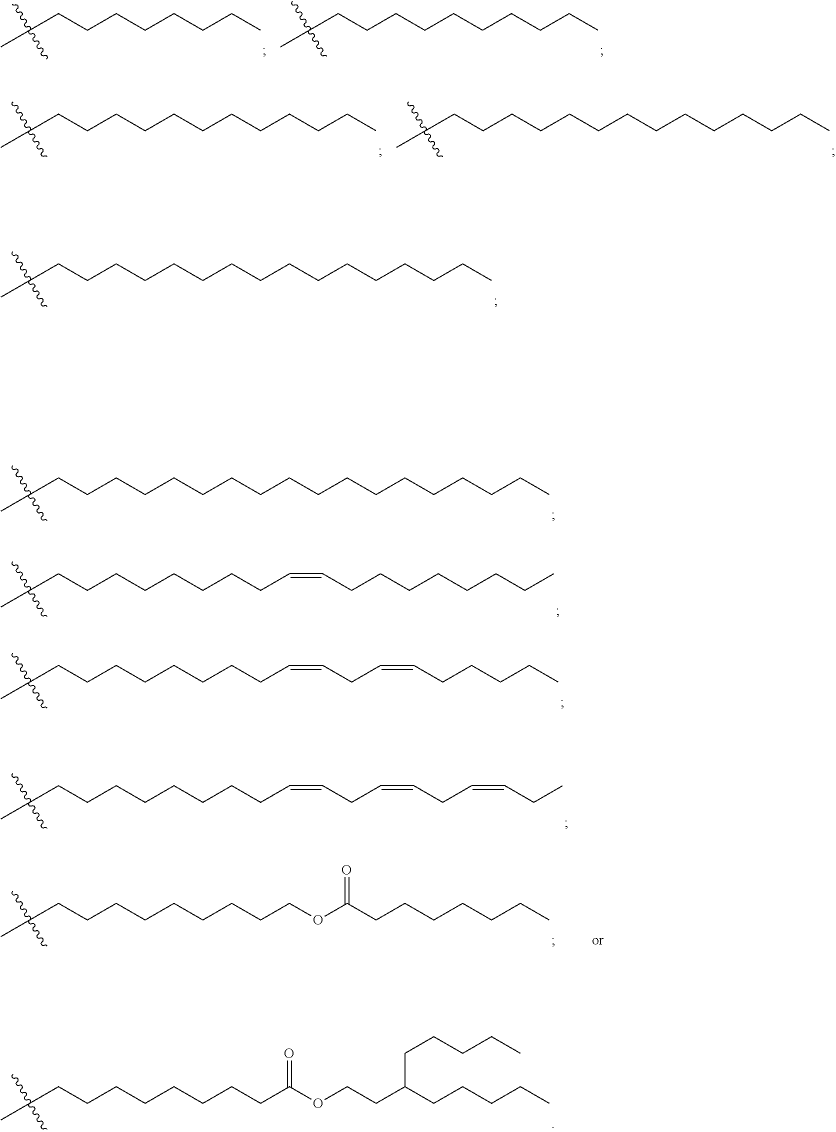

In some embodiments, the LNPs, compositions, pharmaceutical compositions and methods of the present invention include a cationic lipid having a compound structure of:

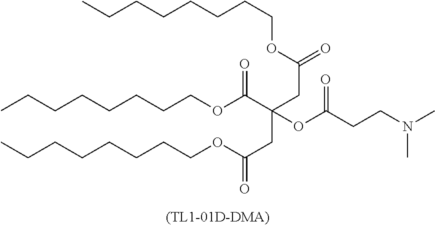 [SMILES: CCCCCCCCOC(=O)CC(CC(=O)OCCCCCCCC)(OC(=O)CCN(C)C)C(=O)OCCCCCCCC]

and pharmaceutically acceptable salts thereof. In some embodiments, the LNPs, compositions, pharmaceutical compositions and methods of the present invention include a cationic lipid having a compound structure of:

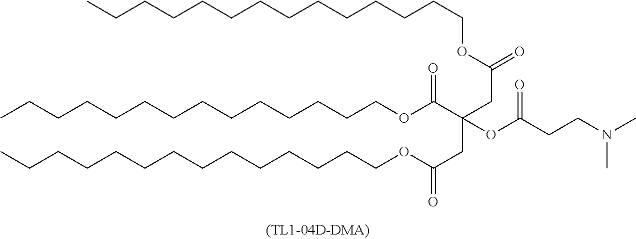 [SMILES: CCCCCCCCCCCCCCOC(=O)CC(CC(=O)OCCCCCCCCCCCCCC)(OC(=O)CCN(C)C)C(=O)OCCCCCCCCCCCCCC]

and pharmaceutically acceptable salts thereof. In some embodiments, the LNPs, compositions, pharmaceutical compositions and methods of the present invention include a cationic lipid having a compound structure of:

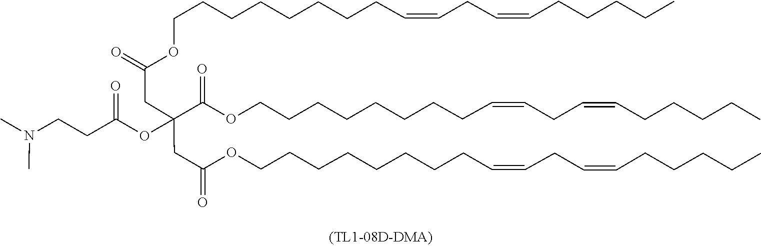 [SMILES: CCCCC/C=C\C/C=C\CCCCCCCCOC(=O)CC(CC(=O)OCCCCCCCC/C=C\C/C=C\CCCCC)(OC(=O)CCN(C)C)C(=O)OCCCCCCCC/C=C\C/C=C\CCCCC]

and pharmaceutically acceptable salts thereof. In some embodiments, the LNPs, compositions, pharmaceutical compositions and methods of the present invention include a cationic lipid having a compound structure of:

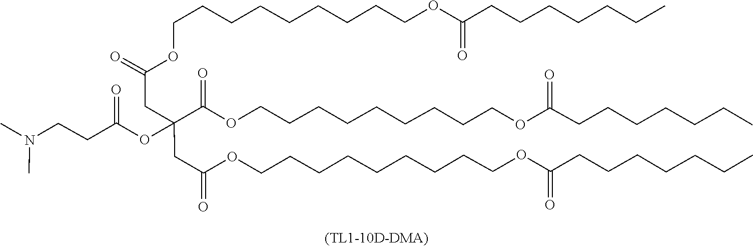 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

and pharmaceutically acceptable salts thereof.

Other suitable cationic lipids for use in the LNPs, compositions, pharmaceutical compositions and methods of the present invention include cleavable cationic lipids as described in International Patent Publication WO 2012/170889, which is incorporated herein by reference. In some embodiments, the LNPs, compositions, pharmaceutical compositions and methods of the present invention include a cationic lipid of the following formula:

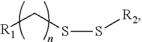

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

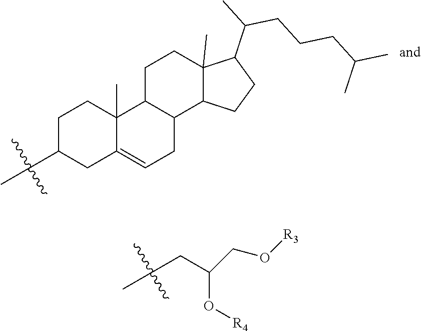

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In some embodiments, the LNPs, compositions, pharmaceutical compositions and methods of the present invention include a cationic lipid, “HGT4001”, having a compound structure of:

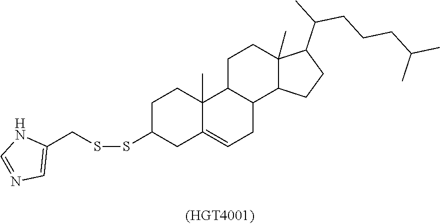 [SMILES: CC(C)CCCC(C)C1CCC2C3CC=C4CC(SSCc5cnc[nH]5)CCC4(C)C3CCC12C]

and pharmaceutically acceptable salts thereof. In some embodiments, the LNPs, compositions, pharmaceutical compositions and methods of the present invention include a cationic lipid, “HGT4002”, having a compound structure of:

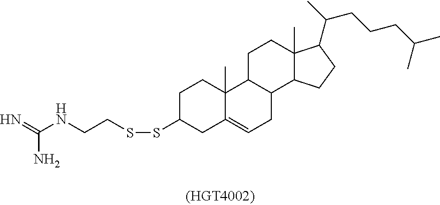 [SMILES: CC(C)CCCC(C)C1CCC2C3CC=C4CC(SSCCNC(=N)N)CCC4(C)C3CCC12C]

and pharmaceutically acceptable salts thereof. In some embodiments, the LNPs, compositions, pharmaceutical compositions and methods of the present invention include a cationic lipid, “HGT4003,” having a compound structure of:

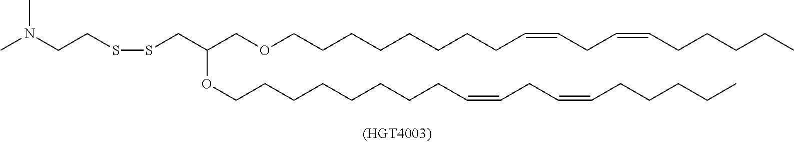 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In some embodiments, the LNPs, compositions, pharmaceutical compositions and methods of the present invention include a cationic lipid, “HGT4004,” having a compound structure of:

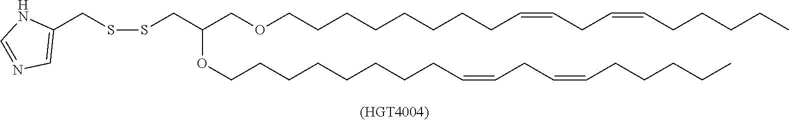 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In some embodiments, the LNPs, compositions, pharmaceutical compositions and methods of the present invention include a cationic lipid “HGT4005,” having a compound structure of:

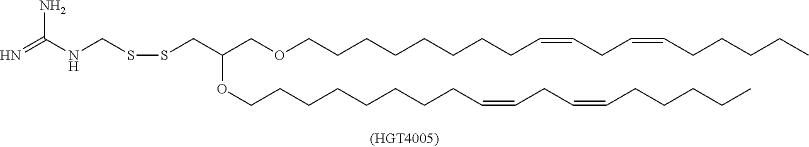 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the LNPs, compositions, pharmaceutical compositions and methods of the present invention include cleavable cationic lipids as described in International Patent Publication WO 2019/222424, and incorporated herein by reference. In some embodiments, the LNPs, compositions, pharmaceutical compositions and methods of the present invention include a cationic lipid that is any of general formulas or any of structures (1a)-(21a) and (1b)-(21b) and (22)-(237) described in International Patent Publication WO 2019/222424. In some embodiments, the LNPs, compositions, pharmaceutical compositions and methods of the present invention include a cationic lipid that has a structure according to Formula (I′),

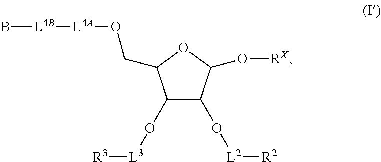

wherein:
R^{X} is independently —H, -L¹-R¹, or -L^{5A}-L^{5B}-B′;
each of L¹, L², and L³ is independently a covalent bond, —C(O)—, —C(O)O—, —C(O)S—, or —C(O)NR^{L}—;
each L^{4A} and L^{5A} is independently —C(O)—, —C(O)O—, or —C(O)NR^{L}—;
each L^{4B} and L^{5B} is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl; and
each R^{L} is independently hydrogen, C₁-C₂₀ alkyl, C₂-C₂₀ alkenyl, or C₂-C₂₀ alkynyl.
   In some embodiments, the LNPs, compositions, pharmaceutical compositions and methods of the present invention include a cationic lipid that is Compound (139) of International Patent Publication No. WO 2019/222424, having a compound structure of:

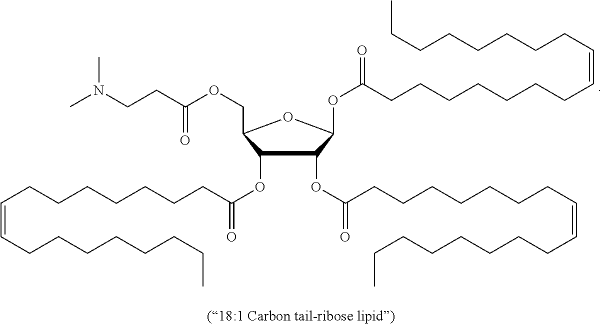

In some embodiments, the LNPs, compositions, pharmaceutical compositions and methods of the present invention include a cationic lipid that is RL3-DMA-07D having a compound structure of:

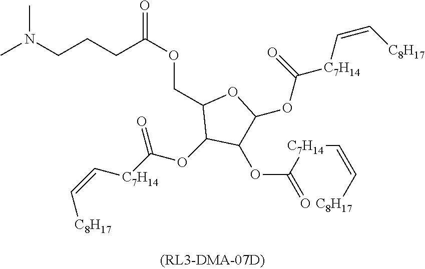

and pharmaceutically acceptable salts thereof.

In some embodiments, the LNPs, compositions, pharmaceutical compositions and methods of the present invention include a cationic lipid that is RL2-DMP-07D having a compound structure of:

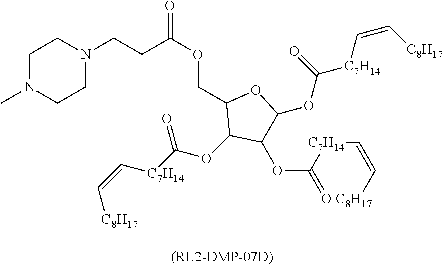

and pharmaceutically acceptable salts thereof.

In some embodiments, the LNPs, compositions, pharmaceutical compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the LNPs, compositions, pharmaceutical compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the LNPs, compositions, pharmaceutical compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′,1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z,12Z)-octadeca-9,12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z,12Z)-octadeca-9,12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z,12Z)-octadeca-9,12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the LNPs, compositions, pharmaceutical compositions and methods of the present invention include 2,2-Dilinoleyl-4-dimethylaminoethyl-[1,3]-dioxolane (“XTC”); (3aR,5s,6aS)-N,N-dimethyl-2,2-di((9Z,12Z)-octadeca-9,12-dienyl)tetrahydro-3aH-cyclopenta[d][1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the LNPs, compositions, pharmaceutical compositions of the present invention include one or more cationic lipids that constitute at least about 5%, 10%, 20%, 30%, 35%, 40%, 45%, 50%, 55%, 60%, 65%, or 70%, measured by weight, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the LNPs, compositions, pharmaceutical composition, e.g., a lipid nanoparticle.

### Non-Cationic Lipids

In some embodiments, the lipid nanoparticles contain one or more non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 1,2-dierucoyl-sn-glycero-3-phosphoethanolamine (DEPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof. In some embodiments, lipid nanoparticles suitable for use with the invention include DOPE as the non-cationic lipid component. In other embodiments, lipid nanoparticles suitable for use with the invention include DEPE as the non-cationic lipid component.

In some embodiments, a non-cationic lipid is a neutral lipid, i.e., a lipid that does not carry a net charge in the conditions under which the LNPs, compositions, pharmaceutical compositions are formulated and/or administered.

### Cholesterol-Based Lipids

In some embodiments, the lipid nanoparticle comprises one or more cholesterol-based lipids. For example, suitable cholesterol-based cationic lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335; all of which are incorporated herein by reference), or imidazole cholesterol ester (ICE), as disclosed in International Patent Publication WO 2011/068810 (incorporated herein by reference), which has the following structure:

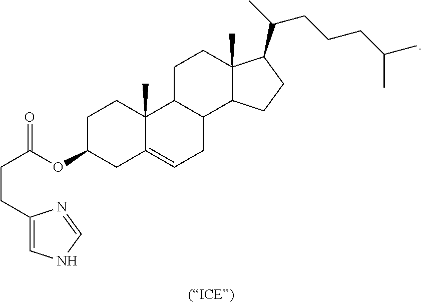 [SMILES: CC(C)CCCC(C)[C@H]1CCC2C3CC=C4C[C@@H](OC(=O)CCc5c[nH]cn5)CC[C@]4(C)C3CC[C@@]21C]

In some embodiments, a cholesterol-based lipid is cholesterol.

### PEG-Modified Lipids

In some embodiments, the lipid nanoparticle comprises one or more PEGylated lipids.

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid pharmaceutical compositions together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C₆-C₂₀ length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid pharmaceutical composition to the target tissues, (Klibanov et al. (1990) FEBS Letters, 268 (1): 235-237; incorporated herein by reference), or they may be selected to rapidly exchange out of the pharmaceutical composition in vivo (see U.S. Pat. No. 5,885,613; incorporated herein by reference). Particularly useful exchangeable lipids are PEG-ceramides having shorter acyl chains (e.g., C₁₄ or C₁₈). Lipid nanoparticles suitable for use with the invention typically include a PEG-modified lipid such as 1,2-dimyristoyl-rac-glycero-3-methoxypolyethylene glycol-2000 (DMG-PEG2K).

In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio. For certain applications, such as pulmonary delivery, lipid nanoparticles in which the PEG-modified lipid component constitutes about 5% of the total lipids by molar ratio have been found to be particularly suitable.

### Exemplary Lipid Formulations

A typical LNP for use with the invention may be composed of one of the following combinations of a cationic lipid, a non-cationic lipid, a PEG-modified lipid and optionally cholesterol: cKK-E12, DOPE, cholesterol and DMG-PEG2K; cKK-E10, DOPE, cholesterol and DMG-PEG2K; OF-Deg-Lin, DOPE, cholesterol and DMG-PEG2K; OF-02, DOPE, cholesterol and DMG-PEG2K; GL-HEPES-E3-E12-DS-4-E10, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; HGT4001, DOPE, cholesterol and DMG-PEG2K; HGT4002, DOPE, cholesterol and DMG-PEG2K; TL1-01D-DMA, DOPE, cholesterol and DMG-PEG2K; TL1-04D-DMA, DOPE, cholesterol and DMG-PEG2K; TL1-08D-DMA, DOPE, cholesterol and DMG-PEG2K; TL1-10D-DMA, DOPE, cholesterol and DMG-PEG2K; ICE, DOPE and DMG-PEG2K; HGT4001, DOPE and DMG-PEG2K; HGT4002, DOPE and DMG-PEG2K; SY-3-E14-DMAPr, DOPE, cholesterol and DMG-PEG2K; RL3-DMA-07D, DOPE, cholesterol and DMG-PEG2K; RL2-DMP-07D, DOPE, cholesterol and DMG-PEG2K; cHse-E-3-E10, DOPE, cholesterol and DMG-PEG2K; cHse-E-3-E12, DOPE, cholesterol and DMG-PEG2K; or cDD-TE-4-E12, DOPE, cholesterol and DMG-PEG2K. In specific embodiments, the LNP may be composed of SY-3-E14-DMAPr, DOPE, cholesterol and DMG-PEG2K. In other specific embodiments, the LNP may be composed of RL3-DMA-07D, DOPE, cholesterol and DMG-PEG2K. In yet other specific embodiments, the LNP may be composed of RL2-DMP-07D, DOPE, cholesterol and DMG-PEG2K. In yet other specific embodiments, the LNP may be composed of cHse-E-3-E10, DOPE, cholesterol and DMG-PEG2K. In yet other specific embodiments, the LNP may be composed of cHse-E-3-E12, DOPE, cholesterol and DMG-PEG2K. In yet other specific embodiments, the LNP may be composed of cDD-TE-4-E12, DOPE, cholesterol and DMG-PEG2K.

In some embodiments, cationic lipids (e.g., cKK-E12, cKK-E10, OF-Deg-Lin, OF-02, GL-HEPES-E3-E12-DS-4-E10, TL1-01D-DMA, TL1-04D-DMA, TL1-08D-DMA, TL1-10D-DMA, ICE, HGT4001, and/or HGT4002) constitute about 30-60% (e.g., about 30-55%, about 30-50%, about 30-45%, about 30-40%, about 35-50%, about 35-45%, or about 35-40%) of the lipid nanoparticle by molar ratio. In some embodiments, the percentage of cationic lipids (e.g., cKK-E12, cKK-E10, OF-Deg-Lin, OF-02, GL-HEPES-E3-E12-DS-4-E10, TL1-01D-DMA, TL1-04D-DMA, TL1-08D-DMA, TL1-10D-DMA, ICE, HGT4001, and/or HGT4002) is or greater than about 30%, about 35%, about 40%, about 45%, about 50%, about 55%, or about 60% of the lipid nanoparticle by molar ratio.

In some embodiments, the ratio of cationic lipid(s) to non-cationic lipid(s) to cholesterol-based lipid(s) to PEG-modified lipid(s) may be between about 30-60:25-35:20-30:1-15 by molar ratio. In some embodiments, the ratio of cationic lipid(s) to non-cationic lipid(s) to cholesterol-based lipid(s) to PEG-modified lipid(s) is approximately 40:30:20:10 by molar ratio. In some embodiments, the ratio of cationic lipid(s) to non-cationic lipid(s) to cholesterol-based lipid(s) to PEG-modified lipid(s) is approximately 40:30:25:5 by molar ratio. In some embodiments, the ratio of cationic lipid(s) to non-cationic lipid(s) to cholesterol-based lipid(s) to PEG-modified lipid(s) is approximately 40:32:25:3 by molar ratio. In some embodiments, the ratio of cationic lipid(s) to non-cationic lipid(s) to cholesterol-based lipid(s) to PEG-modified lipid(s) is approximately 50:25:20:5 by molar ratio.

In certain embodiments, the LNP comprises: a cationic lipid (e.g., OF-02, GL-HEPES-E3-E12-DS-4-E10 or cKK-E10) at a molar ratio of 35% to 55%; a non-cationic lipid (e.g., DOPE) at a molar ratio of 5% to 40%; a cholesterol-based lipid (e.g., cholesterol) at a molar ratio of 20% to 45%; and a PEG-modified lipid (e.g., DMG-PEG2K) at a molar ratio of 1% to 2%.

In some embodiments, the molar ratio of cationic lipid to non-cationic lipid to cholesterol-based lipid to PEG-modified lipid of 40:30:28.5:1.5. In some embodiments, the molar ratio of cationic lipid to non-cationic lipid to cholesterol-based lipid to PEG-modified lipid of 46.3:9.4:42.7:1.6. In some embodiments, the molar ratio of cationic lipid to non-cationic lipid to cholesterol-based lipid to PEG-modified lipid of 50:10:38.5:1.5.

In some embodiments, the LNP comprises: OF-02, GL-HEPES-E3-E12-DS-4-E10 or c-KK-E10 at a molar ratio of 40%; DOPE at a molar ratio of 30%; cholesterol at a molar ratio of 28.5%; and DMG-PEG2K at a molar ratio of 1.5%. In some embodiments, the LNP comprises: ALC-0315 at a molar ratio of 46.3%; DSPC at a molar ratio of 9.4%; cholesterol at a molar ratio of 42.7%; and ALC-0159 at a molar ratio of 1.6%. In some embodiments, the LNP comprises: SM-102 at a molar ratio of 50%; DSPC at a molar ratio of 10%; cholesterol at a molar ratio of 38.5%; and DMG-PEG2K at a molar ratio of 1.5%. Such lipid nanoparticles are particularly suitable for the delivery of mRNA via intramuscular administration.

In typical three-component lipid nanoparticles suitable for use with the invention, the molar ratio of cationic lipid to non-cationic lipid to PEG-modified lipid may be between about 55-65:30-40:1-15, respectively. In some embodiments, a molar ratio of cationic lipid (e.g., a sterol-based lipid) to non-cationic lipid (e.g., DOPE or DEPE) to PEG-modified lipid (e.g., DMG-PEG2K) of 60:35:5 is particularly suitable, e.g., for delivery of lipid nanoparticles via nebulization.

### Polymers

In some embodiments, a suitable LNP delivery vehicle is formulated using a polymer as a carrier, alone or in combination with other carriers including various lipids described herein. Thus, in some embodiments, LNPs, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

### Compositions

Compositions of the invention (e.g., immunogenic compositions or vaccines) may comprise one or more non-naturally occurring mRNAs encoding polyproteins of different serotypes of rhinovirus group A and/or group C and may be capable of eliciting an immune response against a wide variety of serotypes of rhinovirus group A and/or group C.

In some embodiments, an immunogenic composition of the invention comprises a first non-naturally occurring mRNA encoding at least one P2 polyprotein of rhinovirus A and a second non-naturally occurring mRNA encoding at least one VP0 polyprotein of rhinovirus C. In some embodiments, an immunogenic composition of the invention comprises a first non-naturally occurring mRNA encoding at least one P2 polyprotein of rhinovirus C and a second non-naturally occurring mRNA encoding at least one VP0 polyprotein of rhinovirus A.

In some embodiments, an immunogenic composition of the invention comprises a first non-naturally occurring mRNA encoding at least one VP0 polyprotein of rhinovirus A and a second non-naturally occurring mRNA encoding at least one VP0 polyprotein of rhinovirus C.

In some embodiments, an immunogenic composition of the invention comprises a first non-naturally occurring mRNA encoding at least one P2 polyprotein of rhinovirus A and a second non-naturally occurring mRNA encoding at least one P2 polyprotein of rhinovirus C.

In some embodiments, an immunogenic composition of the invention comprises a first non-naturally occurring mRNA encoding at least one VP0 polyprotein of rhinovirus A, a second non-naturally occurring mRNA encoding at least one P2 polyprotein of rhinovirus A, and a third non-naturally occurring mRNA encoding at least one VP0 polyprotein of rhinovirus C

In some embodiments, an immunogenic composition of the invention comprises a first non-naturally occurring mRNA encoding at least one VP0 polyprotein of rhinovirus A, a second non-naturally occurring mRNA encoding at least one P2 polyprotein of rhinovirus A, a third non-naturally occurring mRNA encoding at least one VP0 polyprotein of rhinovirus C, and a fourth non-naturally occurring mRNA encoding at least one P2 polyprotein of rhinovirus C.

In some embodiments, an immunogenic composition of the invention comprises a first non-naturally occurring mRNA encoding a first VP0 polyprotein of rhinovirus C, a second non-naturally occurring mRNA encoding a first P2 polyprotein of rhinovirus C, a third non-naturally occurring mRNA encoding a second VP0 polyprotein of rhinovirus C, and a fourth non-naturally occurring mRNA encoding a second P2 polyprotein from rhinovirus C.

In some embodiments, an immunogenic composition of the invention comprises a first non-naturally occurring mRNA encoding a first VP0 polyprotein of rhinovirus C, a second non-naturally occurring mRNA encoding a first P2 polyprotein of rhinovirus C, a third non-naturally occurring mRNA encoding a second VP0 polyprotein of rhinovirus C, a non-naturally occurring fourth mRNA encoding a second P2 polyprotein from rhinovirus C, a fifth non-naturally occurring mRNA encoding a VP0 polyprotein of rhinovirus A, a sixth non-naturally occurring mRNA encoding a P2 polyprotein of rhinovirus A.

In some embodiments, an immunogenic composition of the invention comprises a first non-naturally occurring mRNA encoding a VP0 polyprotein of rhinovirus A and a P2 polyprotein of rhinovirus A (e.g., as a fusion protein), and a second non-naturally occurring mRNA encoding at least one VP0 polyprotein of rhinovirus C.

In some embodiments, an immunogenic composition of the invention comprises a first non-naturally occurring mRNA encoding a first VP0 polyprotein of rhinovirus C and a first P2 polyprotein of rhinovirus C (e.g., as a fusion protein), and a second non-naturally occurring mRNA encoding a second VP0 polyprotein of rhinovirus C and a second P2 polyprotein from rhinovirus C (e.g., as a fusion protein).

In some embodiments, an immunogenic composition of the invention comprises a first non-naturally occurring mRNA encoding a VP0 polyprotein of rhinovirus A and a P2 polyprotein of rhinovirus A (e.g., as a fusion protein), a second non-naturally occurring mRNA encoding a VP0 polyprotein of rhinovirus C and a P2 polyprotein of rhinovirus C (e.g., as a fusion protein).

In some embodiments, an immunogenic composition of the invention comprises a first non-naturally occurring mRNA encoding a VP0 polyprotein of rhinovirus A and a P2 polyprotein of rhinovirus A (e.g., as a fusion protein), a second non-naturally occurring mRNA encoding a first VP0 polyprotein of rhinovirus C and a first P2 polyprotein of rhinovirus C (e.g., as a fusion protein), and a third non-naturally occurring mRNA encoding a second VP0 polyprotein of rhinovirus C and a second P2 polyprotein of rhinovirus C (e.g., as a fusion protein).

In some embodiments, an immunogenic composition of the invention comprises a non-naturally occurring mRNA encoding a VP0 polyprotein of rhinovirus A, a first VP0 polyprotein of rhinovirus C and a second VP0 polyprotein of rhinovirus C (e.g., as a fusion protein).

mRNA Concentration

The invention provides compositions comprising an mRNA of the invention. In some embodiments, a composition in accordance with the invention comprises an mRNA of the invention at a concentration ranging from about 0.5 mg/mL to about 1.0 mg/mL. In some embodiments, the mRNA is at a concentration of at least 0.5 mg/mL. In some embodiments, the mRNA is at a concentration of at least 0.6 mg/mL. In some embodiments, the mRNA is at a concentration of at least 0.7 mg/mL. In some embodiments, the mRNA is at a concentration of at least 0.8 mg/mL. In some embodiments, the mRNA is at a concentration of at least 0.9 mg/mL. In some embodiments, the mRNA is at a concentration of at least 1.0 mg/mL. In a typical embodiment, the mRNA is at a concentration of about 0.6 mg/mL to about 0.8 mg/mL. Pharmaceutically Acceptable Carriers and Excipients

Typically, the mRNA in the composition is encapsulated in LNPs. To stabilize the mRNA or the LNPs encapsulating it, or to enhance in vivo expression of the mRNAs, the compositions of the invention may be formulated with one or more carrier, stabilizing reagent or other excipients. Such compositions may be pharmaceutical compositions, and as such they may include one more or more pharmaceutically acceptable excipients. The one or more pharmaceutically acceptable excipients may be selected from a buffer, a sugar, a salt, a surfactant or combinations thereof.

In some embodiments, the pharmaceutical composition is formulated with a diluent. In some embodiments, the diluent is selected from a group consisting of ethylene glycol, glycerol, propylene glycol, sucrose, trehalose, or combinations thereof. In some embodiments, the formulation comprises 1%, 2%, 3%, 4%, 5%, 6%, 7%, 8%, 9%, 10%, 11%, 12%, 13%, 14%, 15%, 16%, 17%, 18%, 19% or 20% diluent.

In some embodiments, the LNPs are suspended in an aqueous solution comprising a disaccharide. Suitable disaccharides for use with the invention include trehalose and sucrose. For example, in some embodiments, the LNPs are suspended in an aqueous solution comprising trehalose, e.g., 10% (w/v) trehalose in water. In other embodiments, LNPs are suspended in an aqueous solution comprising sucrose, e.g., 10% (w/v) sucrose in water.

In some embodiments, the aqueous solution further comprises a buffer, a salt, a surfactant or combinations thereof.

In some embodiments, the salt is selected from the group consisting of NaCl, KCl, and CaCl₂). Accordingly, in some embodiments, the salt is NaCl. In some embodiments, the salt is KCl. In some embodiments, the salt is CaCl₂).

In some embodiments, the buffer is selected from the group consisting of a phosphate buffer, a citrate buffer, an imidazole buffer, a histidine buffer, and a Good's buffer. Accordingly, in some embodiments, the buffer is a phosphate buffer. In some embodiments, the buffer is a citrate buffer. In some embodiments, the buffer is an imidazole buffer. In some embodiments, the buffer is a histidine buffer. In some embodiments, the buffer is a Good's buffer. In some embodiments, the Good's buffer is a Tris buffer or HEPES buffer.

In particular embodiments, the buffer is a phosphate buffer (e.g., a citrate-phosphate buffer), a Tris buffer (e.g., TrisHCl), or an imidazole buffer. In some embodiments, the buffer is, or includes, an acetate buffer.

In some embodiments, the composition comprises a buffer and a salt (typically in addition to a suitable diluent such as a disaccharide or optionally a propylene glycol). In some embodiments, the total concentration of the buffer and the salt is selected from about 40 mM Tris buffer and about 75-125 mM NaCl, about 50 mM Tris buffer and about 50 mM-100 mM NaCl, about 100 mM Tris buffer and about 100 mM-200 mM NaCl, about 40 mM imidazole and about 100 mM-125 mM NaCl, and about 50 mM imidazole and 75 mM-100 mM NaCl.

In some embodiments, the composition comprises a buffer (e.g., phosphate or Tris), a salt (e.g., KCl or NaCl, or both), and a sugar (e.g., a disaccharide such as sucrose or trehalose). In particular embodiments, the composition is an aqueous solution (e.g., comprising water for injection) comprising the buffer, salt and sugar. Additional excipients may include NaOH or HCl (e.g., to adjust the pH of the composition).

### Adjuvants

In various embodiments, the immunogenic compositions (e.g., vaccines) described herein further comprise an adjuvant. Adjuvants can include a suspension of minerals (alum, aluminum salts, including, for example, aluminum hydroxide/oxyhydroxide (AlOOH), aluminum phosphate (AlPO₄), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. In some embodiments, the adjuvant is squalene-based. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199, which are incorporated herewith by reference). Adjuvants also include biological molecules, such as lipids and costimulatory molecules. Exemplary biological adjuvants include AS04 (Didierlaurent et al., J Immunol. 2009; 183(10):6186-97, which is incorporated herewith by reference), IL-2, RANTES, GM-CSF, TNF-α, IFN-γ, G-CSF, LFA-3, CD72, B7-1, B7-2, OX-40L and 41 BBL.

In particular embodiments, an immunogenic composition of the invention (e.g., a vaccine) does not include an adjuvant. For example, the composition may include one or more non-naturally occurring mRNAs, e.g., encapsulated in one or more lipid nanoparticles, without any separate adjuvant component.

### Therapeutically Effective Amount

The mRNA in accordance with the invention is provided in a therapeutically effective amount in the pharmaceutical compositions (e.g., an immunogenic composition or vaccine) provided herein. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject.

### Packaging

The immunogenic compositions of the invention (e.g., a vaccine) may be packaged for parenteral (e.g., intramuscular, intradermal, or subcutaneous) administration or mucosal (e.g., nasopharyngeal, pulmonary or intranasal) administration. The vaccine compositions may be in the form of an extemporaneous formulation, e.g., in a lyophilized form that requires reconstitution with a physiological buffer (e.g., PBS) just before use. In some embodiments, an immunogenic composition of the invention (e.g., a vaccine) is provided in the form of an aqueous solution or a frozen aqueous solution and can be directly administered to subjects without reconstitution (after thawing, if previously frozen).

Accordingly, the present disclosure provides an article of manufacture, such as a kit, that provides the immunogenic composition of the invention (e.g., a vaccine) in a single container, or provides the composition (e.g., a vaccine) in one container and a physiological buffer for reconstitution in another container. The container(s) may contain a single-use dosage or multi-use dosage. The containers may be pre-treated glass vials or ampules. The article of manufacture may include instructions for use as well.

In particular embodiments, an immunogenic composition of the invention (e.g., a vaccine) is provided for use in intramuscular injection. The composition can be injected to a subject at, e.g., his/her deltoid muscle in the upper arm. In some embodiments, the immunogenic composition (e.g., a vaccine) is provided in a pre-filled syringe or injector (e.g., single-chambered or multi-chambered). In some embodiments, the immunogenic composition (e.g., a vaccine) is provided for use by mucosal administration (e.g., as an intranasal spray). In some embodiments, the immunogenic composition (e.g., a vaccine) is provided for use by inhalation (e.g., for pulmonary delivery) and is provided in a pre-filled pump, aerosolizer, or inhaler.

In certain embodiments, the immunogenic composition (e.g., a vaccine) are provided for use in skin injection, e.g., in the epidermis, the dermis, or the hypodermis of the skin. In some embodiments, the compositions are provided in a device suitable for skin injection, such as a needle (e.g., an epidermic, dermic or hypodermic needle), a needle free device, a microneedle device, or a microprojection array device. Examples of microneedle or microprojection array devices suitable for the skin injection are described in US20230270842A1, US20220339416A1, US20210085598A1, US20200246450A1, US20220143376A1, US20180264244A1, US20180263641A1, US20110245776A1.

### Therapeutic Uses

In some embodiments, the invention provides a method for eliciting an immune response in a subject, wherein the method comprises administering an effective amount of an immunogenic composition comprising one or more non-naturally occurring messenger RNAs (mRNAs) encoding one or more non-structural and/or one or more structural rhinovirus polypeptides as described herein to the subject. In some embodiments, the invention provides an immunogenic composition comprising one or more non-naturally occurring messenger RNAs (mRNAs) encoding one or more non-structural and/or one or more structural rhinovirus polypeptides as described herein for use in eliciting an immune response in a subject. In some embodiments, the invention provides for the use of a composition comprising one or more non-naturally occurring messenger RNAs (mRNAs) encoding one or more non-structural and/or one or more structural rhinovirus polypeptides as described herein in a method of manufacturing a medicament, wherein the composition is for (e.g., is formulated for) eliciting an immune response in a subject.

In some embodiments, the invention provides a method of reducing or preventing one or more symptoms associated with a rhinovirus infection in a subject, wherein the method comprises administering an effective amount of an immunogenic composition comprising one or more non-naturally occurring messenger RNAs (mRNAs) encoding one or more non-structural and/or one or more structural rhinovirus polypeptides as described herein to the subject. In some embodiments, the invention provides an immunogenic composition comprising one or more non-naturally occurring messenger RNAs (mRNAs) encoding one or more non-structural and/or one or more structural rhinovirus polypeptides as described herein for use in reducing or preventing one or more symptoms associated with a rhinovirus infection in a subject. In some embodiments, the invention provides for the use of a composition comprising one or more non-naturally occurring messenger RNAs (mRNAs) encoding one or more non-structural and/or one or more structural rhinovirus polypeptides as described herein in a method of manufacturing a medicament, wherein the composition is for (e.g., is formulated for) reducing or preventing one or more symptoms associated with a rhinovirus infection in a subject.

In some embodiments, the invention provides a method of reducing the severity of a rhinovirus infection in a subject, the method comprising administering an effective amount of an immunogenic composition comprising one or more non-naturally occurring messenger RNAs (mRNAs) encoding one or more non-structural and/or one or more structural rhinovirus polypeptides as described herein to the subject. In some embodiments, the invention provides an immunogenic composition comprising one or more non-naturally occurring messenger RNAs (mRNAs) encoding one or more non-structural and/or one or more structural rhinovirus polypeptides as described herein for use in reducing the severity of a rhinovirus infection in a subject. In some embodiments, the invention provides for the use of a composition comprising one or more non-naturally occurring messenger RNAs (mRNAs) encoding one or more non-structural and/or one or more structural rhinovirus polypeptides as described herein in a method of manufacturing a medicament, wherein the composition is for (e.g., is formulated for) reducing the severity of a rhinovirus infection in a subject.

In some embodiments, the invention provides a method of preventing a rhinovirus infection in a subject, the method comprising administering an effective amount of an immunogenic composition comprising one or more non-naturally occurring messenger RNAs (mRNAs) encoding one or more non-structural and/or one or more structural rhinovirus polypeptides as described herein to the subject. In some embodiments, the invention provides an immunogenic composition comprising one or more non-naturally occurring messenger RNAs (mRNAs) encoding one or more non-structural and/or one or more structural rhinovirus polypeptides as described herein for use in preventing a rhinovirus infection in a subject. In some embodiments, the invention provides for the use of a composition comprising one or more non-naturally occurring messenger RNAs (mRNAs) encoding one or more non-structural and/or one or more structural rhinovirus polypeptides as described herein in a method of manufacturing a medicament, wherein the composition is for (e.g., is formulated for) preventing a rhinovirus infection in a subject.

In some embodiments, administering an immunogenic composition of the invention boosts a pre-existing rhinovirus T-cell response or re-orients the pre-existing rhinovirus T-cell response towards a T_{H}1 response. In some embodiments, the invention provides a composition for use in boosting a pre-existing rhinovirus T-cell response or re-orienting the pre-existing rhinovirus T-cell response towards a T_{H}1 response in a subject, wherein the composition comprises an effective amount of an immunogenic composition of the invention. In some embodiments, the invention provides a method of manufacturing a composition for boosting a pre-existing rhinovirus T-cell response or re-orienting the pre-existing rhinovirus T-cell response towards a THT response in a subject, wherein the composition is an immunogenic composition of the invention.

In some embodiments of the invention, administering the immunogenic composition induces intracellular antibodies against one or more non-structural polypeptides encoded by the mRNA. In some embodiments, the invention provides a composition for use in inducing intracellular antibodies against one or more non-structural polypeptides encoded by the mRNA in a subject, wherein the composition comprises an effective amount of an immunogenic composition of the invention. In some embodiments, the invention provides a method of manufacturing a composition for inducing intracellular antibodies against one or more non-structural polypeptides encoded by the mRNA in a subject, wherein the composition is an immunogenic composition of the invention.

In some embodiments of the invention, administration of the immunogenic composition provides immunity against a rhinovirus infection caused by a group A strain, a group B strain and/or a group C strain. In some embodiments, administration provides immunity against infection caused by multiple rhinovirus serotypes. In some embodiments, the multiple serotypes are of the same group of rhinovirus (e.g., group A or group C). For example, in some embodiments, immunity is provided against one or more rhinovirus A serotypes. In some embodiments, immunity is provided against about 20 or more, about 30 or more, about 40 or more, or about 50 or more rhinovirus A serotypes. In some embodiments, immunity is provided against one or more rhinovirus A serotypes and one or more rhinovirus C serotypes.

In some embodiments, the immunogenic composition of the invention is administered prophylactically.

In alternative embodiments, the immunogenic composition of the invention is administered following rhinovirus symptoms and/or confirmation that the subject has a rhinovirus infection.

In some embodiments, a composition of the invention is formulated for parenteral administration, such as intramuscular, intravenous, subcutaneous, intraperitoneal, or intradermal administration. In some embodiments, a composition of the invention is formulated for intranasal or inhalation administration. A composition of the invention can also be formulated for any other intended route of administration that is suitable for inducing an immune response.

In some embodiments, the composition of the invention is administered as a single intramuscular dose. In some embodiments, a booster dose is administered intramuscularly about one year or more after the first administration. In some embodiments, the booster dose is administered after 5 years.

### Subjects

In some embodiments, the subject to whom an immunogenic composition of the invention is administered is healthy.

In some embodiments, the subject is an infant (less than 36 months). In some embodiments, the subject is a child or an adolescent (less than 18 years of age). In some embodiments, the subject is elderly (at least 65 years of age). In some embodiments, the subject is a non-elderly adult (at least 18 years of age and less than 65 years of age).

In some embodiments, the subject is at least 40 years of age.

In some embodiments, the subject is at least 65 years old.

In some embodiments, the subject to whom an immunogenic composition of the invention is administered suffers from a respiratory condition. In some embodiments, the subject suffers from asthma. In some embodiments, the subject suffers from chronic obstructive pulmonary disease (COPD). In some embodiments, the subject suffers from COPD and is at least 40 years old.

In some embodiments, the subject is a child. In some embodiments, the subject is a child with asthma.

In some embodiments, the immunogenic composition of the invention is administered to healthy adults who are at least 65 years old, and adults aged 40-64 years old who suffer from COPD.

### Methods of Assessing an Immune Response

Methods of assessing an immune response, e.g., after administration of an immunogenic composition of the invention to a subject, are also provided.

In particular, the provided methods can assess the capacity of a subject for mounting a T-cell-mediated immune response to a rhinovirus. A sample comprising peripheral blood mononuclear cells (PBMCs) obtained from the subject is incubated in the presence of one or more peptides or polypeptides comprising one or more rhinovirus T-cell epitopes identified herein for a time and under conditions sufficient to stimulate the PBMCs to produce one or more effector molecule(s). The presence or level of the one or more effector molecule(s) is indicative of the subject's capacity to mount a T-cell-mediated immune response.

In some embodiments, the sample is a blood sample. PBMCs typically include lymphocytes. In a specific embodiment, the PBMCs are T-lymphocytes.

In some embodiments, the one or more peptides or polypeptides comprising one or more rhinovirus T-cell epitopes are derived from a non-structural rhinovirus protein or polyprotein. In some embodiments, the one or more peptides or polypeptides comprising one or more rhinovirus T-cell epitopes are derived from a non-structural rhinovirus protein or polyprotein. In some embodiments, the one or more peptides or polypeptides comprising one or more rhinovirus T-cell epitopes are derived from non-structural and structural rhinovirus proteins or polyproteins.

In some embodiments, the non-structural rhinovirus polyprotein is P2. In some embodiments, the structural polyprotein is VP0.

In some embodiments, the one or more peptides or polypeptides comprise one or more class-I T-cell epitopes. In some embodiments, the one or more peptides or polypeptides comprise one or more class-II T-cell epitopes. In some embodiments, the one or more peptides or polypeptides comprise one or more class-I T-cell epitopes and one or more class-II T-cell epitopes.

In some embodiments, incubation between the sample and the one or more peptides or polypeptides occurs in a test tube or in the well of a multi-well plate.

In some embodiments, incubation occurs in the presence of heparin.

In some embodiments, incubation occurs in the presence of an added carbohydrate.

In some embodiments, the effector molecule(s) is/are one or more cytokine(s) and/or one or more interleukin(s). In some embodiments, the effector molecule is/are selected from interferon-γ, a cytokine, an interleukin, and TNF-α. In one embodiment, the one or more effector molecules is/are interferon-γ and/or TNF-α. In some embodiments, one or more of the effector molecules is an interleukin. In some embodiments, the one or more interleukins is/are selected from IL-33, IL-25, IL-4, IL-5 and IL-13.

In some embodiments, the subject is a human or a non-human animal. Typically, the subject is a human.

In some embodiments, the subject is healthy. In some embodiments, the subject may be suspected of having a rhinovirus infection. In some embodiments, the subject was previously exposed to or infected with a rhinovirus.

In some embodiments, the subject has a respiratory condition. In some embodiments, the respiratory condition is asthma. In some embodiments, the respiratory condition is chronic obstructive pulmonary disease (COPD). Subjects suffering from respiratory conditions can experience viral-related exacerbations as a consequence of rhinovirus infection.

In some embodiments, a method for assessing an immune response as described herein may be used to diagnose infection by a rhinovirus. In some embodiments, a method for assessing an immune response as described herein may be used to determine whether a subject has a pre-existing rhinovirus T-cell response. In some embodiments, a method for assessing an immune response as described herein may be used to determine whether an immunogenic composition of the invention can re-orient a pre-existing rhinovirus T-cell response towards a T_{H}1 response.

### EXAMPLES

The following examples are included for illustrative purposes only and are not intended to limit the scope of the invention.

### Example 1. Identification of Conserved Regions in the Rhinovirus A Polyprotein

This example illustrates that the P2 region encoding non-structural rhinovirus proteins 2A, 2B and 2C and the VP0 region encoding structural rhinovirus proteins VP4 and VP2 include the largest number of conserved regions of the rhinovirus A polyprotein.

Publicly available sequences were retrieved from the website of ViPR (https://www.viprbrc.org/). It is administered by the NIAID (National Institute of Allergy and Infectious Diseases) and the sequences originally come from GenBank (administered by the NCBI—National Center for Biotechnology Information).

Sequences retrieval was performed on 24 Aug. 2021. A total of 19,361 sequences were obtained at that time. Different types of data were downloaded in parallel, starting from the following request: Taxonomy browser/Enterovirus/Rhinovirus A (and then B, C, plus unclassified Rhinovirus). Only 1,076 protein sequences comprising over 800 amino acids were included in the database, indicating that less than 6% of the entries include the full rhinovirus polyprotein. The majority of published sequences covered the N-terminal part of the polyprotein (VP4-VP2 proteins).

The metadata for each amino acid sequence were enriched by assigning a rhinovirus group to as many sequences as possible, as this information was available only in a minor number of entries. Using strain name information, the virus type was inferred when not properly annotated by default. The metadata were further complemented by results of a global sequence alignment in which the three rhinovirus groups (A, B and C) were perfectly differentiated from each other.

To achieve better quality sequence alignments and phylogenetic tree calculations, virus types were separated and one alignment per rhinovirus type was generated. To further improve quality, subsets of protein sequences were extracted from the database, to only keep the longest ones for protein alignment. The cutoff was set at 800 amino acids in length. The rationale for 800 is that this is the approximate length of the full VP capsid region of the polyprotein. As the VP proteins are the only ones visible on the virus surface, they should describe most if not all differences between serotypes. Low quality sequences exhibiting stretches of X (unknown amino acids) longer than 10 were also eliminated. All sequences selected in this way contained most if not all VP peptides. Under these conditions, the number of sequences used for alignments was drastically reduced:
Type A viruses: 539 sequences
Type B viruses: 159 sequences
Type C viruses: 374 sequences

The initial analysis focused on the rhinovirus A sequences. Phylogenetic trees were calculated in parallel with FastTree and PhyML algorithms. For hundreds of long sequences, PhyML requires a very long calculation time, up to several weeks sometimes, making FastTree a better choice to get results faster. Both are based on the same approach called maximum likelihood. FastTree is a proxy of PhyML, and prior comparison performed in-house showed that generated trees were quasi-identical between both algorithms when applied to virus proteins of the same family.

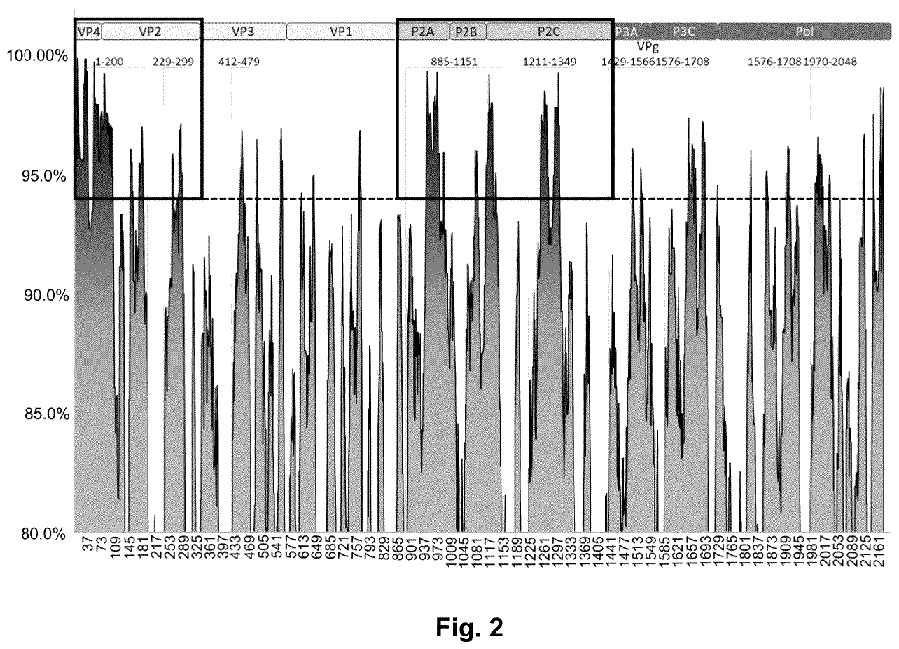

Protein sequence alignments were performed using the MAFFT algorithm and were followed by manual curation to minimize gaps. An in silico consensus sequence was calculated from the sequence alignment. A gap was allocated to a position in which the sum of any amino acid was lower than 50% in frequency. The most frequently occurring amino acid at each position was used all along the alignment of the complete rhinovirus A polyprotein sequence. Then a sliding window of the average value for 10 amino acids was calculated to smooth the curve. The result of this analysis is shown in FIG. 2.

The amino acid sequence of a region was considered conserved if it comprised more than 50 amino acids and had a sequence identity of greater than 80%. The most conserved regions are VP4 and P2A and the N-terminal domains VP2 and P2B. P2C also includes two conserved regions. The conserved regions in P3 proteins are more dispersed. On the basis of this analysis, the VP0 polyprotein and P2 polyprotein of rhinovirus A were identified as including the largest number of conserved regions.

To identify a naturally occurring consensus sequence, the in silico consensus sequence was then used as a probe sequence to identify in the full dataset the closest matching published sequences of naturally occurring rhinovirus A strains. The analysis was performed for the complete rhinovirus protein as well as the VP0 and P2 polyproteins. To this end, a matrix of identity was calculated from the alignment used for generating the in silico consensus after adding it into the alignment. The column of the in silico consensus sequence displayed all pairwise identities between the consensus sequence and each aligned sequence. The highest identity was selected as the naturally occurring consensus sequence with the best match. This analysis yielded the results shown in Table 8.

Each individual sequence alignment generated its own identity percentage list. To select an amino acid sequence for the antigen design, the count of hits among the top 20 best matches was also taken into account for each strain (see Table 8).

TABLE 8
Best matches of naturally occurring
rhinovirus A amino acid sequences

| | | | | SEQ | In silico | #hits |
|---|---|---|---|---|---|---|
| Aligned | | | GenBank | ID | consensus | in |
| seq | Rank | Serotype | ID | NO. | Id % | top20 |
| Polyprotein | 1 | hRVA21 | FJ445121.1 | 45 | 90.1 | 9 |
| | 2 | hRVA24 | JN562727.1 | 67 | 90.3 | 5 |
| | 3 | hRVA57 | KY369874.1 | 68 | 90.1 | 3 |
| VP0 | 1 | hRVA21 | FJ445121.1 | 4 | 92.4 | 13 |
| | 2 | hRVA90 | FJ445167.1 | 5 | 92.7 | 1 |
| | 3 | hRVA96 | FJ445171.1 | 69 | 91.2 | 1 |
| P2 | 1 | hRVA21 | FJ445121.1 | 6 | 95.7 | 9 |
| | 2 | hRVA57 | KY369874.1 | 7 | 95.5 | 5 |
| | 3 | hRVA24 | JN562727.1 | 70 | 95.3 | 3 |

The amino acid sequences of the VP0 and P2 polyprotein from the 7134 nucleotide genome of an A21 serotype of human rhinovirus (GenBank ID: FJ445121.1, strain name: ATCC VR-1131) were identified as the closest ones to the in silico consensus sequence. The complete polyprotein of this virus has the following amino acid sequence:

(SEQ ID NO: 45)
MGTQVSRQNVGTHSTQNSVSNGSSLNYFNINYFKDAASSGASKLE
FSQDPSKFTDPVKDVLEKGIPTLQSPTVEACGYSDRIIQITRGDS
TITSQDVANAVVGYGVWPHYLTPQDATAIDKPSRPDTSSNRFYTL
ESKMWTSDSKGWWWKLPDALKNMGIFGENMFYHFLGRSGYTVHVQ
CNASKFHQGTLIVVMIPEHQLASASTGNVTAGYNLTHPGEQGRDV
GITRVEDLLKQPSDDSWLNEDGILLGNITIFPHQFINLRSNNSAT
IIVPYVNAVPMDSMPRHNNWSLVIIPICPLESDGQTPVPITISIS
PMCAEFSGARAKSQGLPVMLTPGSGQFMTTDDFQSPSALPWFHPT
KEISIPGQVINLIELCQVDTLIPVNNTETNGVTNINMYTVTINRE
VNITPAKEIFAIKVDIASQPLATTLIGEIANYYTHWTGSIRFSFL
FCGTANTTLKLLVAYTPPGIKKPENRKDAMLGTHVVWDVGLQSTI
SMVVPWISASHYRNTTPDKYSSAGYITCWYQTNLVVPPNTPTSAK
MLCFVSGCKDFCLRMARDTSLHKQSAPITQNPVENYIDEVLNEVL
VVPNIRESHGTTSNSAPALDAAETGHTSNVQPEDMVETRYVQTSQ
TRDEMSIESFLGRSGCIHMSKLVVNYDNYNTGENNISTWQINIKE
MAQIRRKFEMFTYTREDSEITLVPSIAARAGDIGHVVMQYMYVPP
GAPIPKTREDFAWQSGTNASIFWQHGQTYPRFSLPFLSIASAYYM
FYDGYDGDQTDSQYGAVVINDMGSLCYRIVTGQHKHKIEVTTRIY
HKAKHVKAWCPRPPRAVEYTHTHVINYKIANHEVTSAVESRRTIV
TVGPSDMYVHVGNLIYRNLHLENSEMHDSILISYSSDLVIYRTNT
TGDDYIPTCDCTEATYYCKHKNRYFPIKVTSHDWYEIQESEYYPK
HIQYNLLIGEGPCEPGDCGGKLLCKHGVIGMITAGGDGHVAFIDL
RHFHCAEEQGITDYIHMLGEAFGSGFVDSVKEQINAINPINNISK
KIIKWLLRIISAMVIIIRNSSDPQTIIATLTLIGCSGSPWRFLKE
KFCKWTQLNYIHKESDSWLKKFTEMCNAARGLEWIGNKISKFIDW
MKSMLPQAQLKVKYLSELKKLNLLEKQIEHLRAADSATQEKIKCE
IDTLHDLSCKFLPLYASEAKRIKVLHNKCNVVIKQKKRCEPVAVV
IHGEPGTGKSMITNFLARMIINDSDIYSLPPDPKYFDGYDQQSVV
IMDDIMQNPSGDDMTLFCQMVSSVTFIPPMADLPDKGKPFDSRFV
LCSTNHSLLTPPTITSLPAMNRRFFMDLDIIVCDKYKDAQGKLNV
SAAFKPCDVDTKIGNARCCPFICGKAVMFKDRNTCRTYTLAQIYN
QILEEDKRRRQVIDVMSAIFQGPISLEGPPPAAISDLLQSVRTPE
VIKYCEANKWIIPAECKVERDLNIANTIITIIANIISISGIIYVI
YKLFCTLQGPYSGEPKPKTKMPERRVVAQGPEEEFGRSLIKHNSC
VVTTQNGKFTGLGIYDRTLIIPTHADPGKEVQIDGIATKVEDSYD
LENKDGVKLEITVLKLKRNEKFKDIRKYIPENEDDYPDCNLALSA
NQPETTIINVGDVISYGNILLSGTQTARMLKYNYPTKSGYCGGIL
YKIGQVLGIHVGGNGRDGFAAMLLRSYFSETQGQIINSKPTDKCG
LPSIHTPSKTKLQPSVFYDIFPGNKEPAVLSNKDPRLEVDFEKAL
FSKYKGNEHCVMNDHINVAISHYSAQLATLDINPQPISIEESVFG
MDGLEALDLNTSAGFPYVTMGIKKRDLINKQTKDITKLKMALDKY
GVDLPMVTFLKDELRKKEKISAGKTRVIEASSVNDTVLFRTTFGN
LFSKFHLNPGIVTGSAVGCDPEVFWSKIPVMLDGDCIMAFDYTNY
DGSIHPIWFQALKQVLINLSFEASLIDRLCKSKHIFKNIYYEVEG
GVPSGCSGTSIFNTMINNVIIRTLVLDAYKNIDLDKLKIIAYGDD
VIFSYKYQLDMEAIANEGTKYGLTITPADKSTCFKQLDYSNVTFL
KRGFKQDEKHQFLIHPTFPIEEIHESIRWTKNPSQMQEHVLSLCH
LMWHNGRDVYKQFEKRIRSVGAGRALYIPPYDLLLHEWYEKF

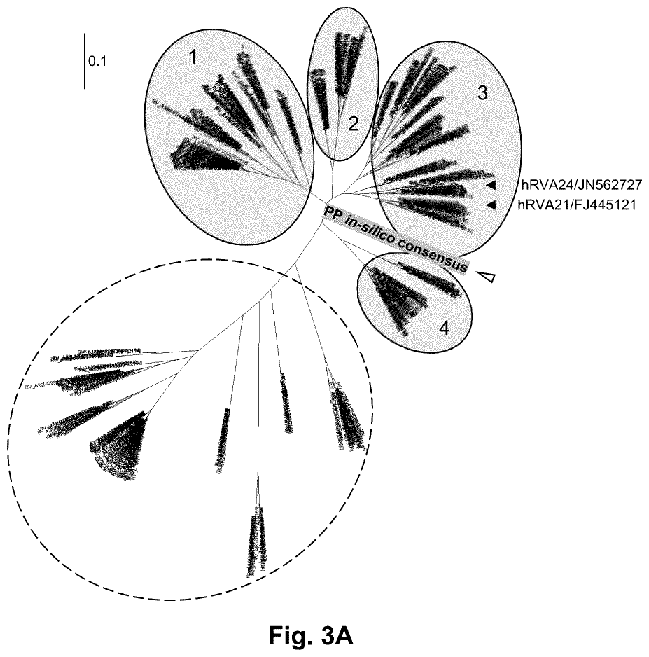

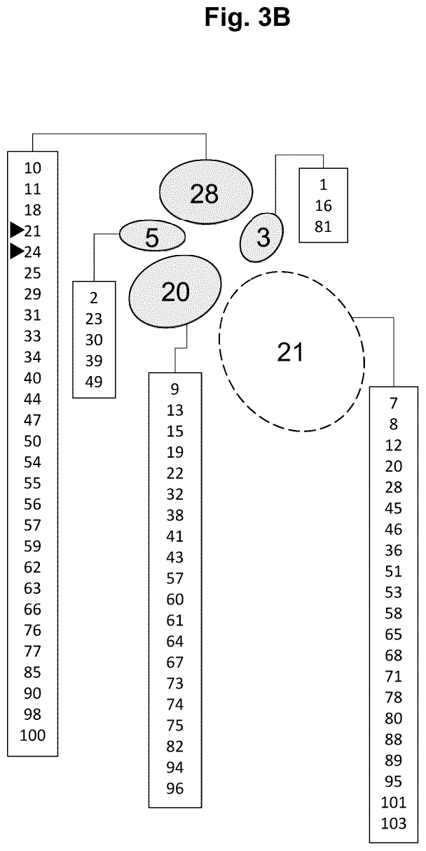

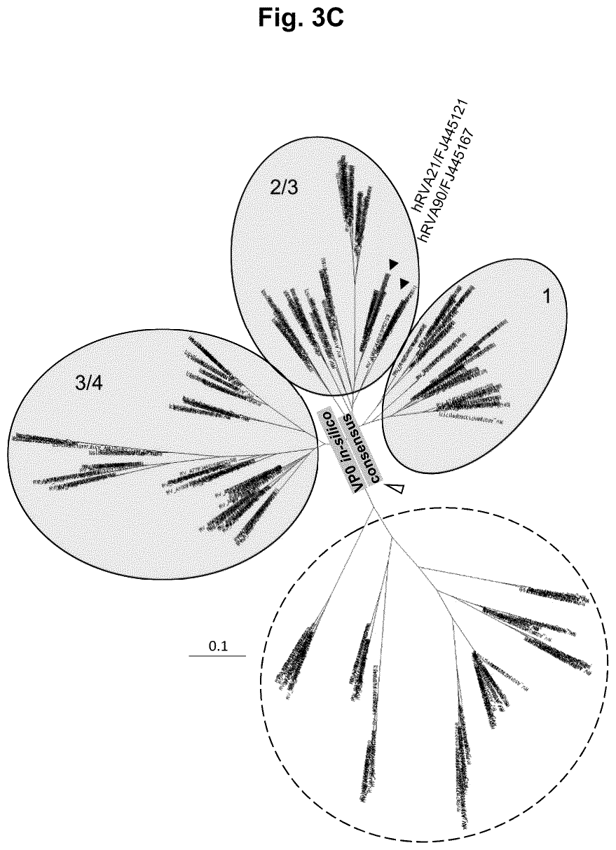

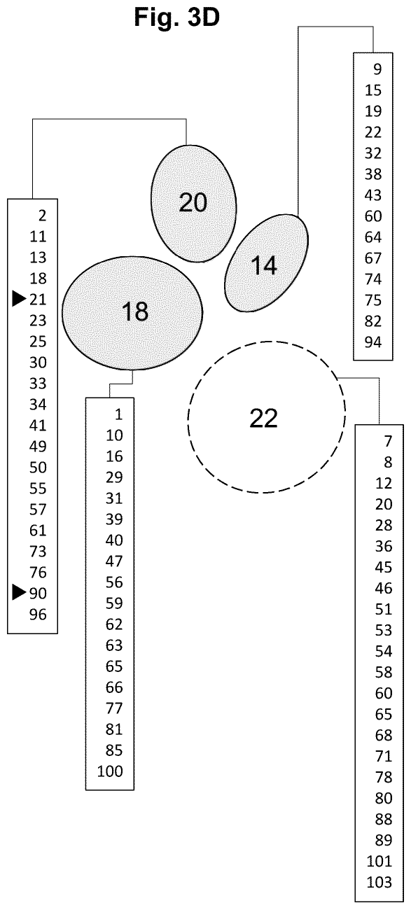

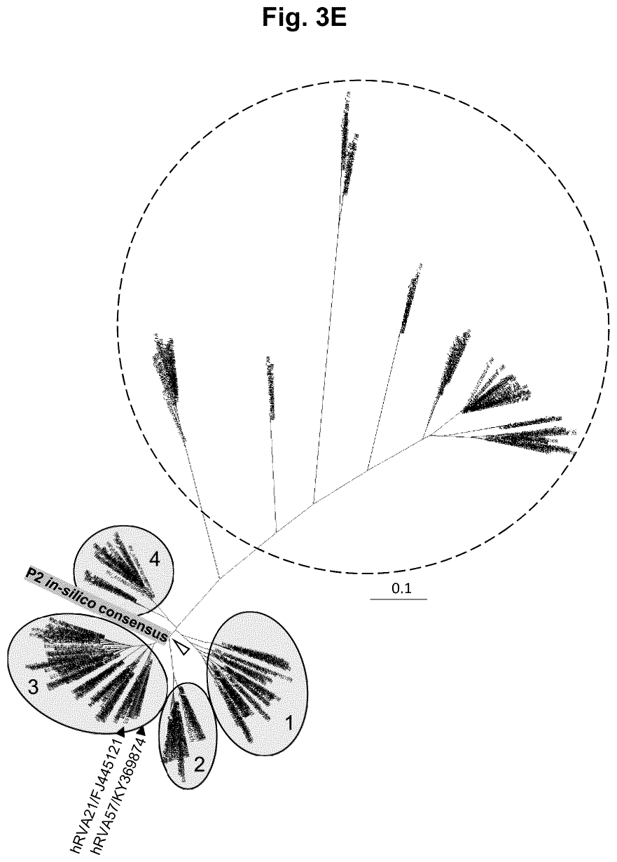

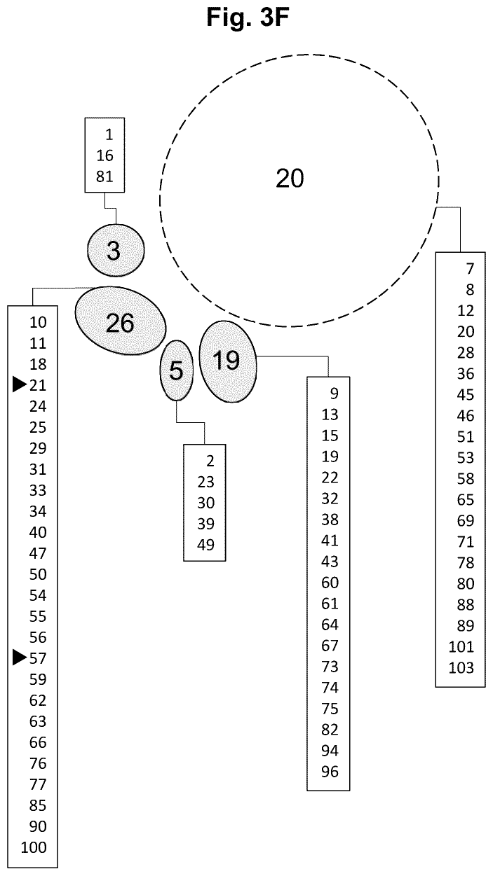

The phylogenetic analysis confirmed the large diversity among rhinovirus A strains. FIGS. 3A-3F shows the phylogenetic trees for each of the complete rhinovirus A polyprotein (FIG. 3A), the rhinovirus A VP0 polyprotein (FIG. 3C), and the rhinovirus A P2 polyprotein (FIG. 3E), respectively. The in silico consensus sequence for each of the polyproteins is shown at the center point from which different phylogenetic clusters branch off. Each of the three phylogenetic trees shows phylogenetic clusters that are very close to the center point. The analysis of amino acid sequences of the complete rhinovirus A polyprotein revealed at least four major phylogenetic clusters (see FIGS. 3A and 3B). As can be seen from FIGS. 3C and D, there are at least three major phylogenetic clusters of VP0 polyproteins with close proximity to the center point representing the in silico consensus sequence. Consistent with the analysis based on complete rhinovirus A polyprotein sequences, FIGS. 3E and 3F indicate that there are at least four major phylogenetic clusters of P2 polyproteins with close proximity to the center point representing the in silico P2 polyprotein consensus sequence. A closer look at the phylogenetic clustering obtained for the complete polyprotein, the VP0 polyprotein and P2 polyprotein sequences revealed that only one of the VP0 polyprotein clusters (cluster 1) corresponds to the clusters identified for the complete polyproteins and the P2 polyproteins. The other two clusters of the VP0 polyproteins were predominantly made up of a mixture of serotypes allocated to clusters 2 and 3 or clusters 3 and 4, respectively, that were identified for the complete polyproteins and the P2 polyproteins.

Further analysis indicated that 333 of 540 amino acid sequences (62%) used for the analysis had an identity of greater than 80% to the amino acid sequence of the complete rhinovirus polyprotein of the selected rhinovirus A serotype 21 strain (GenBank ID: FJ445121.1, strain name: ATCC VR-1131). The proportion was similar for the P2 polyprotein, with 298 out of 461 amino acid sequences (65%) used for the analysis having an identity of greater than 80% to the amino acid sequence of the P2 polyprotein of this strain. Surprisingly, the proportion was greater for the VP0 polyprotein, with 420 out of 517 analyzed amino acid sequences (81%) having an identity of greater than 80% to the amino acid sequence of the VP0 polyprotein of this strain.

Without wishing to be bound by any particular theory, the inventors hypothesize that a naturally occurring polyprotein having an amino acid sequence that has an average identity of at least 80% to the amino acid sequences of corresponding polyproteins from at least two phylogenetic clusters of rhinoviruses of the same group is capable of eliciting an immune response against multiple rhinovirus serotypes of that group. Accordingly, the selected naturally occurring VP0 and P2 polyproteins are likely to elicit an immune response that is broadly effective against multiple rhinovirus serotypes.

In summary, the P2 region encoding non-structural rhinovirus polypeptides and the VP0 region encoding structural rhinovirus polypeptides include the largest number of conserved regions of the rhinovirus A polyprotein. A single naturally occurring P2 polyprotein may be capable of eliciting an immune response against multiple serotypes of rhinovirus A. Similarly, a single naturally occurring VP0 polyprotein may be capable of eliciting an immune response against multiple serotypes of rhinovirus A. Even better coverage may be achieved by an immunogenic composition providing both a VP0 polyprotein and a P2 polyprotein as antigens.

Accordingly, this example indicates that an immunogenic composition comprising an mRNA encoding one or more naturally occurring polyproteins having an average identity of at least 80% to the amino acid sequences of corresponding polyproteins from at least two phylogenetic clusters of rhinoviruses of the same group is likely to elicit an immune response against multiple serotypes of rhinovirus group A strains in a subject. The immune response may reduce or prevent one or more symptoms associated with an infection caused by these serotypes.

### Example 2. Identification of T-Cell Epitope-Rich Regions in Rhinovirus A Polypeptides

This example illustrates that the conserved VP0 region of the rhinovirus A polyprotein, which encodes structural rhinovirus proteins VP4 and VP2, comprises a plurality of T-cell epitope-rich regions.

All published epitopes are made publicly available at the IEDB website (available at www.iedb.org). All such epitopes specific from human rhinoviruses were extracted from the public database on 8 Oct. 2021 and assembled into a database providing epitope sequence, epitope type (B, T-MHC-I or T-MHC-II), target organism and antigen and related publication links. A total of 233 published epitopes were retrieved.

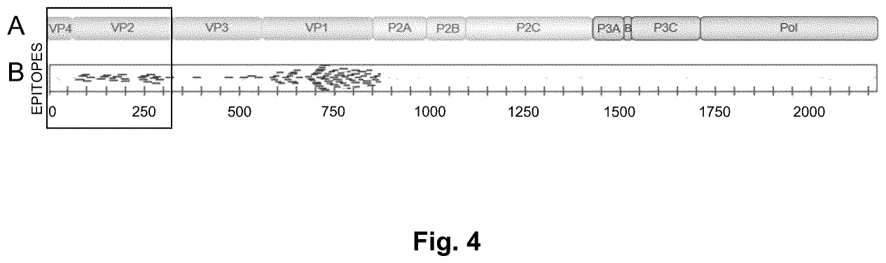

The published epitopes were mapped along the polyprotein, as shown in panel B of FIG. 4. Notably, all class-I published epitopes were found to be concentrated in the capsid proteins. This result is strongly biased by the fact that most research conducted on rhinovirus A is focused on the immune response against the VP proteins. Accordingly, the analysis revealed that VP1 and VP2 are the two privileged targets for published class-I T-cell epitopes. There are only a limited number of published class-II T-cell epitopes. They were found to be distributed all along the polyprotein (see panel B of FIG. 4).

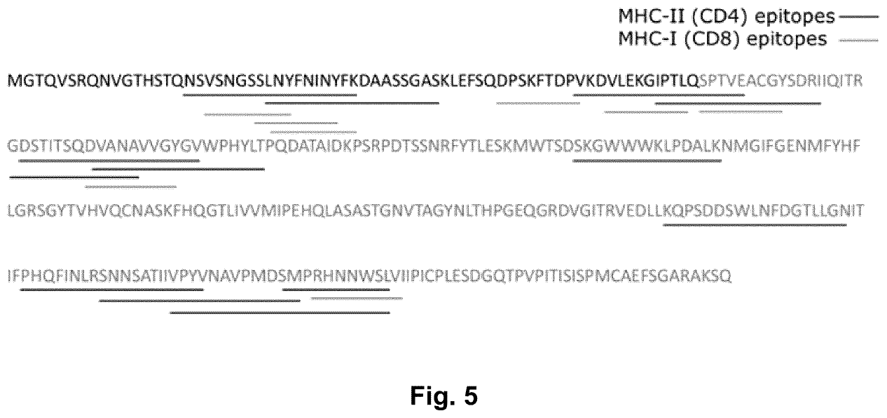

A corresponding analysis was also performed for the naturally occurring VP0 polyprotein of rhinovirus A serotype 21 (Genbank ID FJ445121.1) identified in Example 1, confirming the presence of multiple class-I T-cell epitopes and class-II T-cell epitopes across the length of this polyprotein (see FIG. 5).

This example demonstrates that the conserved VP0 region of the rhinovirus A polyprotein, which encodes structural rhinovirus proteins VP4 and VP2, comprises a plurality of class-I T-cell epitopes. Therefore, an immunogenic composition comprising an mRNA encoding one or more structural proteins with these T-cell epitope-rich regions is likely to elicit a T-cell response in a subject.

### Example 3. T-Cell Epitope Prediction in Rhinovirus A Polypeptides

This example illustrates that the conserved P2 region of the rhinovirus A polyprotein, which encodes non-structural rhinovirus proteins 2A, 2B and 2C, comprises a plurality of class-I and class-II T-cell epitopes. Furthermore, this example illustrates the conserved VP0 region of the rhinovirus A polyprotein, which encodes structural rhinovirus proteins VP4 and VP2, comprises a plurality of class-II T-cell epitopes, in addition to a plurality of class-I T-cell epitopes.

All T-cell epitope predictions were performed using the IEDB website (available at www.iedb.org). Both MHC-I and MHC-II (Major Histocompatibility Complex) affinity predictions were performed for each consensus sequence generated from alignments using parameters recommended by IEDB.

### MHC Class I Epitope Prediction

MHC class I T-cell epitope predictions were conducted with the IEDB recommended method 2020.09 (NetMHCpan EL 4.1).

Results were merged for the full MHC-I reference set of 27 alleles, and also for all possible peptide lengths (from 8 to 14-mers). All hits were entered into an excel table displaying each peptide sequence, coordinates in the target sequence and different scores obtained from the prediction.

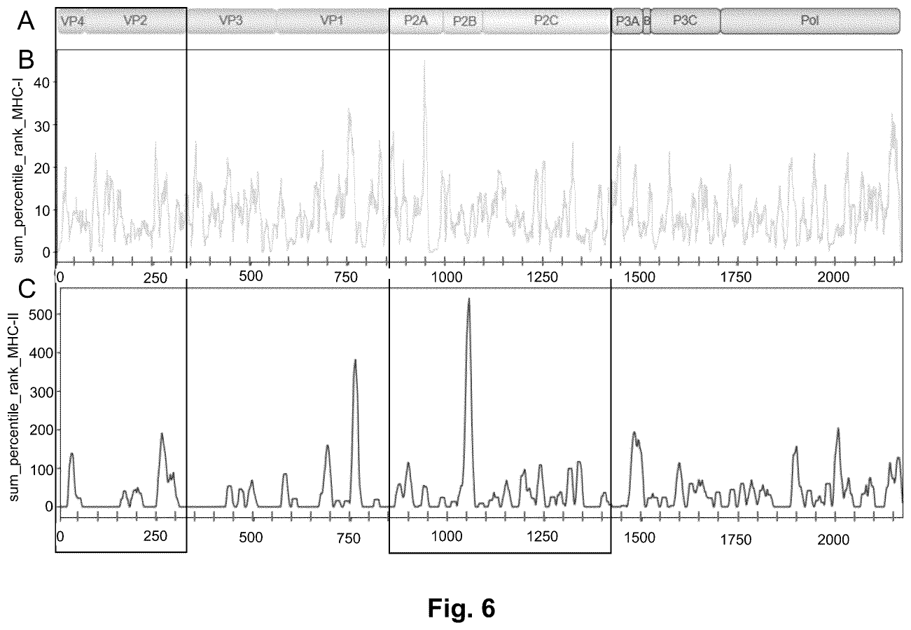

The almost 300,000 hits for the rhinovirus type A consensus polyprotein were filtered by percentile rank. Only hits with a percentile rank lower than 1% were selected for visualizations. Panel B of FIG. 6 displays the sum of percentile rank scores along the polyprotein.

Panel B of FIG. 6 displays only the global count of best hits. Another dimension of high importance is the qualitative content of different HLA alleles in a selected region. The MHC-I and MHC-II HLA alleles set recommended by IEDB has been set up to cover 97% to 99% of the worldwide human population. Any missing HLA in hits would result in a lower population coverage. Accordingly, the qualitative response in terms of potential human population coverage by the predicted hits was also determined.

To this end, hits were counted for each allele from the set of 27 MHC-I reference alleles to ensure the full coverage of the human population. All alleles are well represented in the full polyprotein in class-I predicted epitopes.

### MHC Class II Epitope Prediction

IEDB recommendations were also used for MHC-II epitope binding predictions: IEDB recommended method 2.22, which is a consensus approach from several methods conducted in parallel. The full HLA reference recommended set of 27 MHC-II alleles was used, and all peptide lengths hits were merged in the results table (from 11 to 30-mers). The hit table structure is similar that the one for MHC-I predictions.

The more than 800,000 hits for the rhinovirus type A consensus polyprotein were filtered by applying a threshold of 3% in the case of predicted class-II T-cell epitopes. Panel C of FIG. 6 displays the sum of percentile rank scores along the length of the polyprotein.

The qualitative response in terms of potential human population coverage by the predicted hits was also determined. Hits were counted for each allele from the set of 27 MHC-II reference alleles to ensure the full coverage of the human population. All alleles are well represented in the full polyprotein in class-II predicted epitopes.

This example demonstrates that the conserved P2 region of the rhinovirus A polyprotein, which encodes non-structural rhinovirus proteins 2A, 2B and 2C, comprises a plurality of class-I and class-II T-cell epitopes. Furthermore, this example illustrates the conserved VP0 region of the rhinovirus A polyprotein, which encodes structural rhinovirus proteins VP4 and VP2, comprises a plurality of class-II T-cell epitopes, in addition to a plurality of class-I T-cell epitopes. Moreover, this example demonstrates that the identified T-cell epitope-rich regions in these proteins cover the vast majority of MHC-I and MHC-II alleles present in the human population. An immunogenic composition comprising an mRNA encoding one or more of these non-structural or structural proteins with these T-cell epitope-rich regions is predicted to elicit an effective T-cell response in 97-99% of the human population.

### Example 4. Antigen Design Validation for Rhinovirus A

This example illustrates an antigen design process for two rhinovirus A antigens that integrates the findings of Examples 1-3. One antigen comprises the conserved P2 region of the rhinovirus A polyprotein, which encodes non-structural rhinovirus proteins 2A, 2B and 2C and comprises a plurality of class-I and class-II T-cell epitopes. The other antigen comprises the conserved VP0 region of the rhinovirus A polyprotein which encodes structural rhinovirus proteins VP4 and VP2 and likewise comprises a plurality of class-I and class-II T-cell epitopes.

The antigen design step integrated the different parameters identified in Examples 1-3. The amino acid sequence of the naturally occurring polyprotein of human rhinovirus A serotype 21 strain ATCC VR-1131 was most similar to the consensus sequence generated by the alignment of the human rhinovirus A polyproteins in Example 1. This sequence was used to identify regions of the polyprotein that are highly conserved regions among many rhinovirus A strains. In Example 2, published T-cell epitopes were mapped on the polyprotein sequence. Predicted best hits for putative T-cell epitopes of both class-I and class-II were identified in Example 3. Taking into account all considered parameters, the sequence of human rhinovirus A serotype 21 strain ATCC VR-1131 was selected for the antigen design. The antigen design focused on the two selected regions VP0 and P2.

The first antigen is composed of P2 region of the rhinovirus A polyprotein, which includes the three non-structural proteins 2A, 2B and 2C. As shown in panels A and B of FIG. 6, it is rich in predicted T-cell epitopes. As shown in FIG. 2, it also comprises the second-best conserved region in the polyprotein.

The P2 polyprotein comprises 560 amino acid and has the following ATCC VR-1131-derived sequence:

(SEQ ID NO: 6)
MGPSDMYVHVGNLIYRNLHLENSEMHDSILISYSSDLVIYRINTT
GDDYIPTCDCTEATYYCKHKNRYFPIKVTSHDWYEIQESEYYPKH
IQYNLLIGEGPCEPGDCGGKLLCKHGVIGMITAGGDGHVAFIDLR
HFHCAEEQGITDYIHMLGEAFGSGFVDSVKEQINAINPINNISKK
IIKWLLRIISAMVIIIRNSSDPQTIIATLTLIGCSGSPWRFLKEK
FCKWTQLNYIHKESDSWLKKFTEMCNAARGLEWIGNKISKFIDWM
KSMLPQAQLKVKYLSELKKLNLLEKQIEHLRAADSATQEKIKCEI
DTLHDLSCKFLPLYASEAKRIKVLHNKCNVVIKQKKRCEPVAVVI
HGEPGTGKSMTINFLARMITNDSDIYSLPPDPKYFDGYDQQSVVI
MDDIMQNPSGDDMTLFCQMVSSVTFIPPMADLPDKGKPFDSRFVL
CSTNHSLLTPPTITSLPAMNRRFFMDLDIIVCDKYKDAQGKLNVS
AAFKPCDVDTKIGNARCCPFICGKAVMFKDRNTCRTYTLAQIYNQ
ILEEDKRRRQVIDVMSAIFQ

The second antigen is composed of the VP0 region of the rhinovirus A polyprotein, which includes the structural proteins VP4+VP2. This region is the most conserved in the full polyprotein (see FIG. 2). It also includes a large number of both published and putative class-I and class-II T-cell epitopes (see FIGS. 4-6).

The VP0 polyprotein comprises 329 amino acid and has the following ATCC VR-1131-derived sequence:

(SEQ ID NO: 4)
MGTQVSRQNVGTHSTQNSVSNGSSLNYFNINYFKDAASSGASKLE
FSQDPSKFTDPVKDVLEKGIPTLQSPTVEACGYSDRIIQITRGDS
TITSQDVANAVVGYGVWPHYLTPQDATAIDKPSRPDTSSNRFYTL
ESKMWTSDSKGWWWKLPDALKNMGIFGENMFYHFLGRSGYTVHVQ
CNASKFHQGTLIVVMIPEHQLASASTGNVTAGYNLTHPGEQGRDV
GITRVEDLLKQPSDDSWLNEDGILLGNITIFPHQFINLRSNNSAT
IIVPYVNAVPMDSMPRHNNWSLVIIPICPLESDGQTPVPITISIS
PMCAEFSGARAKSQ

All prior analyses in Examples 1-3 were conducted on the global consensus polyprotein sequence. Therefore, the ATCC VR-1131-derived antigen sequences required independent validation.

### P2 Polyprotein

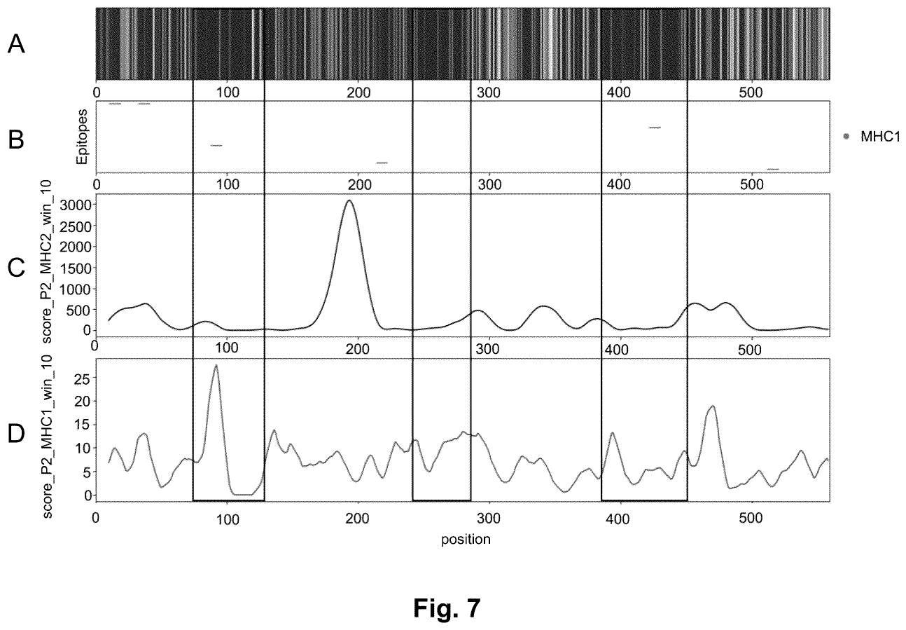

ATCC VR-1131-derived P2 polyprotein was confirmed to have a high degree of sequence conservation, especially around the regions defined by residues 80-120, residues 250-280 and residues 380-450 of SEQ ID NO: 6. A total of six class-I, but no class-II published T-cell epitopes are present in the P2 polyprotein (see FIG. 7). The major region of predicted class-II T-cell epitopes is not in the most conserved sequences, but other regions of predicted class-II T-cell epitopes are distributed all along the sequence. Several of the predicted class-I epitope comprising regions are also located in the highly conserved regions of SEQ ID NO: 6.

Table 9 shows the hit number per MHC-I and MHC-II alleles in the P2 polyprotein.

TABLE 9
P2 MHC-I/-II allele hits count

| MHC-I allele | count | MHC-II allele | count |
|---|---|---|---|
| HLA-A*26:01 | 35 | HLA-DPA1*01:03/DPB1*02:01 | 5 |
| HLA-B*51:01 | 38 | HLA-DPA1*01:03/DPB1*04:01 | 15 |
| HLA-B*07:02 | 26 | HLA-DPA1*02:01/DPB1*01:01 | 12 |
| HLA-B*57:01 | 37 | HLA-DPA1*02:01/DPB1*05:01 | 122 |
| HLA-A*02:06 | 24 | HLA-DPA1*02:01/DPB1*14:01 | 21 |
| HLA-A*68:01 | 21 | HLA-DPA1*03:01/DPB1*04:02 | 2 |
| HLA-A*01:01 | 40 | HLA-DQA1*01:01/DQB1*05:01 | 258 |
| HLA-B*58:01 | 37 | HLA-DQA1*01:02/DQB1*06:02 | 21 |
| HLA-B*40:01 | 30 | HLA-DQA1*03:01/DQB1*03:02 | 2 |
| HLA-A*02:01 | 21 | HLA-DQA1*04:01/DQB1*04:02 | 29 |
| HLA-A*30:02 | 36 | HLA-DQA1*05:01/DQB1*02:01 | 121 |
| HLA-B*53:01 | 42 | HLA-DQA1*05:01/DQB1*03:01 | 12 |
| HLA-A*03:01 | 26 | HLA-DRB1*01:01 | 55 |
| HLA-A*02:03 | 30 | HLA-DRB1*03:01 | 174 |
| HLA-A*24:02 | 45 | HLA-DRB1*04:01 | 426 |
| HLA-B*35:01 | 36 | HLA-DRB1*04:05 | 444 |
| HLA-B*15:01 | 35 | HLA-DRB1*07:01 | 260 |
| HLA-B*08:01 | 39 | HLA-DRB1*08:02 | 192 |
| HLA-A*33:01 | 24 | HLA-DRB1*09:01 | 199 |
| HLA-A*31:01 | 27 | HLA-DRB1*11:01 | 296 |
| HLA-A*23:01 | 46 | HLA-DRB1*12:01 | 305 |
| HLA-A*68:02 | 19 | HLA-DRB1*13:02 | 602 |
| HLA-B*44:03 | 36 | HLA-DRB1*15:01 | 719 |
| HLA-B*44:02 | 41 | HLA-DRB3*01:01 | 256 |
| HLA-A*11:01 | 30 | HLA-DRB3*02:02 | 206 |
| HLA-A*32:01 | 31 | HLA-DRB4*01:01 | 309 |
| HLA-A*30:01 | 29 | HLA-DRB5*01:01 | 40 |

P2 looks optimal to trigger a strong T-cell response. There are no missing alleles in the class-I and class-II predictions. Only three class-II alleles display low number of high score hits (shown in italics in Table 9).

### VP0 Polyprotein

The ATCC VR-1131-derived VP0 polyprotein was confirmed to have a high degree of sequence conservation, especially in its N-terminal part (VP4). A total of three class-I and six class-II published T-cell epitopes are present in the VP0 polyprotein (see FIG. 8). Two major regions of predicted class-II T-cell epitopes are in well-conserved regions (defined by residues 1-100, 150-200 and residues 229-299 of SEQ ID NO: 4), so are likely shared by many other serotypes. Most of the predicted class-I epitope comprising regions are also located in these highly conserved regions.

Table 10 shows the hit number per MHC-I and MHC-II alleles in the VP0 polyprotein.

TABLE 10
VP0 MHC-I/-II allele hits count

| MHC-I allele | count | MHC-II allele | count |
|---|---|---|---|
| HLA-A*26:01 | 29 | HLA-DPA1*01:03/DPB1*02:01 | 12 |
| HLA-B*51:01 | 28 | HLA-DPA1*01:03/DPB1*04:01 | 102 |
| HLA-B*07:02 | 19 | HLA-DPA1*02:01/DPB1*01:01 | 12 |
| HLA-B*57:01 | 29 | HLA-DPA1*02:01/DPB1*05:01 | 41 |
| HLA-A*02:06 | 19 | HLA-DPA1*02:01/DPB1*14:01 | 3 |
| HLA-A*68:01 | 17 | HLA-DPA1*03:01/DPB1*04:02 | 90 |
| HLA-A*01:01 | 25 | HLA-DQA1*01:01/DQB1*05:01 | 219 |
| HLA-B*58:01 | 32 | HLA-DQA1*01:02/DQB1*06:02 | 195 |
| HLA-B*40:01 | 8 | HLA-DQA1*03:01/DQB1*03:02 | 3 |
| HLA-A*02:01 | 14 | HLA-DQA1*04:01/DQB1*04:02 | 4 |
| HLA-A*30:02 | 24 | HLA-DQA1*05:01/DQB1*02:01 | 1 |
| HLA-B*53:01 | 30 | HLA-DQA1*05:01/DQB1*03:01 | 6 |
| HLA-A*03:01 | 10 | HLA-DRB1*01:01 | 32 |
| HLA-A*02:03 | 15 | HLA-DRB1*03:01 | 3 |
| HLA-A*24:02 | 19 | HLA-DRB1*04:01 | 310 |
| HLA-B*35:01 | 29 | HLA-DRB1*04:05 | 229 |
| HLA-B*15:01 | 17 | HLA-DRB1*07:01 | 1 |
| HLA-B*08:01 | 16 | HLA-DRB1*08:02 | 54 |
| HLA-A*33:01 | 16 | HLA-DRB1*09:01 | 0 |
| HLA-A*31:01 | 10 | HLA-DRB1*11:01 | 23 |
| HLA-A*23:01 | 17 | HLA-DRB1*12:01 | 0 |
| HLA-A*68:02 | 26 | HLA-DRB1*13:02 | 248 |
| HLA-B*44:03 | 6 | HLA-DRB1*15:01 | 7 |
| HLA-B*44:02 | 8 | HLA-DRB3*01:01 | 21 |
| HLA-A*11:01 | 15 | HLA-DRB3*02:02 | 241 |
| HLA-A*32:01 | 25 | HLA-DRB4*01:01 | 180 |
| HLA-A*30:01 | 7 | HLA-DRB5*01:01 | 1 |

The qualitative T-cell epitope predictions for the VP0 polyprotein shows two missing MHC-II alleles (shown in bold in Table 10). A frequency estimation of worldwide population coverage does not show any significant drop of the estimated coverage without the two missing alleles, as both are rare HLA alleles. Several other alleles display a low number of hits (4 in class-I and 9 in class-II; shown italics in Table 10).

This example demonstrates the design of two antigens, one comprising the conserved P2 region of the rhinovirus A polyprotein which encodes non-structural rhinovirus proteins 2A, 2B and 2C and comprises a plurality of class-I and class-II T-cell epitopes, and another comprising the conserved VP0 region of the rhinovirus A polyprotein which encodes structural rhinovirus proteins VP4 and VP2 and likewise comprises a plurality of class-I and class-II T-cell epitopes. Both antigens are designed to include conserved T-cell epitope-rich regions that cover the vast majority of MHC-I and MHC-II alleles present in the human population. An immunogenic composition comprising one or more mRNAs encoding either one or both of these antigens is predicted to elicit an effective T-cell response in the vast majority of human subjects.

### Example 5. Fusion Protein Design for Rhinovirus A

This example illustrates a fusion protein comprising the conserved P2 region of the rhinovirus A polyprotein encoding non-structural rhinovirus proteins 2A, 2B and 2C and the conserved VP0 region of the rhinovirus A polyprotein encoding structural rhinovirus proteins VP4 and VP2.

The P2-VP0 fusion protein has the following sequence, wherein P2 is in bold and VP0 is underlined:

(SEQ ID NO: 34)
MGPSDMYVHVGNLIYRNLHLFNSEMHDSILISYSSDLVIYRTNTT
GDDYIPTCDCTEATYYCKHKNRYFPIKVTSHDWYEIQESEYYPKH
IQYNLLIGEGPCEPGDCGGKLLCKHGVIGMITAGGDGHVAFIDLR
HFHCAEEQGITDYIHMLGEAFGSGFVDSVKEQINAINPINNISKK
IIKWLLRIISAMVIIIRNSSDPQTIIATLTLIGCSGSPWRFLKEK
FCKWTQLNYIHKESDSWLKKFTEMCNAARGLEWIGNKISKFIDWM
KSMLPQAQLKVKYLSELKKLNLLEKQIEHLRAADSATQEKIKCEI
DTLHDLSCKFLPLYASEAKRIKVLHNKCNVVIKQKKRCEPVAVVI
HGEPGTGKSMTTNFLARMITNDSDIYSLPPDPKYFDGYDQQSVVI
MDDIMQNPSGDDMTLFCQMVSSVTFIPPMADLPDKGKPFDSRFVL
CSTNHSLLTPPTITSLPAMNRRFFMDLDIIVCDKYKDAQGKLNVS
AAFKPCDVDTKIGNARCCPFICGKAVMFKDRNTCRTYTLAQIYNQ
ILEEDKRRRQVIDVMSAIFQGTQVSRQNVGTHSTQNSVSNGSSLN
YFNINYFKDAASSGASKLEFSQDPSKFTDPVKDVLEKGIPTLQSP
TVEACGYSDRIIQITRGDSTITSQDVANAVVGYGVWPHYLTPQDA
TAIDKPSRPDTSSNRFYTLESKMWTSDSKGWWWKLPDALKNMGIF
GENMFYHFLGRSGYTVHVQCNASKFHQGTLIVVMIPEHQLASAST
GNVTAGYNLTHPGEQGRDVGITRVEDLLKQPSDDSWLNFDGTLLG
VNITIFPHQFINLRSNNSATIIVPYVNAVPMDSMPRHNNWSLVIIP
ICPLESDGQTPVPITISISPMCAEFSGARAKSQ

The fusion protein comprises a starting methionine, P2A in italics and bold, P2B in bold, P2C in italics and bold, VP4 underlined, VP2 underlined and in italics.

To elicit a strong T-cell response, a fusion protein comprising both the VP0 and P2 polyprotein was designed. Table 11 shows the hit number per MHC-I and MHC-II alleles in the VP0+P2 fusion protein.

TABLE 11
VP0 + P2 MHC-I/-II allele hits count

| MHC-I allele | count | MHC-II allele | count |
|---|---|---|---|
| HLA-A*26:01 | 64 | HLA-DPA1*01:03/DPB1*02:01 | 17 |
| HLA-B*51:01 | 66 | HLA-DPA1*01:03/DPB1*04:01 | 117 |
| HLA-B*07:02 | 45 | HLA-DPA1*02:01/DPB1*01:01 | 24 |
| HLA-B*57:01 | 66 | HLA-DPA1*02:01/DPB1*05:01 | 163 |
| HLA-A*02:06 | 43 | HLA-DPA1*02:01/DPB1*14:01 | 24 |
| HLA-A*68:01 | 38 | HLA-DPA1*03:01/DPB1*04:02 | 92 |
| HLA-A*01:01 | 65 | HLA-DQA1*01:01/DQB1*05:01 | 477 |
| HLA-B*58:01 | 69 | HLA-DQA1*01:02/DQB1*06:02 | 216 |
| HLA-B*40:01 | 38 | HLA-DQA1*03:01/DQB1*03:02 | 5 |
| HLA-A*02:01 | 35 | HLA-DQA1*04:01/DQB1*04:02 | 33 |
| HLA-A*30:02 | 60 | HLA-DQA1*05:01/DQB1*02:01 | 122 |
| HLA-B*53:01 | 72 | HLA-DQA1*05:01/DQB1*03:01 | 18 |
| HLA-A*03:01 | 36 | HLA-DRB1*01:01 | 87 |
| HLA-A*02:03 | 45 | HLA-DRB1*03:01 | 177 |
| HLA-A*24:02 | 64 | HLA-DRB1*04:01 | 736 |
| HLA-B*35:01 | 65 | HLA-DRB1*04:05 | 673 |
| HLA-B*15:01 | 52 | HLA-DRB1*07:01 | 261 |
| HLA-B*08:01 | 55 | HLA-DRB1*08:02 | 246 |
| HLA-A*33:01 | 40 | HLA-DRB1*09:01 | 199 |
| HLA-A*31:01 | 37 | HLA-DRB1*11:01 | 319 |
| HLA-A*23:01 | 63 | HLA-DRB1*12:01 | 305 |
| HLA-A*68:02 | 45 | HLA-DRB1*13:02 | 850 |
| HLA-B*44:03 | 42 | HLA-DRB1*15:01 | 726 |
| HLA-B*44:02 | 49 | HLA-DRB3*01:01 | 277 |
| HLA-A*11:01 | 45 | HLA-DRB3*02:02 | 447 |
| HLA-A*32:01 | 56 | HLA-DRB4*01:01 | 489 |
| HLA-A*30:01 | 36 | HLA-DRB5*01:01 | 41 |

The P2-VP0 fusion protein looks optimal to trigger a strong T-cell response. It has no missing alleles in class-I and -II predictions, and only a single class-II allele displays a low number of hits.

This example demonstrates that a fusion protein comprising the conserved P2 region of the rhinovirus A polyprotein encoding non-structural rhinovirus proteins 2A, 2B and 2C and the conserved VP0 region of the rhinovirus A polyprotein encoding structural rhinovirus proteins VP4 and VP2 is likely the optimal design to elicit an effective T-cell response against the majority of known rhinovirus A serotypes. An immunogenic composition comprising an mRNA encoding this fusion protein is predicted to elicit an effective T-cell response in 97-99% of the human population.

### Example 6. Assessing Rhinovirus A Polyprotein Expression

This example illustrates the expression of the P2 polyprotein and the VP0 polyprotein provided in Example 4 and the fusion protein provided in Example 5 using mRNAs comprising optimized nucleotide sequences encoding these proteins.

Using a sequence optimization algorithm as described herein, optimized nucleotide sequences were generated that encode the amino acid sequences of the P2 polyprotein and the VP0 polyprotein provided in Example 4 and the amino acid sequence of the fusion protein provided in Example 5. For both the P2 polyprotein and the VP0 polyprotein, two optimized nucleotide sequences were selected for testing. The optimized nucleotide sequences encoding the fusion protein were constructed from the optimized sequences encoding the P2 polyprotein and the VP0 polyprotein. In addition, optimized nucleotide sequences were prepared in which the HA secretion signal sequence of influenza strain A/California/7/2009 was fused to the N-terminus of the P2 polyprotein, the VP0 polyprotein or the fusion protein. The HA secretion signal sequence had the amino acid sequence of SEQ ID NO: 15 and was encoded by the nucleotide sequence of SEQ ID NOs: 71 or 72. The amino acid sequence DTL was added to the C-terminus of the secretion signal to fuse it to the VP0 polyprotein. This sequence corresponds to the first three amino acids at the N-terminus of the mature influenza HA protein of influenza strain A/California/7/2009.

For ease of detection of the expressed protein, additional optimized nucleotide sequences were prepared that encoded FLAG-tagged versions of the P2 polyprotein, the VP0 polyprotein and the fusion protein. The FLAG-tag with the amino acid sequence DYKDDDDK (SEQ ID NO: 73) was linked to the C-terminus of each protein using a linker with the amino acid sequences GGGGS (SEQ ID NO: 74). The nucleotide sequence encoding the FLAG-tag was GACTACAAGGACGATGACGATAAG (SEQ ID NO: 75), and the nucleotide sequence encoding the linker was GGAGGCGGAGGCAGC (SEQ ID NO: 76), respectively.

mRNAs were prepared by in vitro transcription from a template plasmid comprising a nucleic acid sequence comprising a 5′-UTR of SEQ ID NO: 5, the respective optimized sequence, and a 3′-UTR of SEQ ID NO: 6, operably linked to an RNA polymerase promoter sequence. The resulting mRNAs were capped and tailed in separate multi-step, enzyme-catalyzed process and then purified to remove enzyme reagents. The final mRNAs had a Cap1 structure at the 5′ end and a poly(A) tail of 100-250 nucleotides in length at the 3′ end. The cap structure consisted of a 7-methyl guanosine (m7G) residue linked via an inverted 5′5′ triphosphate bridge to the first nucleoside of the 5′-UTR, which is itself modified by 2′-O-ribose methylation.

LIPOFECTAMINE® MESSENGERMAX™ (Thermo Fisher Scientific) was combined with the mRNAs encoding the FLAG-tagged proteins at a 1:2.5 ratio of mRNA (μg) to Lipofectamine (μL) to prepare transfection mixtures. For each protein, two mRNAs comprising different optimized nucleotide sequences were tested. After 10±5 minutes incubation with Lipofectamine, 15 μL transfection mixtures were added to wells of a multi-well plate. 1 mL of HeLa at 2×10⁵ cells/mL were added to each well. The cells were placed in a humidified tissue culture incubator at 37° C. with 5% CO₂ for 20±4 hours. Supernatants were collected, and 0.25 mL of RIPA buffer containing 1×HALT protease inhibitor and 0.2% OMNICLEAVE™ (Biosearch Technologies) was added to the cells. The plates were incubated on ice for 15 minutes. The lysates were collected and clarified at 10,000 rcf, 4° C., 10 minutes. Supernatants and lysates were used immediately or stored at −80° C. until use.

Samples were combined with 4×LDS loading dye and reducing agent, boiled for 5 minutes at 95° C., and run on an 8-16% PAGE gel. Proteins were transferred to nitrocellulose membranes, total protein was stained with REVERT™ (LI-COR) and blocked in Intercept TBS blocking buffer (LI-COR). Blots were probed overnight with 1:1,000 anti-FLAG (R&D Systems MAB8529), washed three times for 5 minutes in TBST, and then for 1 hour with 1:5,000 anti-rabbit IRDye800. Blots were washed, rinsed in H₂O and then images on an Odyssey CLx scanner.

Both the supernatants and RIPA lysate were analyzed by Western Blot. Expression was scored as high expression +++, medium expression ++, low expression +, and not detected N.D., as set out in Table 12.

TABLE 12
Expression levels of the mRNA-encoded proteins

| | SEQ ID NO. | Expression | Expression |
|---|---|---|---|
| | (protein/ | level in | level in |
| mRNA-encoded protein | nucleotide) | supernatant | lysate |
| VP0 polyprotein | 3/9 | N.D. | ++ |
| VP0 polyprotein | 3/10 | N.D. | ++ |
| P2 polyprotein | 2/7 | N.D. | ++ |
| P2 polyprotein | 2/8 | N.D. | ++ |
| P2-VP0 fusion protein | 4/18 | N.D. | + |
| P2-VP0 fusion protein | 4/19 | N.D. | + |
| VP0 polyprotein with | 12/16 | +++ | +++ |
| secretion signal sequence | | | |
| VP0 polyprotein with | 12/17 | +++ | +++ |
| secretion signal sequence | | | |
| P2 polyprotein with | 11/14 | N.D. | ++ |
| secretion signal sequence | | | |
| P2 polyprotein with | 11/15 | N.D. | ++ |
| secretion signal sequence | | | |
| P2-VP0 fusion protein | 13/22 | N.D. | + |
| with secretion signal | | | |
| sequence | | | |
| P2-VP0 fusion protein | 13/23 | N.D. | + |
| with secretion signal | | | |
| sequence | | | |

Expression of the mRNA-encoded protein was observed in the lysates of all transfected cells, and in the supernatants of cells expressing the VP0 polyprotein linked to a secretion signal sequence. P2 polyprotein was detected as two bands at ˜60 kDa and at ˜45 kDa, indicating that the polyprotein was cleaved.

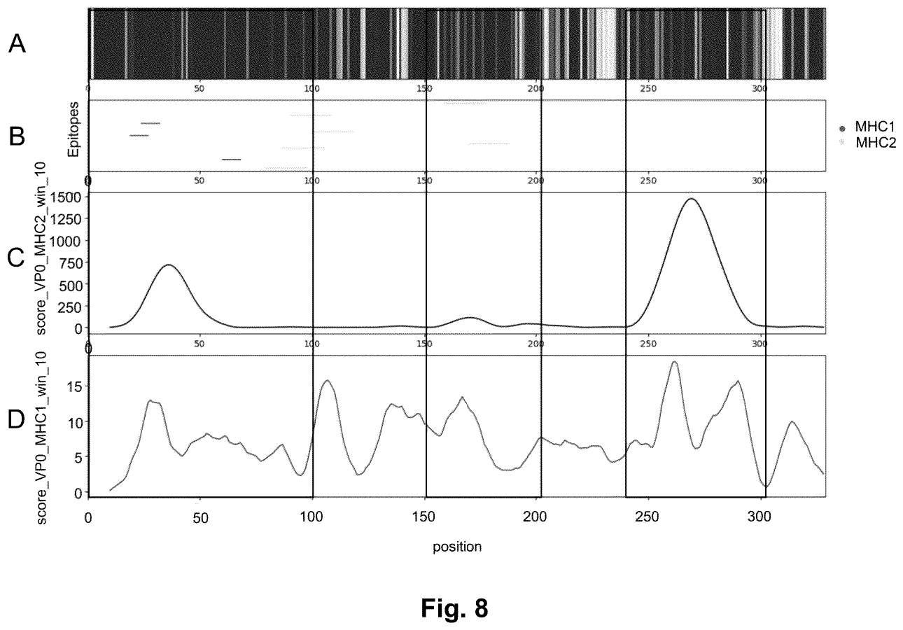

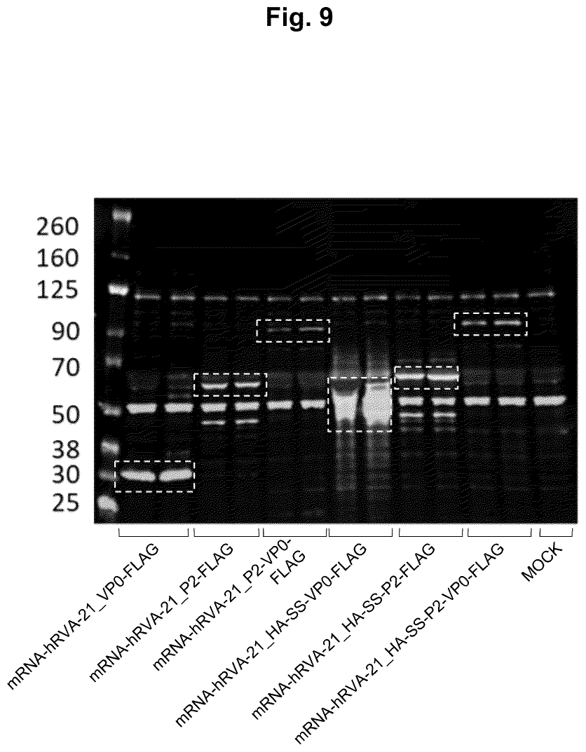

As can be seen from Table 12 and FIG. 9, abundant expression was observed in lysates and supernatants of cells transfected with mRNAs encoding the VP0 polyprotein linked to a secretion signal sequence. Lysates of cells transfected with mRNAs encoding the P2 polyprotein or the fusion protein showed much lower levels of expression. To determine the cause of the reduced expression levels, the previously prepared cell lysates were further analyzed by Western blot using an anti-eIF4g antibody (CST 2948 @1:1,000). Intact eIF4g was observed in lysates from cells transfected with mRNAs encoding the VP0 polyprotein. In cells transfected with mRNA encoding the P2 polyprotein or the fusion protein, eIF4g cleavage was observed. eIF4g cleavage leads to global translational repression. These data indicate that functional P2 is expressed in cells transfected with mRNA encoding the P2 polyprotein or the fusion protein.

This example demonstrates successful expression and activity of the P2 polyprotein and the VP0 polyprotein provided in Example 4 and the fusion protein provided in Example 5 after transfection of cells with mRNAs comprising optimized nucleotide sequences encoding these proteins. Operationally linking a rhinovirus antigen to a non-native secretion signal sequence from a different virus resulted in effective secretion of the rhinovirus antigen in the cell supernatant.

### Example 7. Engineering Rhinovirus A P2 Polyprotein with Reduced or No Proteolytic Activity

This example illustrates that a point mutation can be introduced in the P2 polyprotein to inactivate its proteolytic activity.

The active site of rhinovirus A serotype 21 protein 2A comprises a catalytic triad including His18, Asp35 and Cys106 of the native coding sequence. The catalytic triad of protein 2A is not always in the same position across all serotypes of rhinovirus A. This is due to the different lengths of the native 2A proteins across rhinovirus A. For example, the active site of rhinovirus A serotype 8 protein 2A comprises a catalytic triad including His18, Asp36 and Cys107 of the native coding sequence. The proteolytic activity can be reduced or abolished by substituting the cysteine which acts as the nucleophile in the catalytic triad with a serine or alanine. The inventors found that substituting the cysteine residue in rhinovirus A serotype 21 with alanine resulted in a more drastic effect of abolishing the proteolytic activity.

The ATCC VR-1131-derived P2 polyprotein (serotype 21) comprising a cysteine to alanine (C>A) point mutation has the following sequence (the C>A substitution is shown in bold and underlined):

(SEQ ID NO: 77)
MGPSDMYVHVGNLIYRNLHLENSEMHDSILISYSSDLVIYRTNTT
GDDYIPTCDCTEATYYCKHKNRYFPIKVTSHDWYEIQESEYYPKH
IQYNLLIGEGPCEPGDAGGKLLCKHGVIGMITAGGDGHVAFIDLR
HFHCAEEQGITDYIHMLGEAFGSGFVDSVKEQINAINPINNISKK
IIKWLLRIISAMVIIIRNSSDPQTIIATLTLIGCSGSPWRFLKEK
FCKWTQLNYIHKESDSWLKKFTEMCNAARGLEWIGNKISKFIDWM
KSMLPQAQLKVKYLSELKKLNLLEKQIEHLRAADSATQEKIKCEI
DTLHDLSCKFLPLYASEAKRIKVLHNKCNVVIKQKKRCEPVAVVI
HGEPGTGKSMTTNFLARMITNDSDIYSLPPDPKYFDGYDQQSVVI
MDDIMQNPSGDDMTLFCQMVSSVTFIPPMADLPDKGKPFDSRFVL
CSTNHSLLTPPTITSLPAMNRRFFMDLDIIVCDKYKDAQGKLNVS
AAFKPCDVDTKIGNARCCPFICGKAVMFKDRNTCRTYTLAQIYNQ
ILEEDKRRRQVIDVMSAIFQ

Similarly, a correspondingly mutated rhinovirus A serotype 8 derived from ATCC VR-1118 (SEQ ID NO: 81) has reduced or no proteolytic activity.

### Example 8. Identification of Rhinovirus C Polyproteins Suitable as Antigens

This example illustrates that a single VP0 polyprotein comprising the structural rhinovirus proteins VP4 and VP2 is better suited than a single P2 polyprotein comprising the non-structural rhinovirus proteins 2A, 2B and 2C as an antigen capable of inducing protection against a large number of rhinovirus C strains.

Publicly available sequences were retrieved from the ViPR website as described in Example 1. Sequence retrieval was performed on 9 Sep. 2022. At this time, the database contained a total of 22,589 human rhinovirus sequences. 7,021 of these were annotated as human rhinovirus C sequences.

The first step was to serotype sequences by clustering them into phylogenetic trees as described in Example 1. Sequences encoding proteins shorter than 800 amino acids in length or comprising X stretches longer than 10 were excluded from the analysis. This reduced the number of human rhinovirus C sequences used for alignments to 463.

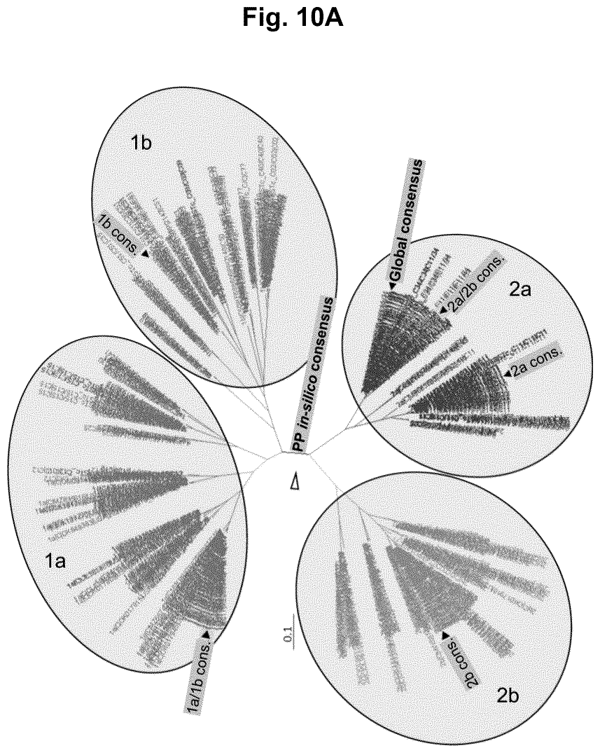

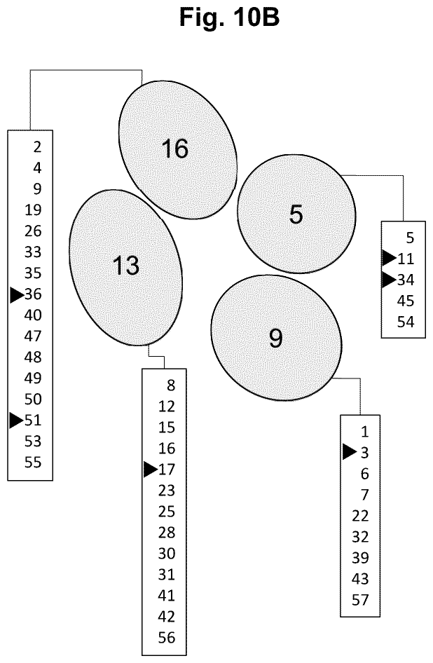

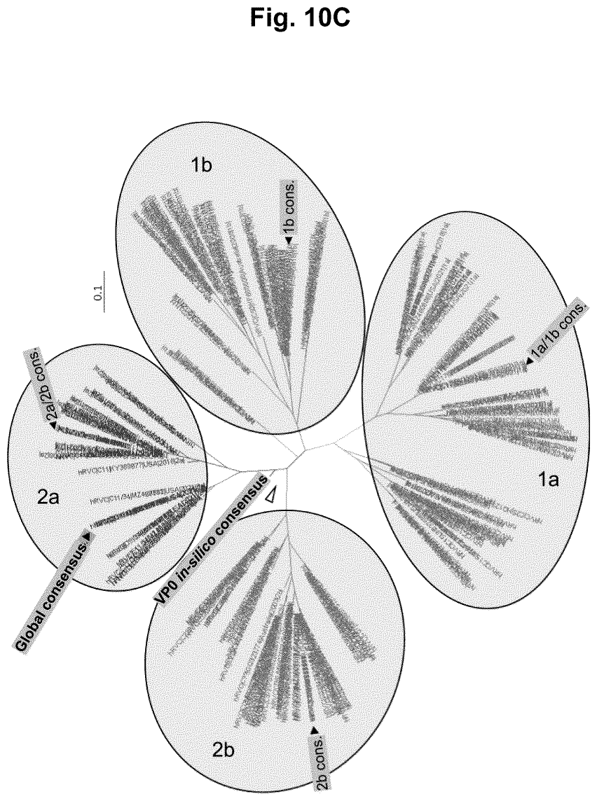

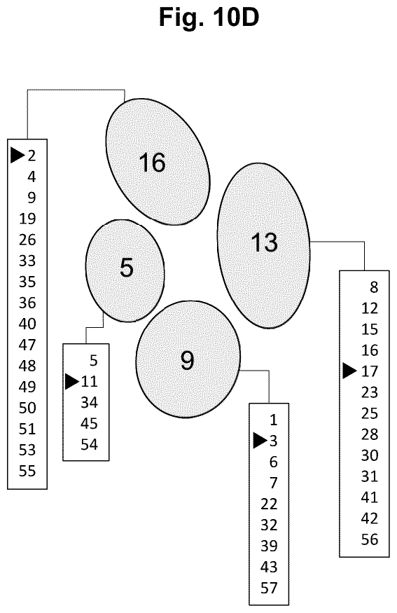

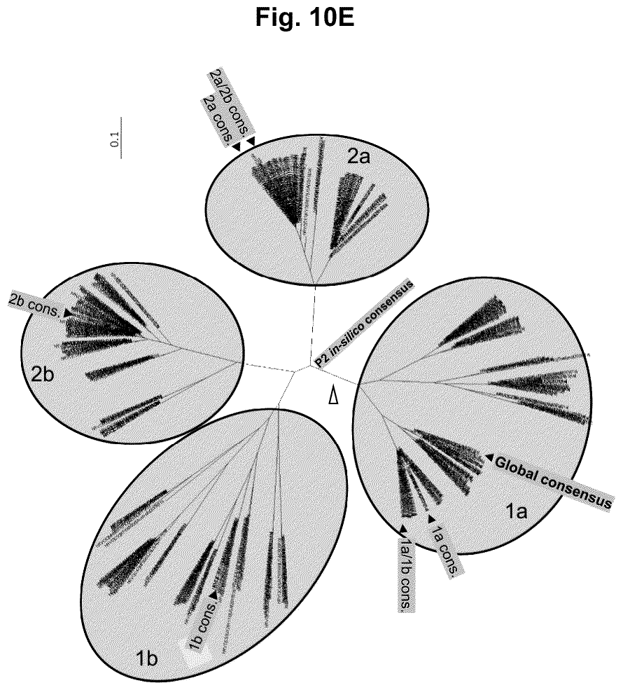

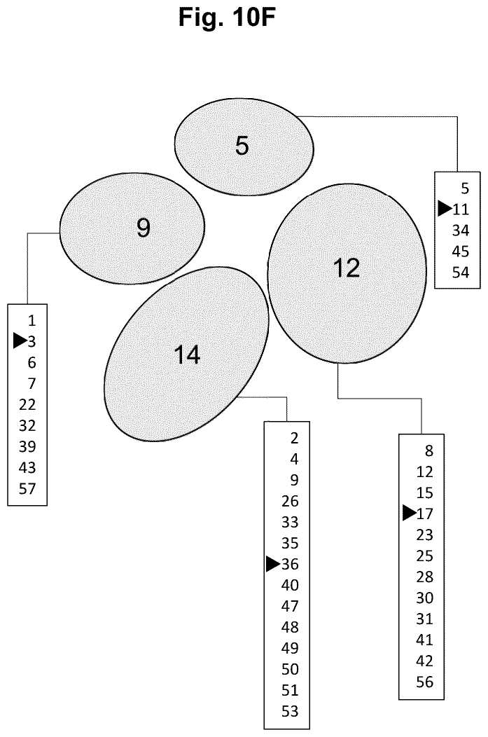

Using FastTree as described in Example 1, the retrieved polyprotein sequences could be subdivided into two large phylogenetic clusters, which were subdivided into two phylogenetic clusters each (see FIG. 10A), designated 1ab and 2ab. The four potential clusters were designated 1a, 1b, 2a and 2b, comprising of 13, 16, 5 and 9 serotypes, respectively (see FIGS. 10B, 10D and 10F, respectively). The same clustering was also observed, when the FastTree analysis was repeated for the VP0 polyprotein (see FIG. 10C) and the P2 polyprotein (see FIG. 10E) of rhinovirus C.

Based on the results obtained in Example 1, it was determined that a naturally occurring rhinovirus polypeptide, protein or polyprotein having both an average identity and a median identity of greater than 80% to corresponding rhinovirus polypeptides, proteins or polyproteins of multiple rhinovirus serogroups would be suitable to provide broad protection. Using the same approach as described in Example 1, rhinovirus C polyprotein consensus sequences were determined. Consistent with the FastTree analysis discussed above, two consensus sequences—one for the 1ab cluster and one for the 2ab cluster—were required to meet the average/median identity criterion (see Table 13).

TABLE 13
Average percent identity of the complete rhinovirus C polyprotein
consensus sequence relative to complete rhinovirus C polyproteins
of different phylogenetic clusters

Polyprotein consensus

| | All | 1ab | 1a | 1b | 2ab | 2a | 2b |
|---|---|---|---|---|---|---|---|
| Average | 78 | 81 | 86 | 82 | 82 | 90 | 92 |
| Median | 79 | 81 | 87 | 83 | 82 | 91 | 92 |
| Min. | 71 | 69 | 71 | 73 | 74 | 81 | 76 |
| Max. | 81 | 90 | 94 | 85 | 87 | 94 | 99 |

A similar finding was also made, when the analysis was repeated for the P2 polyprotein (see Table 14).

TABLE 14
Average percent identity of the rhinovirus C P2 polyprotein
consensus sequence relative to rhinovirus C P2 polyproteins
of different phylogenetic clusters

P2 consensus

| | All | 1ab | 1a | 1b | 2ab | 2a | 2b |
|---|---|---|---|---|---|---|---|
| Average | 78 | 81 | 86 | 81 | 82 | 92 | 94 |
| Median | 79 | 83 | 90 | 82 | 85 | 96 | 96 |
| Min. | 71 | 68 | 73 | 75 | 69 | 85 | 74 |
| Max. | 83 | 91.0 | 95 | 85 | 89 | 98 | 100 |

Surprisingly, despite encoding surface-exposed capsid proteins VP2 and VP4, a single consensus sequence for the VP0 polyprotein could be identified that had both an average identity and a median identity of greater than 80% to the analyzed rhinovirus sequences (see Table 15).

TABLE 15
Average percent identity of the rhinovirus C VP0 polyprotein
consensus sequence relative to rhinovirus C VP0 polyproteins
of different phylogenetic clusters

VP0 consensus

| | All | 1ab | 1a | 1b | 2ab | 2a | 2b |
|---|---|---|---|---|---|---|---|
| Average | 83 | 84 | 88 | 86 | 87 | 93 | 93 |
| Median | 82 | 85 | 88 | 85 | 87 | 94 | 91 |
| Min. | 75 | 73 | 74 | 81 | 81 | 87 | 84 |
| Max. | 87 | 93 | 96 | 91 | 91 | 96 | 98 |

This result indicates that a single VP0 polyprotein is the most suitable antigen to induce an immune response against rhinovirus C serotypes of all four phylogenetic clusters 1a, 1b, 2a and 2b. Using the approach described in Example 1, the available rhinovirus C sequences were screened to identify a naturally occurring VP0 polyprotein that met the average/median identity criterion. The results of this analysis are summarized in Table 16. The percent identity of the naturally occurring VP0 polyprotein to the in silico generated VP0 polyprotein consensus sequence is indicated in the column labelled “Id % to in silico consensus”. The number of distinct serotypes in each phylogenetic cluster (including the smaller clusters 1a, 1b, 2a and 2b) is indicated in the column labelled “#ST”.

Human rhinovirus C serotype 34 strain (GenBank ID: MZ322913.1, strain name: 7H8M5V) was the best match (see Table 16) and was selected to design an expression-optimized coding sequence for an mRNA-based vaccine. The GenBank ID: MZ322913.1 has been annotated to indicate that the VP0 polyprotein is derived from human rhinovirus C serotype 20. A sequence search revealed that the amino acid sequence of the VP0 polyprotein of MZ322913 is identical to the amino acid sequences of VP0 polyproteins annotated as deriving from rhinovirus C serotypes 20 and 34.

TABLE 16
Naturally occurring protein closest to in silico VP0 polyprotein consensus

| Best | | Id % to in silico | | | | | |
|---|---|---|---|---|---|---|---|
| match | Strain | consensus | #ST | Average | Median | Min | Max |
| hRVC | hRVC\|C11/34\|MZ322913\| | 87.0 | | 82.8 | 82.4 | 75.4 | 87.0 |
| | USA\|2021\|2a | | | | | | |
| 1ab | hRVC\|C17\|MZ153277\| | 93.0 | 29 | 84.2 | 85.0 | 72.9 | 93.0 |
| | USA\|2021\|1a | | | | | | |
| 1a | hRVC\|C17\|MZ153277\| | 95.8 | 13 | 88.2 | 88.0 | 73.9 | 95.8 |
| | USA\|2021\|1a | | | | | | |
| 1b | hRVC\|C02\|MK989752\| | 90.6 | 16 | 85.6 | 84.5 | 81.1 | 90.6 |
| | Kenya\|2015\|1b | | | | | | |
| 2ab | hRVC\|C11\|MZ268689\| | 91.2 | 14 | 87.4 | 86.7 | 80.7 | 91.2 |
| | USA\|2021\|2a | | | | | | |
| 2a | hRVC\|C11\|MZ268689\| | 96.4 | 5 | 92.5 | 94.3 | 87.3 | 96.4 |
| | USA\|2021\|2a | | | | | | |
| 2b | hRVC\|C03\|MZ268693\| | 98.2 | 9 | 92.8 | 91.3 | 84.3 | 98.2 |
| | USA\|2021\|2b | | | | | | |

The VP0 polyprotein of the selected strain (serotype 34) has the following amino acid sequence:

(SEQ ID NO: 3)
MGAQVSKQNVGSHESGISASSGSVIKYFNINYYKDSASSGLSKQD
ESMDPEKFTKPIAETLINPALMSPSIEACGFSDRLKQITIGNSTI
TTQDALNTVVAYGEWPQYLSDMDASAIDKPTHPETSTDRFYTLTS
VIWDTTSKGWWWKIPDCLKEMGMFGQNMYHHALGRSGFIIHVQCN
ATKFHSGLLIVAVVPEHQLAYIGGTNVSVGYNHTHPGENGHTIGL
NDQRGDRQPDEDPFFNCNGTLLGNLTIFPHQLINLRINNSATIVV
PYINCVPMDSMLRHNNLSLVIIPMVDLRFGTTGVTTLPITISIAP
VKSEFSGARQSRTQ

To determine whether the antigen design could be improved by providing separate VP0 sequences for each of the two larger phylogenetic clusters 1ab and 2ab, the analysis was repeated with two separate VP0 consensus sequences for each cluster. Indeed, consistent with the FastTree analysis, this resulted in a better match, identifying a consensus sequence with both an average identity and a median identity of greater than 85% for phylogenetic clusters 1a and Tb, and an average identity and a median identity of greater than 90% for clusters 2a and 2b (see Table 16). Once again, the available rhinovirus C sequences were screened to identify a naturally occurring VP0 polyprotein that met these revised average/median identity criteria. The VP0 polyprotein of a human rhinovirus C serotype 17 strain (GenBank ID: MZ153277.1, strain name: RvC17/USA/2021/368038-4) was the best match for cluster 1ab, and the VP0 polyprotein of a human rhinovirus C serotype 11 strain (GenBank ID: MZ268689.1, isolate: 469843) was the best match for cluster 2ab (see Table 14).

Notably, the VP0 polyprotein of the selected rhinovirus C serotype 11 strain was a significantly better match across the phylogenic clusters 2a and 2b. The two identified VP0 polyprotein sequences were selected to design an expression-optimized coding sequence for an mRNA-based vaccine.

The VP0 polyprotein of the selected serotype 17 strain (GenBank ID: MZ153277.1, strain name: RvC17/USA/2021/368038-4) has the following amino acid sequences:

(SEQ ID NO: 2)
MGAQVSRQTTGSHESAVNATNGGIIKYFNINYYRDSASSGLTKQD
FSQDPSKFTQPLVDTLINPALMSPSVEACGFSDRLKQITMGNSTI
TTQDALHTVLAYGEWPQYLSDLDATSVDKPTHPETSSDRFYTLSS
VSWTNTSKGWWWKLPDALKDMGVFGQNLYYHAMGRAGYIIHTQCN
ATKFHSGALLVVLIPEHQLAYIGAEKVNIAYDLTHPGETGHVIGR
NTSRGNNNPDEDPFFNCNGTLFGNLTIFPHQIINLRINNSSTIIT
PYINCQPMDNMLKHNNLTLLIVPLVRLRFGTEASPTVSITVTIAP
YKSEFSGAMETQKHQ

The VP0 polyprotein of the selected serotype 11 strain (GenBank ID: MZ268689.1, isolate: 469843) has the following amino acid sequences:

(SEQ ID NO: 1)
MGAQVSKQNVGSHESGISASSGSVIKYFNINYYKDSASSGLSKQD
ESMDPEKFTKPLADVMINPALMSPSIEACGFSDRLKQITIGSSTI
TTQDTLNTVVAYGEWPEYLRDTDASAVDKPTHPETSTDRFYTLTS
VIWNGSSKGWWWKIPDCLKDMGMFGQNMYHHALGRSGYIFHIQCN
ATKFHSGLLLVAIVPEHQLAYVGGTYANVGYNHTHPGEGGHEIRE
PTGRDDKKPDEDPLFNCNGTLLGNLTIFPHQLINLRTNNSVTIVV
PYINCVPMDSMLKHNNISLVIIPLVPLRSGSTQAPQTLPITISIA
PDKSEFSGARQSNKTQ

This example demonstrates that a single rhinovirus C VP0 polyprotein is better suited than a single rhinovirus C P2 polyprotein as an antigen capable of inducing protection against multiple rhinovirus C serotypes. A single naturally occurring VP0 polyprotein with an average and median identity of at least 80% to the amino acid sequences of VP0 polyproteins from at least two phylogenetic clusters of rhinovirus C may be able to provide broad protection. Slightly better coverage for at least three of the four phylogenetic clusters (including 2a and 2b) may be achieved by an immunogenic composition that provides two rhinovirus C VP0 polyproteins as antigens.

### Example 9. Identification of Rhinovirus C P2 Polyproteins Suitable as Antigens

This example provides rhinovirus C P2 polyproteins that are suitable as antigens to provide immunity against multiple serotypes of rhinovirus group C.

Example 1 identified the rhinovirus A P2 polyprotein as containing highly conserved T-cell epitope-rich regions. The non-structural rhinovirus proteins 2A, 2B and 2C are subject to different evolutionary pressures than the structural rhinovirus proteins VP2 and VP4 comprised in the VP0 polyprotein. Accordingly, providing a rhinovirus P2 polyprotein as an additional antigen may improve the immune response and result in a wider protection against infection with various rhinovirus C serotypes.

Based on the analysis described in Example 8 (see Table 14), the available rhinovirus C sequences were screened to identify naturally occurring P2 polyproteins that had both an average identity and a median identity of greater than 80% to the analyzed rhinovirus P2 polyprotein sequences in each of the two phylogenetic clusters 1ab and 2ab. The results of this analysis in summarized in Table 17. The percent identity of the naturally occurring P2 polyprotein to the in silico generated P2 polyprotein consensus sequence is indicated in the column labelled “Id % to in silico consensus”. The number of distinct serotypes in each phylogenetic cluster (including the smaller clusters 1a, 1b, 2a and 2b) is indicated in the column labelled “#ST”.

The P2 polyprotein of a human rhinovirus C serotype 17 strain (GenBank ID: MZ153245.1, strain name: RvC17/USA/2021/RCC55) was identified as the best match for cluster 1ab, and the P2 polyprotein of a human rhinovirus serotype 11 strain (GenBank ID: OK254863.1, strain name: RvC11/USA/2021/L2PJH9) was identified as the best match for cluster 2ab (see Table 17). Notably, these strains were different from the serogroup 11 and serogroup 17 strains identified as the source of suitable VP0 polyproteins. Therefore, additionally including P2 polyproteins in the design of an mRNA-based vaccine may indeed result in broader protection against rhinovirus C infection.

TABLE 17
Naturally occurring protein closest to in silico P2 polyprotein consensus

| Best | | Id % to in silico | | | | | |
|---|---|---|---|---|---|---|---|
| match | Strain | consensus | #ST | Average | Median | Min | Max |
| hRVC | hRVC\|C28\|JN798569\| | 82.7 | | 72.5 | 67.0 | 64.4 | 99.5 |
| | USA\|2000\|1a\| | | | | | | |
| 1ab | hRVC\|C17\|MZ153245\| | 91.0 | 29 | 79.3 | 77.1 | 63.8 | 99.8 |
| | USA\|2021\|1a\| | | | | | | |
| 1a | hRVC\|C17\|MZ153258\| | 95.4 | 13 | 85.5 | 87.3 | 70.7 | 100.0 |
| | USA\|2021\|1a\| | | | | | | |
| 1b | hRVC\|C36/C51\|MZ221147\| | 85.2 | 16 | 77.5 | 72.1 | 69.4 | 100.0 |
| | USA\|2021\|1b\| | | | | | | |
| 2ab | hRVC\|C11\|OK254863\| | 89.4 | 14 | 79.4 | 84.6 | 64.7 | 100.0 |
| | USA\|2021\|2a\| | | | | | | |
| 2a | hRVC\|C11\|EU840952\| | 97.5 | 5 | 91.9 | 93.1 | 82.7 | 99.6 |
| | Switzerland\|2008\|2a | | | | | | |
| 2b | hRVC\|C03\|MN228693\| | 99.5 | 9 | 93.6 | 95.4 | 74.0 | 100.0 |
| | USA\|2019\|2b | | | | | | |

Example 6 identified the proteolytic activity of the rhinovirus A protein 2A as an obstacle to high levels of antigen expression. Therefore, for the antigen design, both rhinovirus C P2 polyprotein sequences were modified by replacing the cysteine in the active site with alanine as described in Example 7. The active site of rhinovirus C protein 2A comprises a catalytic triad including His18, Asp34 and Cys105. The proteolytic activity can be reduced or abolished by substituting the cysteine which acts as the nucleophile in the catalytic triad with a serine or alanine. As described previously, the inventors found that substituting the cysteine residue with alanine resulted in a more drastic effect of abolishing the proteolytic activity.

The modified P2 polyprotein of a human rhinovirus C serotype 17 strain (GenBank ID: MZ153245.1, strain name: RvC17/USA/2021/RCC55) has the following amino acid sequence (the C>A substitution is marked in bold and underlined):

(SEQ ID NO: 78)
GPSDQCVHTKDAIYTCAHLTEPNSNTILLAITADLQVDSTDTPGP
DFIPTCDCVQACYYAKHAQRYYPITVTPHDWYEIQESQYYPKHIQ
YNILIGEGPCEPGDAGGKLLCRHGVIGIITAGGDGHVAFTDLRPY
ACLSTHQGLVSDYVNQLGAAFGDGFSSNIKDHLTGLCTTVSDKIT
GKVIKWLVRVISALTIMIRNSSDTATVLATLALLGCHGSPWSFLK
EKICQWLGIPRPPTRQGESWLKKFTECCNAAKGLEWVAQKIGKFI
DWLKEKLIPTVQRKKETLDQCKKIGLYEEQTKGFSHSEAEAQQSL
ILEVAKLKRGLDDLAPLYASENKRVTIIQKELQRLSAYQKTHRHE
PVCCLLRGPPGCGKSLVTSIIAHGLTNEANIYSLPPDPKHFDGYN
QQTVVIMDDVGQNPDGKDLSMFCQMVSTTEFIVPMASIEDKGRAF
TSQYVLASTNLDSLSPPTVTIPEAISRRFYLDADLQVTSKFKAHN
GLLDVAKALQPCAKCPKPNHYKQCCPILCGQALVLRDRRTSANYP
LLAVVEQLRMENNTRDKVKSNLRAIFQ

The P2 polyprotein of a human rhinovirus serotype 11 strain (GenBank ID: OK254863.1, strain name: RvC11/USA/2021/L2PJH9) has the following amino acid sequence (the C>A substitution is marked in bold and underlined):

(SEQ ID NO: 79)
GPSDMYVHTKEAIYKNAHLISANEQTILIALTADLQVDAADHPGD
DVIPDCDCTTGTYYCKSKDRYYPVEVVSHAWYPIEETCYYPKHIQ
YNILLGEGPCVPGDAGGKLLCKHGVIGIVTAGGENHVAFTDLRPY
SNLAHTQGPISDYVTQLGNAFGTGFTQTLETNLRETCSGMFDAIT
SKTVKWVVRIISALTIVIRNSSDIPTILATMALLGCTGSPWQYLK
SKLCNWLGVQKPPSKQSDSWLKKFTEWCNAAKGLEWIGYKISKFI
DWLKEKLIPTVQRKKDTLLECKKLTLYEDQVRAFPQSPEAFQNEL
TTKLQILKKNLDDMCPLYAAENKRVINMLRDIKTMTAYKKTHRTE
PVCVLIHGGPGCGKSLATTVIARGLTDSGNVYSLPPSPKHFDGYC
QQQVVMMDDLGQNPDGQDLAMFCQMVSTTDFIVPMAALEDKGKSF
TSDFVLASTNLNQLSPPTVTIPEAITRRFFLDVDLKIMSGYRTHA
GLLDTAKALQACPDCAKPPYYKQCCPLLCGKAVVLQNRRTSASLS
LNMVVSQLREESNTRKRIHTNLNAIFQ

This example identifies naturally occurring rhinovirus C P2 polyproteins that are suitable as antigens to provide immunity against multiple serotypes of rhinovirus group C. To design the antigens, the naturally occurring amino acid sequences were modified by a single amino acid substitution to reduce or abolish the proteolytic activity of the rhinovirus C protein 2A.

### Example 10. Preparation of mRNA-Encapsulating Lipid Nanoparticles

This example illustrates the preparation of immunogenic compositions in which the mRNAs prepared in Example 6 are encapsulated in lipid nanoparticles (LNPs) comprising a cationic lipid, a non-cationic lipid, a PEG-modified lipid, and a cholesterol-based lipid.

mRNAs encoding either the VP0 polyprotein or the P2 polyprotein from Example 4, or the fusion protein from Example 5 were synthesized in vitro as described in Example 6. The purified mRNAs were separately encapsulated in LNPs comprising the cationic lipid OF-02, the non-cationic lipid DOPE, cholesterol and the PEG-modified lipid DMG-PEG-2K at molar ratios of 40:30:28.5:1.5. The final mRNA-LNP formulations were provided in an aqueous suspension.

### Example 11. Eliciting an Immune Response to Rhinovirus A In Vivo

This example illustrates that an immunogenic composition comprising mRNA encoding the VP0 polyprotein of Example 4 is effective in eliciting an immune response in vivo against corresponding polyproteins from other phylogenetic clusters of rhinoviruses of the same group. In particular, the immunogenic compositions are effective in inducing a T-cell response in the test subjects.

An immunogenic composition was prepared as described in Example 10. This composition included an LNP encapsulating an mRNA encoding a rhinovirus A serotype 21 VP0 polyprotein as described in Example 4. In addition, an LNP was prepared without mRNA. The empty LNP served as a negative control. A recombinant VP0 polyprotein of a rhinovirus A serotype 16 strain formulated with the T_{H}1 adjuvant SPA09 served as a positive control. Each of the three compositions were separately administered to groups of wild-type C57BL/6 mice (n=6) via intramuscular injection in a 2-dose immunization series (day 0 and day 21). mRNA was administered at a dose of 2 μg of mRNA. The animals were sacrificed on day 35, and serum and spleen samples were collected.

Cross-reactivity against VP0 polyproteins from rhinovirus A serotypes belonging to different phylogenetic clusters than the rhinovirus A strain used for the antigen design (serotype 21) was assessed. Splenocytes were stimulated for 6 hours with different overlapping peptides libraries derived from the VP0 polyproteins of rhinovirus A strains representing serotypes 21, 1b and 8. VP0 from rhinovirus A serotypes 1b and 8 were selected based on their percent sequence identity relative to the VP0 from rhinovirus A serotype 21, which was present in the VP0 polyprotein encoded by the mRNA used for immunization. As shown in Example 1, the VP0 polyprotein of rhinovirus A serotype 21 has the highest percent sequence identity to the consensus sequence determined in that example. The VP0 polyprotein of rhinovirus A serotype 8 has the lowest percent sequence identity to the consensus sequence amongst all tested rhinovirus A serotypes, whereas rhinovirus A serotype 1b falls into the middle. Both serotypes 1b and 8 are in phylogenetic clusters that are different from that including serotype 21. The serotype 1b and 8 VP0 peptides had 85% and 76% identity to the serotype 21 VP0 peptide, respectively.

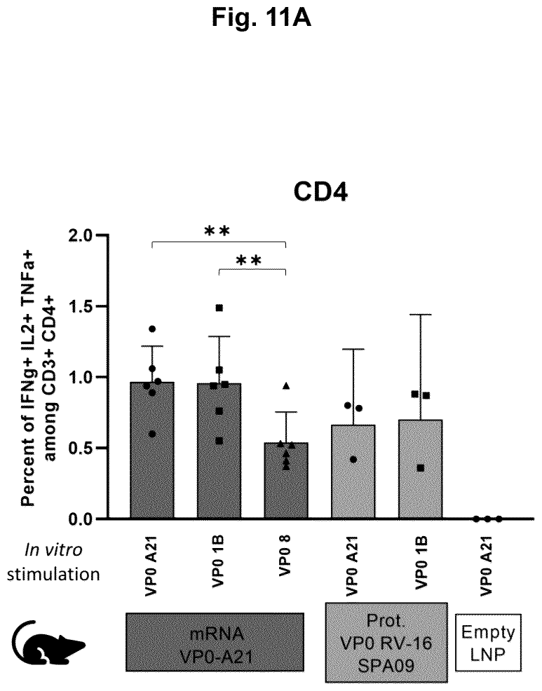

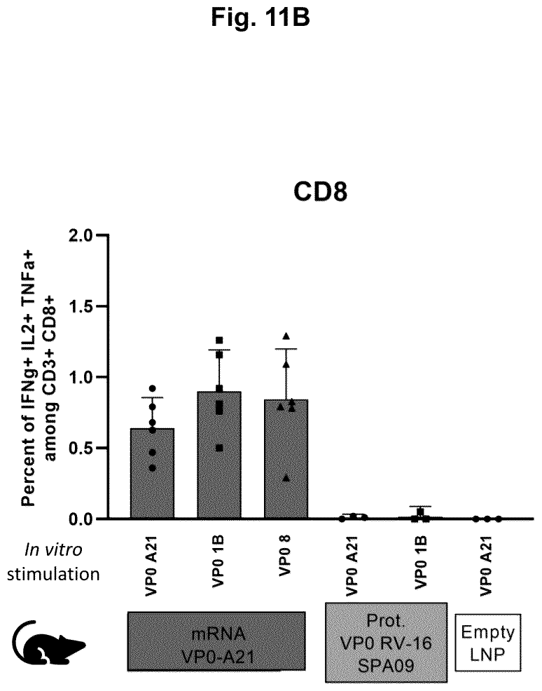

Following in vitro stimulation with overlapping peptide libraries, the percentages of polyfunctional IFN-γ, IL-2 and TNF-α-positive CD4+ and polyfunctional IFN-γ, IL-2 and TNF-α-positive CD8+ T-cells were analyzed using intracellular cytokine staining (ICS) and flow cytometry analysis. Immunization of mice with an mRNA encoding the VP0 polyprotein of rhinovirus A serotype 21 induced cross-reactive polyfunctional CD4+ and CD8+ T-cells in mice, as shown in FIG. 11A and FIG. 11B. About 1% of the CD4+ T-cells stimulated with the serotype 21 VP0 peptide were polyfunctional (see FIG. 11A). This was similar to the percentage observed with the positive control. This is notable because the mRNA-LNP composition did not include a separate adjuvant, unlike the recombinant VP0 polyprotein that was used as a positive control. A similar percentage of about 1% of polyfunctional CD4+ T-cells was also observed after stimulation with the serotype 1b VP0 peptide, confirming that the immune response elicited after immunization with the VP0-encoding mRNA extends to serotypes of other phylogenetic clusters that have VP0 polyproteins with at least 80% identity to the VP0 polyprotein encoded by the mRNA used for immunization. Notably, slightly fewer polyfunctional CD4+ T-cells (0.5%) were detected after stimulation with the serotype 8 VP0 peptide, which has a lower percent sequence identity to the serotype 21 VP0 peptide. The observed difference was statistically significant.

Surprisingly, no statistical difference was observed in the percentage of polyfunctional CD8+ T-cells. In comparison to the recombinant protein that was used as a positive control, a very high number of polyfunctional CD8+ T-cells (0.6-0.9%) was observed after immunization with the VP0-encoding mRNA and stimulation with any of the three VP0 peptides from serotypes 21, 1b and 8 (see FIG. 11B). The stimulation of a broadly cross-reactive CD8+ T-cell response is particular impressive as it indicates that the immune response elicited by the mRNA-encoded VP0 polyprotein may extend to phylogenetically more distant rhinoviruses of the same group than expected.

In summary, this example demonstrates that an immunogenic composition of the invention comprising an mRNA encoding a naturally occurring rhinovirus polyprotein is effective in eliciting an effective T-cell response in vivo against corresponding polyproteins from other phylogenetic clusters of rhinoviruses of the same group. The data also indicate that broader immune protection against rhinoviruses of the same group may be achieved by providing an immunogenic composition that comprises mRNAs encoding polyproteins from phylogenetically distant rhinovirus serotypes of the same group.

### Example 12. Immunization with an mRNA Encoding a VP0, P2 or VP0-P2 Fusion Protein

This example illustrates that an immunogenic composition comprising an mRNA encoding either the VP0 polyprotein or the P2 polyprotein of Example 4 is effective in eliciting an immune response in vivo against corresponding polyproteins from other phylogenetic clusters of rhinoviruses of the same group. The example also shows that mRNA encoding the VP0 protein can induce IgG titers against whole rhinovirus that are comparable to those induced by immunization with an adjuvanted recombinant VP0 protein.

A portion of the data from the mouse immunization experiments described in this example has already been described in Example 11.

### Mouse Immunization

Seven different immunogenic compositions were prepared as described in Example 10. The first and second composition included an LNP encapsulating an mRNA encoding a rhinovirus A serotype 21 VP0 polyprotein as described in Example 4, with or without an HA signal sequence, respectively. The third and fourth compositions included an LNP encapsulating an mRNA encoding a rhinovirus A serotype 21 P2 polyprotein as described in Example 4, with or without an HA signal sequence, respectively. The fifth composition included an LNP encapsulating an mRNA encoding a rhinovirus A serotype 21 P2 polyprotein as described in Example 7, which had been mutated at the catalytic site to prevent cleavage of translation factor eIF4g. In vitro studies confirmed that expression was comparable for mRNAs encoding the mutated P2 polyprotein and the corresponding wild-type protein. The sixth and seventh compositions included an LNP encapsulating an mRNA encoding a rhinovirus A serotype 21 P2-VP0 polyprotein as described in Example 5, with and without an HA signal sequence.

In addition, a corresponding LNP was prepared without mRNA. The empty LNP served as a negative control. A recombinant VP0 polyprotein of a rhinovirus A serotype 16 strain formulated with the T_{H}1 adjuvant SPA09 served as a positive control.

Each composition was separately administered to groups of 8-week-old wild-type C₅₇BL/6J mice (n=6) via injection in the quadriceps muscle at a dose of either 0.2 or 2 μg in a 2-dose immunization series (day 0 and day 21). The positive control group received a 10 μg dose of the adjuvanted recombinant VP0 polyprotein. Administration route and regimen was otherwise the same. The animals were sacrificed at week 5 post immunization, and serum and spleen samples were collected.

### Intracellular Cytokine Staining

Spleens were harvested from vaccinated or control mice and passed through 70 μm filters. Cells were stimulated for 6 hours with peptide libraries of 15-amino-acid (aa) peptides with 11 aa overlaps covering VP0 or P2 full length protein (GenScript, Piscataway, NJ) at 1 μg/ml in complete medium (RPMI 1640 supplemented with penicillin, streptomycin, L-glutamine, 10% fetal bovine serum (FBS)) in 96-well plates. Brefeldin A (10 μg/ml) was added during the final 5 hours of culture.

Cells were stained with Live/dead reagent, APC Fire-anti-CD14, APC Fire anti-CD19, Alexa Fluor 700 anti-CD3, Kiravia Blue 520 anti-CD4, BV510 anti-CD8. Cells were fixed and permeabilized with Cytofix/Cytoperm according to manufacturer protocol (BD Biosciences, Pont de Claix, Fr). Intracellular cytokines were stained with BV421 anti-IFN-γ, BV711 anti-TNF-μ, APC anti-IL2, and PE anti-IL-5 antibodies. All antibodies and Live/Dead were purchased from Biolegend (San Diego, CA).

Flow cytometry was performed on a BD Fortessa X-20. Data were analyzed using FlowJo 10.8.1 software. Dead cells, monocytes and B cells were excluded based on Live/dead staining, CD14+ and CD19+ cells, respectively. T cells were positively gated by CD3+, and CD4+ and CD8+ T cells were then positively gated. Cytokine expressions were evaluated in each CD4 and CD8 T cell sub-populations. Boolean analysis was performed for IFN-γ, IL-2 and TNF-β expression for both CD4 and CD8 T cells to analyze the single-, double- and triple-cytokine positive T cells.

### IFN-γ FluoroSpot

IFN-γ FluoroSpot assay was performed using Mouse Fluorospot kit (Mabtech, Nacka Strand, Sweden Cat #FS-4143-10). Anti-mouse IFN-γ pre-coated Multiscreen™ 96-well IPFL plates (AN18) were washed with 1×PBS and blocked for 30 minutes at room temperature with RPMI 1640 supplemented with penicillin, streptomycin, L-glutamine, 10% FBS. After elimination of complete medium, 2×10⁵ freshly isolated splenocytes were added per well and incubated for 24 hours with peptide pools (1 μg/mL), medium alone as negative control or concanavalin A (2.5 μg/mL) as a positive control.

After 6 washes with 0.1% bovine serum albumin (BSA) in 1×PBS, 100 μL of anti-mouse IFN-γ BAM-conjugated antibody (R4-6A2) diluted in PBS-0.1% BSA were added per well for 2 hours at room temperature in the dark. After washes in PBS-0.1% BSA, 100 μL of anti-BAM 490 antibody (1:200 dilution) in PBS-0.1% BSA were added per well and incubated for 1 hour at room temperature in the dark. After 5 washes with PBS and incubation with a FluoroSpot enhancer, fluorescent spots were counted with an automated fluorospot plate reader (Microvision, Instruments, Evry, France).

Results were expressed as number of specific IFN-γ-secreting cells per 10⁶ spleen cells after subtracting fluorospot counts from negative control wells.

### Enzyme-Linked Immunosorbent Assay for the Detection of Rhinovirus-Specific IgGs

Rhinovirus-specific IgGs were assayed using indirect Enzyme-linked immunosorbent assay (ELISA). Nunc microwell plates were coated overnight at 4° C. with (i) 2 μg/mL VP2 or 1 μg/mL VP4 proteins (Sanofi) from rhinovirus A serotype 21, respectively, in carbonate bicarbonate buffer; or (ii) purified rhinovirus A serotype 1b or rhinovirus A serotype 21 at 9.3 log₁₀ Geq/mL in 1×PBS.

After three washes with PBS-Tween 0.05%, plates were blocked with 1% milk in PBS-Tween 0.05% for 1 hour. Serum samples were diluted two-fold and added to the first wells followed by two-fold serial dilutions in blocking buffer. After a 1-hour incubation at room temperature, plates were washed three times in 1×PBS before adding a secondary antibody.

HRP goat anti-mouse (Jackson) was added diluted at 1:5,000 (VP2 wells), 1:2,500 (VP4 wells), or at 1:20,000 for virus coating. Plates were incubated at room temperature for 1 hour and washed three times. Plates were developed using tetramethylbenzidine (TMB) substrate solution for 30 minutes and stopped by HCL solution. Plates were read at 450 nm in a SpectraMax plate reader, and the data analyzed using Softmax Pro 6.5.1 GxP software.

Antibody titers were calculated as the reciprocal dilution given an optical density (OD) of 1.

### Statistical Analysis

Statistical analyses were performed with SAS® SEG software in a Wise 4 Environment. The nominal level of statistical significance was set to α=0.05 for effect estimates and α=0.01 for normality tests.

Analysis for ICS and ELISPOT readouts were performed on log₁₀-transformed data. A mixed model with vaccine and stimulation as fixed factors was used for ICS analysis. The data were paired among stimulation, and a Variance Component (VC) matrix of variance/co-variance was used. The heterogeneity of the results was considered using the “group=” option in the model. For ELISPOT readouts, a mixed model by stimulation with vaccine as fixed factor was used. For ELISA IgG readouts, the data did not follow a normal distribution, so non-parametric statistics were used. When data was paired, Wilcoxon and Wilcoxon signed rank test were used.

T-Cell Populations Elicited by mRNA Encoding a VP0 or P2 Polyprotein

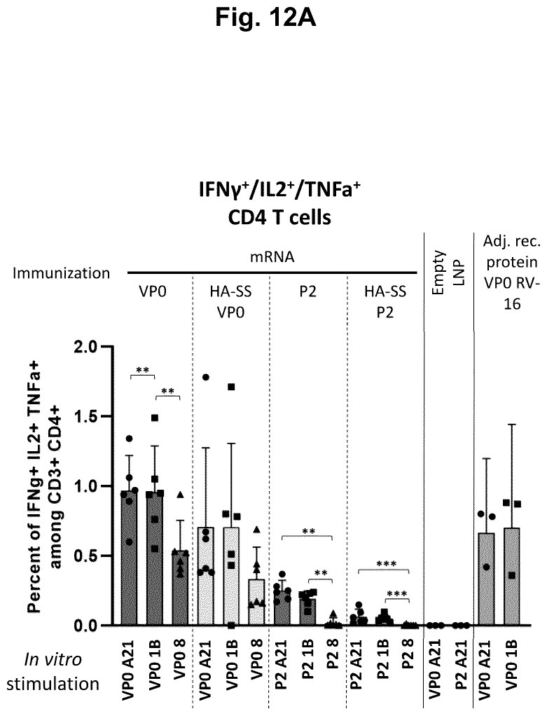

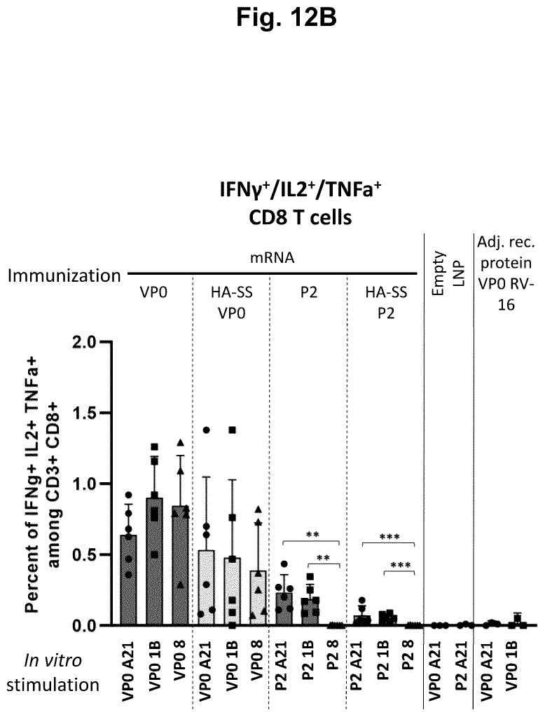

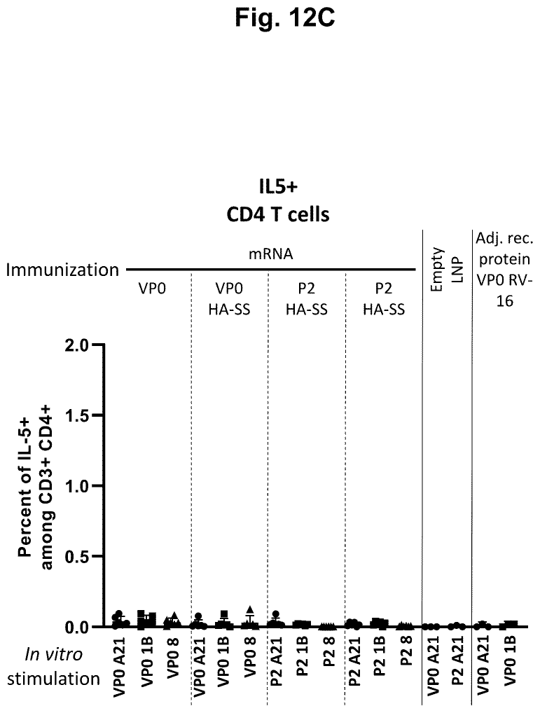

Intracellular cytokine staining (ICS) was used to assess the T-cell response as a result of immunization with LNP-encapsulated mRNA encoding the rhinovirus A serotype 21 VP0 polyprotein or the P2 polyprotein as described in Example 4, either with or without an HA signal sequence. The results are summarized in FIGS. 12A and 12B, respectively.

When administered two 2-μg doses, the LNP-encapsulated mRNA encoding the VP0 polyprotein of the rhinovirus A serotype 21 strain induced a robust cross-reactive polyfunctional CD4⁺ and CD8⁺ T-cell response (see FIGS. 12A and 12B, respectively). The T-cells were reactive against the VP0 polyprotein from rhinovirus A serotype 21 strain (RV-A21) as well as the VP0 polyproteins belonging to different phylogenetic clusters (namely rhinovirus A serotype 1b (RV-Alb) and rhinovirus A serotype 8 (RV-A8). The means were between 0.5% and 1%. The percentage of IFN-γ+ IL2+ TNFα+ CD4+ T cells of all CD3+ CD4+ T cells was significantly higher in the samples stimulated with VP0 A21 or VP0 1b peptide pools compared to samples stimulated with the VP0 8 peptide pool (p<0.01; FIG. 12A). In contrast, a similar CD8+ response was observed across all samples in response to stimulation with VP0 peptide libraries from rhinovirus A serotypes 21, 1b and 8 (FIG. 12B).

Similar observations were also made after immunization with the LNP-encapsulated mRNA encoding the P2 polyprotein. As can be seen in FIGS. 12A and 12B, when administered at two doses (2 μg/dose), the mRNA encoding the P2 polyprotein of the rhinovirus A serotype 21 also induced a P2-specific cross-reactive polyfunctional CD4+ and CD8+ T-cell response. The T-cells were reactive against the P2 peptide library from rhinovirus A serotype 21 and from rhinovirus A serotype 1b. The percentage of both IFN-γ+ IL2+ TNFα+ CD4+ or CD8+ T cells was significantly higher in the samples stimulated with P2 peptide pools from rhinovirus A serotype 21 and 1b, compared to samples stimulated with the P2 peptide pools from rhinovirus serotype 8 (p<0.01; FIGS. 12A and 12B).

In the ICS assay, adding the hemagglutinin secretion signal (HA-SS) to VP0 and P2 did not have a substantial impact on T-cell immunogenicity.

In contrast to immunization with mRNA, the recombinant VP0 protein used as a positive control induced only CD4⁺ T cells but no CD8⁺ T cells. The negative control “empty LNPs” did not induce any Tcell responses.

IL-5 production by CD4⁺ T cells was assessed to determine whether the T-cell response elicited by the VP0- and P2-encoding mRNAs can induce a T_{H}1-biased response (IFN-γ-, IL-2- and TNF-α secreting T-cells). The results of an intracellular cytokine staining assay are shown in FIG. 12 C. They confirm that the T-cell response is T_{H}1-biased, as desired.

### Non-Catalytic P2 Polyprotein Induces a Greater Number of CD4⁺ T-Cells

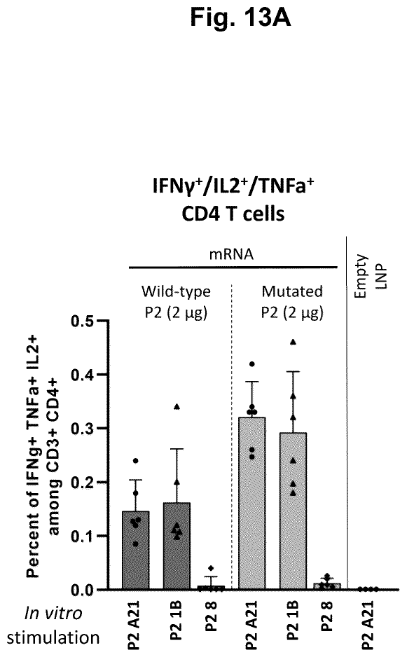

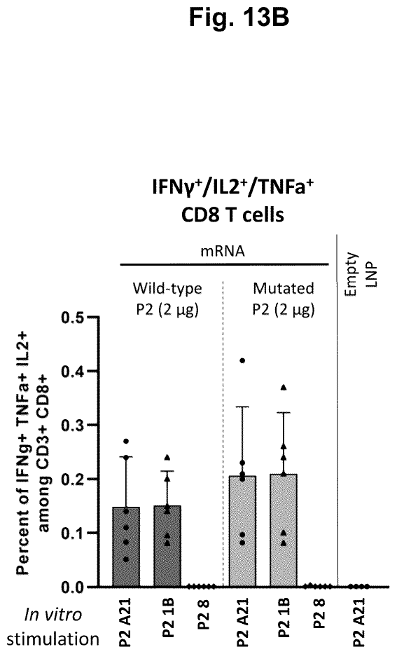

The ICS assay was also used to compare the effect of inactivating the proteolytic activity of the P2 polyprotein on the T-cell populations elicited in response to immunization. Cross-reactive polyfunctional CD4⁺ and CD8⁺ T-cells responses (see FIGS. 13A and 13B, respectively) were observed in response to immunization with LNP-encapsulated mRNAs encoding either wild-type or mutated P2 polyprotein.

mRNA encoding the mutated P2 polyprotein induced about double the percentage of polyfunctional CD4⁺ T-cells (IFN-γ⁺/IL2⁺/TNF-α⁺) than the wild-type P2 polyprotein. In contrast, mRNA encoding for the wild-type and mutated P2 polyprotein respectively, induced similar levels of polyfunctional CD8⁺ T cells (IFN-γ⁺/IL2⁺/TNF-α⁺).

Both CD4⁺ and CD8⁺ T cells were reactive against the P2 peptide pool from rhinovirus A serotype 21 and serotype 1b which belongs to different phylogenetic clusters. These data confirm that an appropriately selected P2 polyprotein can induce a cross-reactive T-cell response against multiple rhinoviruses from different phylogenetic clusters. Cross-reactivity with the P2 peptide pool from rhinovirus serotype 8 was not observed in this experiment.

IFN-γ Secretion after Immunization with mRNA Encoding VP0, P2 or a P2-VP0 Fusion Protein

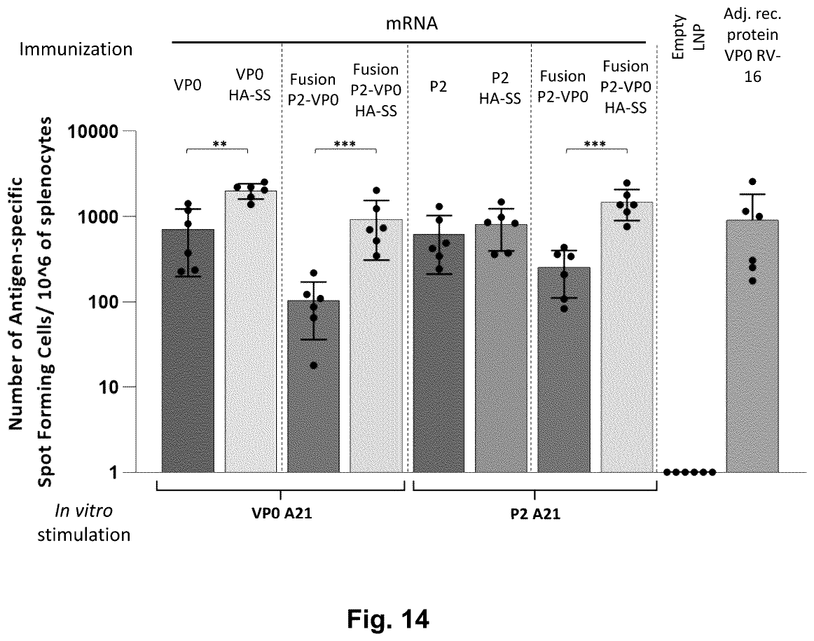

T-cell function after immunization with two 0.2 μg doses of mRNA encoding a VP0 polyprotein, a P2 polyprotein and a P2-VP0 fusion protein, respectively, was assessed using an IFN-γ FluoroSpot assay (ELISPOT). mRNAs encoded polyproteins with or without the HA secretion signal. Spleen cells were stimulated with peptide pools representative of the VP0 and P2 polyproteins encoded by the administered mRNAs. IFN-γ secretion in response to stimulation was assayed as described above. The results are summarized in FIG. 14.

As can be seen from FIG. 14, the lower mRNA dose was sufficient to induce robust IFN-γ responses, confirming the results obtained by ICS. The presence of the HA secretion signal significantly increased IFN-γ secretion in response to stimulation with the VP0 from rhinovirus A serotype 21 peptide pool, in particular after immunization with the P2-VP0 fusion protein (p<0.001). Notably, an extraordinary and statistically significant (p<0.01) increase in the number of antigen specific spot forming cells was observed with splenocytes isolated from mice immunized with an mRNA encoding the VP0 polyprotein with the HA secretion signal compared to splenocytes isolated from mice immunized with an mRNA encoding the VP0 polyprotein without the HA secretion signal. A similar observation was made in response to stimulation with the P2 peptide pool from rhinovirus A serotype 21 after immunization with the P2-VP0 fusion protein linked to the HA secretion signal (p<0.001). The mRNA-encoding the P2-VP0 fusion protein was effective in inducing IFN-γ secreting T-cells specific to VP0 and P2, respectively.

These results indicate that immunization with an mRNA encoding the P2-VP0 fusion protein is a suitable alternative to immunization with two individual mRNAs encoding the VP0 and P2 polyproteins.

IgG Response Elicited by mRNA Encoding a VP0 Polyprotein

An ELISA assay was used to assess IgG antibody titers directed against the surface-exposed VP2 protein and the intra-virion VP4. Both VP2 and VP4 are encoded by the VP0 polyprotein. The results are summarized in FIGS. 15A and 15B.

The IgG ELISA showed that immunizations with an mRNA encoding the VP0 polyprotein from rhinovirus A serotype 21 induced high IgG titers directed against both subunits of immunogen. Animals receiving two 2-μg doses had higher anti-VP2 and anti-VP4 IgG titers than animal receiving two 0.2-μg doses, confirming a dose-dependent increase in the antibody response.

Adding the HA secretion signal improved immunogenicity of the mRNA-encoded VP0 polyprotein. A significant increase in anti-VP2 and anti-VP4 was observed after immunization with an mRNA encoding a VP0 polyprotein with the HA secretion signal at both the 0.2-μg (p<0.01) and 2-μg (VP2: p<0.001; VP4: p<0.01) doses when compared to immunization with an mRNA encoding a VP0 polyprotein without the HA secretion signal. At the 2-μg dose, anti-VP2 and anti-VP4 IgG antibody titers elicited by an mRNA encoding the VP0 polyprotein from rhinovirus A serotype 21 with an HA secretion signal were equivalent to those observed with the adjuvanted recombinant protein that was used as a positive control.

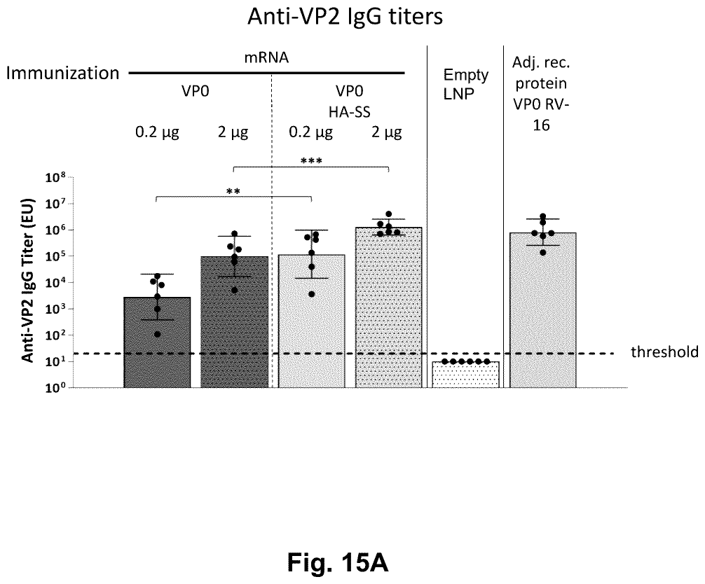

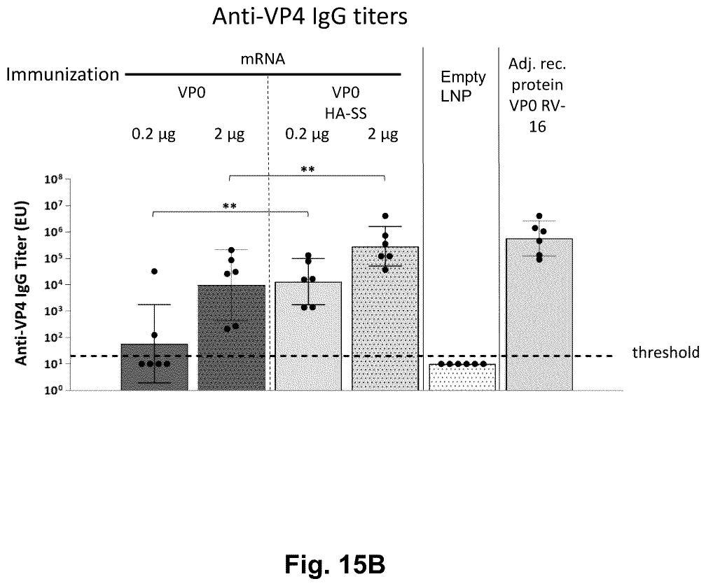

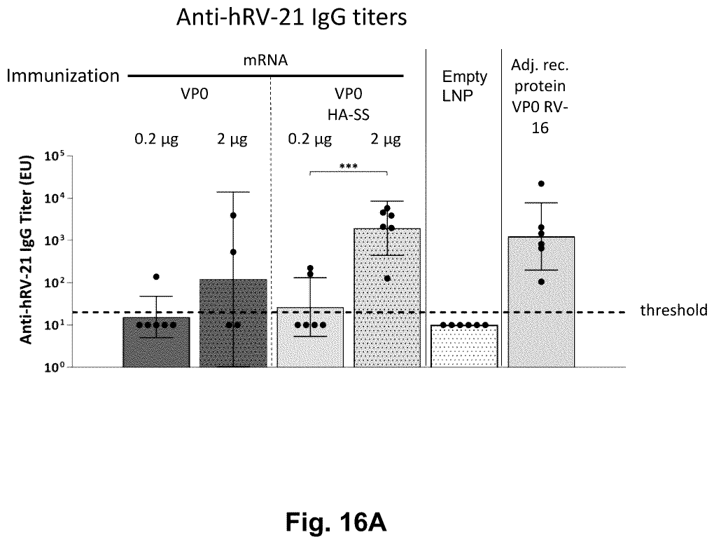

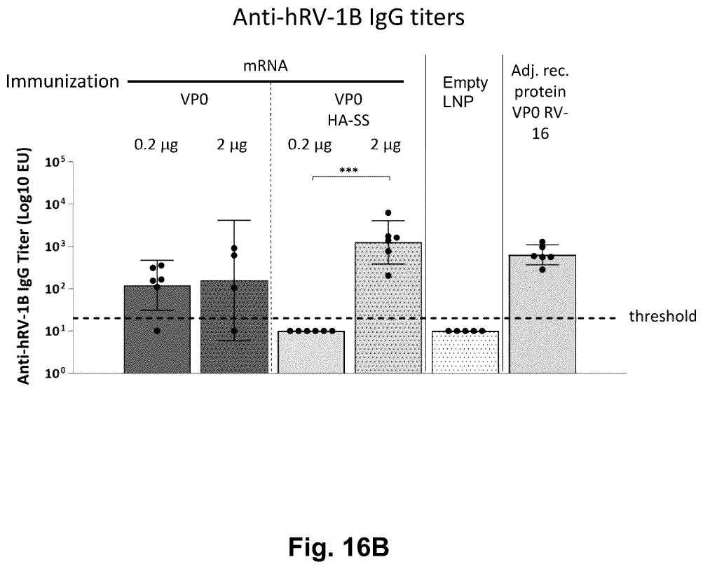

A whole virus ELISA was performed to determine whether the elicited antibodies could bind effectively both rhinovirus A serotype 21, which is source of the mRNA-encoded immunogen, as well as rhinovirus A serotype 1b, which belongs to a different phylogenetic cluster. The ELISA data are summarized in FIG. 16A and FIG. 16B, respectively.

The whole virus assay confirmed that the IgG antibodies elicited by the mRNA-encoded VP0 polyprotein from rhinovirus serotype A 21 could bind effectively both from rhinovirus serotype A 21 (FIG. 16A) and from rhinovirus serotype A 1b virions (FIG. 16B). RNA-encoded VP0 polyprotein from rhinovirus serotype A 21 with an HA secretion signal, with the 2-μg dose induced significantly higher antibody titers of anti-rhinovirus serotype A 21A21 or anti-rhinovirus serotype 1b than the 0.2-μg dose (p<0.001). The titers of antibodies binding rhinovirus serotypes A 21 and 1b antibody titers were equivalent to corresponding antibody titers elicited by the adjuvanted recombinant protein that was used as a positive control.

Overall, these data indicate that a VP0 polyprotein which is selected as an immunogen on the basis that it has an average identity of at least 80% to the amino acid sequences of corresponding polyproteins from at least two phylogenetic clusters of rhinoviruses can induce an IgG response that is broadly effective against multiple rhinovirus of different phylogenetic clusters. Moreover, inclusion of an HA secretion signal in the mRNA sequence encoding the immunogen results in improved immunogenicity, with the resulting IgG response being non-inferior to that elicited by an adjuvanted recombinant protein.

The data also indicate that an immunogenic composition that comprises non-naturally occurring mRNAs encoding naturally occurring polyproteins from phylogenetically distant rhinovirus serotypes of the same group may achieve even broader protection. For example, it may be beneficial to include a polyprotein (e.g., a P2 polyprotein) from a phylogenetically distant virus (e.g., rhinovirus A serogroup 8) to ensure broader coverage (e.g., improved T cell immunity) against a majority of rhinovirus in group A.

### Example 13. Natural Rhinovirus Infection Induces a T_{H}1 CD4 T-Cell Response Specific to Rhinovirus A and C VP0 in Healthy Human Subjects

This example illustrates that healthy human subjects exhibit a VP0-specific T_{H}1 response following stimulation with overlapping peptide libraries derived from VP0 polyproteins of either rhinovirus A or C.

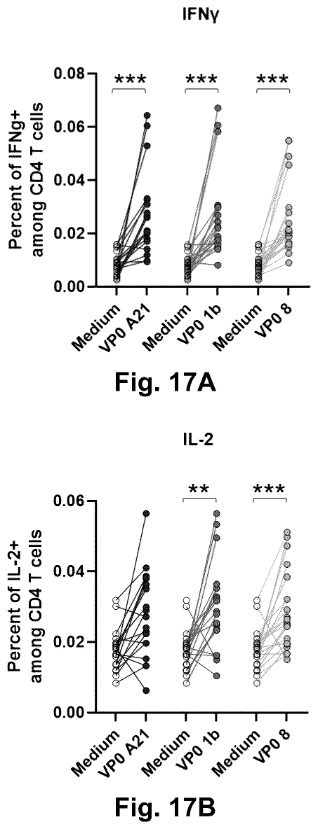

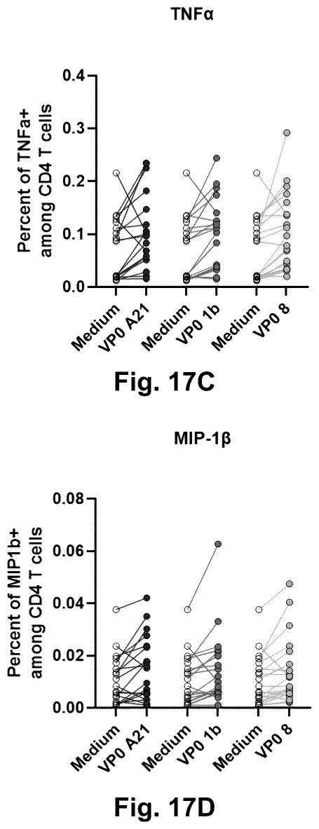

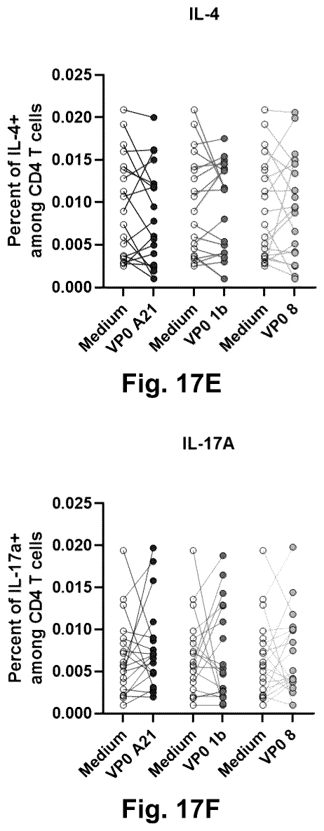

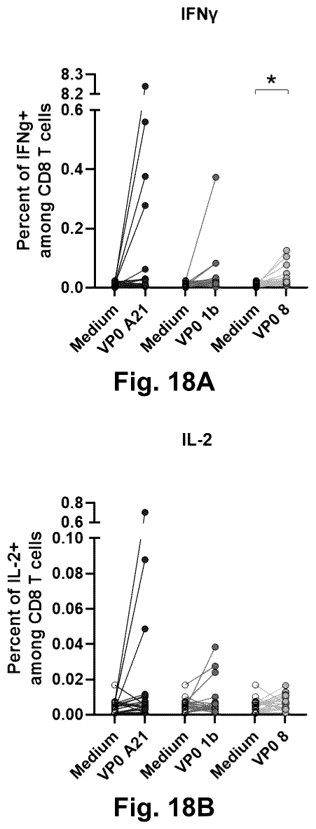

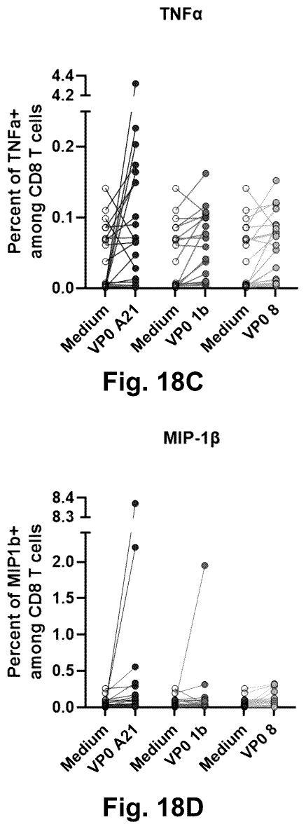

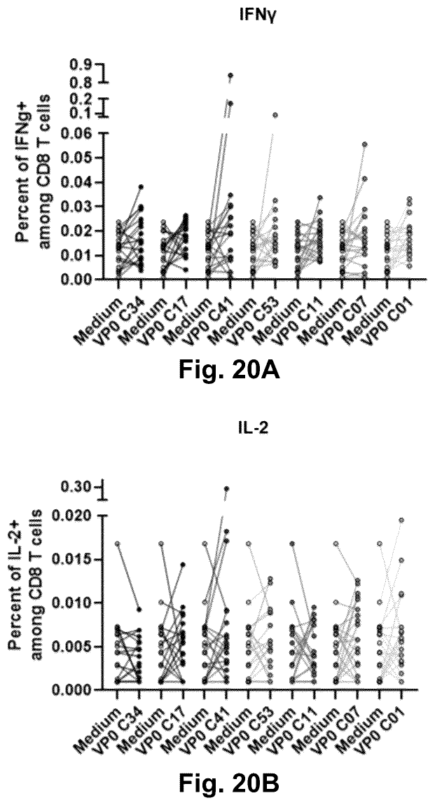

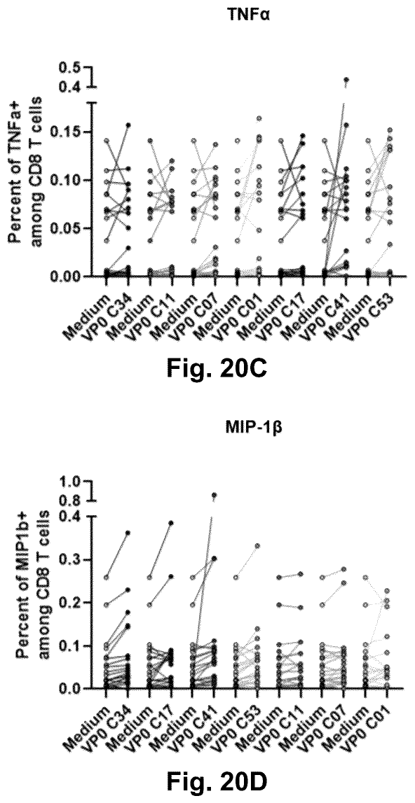

Example 12 shows that an mRNA-LNP coding for a rhinovirus A VP0 polyprotein which was selected as an immunogen on the basis that it has an average identity of at least 80% to the amino acid sequences of corresponding polyproteins from at least two phylogenetic clusters of rhinoviruses can induce T_{H}1 responses in mice that is effective against phylogenetically distant serotypes of the same rhinovirus group. To determine if VP0-specific CD4 and CD8 T-cell responses are elicited after rhinovirus natural exposure, peripheral blood mononuclear cells (PBMCs) isolated from healthy volunteers (n=20) were stimulated for 6 hours with 15-amino acid peptide libraries with 11 amino acid overlaps covering the full-length VP0 polyproteins of several rhinovirus group A and C. The results of these experiments are summarized in FIGS. 17A-20D.

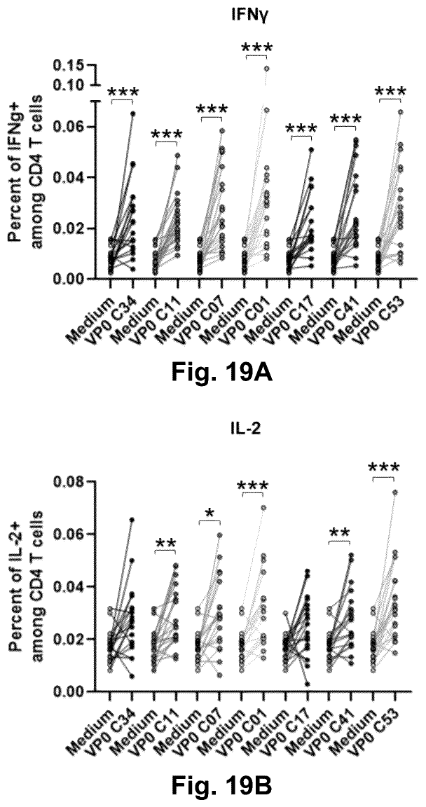

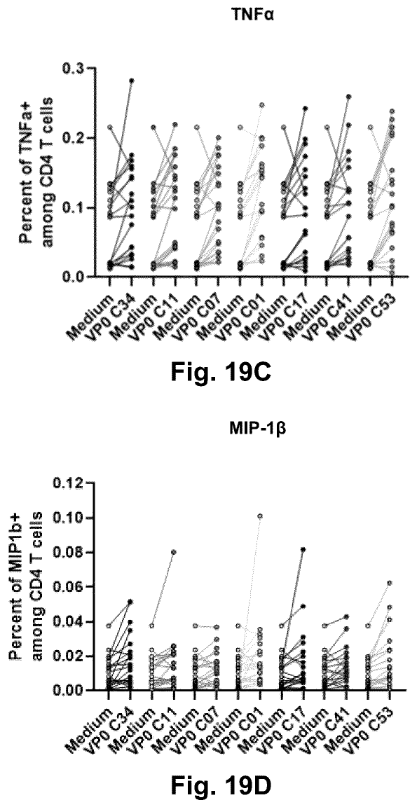

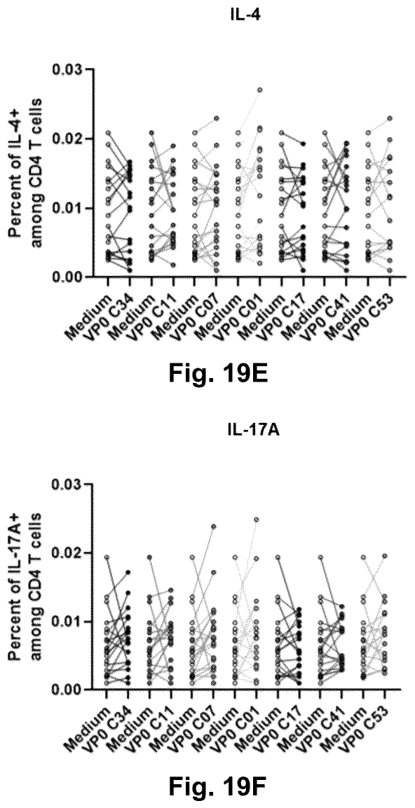

FIGS. 17A-17F and 19A-19F show the percentages of CD4+ T-cells secreting cytokines after stimulation of the PBMCs with overlapping peptide libraries representative of VP0 polyproteins of various rhinovirus A serotypes (FIGS. 17A-17F) and rhinovirus C serotypes (FIGS. 19A-19F). In response to peptide stimulation, a significant percentage of VP0-specific CD4+ T cells that secreted cytokines such as IFN-γ and IL-2 (see FIGS. 17A, 17B, 19A, and 19B) were detected, indicating that rhinovirus natural infection elicits a VP0-specific T_{H}1 response against rhinovirus A serotypes 21, 1b, 8 and C serotypes C34, C11, C07, C01, C17, C41 and C53. The percentage of VP0-specific CD4+ cells secreting TNF-α and MIP-1β was not significantly different between VP0-specific stimulated PBMCs and medium-stimulated PBMCs (see FIGS. 17C, 17D, 19C and 19D). Similarly, the percentage of VP0-specific CD4+ T cells secreting either IL-4 or IL-17A was not significantly different between VP0-specific stimulated PBMCs and medium-stimulated PBMCs (see FIGS. 17E, 17F, 19E and 19F), providing further support that a rhinovirus natural infection elicits a VP0-specific T_{H}1-biased response in healthy humans.

No statistically significant percentage of VP0-specific CD8+ T-cell responses was observed in PBMCs from healthy humans after VP0 peptide stimulation against rhinovirus groups A and C.

This example demonstrates that rhinovirus natural infection induced VP0-specific T_{H}1 responses against rhinovirus A serotypes 21, 1b, 8 and C serotypes C34, C11, C07, C01, C17, C41 and C53 in healthy humans.

### Example 14. Eliciting an Immune Response to Rhinovirus C In Vivo

This example illustrates that an immunogenic composition comprising mRNA encoding the VP0 polyprotein of human rhinovirus C serotype 34 strain (GenBank ID: MZ322913.1) described in Example 8 can elicit a T_{H}1-directed CD4+ T-cell response in vivo that is broadly reactive to VP0 polyproteins from other phylogenetic clusters of rhinoviruses of the same group.

Previous research suggests that human subjects respond rapidly with an adaptive immune response to an infection with rhinovirus. It has been hypothesized that this rapid response is due to an engagement of existing effector memory T cells which are cross-reactive with T-cell epitopes conserved across multiple rhinoviruses. The results of Example 12 indicate that an mRNA encoding rhinovirus A VP0 polyprotein rich in conserved T-cell epitopes is effective in inducing a T_{H}1-directed T-cell response in mice that is cross-reactive against multiple serotypes of group A rhinoviruses.

To test whether the approach in Example 12 could be used to similarly induce a cross-reactive T_{H}1-directed T-cell response against group C rhinoviruses, an immunogenic composition was prepared by encapsulating an mRNA encoding the rhinovirus C serotype 34 VP0 polyprotein identified in Example 8 using the same LNP formulation as described in Example 10. Empty LNPs (i.e., without mRNA) were prepared to serve as a negative control.

Each of the two compositions was separately administered to groups of wild-type C57BL/6 mice (n=10) via intramuscular injection in a 2-dose immunization series (day 0 and day 21). The mRNA encapsulated with LNPs were administered at a dose of 2 μg of mRNA. Mock-treated and immunized animals were sacrificed on day 35 after the first injection, and spleen samples were collected.

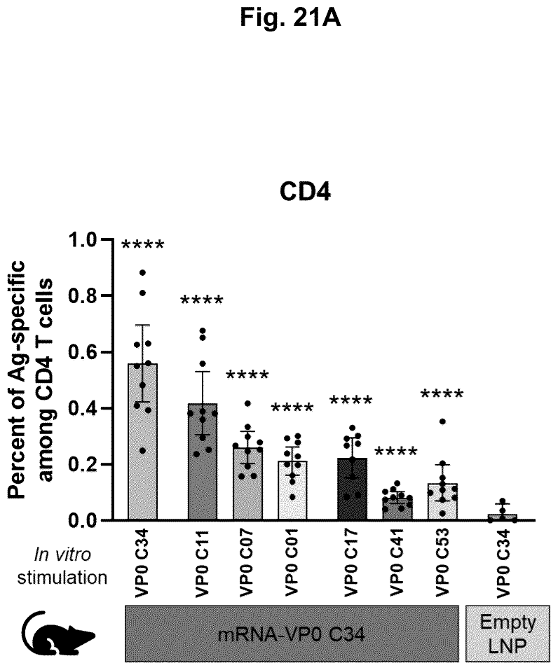

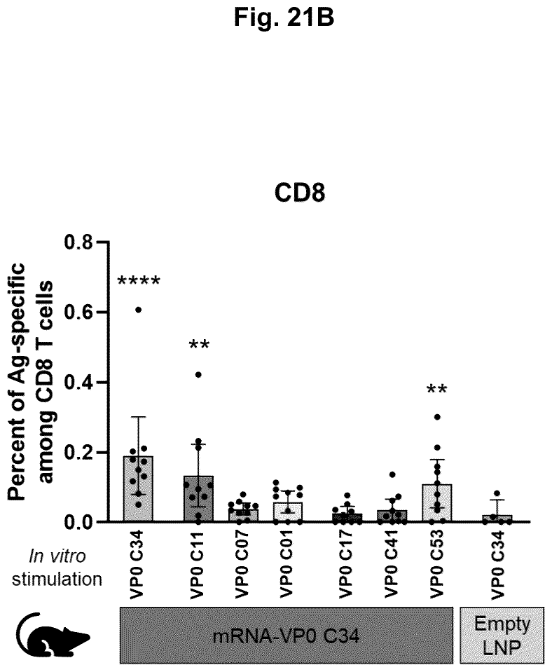

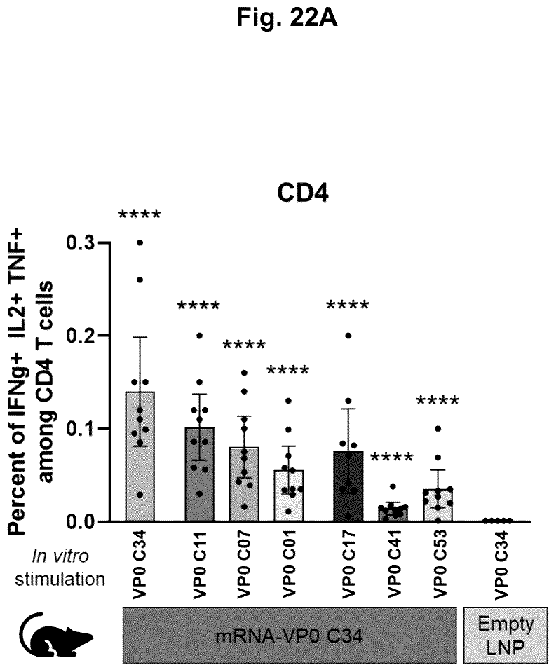

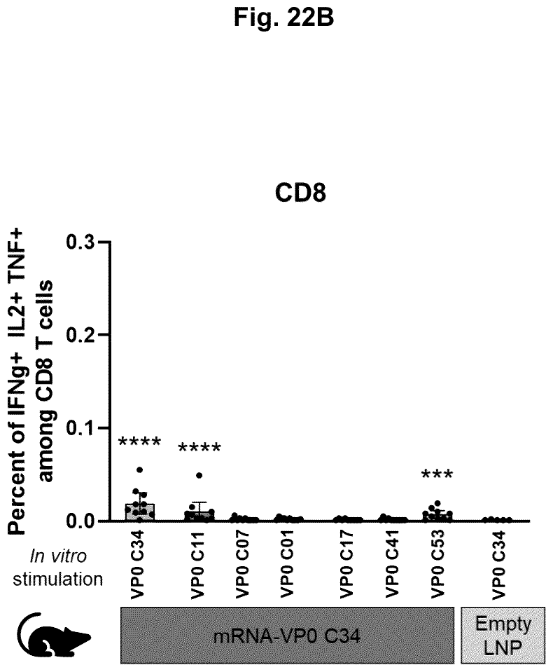

Cross-reactivity against VP0 polyproteins from rhinovirus C serotypes belonging to different phylogenetic clusters than the rhinovirus C strain used for the antigen design (serotype 34) was assessed. Splenocytes were stimulated for 6 hours with different peptide libraries derived from the VP0 polyproteins of rhinovirus C serotypes C34, C11, C7, C1, C17, C41 and C53. Table 18 provides these rhinovirus C serotype sequences. The VP0 polyproteins of serotypes C11, C7, C1, C17, C41 and C53 were selected based on their percentage of sequence identity relative to the serotype C34 VP0 polyprotein encoded by the mRNA used for immunization. The VP0 polyproteins of serotypes C11, C7, C1, C17, C41 and C53 have between 84% and 77% identity to the serotype C34 VP0 polyprotein, with the serotype C₅₃ VP0 polyprotein having the lowest percent sequence identity. The VP0 polyprotein of serotype C11 is in the same phylogenetic cluster as the VP0 polyprotein of serotype C34, whereas the other polyproteins are in different phylogenetic clusters. The VP0 polyproteins of serotypes C17 and C41 belong to the same phylogenetic cluster, and so do the VP0 polyproteins of serotypes C1 and C7. Following stimulation, the percentages of antigen-specific CD4+ and CD8+ T-cells were determined using intracellular cytokine staining and flow cytometry as described in Example 12. CD4+ and CD8+ cells were identified as antigen-specific T-cells if they stained positively for any combination of IFN-γ, IL-2, and/or TNF-α, i.e., they include single, double, or triple positive T-cells. T-cells staining positive for all three cytokines were characterized as polyfunctional. The results are summarized in FIGS. 21A-21B and 22A-22B, respectively. Negative control data is not shown for splenocytes isolated from mice mock-immunized with empty LNPs and stimulated with peptides pools deriving from the VP0 polyproteins of rhinovirus C serotypes C11, C7, C1, C17, C41 or C53. Statistical analysis was performed using the negative control data resulting from stimulation of T-cells of mock-treated mice with the corresponding peptide pool.

TABLE 18
Rhinovirus C serotype sequences for
VPO polyproteins

| SEQ | | | |
|---|---|---|---|
| ID | Sero- | GenBank | |
| NO | type | ID | Sequence |
| 83 | C1 | MZ268720 | MGAQVSRQSVGSHETMIHAGTGAVV |
| | | | KYFNVNYYKDAASSGLTKQDFSQDP |
| | | | SKFTQPVADVLTNPALMSPSVEACG |
| | | | FSDRLKQITIGNSTITTQDAVNTIV |
| | | | AYGEWPSYLSDLDATSVDKPTHPET |
| | | | SSDRFYTLKSVEWQSSSEGWWWKLP |
| | | | DCLKDMGIFGQNMFHHAMGRSGYII |
| | | | HTQCNATKFHSGCLLVAVVPEHQLA |
| | | | YIGADARVSYEHTHPGEKGHVIGSN |
| | | | SDRNNHQPDESAFFNCNGTLLGNLT |
| | | | IFPHQLINLRTNNSSTIVVPYINCT |
| | | | PMDSMLRHNNVSLVVIPICPLRPPN |
| | | | PGQQTLPITISIAPIKSEFSGARQA |
| | | | IKATAQ |
| 84 | C7 | OM001451 | MGAQVSKQSVGAHETMVHAGSGAVV |
| | | | KYFNINYYKDAASSGLTKQDFSQDP |
| | | | SKFTQPVADLLTNPALMSPSVEACG |
| | | | YSDRLKQITIGSSTITTQDSVNTIV |
| | | | AYGEWPSYLSDLDASSVDKPTHPET |
| | | | SADRFYTLDSVRWGGSSKGWWWKLP |
| | | | DCLKNMGIFGQNMYYHAMGRSGYII |
| | | | HTQCNATKFHSGCLLVAVVPEHQLA |
| | | | YIGGTNAQVSYKHTHPGESGHEIGL |
| | | | NTSRGDNRPDEDPFFNCNGTLLGNL |
| | | | TIFPHQLINLRTNNSSTIVVPYINC |
| | | | TPMDSMLRHNNVSLVIIPICPLRAS |
| | | | SGAPTTLPITVSIAPDRSEFSGARQ |
| | | | SATRQ |
| 85 | C11 | MZ268689 | MGAQVSKQNVGSHESGISASSGSVI |
| | | | KYFNINYYKDSASSGLSKQDFSMDP |
| | | | EKFTKPLADVMTNPALMSPSIEACG |
| | | | FSDRLKQITIGSSTITTQDTLNTVV |
| | | | AYGEWPEYLRDTDASAVDKPTHPET |
| | | | STDRFYTLTSVIWNGSSKGWWWKIP |
| | | | DCLKDMGMFGQNMYHHALGRSGYIF |
| | | | HIQCNATKFHSGLLLVAIVPEHQLA |
| | | | YVGGTYANVGYNHTHPGEGGHEIRE |
| | | | PTGRDDKKPDEDPLFNCNGTLLGNL |
| | | | TIFPHQLINLRTNNSVTIVVPYINC |
| | | | VPMDSMLKHNNISLVIIPLVPLRSG |
| | | | STQAPQTLPITISIAPDKSEFSGAR |
| | | | QSNKTQ |
| 86 | C17 | MZ153277 | MGAQVSRQTTGSHESAVNATNGGII |
| | | | KYFNINYYRDSASSGLTKQDFSQDP |
| | | | SKFTQPLVDTLTNPALMSPSVEACG |
| | | | FSDRLKQITMGNSTITTQDALHTVL |
| | | | AYGEWPQYLSDLDATSVDKPTHPET |
| | | | SSDRFYTLSSVSWTNTSKGWWWKLP |
| | | | DALKDMGVFGQNLYYHAMGRAGYII |
| | | | HTQCNATKFHSGALLVVLIPEHQLA |
| | | | YIGAEKVNIAYDLTHPGETGHVIGR |
| | | | NTSRGNNNPDEDPFFNCNGTLFGNL |
| | | | TIFPHQIINLRTNNSSTIITPYINC |
| | | | QPMDNMLKHNNLTLLIVPLVRLRFG |
| | | | TEASPTVSITVTIAPYKSEFSGAME |
| | | | TQKHQ |
| 87 | C41 | KF958311 | MGAQVSKQSNGTHENIVSASNGAVV |
| | | | KYFNINYYKDSASSGLSRQDFSQDP |
| | | | SKFTQPLVDTLTNPALMSPTVEACG |
| | | | FSDRLKQITIGNSTITTQDSLNSVL |
| | | | AYGEWPTYLSDIDATSVDKPTHPET |
| | | | SADRFYTLRSVNWGATSKGWWWKFP |
| | | | DALSEMGVFGQNMYYHAMGRSGYII |
| | | | HTQCNATKFHSGAIIVAVVPEHQLA |
| | | | YIGGTKANVSYGHTHPGENGHEIRG |
| | | | PDSGHDRGNNNPDEDPLFNCNGTLL |
| | | | GNITIFPHQIINLRTNNSSTIIVPY |
| | | | INCTPMDSMLKHNNVSLVIIPIVPL |
| | | | RVNGTGPTTIPITVSVAPYKSEFSG |
| | | | AMEAQRQ |
| 88 | C53 | MF775367 | MGAQVTKQKVGSHDNTIAAQSGSVV |
| | | | KYFNINYYKDAASSGLSKQDFSQDP |
| | | | SKFTQPLADVLTNPALMSPSIEACG |
| | | | YSDRLKQITIGDSTITTQDTLNSVV |
| | | | AYGEWPEYLSDIDASSIDKPTHPET |
| | | | SADRFYTLDSVDWSENSTGWWWKLP |
| | | | DCLRDMGLFGQNMYYHAMGRTGYIV |
| | | | HVQCNATKFHSGCLMVAAIPEHQLA |
| | | | YIGGGNANVKYKHTHPGDRGHTMRP |
| | | | SDVRGDNNPDEDPFYLCNGTLFGNI |
| | | | QVYPHQMINLRTNNSATLIIPYINC |
| | | | LPMDSMLRHNNISLVIIPLVRLKTG |
| | | | ATGSTTLPITITIAPDKSEFSGPMQ |
| | | | NQKQ |

Immunization with an mRNA encoding the VP0 polyprotein of rhinovirus C serotype 34 induced antigen-specific CD4+ and CD8+ T-cells in mice, as shown in FIG. 21A and FIG. 21B, respectively. Generally, the percentage of detected antigen-specific CD4+ and CD8+ T-cells was smaller, the lower the percent sequence identity of the VP0 polyprotein was to the serotype C34 VP0 polyprotein (see FIGS. 21A and 21B). As illustrated by FIG. 21B, the CD8+ T cell response extended only to VP0 polyproteins of other phylogenetic clusters that had at least 80% identity to the VP0 polyprotein encoded by the mRNA used for immunization.

About a quarter to a third of the antigen-specific CD4+ T-cells was polyfunctional (see FIG. 22A). Similar to the data observed with rhinovirus A in Example 11, only very few cross-reactive polyfunctional CD8+ T-cells were observed (see FIG. 22B).

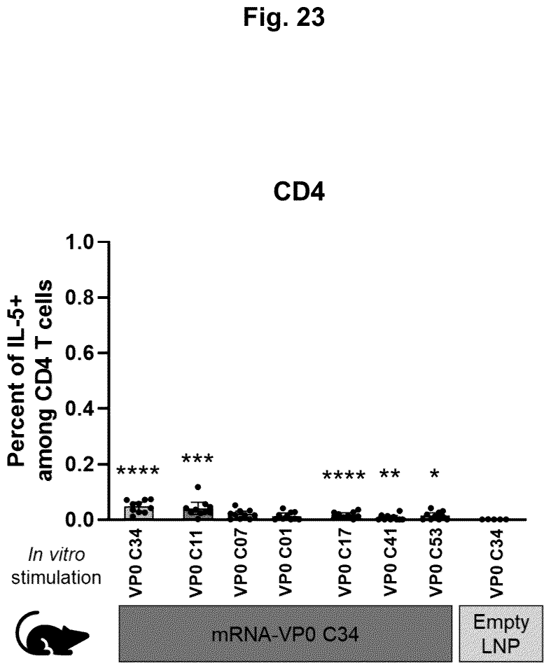

IL-5 production by CD4+ T-cells was assessed to determine whether the T-cell response elicited by the VP0-encoding mRNA is a T_{H}2 biased response. The results of an intracellular cytokine staining (ICS) assay are shown in FIG. 23. No IL-5 secreting CD4+ T cell were evidenced.

In summary, this example demonstrates that an immunogenic composition of the invention comprising an mRNA encoding a naturally occurring rhinovirus polyprotein is effective in eliciting a cross-reactive T-cell response in vivo against corresponding polyproteins from other phylogenetic clusters of rhinoviruses of the same group. The data also indicate that broader immune protection against rhinoviruses of the same group may be achieved by providing an immunogenic composition that comprises mRNAs encoding polyproteins from phylogenetically distant rhinovirus serotypes of the same group.

1. A method of identifying a rhinovirus polyprotein for use as an immunogen that is capable of eliciting an immune response against rhinoviruses from multiple serotypes within a group, said method comprising:
(a) retrieving a plurality of amino acid sequences from a database comprising amino acid sequences from naturally occurring rhinovirus isolates;
(b) removing from the plurality of amino acid sequences retrieved in step (a) amino acid sequences shorter than 800 amino acids;
(c) assigning the amino acid sequences remaining after step (b) into different phylogenetic clusters;
(d) aligning the amino acid sequences to determine a consensus amino acid sequence for a complete rhinovirus polyprotein for one or more phylogenetic clusters identified in step (c);
(e) aligning the consensus amino acid sequence obtained in step (d) with complete polyproteins of naturally occurring rhinovirus isolates; and
(f) selecting a rhinovirus polyprotein as an immunogen that has an average identity of at least 80% to the corresponding amino acid sequences of rhinoviruses from at least two phylogenetic clusters identified in step (c).

2. The method of claim 1, wherein:
(i) the rhinovirus polyprotein selected in step (f) is a VP0 polyprotein or a P2 polyprotein; and/or
(ii) the group is rhinovirus group A or rhinovirus group C; and/or
(iii) the one or more phylogenetic clusters each comprises at least 5 different serotypes or at least 10, 15, 20, or 25 different serotypes.

3-8. (canceled)

9. The method of claim 1, wherein
(i) determining the consensus sequence in step (d) comprises:
(i) selecting the most frequent amino acid at each position; and/or
(ii) creating a gap when the sum of amino acids for a given position is lower than 50% of the number of retrieved sequences, or selecting the most frequent amino acid when the sum of amino acids for a given position is equal to or greater than 50% of the number of retrieved sequences.

10-11. (canceled)

12. The method of claim 1, further comprising a step of generating an optimized nucleic acid sequence encoding the rhinovirus polyprotein selected in step (f).

13. An immunogenic composition comprising at least one messenger RNA (mRNA) comprising a first non-naturally occurring optimized nucleic acid sequence encoding a first polyprotein from a group A or C rhinovirus, wherein said first polyprotein has an amino acid sequence that:
(a) has an average identity of at least 80% to the amino acid sequences of corresponding polyproteins from at least two, at least three, or at least four phylogenetic clusters of rhinoviruses of the same group; and
(b) is naturally occurring aside from an optional single amino acid substitution.

14. (canceled)

15. The immunogenic composition of claim 13, wherein the first polyprotein is:
(i) a VP0 polyprotein comprising proteins VP2 and VP4; or
(ii) a P2 polyprotein comprising proteins 2A, 2B, and 2C.

16-25. (canceled)

26. The immunogenic composition of claim 15, wherein the first polyprotein is a P2 polyprotein and the single amino acid substitution is in the 2A protein and reduces or abolishes the proteolytic activity of the P2 polyprotein, optionally wherein the single amino acid substitution is C>A substitution or C>S in the catalytic triad of the active site of the 2A protein.

27. (canceled)

28. The immunogenic composition of claim 15, wherein:
(i) the VP0 polyprotein is from a group C rhinovirus or a group A rhinovirus; or
(ii) the P2 polyprotein is from a group A rhinovirus or a group C rhinovirus.

29. The immunogenic composition of claim 28, wherein:
(i) the VP0 polyprotein from the group C rhinovirus is of serotype 11, 17, or 34; or
(ii) the VP0 polyprotein from the group A rhinovirus is of serotype 21 or 90; or
(iii) the P2 polyprotein from the group A rhinovirus is of serotype 21 or 57; or
(iv) the P2 polyprotein from the group C rhinovirus is of serotype 11 or 17.

30-39. (canceled)

40. The immunogenic composition of claim 13, further comprising
(ii) a second non-naturally occurring optimized nucleic acid sequence encoding a second polyprotein from a group A or C rhinovirus,
wherein said second polyprotein is different from the first polyprotein and the second polyprotein has an amino acid sequence that:
(a) has an average identity of at least 80% to the amino acid sequences of corresponding polyproteins from at least two, at least three, or at least four phylogenetic clusters of rhinoviruses of the same group; and
(b) is naturally occurring aside from an optional single amino acid substitution.

41. (canceled)

42. The immunogenic composition of claim 40, wherein the first polyprotein is a VP0 polyprotein comprising proteins VP2 and VP4, optionally wherein VP0 polyprotein is from a group A rhinovirus, optionally wherein the group A rhinovirus is of serotype 21 or 90.

43-46. (canceled)

47. The immunogenic composition of claim 40, wherein:
(i) the second polyprotein is a P2 polyprotein comprising proteins 2A, 2B and 2C, optionally wherein the single amino acid substitution is in the 2A protein and reduces or abolishes the proteolytic activity of the P2 polyprotein, optionally wherein the single amino acid substitution is C>A substitution or C>S in the catalytic triad of the active site of the 2A protein; and/or
(ii) the P2 polyprotein is from a group A rhinovirus, optionally wherein the group A rhinovirus is of serotype 21 or 57; or
wherein the second polyprotein is a VP0 polyprotein from a group C rhinovirus, optionally wherein the group C rhinovirus is of serotype 11, 17, or 34.

48-58. (canceled)

59. The immunogenic composition of claim 40, wherein:
(i) the first polyprotein is a VP0 polyprotein and the second polyprotein is a VP0 polyprotein, wherein the at least two, at least three, or at least four phylogenetic clusters referred to in option (a) are different for the first and second polyproteins; or
(ii) the first polyprotein is a P2 polyprotein and the second polyprotein is a P2 polyprotein, wherein the at least two, at least three, or at least four phylogenetic clusters referred to in option (a) are different for the first and second polyproteins,
optionally wherein the first and second polyproteins are from rhinovirus C.

60-72. (canceled)

73. The immunogenic composition of claim 40, further comprising
a third non-naturally occurring optimized nucleic acid sequence encoding a third polyprotein from a group A or C rhinovirus,
wherein said first, second, and third polyproteins are different from each other and the third polyprotein has an amino acid sequence that:
(a) has an average identity of at least 80% to the amino acid sequences of corresponding polyproteins from at least two phylogenetic clusters of rhinoviruses of the same group; and
(b) is naturally occurring aside from an optional single amino acid substitution.

74. The immunogenic composition of claim 73, wherein:
(i) the first polyprotein is a VP0 polyprotein comprising proteins VP2 and VP4, optionally wherein VP0 polyprotein is from a group A rhinovirus, optionally wherein group A rhinovirus is of serotype 21 or 90; and/or
(ii) the second polyprotein is a P2 polyprotein comprising proteins 2A, 2B, and 2C, optionally wherein the single amino acid substitution is in the 2A protein and reduces or abolishes the proteolytic activity of the P2 polyprotein, optionally wherein the single amino acid substitution is C>A substitution or C>S in the catalytic triad of the active site of the 2A protein, optionally wherein the P2 polyprotein is from a group A rhinovirus, optionally wherein the group A rhinovirus is of serotype 21 or 57.

75-85. (canceled)

86. The immunogenic composition of claim 73, wherein:
(i) the at least one mRNA encodes a fusion protein comprising the first polyprotein and the second polyprotein, and optionally the third polyprotein; or
(ii) the first, second, and optionally the third non-naturally occurring optimized nucleic acid sequences are encoded by separate mRNAs.

87. (canceled)

88. The immunogenic composition of claim 73, wherein the third polyprotein is a VP0 polyprotein from a group C rhinovirus, optionally wherein the group C rhinovirus is of serotype 11, 17, or 34.

89-92. (canceled)

93. The immunogenic composition of claim 73, wherein:
(i) said composition is capable of eliciting a T-cell response in at least 95% of the human population, optionally wherein said composition is capable of eliciting a T-cell response in at least 96%, at least 97%, at least 98%, or at least 99% of the human population; and/or
(ii) the VP0 polyprotein and the P2 polyprotein comprise T-cell epitope-rich regions that cover at least 95% of the MHC class-I alleles in Table 4 and/or 95% of the MHC-II alleles in Table 5, optionally wherein the T-cell epitope-rich regions cover at least 96%, at least 97%, at least 98%, or at least 99% of the MHC class-I alleles in Table 4 and/or at least 96%, at least 97%, at least 98%, or at least 99% of the MHC class-II alleles in Table 5.

94-96. (canceled)

97. The immunogenic composition of claim 73, wherein the first, second, and optionally the third non-naturally occurring optimized nucleic acid sequences are optimized to (a) improve the yield of full-length mRNAs during in vitro synthesis, and/or (b) to maximize expression of the encoded polypeptide after delivery of the mRNA to a target cell in vivo.

98. The immunogenic composition of claim 86, wherein the at least one mRNA of (i) or the separate mRNAs of (ii) comprise:
(i) a 5′ untranslated region (UTR); and/or
(ii) a 3′ untranslated region (UTR); and/or
(iii) a 5′ cap; and/or
(iv) a polyadenylation (polyA) sequence comprising at least 90 nucleotides; and/or
(v) N-1-methylpseudouridine in place of uridine.

99-104. (canceled)

105. The immunogenic composition of claim 86, further comprising a plurality of lipid nanoparticles (LNPs) encapsulating the at least one mRNA of (i) or the separate mRNAs of (ii).

106-110. (canceled)

111. A vaccine composition comprising the immunogenic composition of claim 105 and a pharmaceutical acceptable carrier.

112. A method for eliciting an immune response, reducing or preventing one or more symptoms associated with a rhinovirus infection, or reducing the severity of or preventing a rhinovirus infection in a subject, the method comprising administering an effective amount of the vaccine composition of claim 111 to the subject.

113-126. (canceled)

127. The method of claim 112, wherein the administration of the vaccine composition provides immunity against a rhinovirus infection caused by a group A strain and/or group C strain, optionally wherein immunity is provided against multiple serotypes of the same rhinovirus group or against multiple serotypes of different rhinovirus groups.

128-129. (canceled)
